SEQUENCE LISTING (316 sequences)

SEQ ID NO: 1 - protein, 293 aa, Unknown
  Shiga-like toxin 1 Subunit A (SLT-1A)
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 2 - protein, 293 aa, Unknown
  Shiga toxin Subunit A (StxA)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 3 - protein, 297 aa, Unknown
  Shiga-like toxin 2 Subunit A (SLT-2A)
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 4 - protein, 251 aa, Unknown
  Shiga toxin subtype c Subunit A (Stx1cA)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVAR

SEQ ID NO: 5 - protein, 251 aa, Unknown
  Shiga toxin subtype d Subunit A (Stx1dA)
KEFTLDFSTAKKYVDSLNVIRSAIGTPLQTISSGGTSLLMIDSGTGDNLFAVDIMGLEPEEERFNNLRLIVERNNLYVTGFVNRTNNVFYRFADFSHVTFPGTRAVTLSGDSSYTTLQRVAGISRTGMQINRHSLTTSYLDLMSYSGTSLTQSVARAMLRFVTVTAEALRFRQIQRGFRTTLDDLSGRSYVMTAEDVDLTLNWGRLSSILPDYHGQDSVRVGRISFGSINAILGSVALILNCHHHASRVAR

SEQ ID NO: 6 - protein, 251 aa, Unknown
  Shiga toxin subtype e Subunit A (Stx1eA)
QDFTVDFSTAKKYVDSLNAIRSAIGTPLHSISSGGTSLLMIDNGTGDNLFAVDIRGLDPEEERFDNLRLIIERNNLYVTGFVNRTSNIFYRFADFSHVTFPGTRAVTLSGDSSYTTLQRVAGIGRTGMQINRHSLTTSYLDLMSYSGSSLTQPVARAMLRFVTVTAEALRFRQIQRGFRTTLDDVSGHSYTMTVEDVDLTLNWGRLSSVLPDYHGQDSVRVGRISFGGVNAILGSVALILNCHHHTSRVSR

SEQ ID NO: 7 - protein, 250 aa, Artificial Sequence
  Shiga toxin subtype 2c Subunit A (Stx2cA) variant 1
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 8 - protein, 250 aa, Artificial Sequence
  Shiga toxin subtype 2c Subunit A (Stx2cA) variant 2
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 9 - protein, 250 aa, Artificial Sequence
  Shiga toxin subtype 2c Subunit A (Stx2cA) variant 3
REFTIDFSTQQSYVSSLNSIRTEISTPLEHISQGTTSVSVINHTPPGSYFAVDIRGLDIYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 10 - protein, 250 aa, Artificial Sequence
  Shiga toxin subtype 2c Subunit A (Stx2cA) variant 4
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPSVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 11 - protein, 250 aa, Artificial Sequence
  Shiga toxin subtype 2c Subunit A (Stx2cA) variant 5
REFTIDFSTQQSYVSSLNSIRTEISTPLEHISQGTTSVSVINHTPPGSYFAVDIRGLDVYQARFDHLRLIIEQNNLYMAGFVNTATNTFYRFSDFTHISVPSVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 12 - protein, 250 aa, Artificial Sequence
  Shiga toxin subtype 2c Subunit A (Stx2cA) variant 6
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQVLSETAPVYTMTPGDVDLTLNWGRISNVLPEYRGEDGVRVGRISFNNISAILSTVAVILNCHHQGARSVR

SEQ ID NO: 13 - protein, 250 aa, Artificial Sequence
  Shiga toxin subtype 2d Subunit A (Stx2dA) variant 1
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVIPEYRGEDGVRVGRISFNNISAILGTVAVILNCHHQGARSVR

SEQ ID NO: 14 - protein, 250 aa, Artificial Sequence
  Shiga toxin subtype 2d Subunit A (Stx2dA) variant 2
REFMIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPEEVDLTLNWGRISNVLPEFRGEGGVRVGRISFNNISAILGTVAVILNCHHQGARSVR

SEQ ID NO: 15 - protein, 250 aa, Artificial Sequence
  Shiga toxin subtype 2d Subunit A (Stx2dA) variant 3
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVIPEYRGEDGVRVGRISFNNISAILSTVAVILNCHHQGARSVR

SEQ ID NO: 16 - protein, 250 aa, Artificial Sequence
  Shiga toxin subtype 2e Subunit A (Stx2eA) variant 1
QEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVR

SEQ ID NO: 17 - protein, 250 aa, Artificial Sequence
  Shiga toxin subtype 2e Subunit A (Stx2eA) variant 2
QEFTIDFSTQQSYVSSLNSIRTAISTPLEHISQGATSVSVINHTPPGSYISVGIRGLDVYQAHFDHLRLIIEQNNLYVAGFVNTATNTFYRFSDFAHISLPGVTTISMTTDSSYTTLQRVAALERSGMQISRHSLVSSYLALMEFSGNTMTREASRAVLRFVTVTAEALRFRQIQREFRQALSETAPVYTMTPEDVDLTLNWGRISNVLPEYRGEDGVRVGRISFNNISAILGTVAVILNCHHQGARSVR

SEQ ID NO: 18 - protein, 250 aa, Artificial Sequence
  Shiga toxin subtype 2f Subunit A (Stx2fA)
DEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVR

SEQ ID NO: 19 - protein, 10 aa, Unknown
  light chain HVR CDR1
SASSSVSYMY

SEQ ID NO: 20 - protein, 7 aa, Unknown
  light chain HVR CDR2
LTSNLAS

SEQ ID NO: 21 - protein, 9 aa, Unknown
  light chain HVR CDR3
QQWSSYPPT

SEQ ID NO: 22 - protein, 5 aa, Unknown
  heavy chain HVR CDR1
ENSMH

SEQ ID NO: 23 - protein, 17 aa, Unknown
  heavy chain HVR CDR2
GINPNNGGTSYNQKFKG

SEQ ID NO: 24 - protein, 13 aa, Unknown
  heavy chain HVR CDR3
PYYYGYREDYFDY

SEQ ID NO: 25 - protein, 10 aa, Unknown
  light chain HVR CDR1
RASSSVSYMY

SEQ ID NO: 26 - protein, 9 aa, Unknown
  light chain HVR CDR3
QQWSSNPPT

SEQ ID NO: 27 - protein, 5 aa, Unknown
  heavy chain HVR CDR1
EYTMH

SEQ ID NO: 28 - protein, 17 aa, Unknown
  heavy chain HVR CDR2
GIDPNNGGTWYNQKFKG

SEQ ID NO: 29 - protein, 17 aa, Unknown
  heavy chain HVR CDR2
GINPNNGGTWYNQKFKG

SEQ ID NO: 30 - protein, 17 aa, Unknown
  heavy chain HVR CDR2
GINPNNGGTWYNRKFKG

SEQ ID NO: 31 - protein, 17 aa, Unknown
  heavy chain HVR CDR2
GINPNNGGTSYNQKFQG

SEQ ID NO: 32 - protein, 13 aa, Unknown
  heavy chain HVR CDR3
PYYYGSREDYFDY

SEQ ID NO: 33 - protein, 106 aa, Artificial Sequence
  light chain 1
DIVMTQSPAIMSASPGEKVTMTCSASSSVSYMYWYQQKPRSSPKPWIYLTSNLASGVPARFSGSGSGTSYSLTISSMEAEDAATYYCQQWSSNPPTFGGGTKLELK

SEQ ID NO: 34 - protein, 122 aa, Artificial Sequence
  heavy chain 1
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSS

SEQ ID NO: 35 - protein, 106 aa, Artificial Sequence
  light chain 2
DIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 36 - protein, 106 aa, Artificial Sequence
  light chain 3
DIVLNQSPALMSASPGEKVTMTCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 37 - protein, 122 aa, Artificial Sequence
  heavy chain 2
EVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSS

SEQ ID NO: 38 - protein, 106 aa, Artificial Sequence
  light chain 4
DIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 39 - protein, 122 aa, Artificial Sequence; identical to SEQ ID NO: 37
  heavy chain 3

SEQ ID NO: 40 - protein, 251 aa, Artificial Sequence
  Shiga toxin effector polypeptide 1
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAA

SEQ ID NO: 41 - protein, 251 aa, Artificial Sequence
  Shiga toxin effector polypeptide 2
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 42 - protein, 251 aa, Artificial Sequence
  Shiga toxin effector polypeptide 3
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 43 - protein, 251 aa, Artificial Sequence
  Shiga toxin effector polypeptide 4
KEFTLDFSTAKTYVDSLNVIRSAIGTPLQTISSGGTSLLMIDSGIGDNLFAVDILGFDFTLGRFNNLRLIVERNNLYVTGFVNRTNNAFYRFADFSHVTFPGTTAVTLSADSSYTTLQRVAGISRTGMQINRHSLTTSYLALMSHSGTSLTQSAARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 44 - protein, 251 aa, Artificial Sequence
  Shiga toxin effector polypeptide 5
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 45 - protein, 251 aa, Artificial Sequence
  Shiga toxin effector polypeptide 6
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTADALRFRQIQRGFRTTLDDLSGRSYVMTAEDVDLTLNWGRLSSVLPDYHGQDSVRVGRISFGSINAILGSVALILNSHHHASAVAA

SEQ ID NO: 46 - protein, 251 aa, Artificial Sequence
  Shiga toxin effector polypeptide 7
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAA

SEQ ID NO: 47 - protein, 251 aa, Artificial Sequence
  Shiga toxin effector polypeptide 8
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAA

SEQ ID NO: 48 - protein, 251 aa, Artificial Sequence
  Shiga toxin effector polypeptide 9
KEFTLDFSTAKTYVDSLNVIRSAIGTPLQTISSGGTSLLMIDSGIGDNLFAVDILGFDFTLGRFNNLRLIVERNNLYVTGFVNRTNNAFYRFADFSHVTFPGTTAVTLSADSSYTTLQRVAGISRTGMQINRHSLTTSYLALMSHSGTSLTQSAARAMLRFVTVTADALRFRQIQRGFRTTLDDLSGASYVMTAEDVDLTLNWGRLSSVLPDYHGQDSVRVGRISFGSINAILGSVALILNSHHHASAVAA

SEQ ID NO: 49 - protein, 251 aa, Artificial Sequence
  Shiga toxin effector polypeptide 10
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNCHHHASAVAA

SEQ ID NO: 50 - protein, 251 aa, Artificial Sequence
  Shiga toxin effector polypeptide 11
KEFILRFSVAHKYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 51 - protein, 251 aa, Artificial Sequence
  Shiga toxin effector polypeptide 12
KEFTLDFSTAKTYVDSLNVIRSAIGTPLQTISSGGTSLLMIDNLVPMVATVVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 52 - protein, 251 aa, Artificial Sequence
  Shiga toxin effector polypeptide 13
KEFTLDFSTAKTYVDSLNVIRSAIGTPLQTISSGGTSLLMIDSNLVPMVATVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 53 - protein, 251 aa, Artificial Sequence
  Shiga toxin effector polypeptide 14
KEFTLDFSTAKTYVDSLNVIRSAIGTPLQTISSGGTSLLMIDSGILGFVFTLDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 54 - protein, 251 aa, Artificial Sequence
  Shiga toxin effector polypeptide 15
KEFTLDFSTAKTYVDSLNVIRSAIGTPLQTISSGGTSLLMIDSGSGDNLFAVGILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 55 - protein, 251 aa, Artificial Sequence
  Shiga toxin effector polypeptide 16
KEFTLDFSTAKTYVDSLNVIRSAIGTPLQTISSGGTSLLMIDSGSGDNLFAVDILGFVFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 56 - protein, 251 aa, Artificial Sequence
  Shiga toxin effector polypeptide 17
KEFTLDFSTAKTYVDSLNVIRSAIGTPLQTISSGGTSLLMIDSGSGDNLFAVDILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 57 - protein, 251 aa, Artificial Sequence
  Shiga toxin effector polypeptide 18
KEFTLDFSTAKTYVDSLNVIRSAIGTPLQTISSGGTSLLMIDSGSGDNLFAVGILGFVFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 58 - protein, 251 aa, Artificial Sequence
  Shiga toxin effector polypeptide 19
KEFILDFSTAKTYVDSLNVIRSAIGTPLQTISSGGTSLLMIDSGIGDNLFAVDVRGIAPIEARFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLAALSGASYVMTAEDVDLTLNWGRLSSVLPDYHGQDSVRVGRISFGSINAILGSVALILNSHHHASAVAA

SEQ ID NO: 59 - protein, 252 aa, Artificial Sequence
  Shiga toxin effector polypeptide 20
KEFTLDFSTAKTYVDSLNVIRSAIGTPLQTISSGGTSLLMIDSGSGDNLFAVGILGFVFTLEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 60 - protein, 251 aa, Artificial Sequence
  Shiga toxin effector polypeptide 21
KEFTLDFSTAKTYVDSLNVIRSAIGTPLQTISSGGTSLLMIDSGSGDNLFAVNLVPMVATVGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 61 - protein, 251 aa, Artificial Sequence
  Shiga toxin effector polypeptide 22
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTNLVPMVATVSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 62 - protein, 251 aa, Artificial Sequence
  Shiga toxin effector polypeptide 23
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRGILGDVFTLSYVMTAEDVDLTLNWGRLSSVLPDYHGQDSVRVGRISFGSINAILGSVALILNSHHHASAVAA

SEQ ID NO: 63 - protein, 260 aa, Artificial Sequence
  Shiga toxin effector polypeptide 24
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHILRFSVAHKASAVAA

SEQ ID NO: 64 - protein, 262 aa, Artificial Sequence
  Shiga toxin effector polypeptide 25
KEFTLDFSTAKTYVDSLNVIRSAIGTPLQTISSGGTSLLMIDSGSGDNLFAVDILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHARNLVPMVATVASAVAA

SEQ ID NO: 65 - protein, 251 aa, Artificial Sequence; identical to SEQ ID NO: 61
  Shiga toxin effector polypeptide 26

SEQ ID NO: 66 - protein, 251 aa, Artificial Sequence; identical to SEQ ID NO: 62
  Shiga toxin effector polypeptide 27

SEQ ID NO: 67 - protein, 260 aa, Artificial Sequence; identical to SEQ ID NO: 63
  Shiga toxin effector polypeptide 28

SEQ ID NO: 68 - protein, 262 aa, Artificial Sequence; identical to SEQ ID NO: 64
  Shiga toxin effector polypeptide 29

SEQ ID NO: 69 - protein, 25 aa, Artificial Sequence
  linker 1
GGGGSGGGGSGGGGSGGGGSGGGGS

SEQ ID NO: 70 - protein, 20 aa, Artificial Sequence
  linker 2
GGGGSGGGGSGGGGSGGGGS

SEQ ID NO: 71 - protein, 18 aa, Artificial Sequence
  linker 3
GSTSGSGKPGSGEGSTKG

SEQ ID NO: 72 - protein, 5 aa, Artificial Sequence
  linker 4
GGGGS

SEQ ID NO: 73 - protein, 16 aa, Artificial Sequence
  linker 5
EFPKPSTPPGSSGGAP

SEQ ID NO: 74 - protein, 25 aa, Artificial Sequence
  linker 6
EFPKPSTPPGSSGGAPGILGFVFTL

SEQ ID NO: 75 - protein, 15 aa, Artificial Sequence
  linker 7
GSTSGSGKPGSGEGS

SEQ ID NO: 76 - protein, 16 aa, Artificial Sequence
  linker 8
SPSTPPTPSPSTPPAS

SEQ ID NO: 77 - protein, 15 aa, Artificial Sequence
  linker 9
AHHSEDPSSKAPKAP

SEQ ID NO: 78 - protein, 9 aa, Unknown
  T-cell epitope-peptide C1
NLVPMVATV

SEQ ID NO: 79 - protein, 9 aa, Unknown
  T-cell epitope-peptide C2
VTEHDTLLY

SEQ ID NO: 80 - protein, 9 aa, Unknown
  T-cell epitope-peptide C4
SIINFEKYL

SEQ ID NO: 81 - protein, 9 aa, Unknown
  T-cell epitope-peptide C1-2
GLDRNSGNY

SEQ ID NO: 82 - protein, 9 aa, Unknown
  T-cell epitope-peptide C1-3
GVMTRGRLK

SEQ ID NO: 83 - protein, 9 aa, Unknown
  T-cell epitope-peptide F2
GILGFVFTL

SEQ ID NO: 84 - protein, 9 aa, Unknown
  T-cell epitope-peptide F3
ILRGSVAHK

SEQ ID NO: 85 - protein, 233 aa, Artificial Sequence
  single-chain variable fragment I
DIQMTQSPSSLSASVGDRVTITCSASSSVSYMYWYQQKPRSSPKPWIYLTSNLASGVPARFSGSGSGTSYSLTISSMEAEDAATYYCQQWSSYPPTFGGGTKLEIKGGGGSEVQLQESGPELVKPGASVKISCKTSGYTFTENSMHWVKQSHGKSLEWIGGINPNNGGTSYNQKFKGKATLTVDKSSSTAYMEFRSLTSEDSAVYYCARPYYYGYREDYFDYWGQGTTLTVSS

SEQ ID NO: 86 - protein, 233 aa, Artificial Sequence
  single-chain variable fragment II
DIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKGGGGSEVHLVESGPELVKPGASVKISCKTSGYTFTEYTMHWVKQSHGKSLEWIGGIDPNNGGTWYNQKFKGKATLTVDKSSSTAYMELRSLTSEDSAVYFCARPYYYGSREDYFDYWGQGTTLTVSS

SEQ ID NO: 87 - protein, 233 aa, Artificial Sequence
  single-chain variable fragment III
DIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKGGGGSEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSS

SEQ ID NO: 88 - protein, 233 aa, Artificial Sequence
  single-chain variable fragment IV
DIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKGGGGSEVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSS

SEQ ID NO: 89 - protein, 253 aa, Artificial Sequence
  single-chain variable fragment V
EVQLQESGPELVKPGASVKISCKTSGYTFTENSMHWVKQSHGKSLEWIGGINPNNGGTSYNQKFKGKATLTVDKSSSTAYMEFRSLTSEDSAVYYCARPYYYGYREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSYPPTFGGGTKLEIK

SEQ ID NO: 90 - protein, 253 aa, Artificial Sequence
  single-chain variable fragment VI
EVHLVESGPELVKPGASVKISCKTSGYTFTEYTMHWVKQSHGKSLEWIGGIDPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 91 - protein, 253 aa, Artificial Sequence
  single-chain variable fragment VII
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 92 - protein, 253 aa, Artificial Sequence
  single-chain variable fragment VIII
EVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 93 - protein, 233 aa, Artificial Sequence
  single-chain variable fragment IX
DIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGPEVKKPGASVKVSCKASGYTFTENSMHWVRQSHGKSLEWMGGINPNNGGTSYNQKFKGKVTMTTDKSTSTAYMELRSLTSDDTAVYYCARPYYYGYREDYFDYWGQGTTLTVSS

SEQ ID NO: 94 - protein, 233 aa, Artificial Sequence
  single-chain variable fragment X
DIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKASGYTFTENSMHWVRQAHGQRLEWIGGINPNNGGTWYNRKFKGKAAITVDTSASTAYMELSSLRSEDTAVYYCARPYYYGYREDYFDYWGQGTTLTVSS

SEQ ID NO: 95 - protein, 233 aa, Artificial Sequence
  single-chain variable fragment XI
DIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKASGYTFTENSMHWVRQAHGQRLEWIGGINPNNGGTSYNQKFKGKATITVDTSASTAYMELSSLRSEDTAVYYCARPYYYGYREDYFDYWGQGTTLTVSS

SEQ ID NO: 96 - protein, 233 aa, Artificial Sequence
  single-chain variable fragment XII
DIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKTSGYTFTENSMHWVRQAPGQRLEWMGGINPNNGGTSYNQKFQGRVTLTVDKSSSTAYMELSSLRSEDTAVYYCARPYYYGYREDYFDYWGQGTTLTVSS

SEQ ID NO: 97 - protein, 253 aa, Artificial Sequence
  single-chain variable fragment XIII
QVQLVQSGPEVKKPGASVKVSCKASGYTFTENSMHWVRQSHGKSLEWMGGINPNNGGTSYNQKFKGKVTMTTDKSTSTAYMELRSLTSDDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIK

SEQ ID NO: 98 - protein, 253 aa, Artificial Sequence
  single-chain variable fragment XIV
QVQLVQSGAEVKKPGASVKVSCKASGYTFTENSMHWVRQAHGQRLEWIGGINPNNGGTWYNRKFKGKAAITVDTSASTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIK

SEQ ID NO: 99 - protein, 253 aa, Artificial Sequence
  single-chain variable fragment XV
QVQLVQSGAEVKKPGASVKVSCKASGYTFTENSMHWVRQAHGQRLEWIGGINPNNGGTSYNQKFKGKATITVDTSASTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIK

SEQ ID NO: 100 - protein, 253 aa, Artificial Sequence
  single-chain variable fragment XVI
QVQLVQSGAEVKKPGASVKVSCKTSGYTFTENSMHWVRQAPGQRLEWMGGINPNNGGTSYNQKFQGRVTLTVDKSSSTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIK

SEQ ID NO: 101 - protein, 253 aa, Artificial Sequence
  single-chain variable fragment XVII
EVQLQQSGPELVKPGASVKISCKTSGYTFTEYTMHWVKSRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 102 - protein, 253 aa, Artificial Sequence
  single-chain variable fragment XVIII
EVQLQQSGPELVKPGASVKISCKTSGYTFTEYTMHWVKQRHGKSLEWIGGINPNNGGTWYNRKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 103 - protein, 253 aa, Artificial Sequence
  single-chain variable fragment XIX
EVQLQQSGPELVKPGASVKISCKTSGYTFTEYTMHWVKQRHGKSLEWIGGINPNNGGTWYNQKFKGKTTLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 104 - protein, 253 aa, Artificial Sequence
  single-chain variable fragment XX
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 105 - protein, 233 aa, Artificial Sequence
  single-chain variable fragment XXI
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 106 - protein, 233 aa, Artificial Sequence
  single-chain variable fragment XXII
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 107 - protein, 238 aa, Artificial Sequence
  single-chain variable fragment XXIII
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 108 - protein, 510 aa, Artificial Sequence
  PD-L1 binding protein 115744
MKEFTLDFSTAKTYVDSLNVIRSAIGTPLQTISSGGTSLLMIDSGCGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKGGGGSEVHLVESGPELVKPGASVKISCKTSGYTFTEYTMHWVKQSHGKSLEWIGGIDPNNGGTWYNQKFKGKATLTVDKSSSTAYMELRSLTSEDSAVYFCARPYYYGSREDYFDYWGQGTTLTVSSNLVPMVATV

SEQ ID NO: 109 - protein, 510 aa, Artificial Sequence
  PD-L1 binding protein 115745
MKEFTLDFSTAKTYVDSLNVIRSAIGTPLQTISSGGTSLLMIDSGCGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKGGGGSEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSNLVPMVATV

SEQ ID NO: 110 - protein, 510 aa, Artificial Sequence
  PD-L1 binding protein 115746
MKEFTLDFSTAKTYVDSLNVIRSAIGTPLQTISSGGTSLLMIDSGCGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKGGGGSEVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSSNLVPMVATV

SEQ ID NO: 111 - protein, 534 aa, Artificial Sequence
  PD-L1 binding protein 115747
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQESGPELVKPGASVKISCKTSGYTFTENSMHWVKQSHGKSLEWIGGINPNNGGTSYNQKFKGKATLTVDKSSSTAYMEFRSLTSEDSAVYYCARPYYYGYREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSYPPTFGGGTKLEIKHHAANLVPMVATV

SEQ ID NO: 112 - protein, 534 aa, Artificial Sequence
  PD-L1 binding protein 115748
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVHLVESGPELVKPGASVKISCKTSGYTFTEYTMHWVKQSHGKSLEWIGGIDPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKHHAANLVPMVATV

SEQ ID NO: 113 - protein, 534 aa, Artificial Sequence
  PD-L1 binding protein 115749
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 114 - protein, 534 aa, Artificial Sequence
  PD-L1 binding protein 115750
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKHHAANLVPMVATV

SEQ ID NO: 115 - protein, 510 aa, Artificial Sequence
  PD-L1 binding protein 115751
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGPEVKKPGASVKVSCKASGYTFTENSMHWVRQSHGKSLEWMGGINPNNGGTSYNQKFKGKVTMTTDKSTSTAYMELRSLTSDDTAVYYCARPYYYGYREDYFDYWGQGTTLTVSSNLVPMVATV

SEQ ID NO: 116 - protein, 510 aa, Artificial Sequence
  PD-L1 binding protein 115752
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKASGYTFTENSMHWVRQAHGQRLEWIGGINPNNGGTWYNRKFKGKAAITVDTSASTAYMELSSLRSEDTAVYYCARPYYYGYREDYFDYWGQGTTLTVSSNLVPMVATV

SEQ ID NO: 117 - protein, 510 aa, Artificial Sequence
  PD-L1 binding protein 115753
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKASGYTFTENSMHWVRQAHGQRLEWIGGINPNNGGTSYNQKFKGKATITVDTSASTAYMELSSLRSEDTAVYYCARPYYYGYREDYFDYWGQGTTLTVSSNLVPMVATV

SEQ ID NO: 118 - protein, 510 aa, Artificial Sequence
  PD-L1 binding protein 115754
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKTSGYTFTENSMHWVRQAPGQRLEWMGGINPNNGGTSYNQKFQGRVTLTVDKSSSTAYMELSSLRSEDTAVYYCARPYYYGYREDYFDYWGQGTTLTVSSNLVPMVATV

SEQ ID NO: 119 - protein, 534 aa, Artificial Sequence
  PD-L1 binding protein 115755
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGPEVKKPGASVKVSCKASGYTFTENSMHWVRQSHGKSLEWMGGINPNNGGTSYNQKFKGKVTMTTDKSTSTAYMELRSLTSDDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 120 - protein, 534 aa, Artificial Sequence
  PD-L1 binding protein 115756
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGAEVKKPGASVKVSCKASGYTFTENSMHWVRQAHGQRLEWIGGINPNNGGTWYNRKFKGKAAITVDTSASTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 121 - protein, 534 aa, Artificial Sequence
  PD-L1 binding protein 115757
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGAEVKKPGASVKVSCKASGYTFTENSMHWVRQAHGQRLEWIGGINPNNGGTSYNQKFKGKATITVDTSASTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 122 - protein, 534 aa, Artificial Sequence
  PD-L1 binding protein 115758
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGAEVKKPGASVKVSCKTSGYTFTENSMHWVRQAPGQRLEWMGGINPNNGGTSYNQKFQGRVTLTVDKSSSTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 123 - protein, 521 aa, Artificial Sequence
  PD-L1 binding protein 115961
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 124 - protein, 521 aa, Artificial Sequence
  PD-L1 binding protein 115962
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 125 - protein, 501 aa, Artificial Sequence
  PD-L1 binding protein 116187
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 126 - protein, 514 aa, Artificial Sequence
  PD-L1 binding protein 116188
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKHHAANLVPMVATV

SEQ ID NO: 127 - protein, 501 aa, Artificial Sequence
  PD-L1 binding protein 116296
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 128 - skipped

SEQ ID NO: 129 - protein, 519 aa, Artificial Sequence
  PD-L1 binding protein 116299
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 130 - protein, 534 aa, Artificial Sequence
  PD-L1 binding protein 116324
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIVMTQSPAIMSASPGEKVTMTCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 131 - protein, 514 aa, Artificial Sequence
  PD-L1 binding protein 116325
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIVMTQSPAIMSASPGEKVTMTCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 132 - protein, 509 aa, Artificial Sequence
  PD-L1 binding protein #1
KEFTLDFSTAKTYVDSLNVIRSAIGTPLQTISSGGTSLLMIDSGCGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKGGGGSEVHLVESGPELVKPGASVKISCKTSGYTFTEYTMHWVKQSHGKSLEWIGGIDPNNGGTWYNQKFKGKATLTVDKSSSTAYMELRSLTSEDSAVYFCARPYYYGSREDYFDYWGQGTTLTVSSNLVPMVATV

SEQ ID NO: 133 - protein, 509 aa, Artificial Sequence
  PD-L1 binding protein #2
KEFTLDFSTAKTYVDSLNVIRSAIGTPLQTISSGGTSLLMIDSGCGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKGGGGSEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSNLVPMVATV

SEQ ID NO: 134 - protein, 509 aa, Artificial Sequence
  PD-L1 binding protein #3
KEFTLDFSTAKTYVDSLNVIRSAIGTPLQTISSGGTSLLMIDSGCGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKGGGGSEVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSSNLVPMVATV

SEQ ID NO: 135 - protein, 533 aa, Artificial Sequence
  PD-L1 binding protein #4
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQESGPELVKPGASVKISCKTSGYTFTENSMHWVKQSHGKSLEWIGGINPNNGGTSYNQKFKGKATLTVDKSSSTAYMEFRSLTSEDSAVYYCARPYYYGYREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSYPPTFGGGTKLEIKHHAANLVPMVATV

SEQ ID NO: 136 - protein, 533 aa, Artificial Sequence
  PD-L1 binding protein #5
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVHLVESGPELVKPGASVKISCKTSGYTFTEYTMHWVKQSHGKSLEWIGGIDPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKHHAANLVPMVATV

SEQ ID NO: 137 - protein, 533 aa, Artificial Sequence
  PD-L1 binding protein #6
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 138 - protein, 533 aa, Artificial Sequence
  PD-L1 binding protein #7
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKHHAANLVPMVATV

SEQ ID NO: 139 - protein, 509 aa, Artificial Sequence
  PD-L1 binding protein #8
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGPEVKKPGASVKVSCKASGYTFTENSMHWVRQSHGKSLEWMGGINPNNGGTSYNQKFKGKVTMTTDKSTSTAYMELRSLTSDDTAVYYCARPYYYGYREDYFDYWGQGTTLTVSSNLVPMVATV

SEQ ID NO: 140 - protein, 509 aa, Artificial Sequence
  PD-L1 binding protein #9
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKASGYTFTENSMHWVRQAHGQRLEWIGGINPNNGGTWYNRKFKGKAAITVDTSASTAYMELSSLRSEDTAVYYCARPYYYGYREDYFDYWGQGTTLTVSSNLVPMVATV

SEQ ID NO: 141 - protein, 509 aa, Artificial Sequence
  PD-L1 binding protein #10
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKASGYTFTENSMHWVRQAHGQRLEWIGGINPNNGGTSYNQKFKGKATITVDTSASTAYMELSSLRSEDTAVYYCARPYYYGYREDYFDYWGQGTTLTVSSNLVPMVATV

SEQ ID NO: 142 - protein, 509 aa, Artificial Sequence
  PD-L1 binding protein #11
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKGGGGSQVQLVQSGAEVKKPGASVKVSCKTSGYTFTENSMHWVRQAPGQRLEWMGGINPNNGGTSYNQKFQGRVTLTVDKSSSTAYMELSSLRSEDTAVYYCARPYYYGYREDYFDYWGQGTTLTVSSNLVPMVATV

SEQ ID NO: 143 - protein, 533 aa, Artificial Sequence
  PD-L1 binding protein #12
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGPEVKKPGASVKVSCKASGYTFTENSMHWVRQSHGKSLEWMGGINPNNGGTSYNQKFKGKVTMTTDKSTSTAYMELRSLTSDDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 144 - protein, 533 aa, Artificial Sequence
  PD-L1 binding protein #13
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGAEVKKPGASVKVSCKASGYTFTENSMHWVRQAHGQRLEWIGGINPNNGGTWYNRKFKGKAAITVDTSASTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 145 - protein, 533 aa, Artificial Sequence
  PD-L1 binding protein #14
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGAEVKKPGASVKVSCKASGYTFTENSMHWVRQAHGQRLEWIGGINPNNGGTSYNQKFKGKATITVDTSASTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 146 - protein, 533 aa, Artificial Sequence
  PD-L1 binding protein #15
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASQVQLVQSGAEVKKPGASVKVSCKTSGYTFTENSMHWVRQAPGQRLEWMGGINPNNGGTSYNQKFQGRVTLTVDKSSSTAYMELSSLRSEDTAVYYCARPYYYGYREDYFDYWGQGTTLTVSSGGGGSGGGGSGGGGSGGGGSGGGGSDIQMTQSPSSLSASVGDRVTITCRASSSVSYMYWYQQKPGQAPRPLIYLTSNLASGIPDRFSGSGSGTDYTLTISRLEPEDFAVYYCQQWSSYPPTFGQGTKVEIKHHAANLVPMVATV

SEQ ID NO: 147 - protein, 520 aa, Artificial Sequence
  PD-L1 binding protein #16
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 148 - protein, 520 aa, Artificial Sequence
  PD-L1 binding protein #17
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQSHGKSLEWIGGINPNNGGTWYNRKFKGKAALTVDKSSSTAYMELRSLTSEDSAVYFCARPYYYGSREDYFDYWGQGTTLTVSSGGGGSGGGGSGGGGSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 149 - protein, 500 aa, Artificial Sequence
  PD-L1 binding protein #18
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 150 - protein, 513 aa, Artificial Sequence
  PD-L1 binding protein #19
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLEIKHHAANLVPMVATV

SEQ ID NO: 151 - protein, 500 aa, Artificial Sequence
  PD-L1 binding protein #20
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 152 - protein, 513 aa, Artificial Sequence
  PD-L1 binding protein #21
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 153 - protein, 518 aa, Artificial Sequence
  PD-L1 binding protein #22
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 154 - protein, 533 aa, Artificial Sequence
  PD-L1 binding protein #23
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIVMTQSPAIMSASPGEKVTMTCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 155 - protein, 513 aa, Artificial Sequence
  PD-L1 binding protein #24
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIVMTQSPAIMSASPGEKVTMTCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 156 - protein, 253 aa, Artificial Sequence
  single-chain variable fragment XXIV
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSGGGGSGGGGSGGGGSGGGGSDIVMTQSPAIMSASPGEKVTMTCSASSSVSYMYWYQQKPRSSPKPWIYLTSNLASGVPARFSGSGSGTSYSLTISSMEAEDAATYYCQQWSSNPPTFGGGTKLELK

SEQ ID NO: 157 - protein, 233 aa, Artificial Sequence
  single-chain variable fragment XXV
EVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIVMTQSPAIMSASPGEKVTMTCSASSSVSYMYWYQQKPRSSPKPWIYLTSNLASGVPARFSGSGSGTSYSLTISSMEAEDAATYYCQQWSSNPPTFGGGTKLELK

SEQ ID NO: 158 - protein, 513 aa, Artificial Sequence
  PD-L1 binding protein #25
KEFTLDFSTAKTYVDSLNVIRSAIGTPLQTISSGGTSLLMIDSGIGDNLFAVDILGFDFTLGRFNNLRLIVERNNLSVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 159 - protein, 532 aa, Artificial Sequence
  PD-L1 binding protein #26
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCTGTSSDVGSYNRVSWYQQPPGTAPKLMIYEVSNRPSGVSNRFSGSKSGNTASLTISGLQAEDEADYYCSSHTTSGTYVFGTGTKLTVLGGGGSEVQLVQSGAEVKKPGASVKVSCKASGGTFSSYAISWVRQAPGQGLEWMGGIIPIFGTANYAQKFQGRVTITADESTSTAYMELSSLRSEDTAVYYCARDQGYAHAFDIWGRGTTVTVSSHHAANLVPMVATVRRNLVPMVATVRRNLVP

SEQ ID NO: 160 - protein, 514 aa, Artificial Sequence
  Binding Molecule 116555
MKEFTLDFSTAKTYVDSLNVIRSAIGTPLQTISSGGTSLLMIDSGIGDNLFAVDILGFDFTLGRFNNLRLIVERNNLSVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATV

SEQ ID NO: 161 - protein, 533 aa, Artificial Sequence
  Binding Molecule 115765
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCTGTSSDVGSYNRVSWYQQPPGTAPKLMIYEVSNRPSGVSNRFSGSKSGNTASLTISGLQAEDEADYYCSSHTTSGTYVFGTGTKLTVLGGGGSEVQLVQSGAEVKKPGASVKVSCKASGGTFSSYAISWVRQAPGQGLEWMGGIIPIFGTANYAQKFQGRVTITADESTSTAYMELSSLRSEDTAVYYCARDQGYAHAFDIWGRGTTVTVSSHHAANLVPMVATVRRNLVPMVATVRRNLVP

SEQ ID NO: 162 - protein, 503 aa, Artificial Sequence
  Binding Molecule 115695
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCTGTSSDVGSYNRVSWYQQPPGTAPKLMIYEVSNRPSGVSNRFSGSKSGNTASLTISGLQAEDEADYYCSSHTTSGTYVFGTGTKLTVLGGGGSEVQLVQSGAEVKKPGASVKVSCKASGGTFSSYAISWVRQAPGQGLEWMGGIIPIFGTANYAQKFQGRVTITADESTSTAYMELSSLRSEDTAVYYCARDQGYAHAFDIWGRGTTVTVSS

SEQ ID NO: 163 - protein, 508 aa, Artificial Sequence
  Binding Molecule 114895
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIVLTQSPASLAVSLGQRATISCRATESVEYYGTSLVQWYQQKPGQPPKLLIYAASSVDSGVPARFSGSGSGTDFSLTIHPVEEDDIAMYFCQQSRRVPYTFGGGTKLEIKGGGGSEVQLQQSGPELVKPGASVKMSCKASGYTFTSYVMHWVKQKPGQGLEWIGYVNPFNDGTKYNEMFKGKATLTSDKSSSTAYMELSSLTSEDSAVYYCARQAWGYPWGQGTLVTVSANLVPMVATV

SEQ ID NO: 164 - protein, 532 aa, Artificial Sequence
  Binding molecule V1
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKNLVPMVATVRRNLVPMVATVRRNLVPMVATV

SEQ ID NO: 165 - protein, 527 aa, Artificial Sequence
  Binding molecule V2
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKNLVPMVATVRRNLVPMVATVRRNLVP

SEQ ID NO: 166 - protein, 502 aa, Artificial Sequence
  PD-L1 Binding protein #27
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAASPSTPPTPSPSTPPASDIQMTQSPSSLSASVGDRVTITCTGTSSDVGSYNRVSWYQQPPGTAPKLMIYEVSNRPSGVSNRFSGSKSGNTASLTISGLQAEDEADYYCSSHTTSGTYVFGTGTKLTVLGGGGSEVQLVQSGAEVKKPGASVKVSCKASGGTFSSYAISWVRQAPGQGLEWMGGIIPIFGTANYAQKFQGRVTITADESTSTAYMELSSLRSEDTAVYYCARDQGYAHAFDIWGRGTTVTVSS

SEQ ID NO: 167 - protein, 507 aa, Artificial Sequence
  PD-L1 Binding Protein #28
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIVLTQSPASLAVSLGQRATISCRATESVEYYGTSLVQWYQQKPGQPPKLLIYAASSVDSGVPARFSGSGSGTDFSLTIHPVEEDDIAMYFCQQSRRVPYTFGGGTKLEIKGGGGSEVQLQQSGPELVKPGASVKMSCKASGYTFTSYVMHWVKQKPGQGLEWIGYVNPFNDGTKYNEMFKGKATLTSDKSSSTAYMELSSLTSEDSAVYYCARQAWGYPWGQGTLVTVSANLVPMVATV

SEQ ID NO: 168 - protein, 531 aa, Artificial Sequence
  Binding Molecule V3
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATVRRNLVPMVATVRRNLVP

SEQ ID NO: 169 - protein, 251 aa, Artificial Sequence
  Shiga toxin effector polypeptide 30
KEFTLDFSTAKTYVDSLNVIRSAIGTPLQTISSGGTSLLMIDSGIGDNLFAVDILGFDFTLGRFNNLRLIVERNNLSVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAA

SEQ ID NO: 170 - protein, 251 aa, Artificial Sequence
  Shiga toxin effector polypeptide 31
KEFTLDFSTAKTYVDSLNVIRSAIGTPLQTISSGGTSLLMIDSGCGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 171-172 - skipped (2 entries)

SEQ ID NO: 173 - protein, 251 aa, Artificial Sequence
  Shiga toxin effector polypeptide 30
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTADALSFRQIQRGFRTTLDDLSGASYVMTAEDVDLTLNWGRLSSVLPDYHGQDSVRVGRISFGSINAILGSVALILNSHHHASAVAA

SEQ ID NO: 174 - protein, 26 aa, Artificial Sequence
  Carboxy-terminal moiety
NLVPMVATVRRNLVPMVATVRRNLVP

SEQ ID NO: 175 - protein, 31 aa, Artificial Sequence
  Carboxy-terminal moiety
NLVPMVATVRRNLVPMVATVRRNLVPMVATV

SEQ ID NO: 176 - protein, 13 aa, Artificial Sequence
  Carboxy-terminal moiety
HHAANLVPMVATV

SEQ ID NO: 177 - protein, 30 aa, Artificial Sequence
  Carboxy-terminal moiety
HHAANLVPMVATVRRNLVPMVATVRRNLVP

SEQ ID NO: 178 - protein, 20 aa, Unknown
  Carboxy-terminal moiety
NLVPMVATVRRNLVPMVATV

SEQ ID NO: 179 - protein, 22 aa, Unknown
  Carboxy-terminal moiety
NLVPMVATVHHAANLVPMVATV

SEQ ID NO: 180 - protein, 11 aa, Unknown
  Carboxy-terminal moiety
RRNLVPMVATV

SEQ ID NO: 181 - protein, 28 aa, Unknown
  Carboxy-terminal moiety
RRNLVPMVATVRRNLVPMVATVRRNLVP

SEQ ID NO: 182 - protein, 33 aa, Unknown
  Carboxy-terminal moiety
NLVPMVATVRRNLVPMVATVHHAANLVPMVATV

SEQ ID NO: 183 - protein, 30 aa, Unknown
  Carboxy-terminal moiety
NLVPMVATVRRAANLVPMVATVHHAANLVP

SEQ ID NO: 184 - protein, 33 aa, Unknown
  Carboxy-terminal moiety
NLVPMVATVHHAANLVPMVATVRRNLVPMVATV

SEQ ID NO: 185 - protein, 28 aa, Unknown
  Carboxy-terminal moiety
NLVPMVATVHHAANLVPMVATVRRNLVP

SEQ ID NO: 186 - protein, 35 aa, Unknown
  Carboxy-terminal moiety
NLVPMVATVHHAANLVPMVATVHHAANLVPMVATV

SEQ ID NO: 187 - protein, 30 aa, Unknown
  Carboxy-terminal moiety
NLVPMVATVHHAANLVPMVATVHHAANLVP

SEQ ID NO: 188 - protein, 12 aa, Unknown
  proteinaceous linker
GKSSGSGSESKS

SEQ ID NO: 189 - protein, 14 aa, Unknown
  proteinaceous linker
EGKSSGSGSESKEF

SEQ ID NO: 190 - protein, 14 aa, Unknown
  proteinaceous linker
GSTSGSGKSSEGKG

SEQ ID NO: 191 - protein, 18 aa, Unknown
  proteinaceous linker
GSTSGSGKSSEGSGSTKG

SEQ ID NO: 192 - protein, 18 aa, Unknown
  proteinaceous linker
GSTSGSGKPGSGEGSTKG

SEQ ID NO: 193 - protein, 5 aa, Unknown
  proteinaceous linker
SRSSG

SEQ ID NO: 194 - protein, 5 aa, Unknown
  proteinaceous linker
SGSSC

SEQ ID NO: 195 - protein, 28 aa, Unknown
  proteinaceous linker
AMGRSGGGCAGNRVGSSLSCGGLNLQAM

SEQ ID NO: 196 - protein, 4 aa, Artificial Sequence
  scFv linker
GGGS

SEQ ID NO: 197 - protein, 8 aa, Artificial Sequence
  scFv linker
GGGGSGGG

SEQ ID NO: 198 - protein, 7 aa, Artificial Sequence
  scFv linker
GGSGGGG

SEQ ID NO: 199 - protein, 18 aa, Artificial Sequence
  scFv linker
GSTSGGGSGGGSGGGGSS

SEQ ID NO: 200 - protein, 18 aa, Artificial Sequence
  scFv linker
GSTSGSGKPGSSEGSTKG

SEQ ID NO: 201 - protein, 10 aa, Artificial Sequence
  scFv linker
  MISC_FEATURE (3)..(4): may be absent
  MISC_FEATURE (5)..(6): may be absent
  MISC_FEATURE (7)..(8): may be absent
  MISC_FEATURE (9)..(10): may be absent
GSGSGSGSGS

SEQ ID NO: 202 - protein, 25 aa, Artificial Sequence
  scFv linker
  MISC_FEATURE (6)..(10): may be absent
  MISC_FEATURE (11)..(15): may be absent
  MISC_FEATURE (16)..(20): may be absent
  MISC_FEATURE (21)..(25): may be absent
GSGGSGSGGSGSGGSGSGGSGSGGS

SEQ ID NO: 203 - protein, 25 aa, Artificial Sequence
  scFv linker
  MISC_FEATURE (6)..(10): may be absent
  MISC_FEATURE (11)..(15): may be absent
  MISC_FEATURE (16)..(20): may be absent
  MISC_FEATURE (21)..(25): may be absent
GGGGSGGGGSGGGGSGGGGSGGGGS

SEQ ID NO: 204 - protein, 20 aa, Artificial Sequence
  scFv linker
  MISC_FEATURE (5)..(8): may be absent
  MISC_FEATURE (9)..(12): may be absent
  MISC_FEATURE (13)..(16): may be absent
  MISC_FEATURE (17)..(20): may be absent
GGGSGGGSGGGSGGGSGGGS

SEQ ID NO: 205 - protein, 4 aa, Artificial Sequence
  endoplasmic reticulum retention/retrieval signal motif
KDEL

SEQ ID NO: 206 - protein, 4 aa, Artificial Sequence
  endoplasmic reticulum retention/retrieval signal motif
HDEF

SEQ ID NO: 207 - protein, 4 aa, Artificial Sequence
  endoplasmic reticulum retention/retrieval signal motif
HDEL

SEQ ID NO: 208 - protein, 4 aa, Artificial Sequence
  endoplasmic reticulum retention/retrieval signal motif
RDEF

SEQ ID NO: 209 - protein, 4 aa, Artificial Sequence
  endoplasmic reticulum retention/retrieval signal motif
RDEL

SEQ ID NO: 210 - protein, 4 aa, Artificial Sequence
  endoplasmic reticulum retention/retrieval signal motif
WDEL

SEQ ID NO: 211 - protein, 4 aa, Artificial Sequence
  endoplasmic reticulum retention/retrieval signal motif
YDEL

SEQ ID NO: 212 - protein, 4 aa, Artificial Sequence
  endoplasmic reticulum retention/retrieval signal motif
HEEF

SEQ ID NO: 213 - protein, 4 aa, Artificial Sequence
  endoplasmic reticulum retention/retrieval signal motif
HEEL

SEQ ID NO: 214 - protein, 4 aa, Artificial Sequence
  endoplasmic reticulum retention/retrieval signal motif
KEEL

SEQ ID NO: 215 - protein, 4 aa, Artificial Sequence
  endoplasmic reticulum retention/retrieval signal motif
REEL

SEQ ID NO: 216 - protein, 4 aa, Artificial Sequence
  endoplasmic reticulum retention/retrieval signal motif
KAEL

SEQ ID NO: 217 - protein, 4 aa, Artificial Sequence
  endoplasmic reticulum retention/retrieval signal motif
KCEL

SEQ ID NO: 218 - protein, 4 aa, Artificial Sequence
  endoplasmic reticulum retention/retrieval signal motif
KFEL

SEQ ID NO: 219 - protein, 4 aa, Artificial Sequence
  endoplasmic reticulum retention/retrieval signal motif
KGEL

SEQ ID NO: 220 - protein, 4 aa, Artificial Sequence
  endoplasmic reticulum retention/retrieval signal motif
KHEL

SEQ ID NO: 221 - protein, 4 aa, Artificial Sequence
  endoplasmic reticulum retention/retrieval signal motif
KLEL

SEQ ID NO: 222 - protein, 4 aa, Artificial Sequence
  endoplasmic reticulum retention/retrieval signal motif
KNEL

SEQ ID NO: 223 - protein, 4 aa, Artificial Sequence
  endoplasmic reticulum retention/retrieval signal motif
KQEL

SEQ ID NO: 224 - protein, 4 aa, Artificial Sequence
  endoplasmic reticulum retention/retrieval signal motif
KREL

SEQ ID NO: 225 - protein, 4 aa, Artificial Sequence
  endoplasmic reticulum retention/retrieval signal motif
KSEL

SEQ ID NO: 226 - protein, 4 aa, Artificial Sequence
  endoplasmic reticulum retention/retrieval signal motif
KVEL

SEQ ID NO: 227 - protein, 4 aa, Artificial Sequence
  endoplasmic reticulum retention/retrieval signal motif
KWEL

SEQ ID NO: 228 - protein, 4 aa, Artificial Sequence
  endoplasmic reticulum retention/retrieval signal motif
KYEL

SEQ ID NO: 229 - protein, 4 aa, Artificial Sequence
  endoplasmic reticulum retention/retrieval signal motif
KEDL

SEQ ID NO: 230 - protein, 4 aa, Artificial Sequence
  endoplasmic reticulum retention/retrieval signal motif
KIEL

SEQ ID NO: 231 - protein, 4 aa, Artificial Sequence
  endoplasmic reticulum retention/retrieval signal motif
DKEL

SEQ ID NO: 232 - protein, 4 aa, Artificial Sequence
  endoplasmic reticulum retention/retrieval signal motif
FDEL

SEQ ID NO: 233 - protein, 4 aa, Artificial Sequence
  endoplasmic reticulum retention/retrieval signal motif
KDEF

SEQ ID NO: 234 - protein, 4 aa, Artificial Sequence
  endoplasmic reticulum retention/retrieval signal motif
KKEL

SEQ ID NO: 235 - protein, 4 aa, Artificial Sequence
  endoplasmic reticulum retention/retrieval signal motif
HADL

SEQ ID NO: 236 - protein, 4 aa, Artificial Sequence
  endoplasmic reticulum retention/retrieval signal motif
HAEL

SEQ ID NO: 237 - protein, 4 aa, Artificial Sequence
  endoplasmic reticulum retention/retrieval signal motif
HIEL

SEQ ID NO: 238 - protein, 4 aa, Artificial Sequence
  endoplasmic reticulum retention/retrieval signal motif
HNEL

SEQ ID NO: 239 - protein, 4 aa, Artificial Sequence
  endoplasmic reticulum retention/retrieval signal motif
HTEL

SEQ ID NO: 240 - protein, 4 aa, Artificial Sequence
  endoplasmic reticulum retention/retrieval signal motif
KTEL

SEQ ID NO: 241 - protein, 4 aa, Artificial Sequence
  endoplasmic reticulum retention/retrieval signal motif
HVEL

SEQ ID NO: 242 - protein, 4 aa, Artificial Sequence
  endoplasmic reticulum retention/retrieval signal motif
NDEL

SEQ ID NO: 243 - protein, 4 aa, Artificial Sequence
  endoplasmic reticulum retention/retrieval signal motif
QDEL

SEQ ID NO: 244 - protein, 4 aa, Artificial Sequence
  endoplasmic reticulum retention/retrieval signal motif
REDL

SEQ ID NO: 245 - protein, 4 aa, Artificial Sequence
  endoplasmic reticulum retention/retrieval signal motif
RNEL

SEQ ID NO: 246 - protein, 4 aa, Artificial Sequence
  endoplasmic reticulum retention/retrieval signal motif
RTDL

SEQ ID NO: 247 - protein, 4 aa, Artificial Sequence
  endoplasmic reticulum retention/retrieval signal motif
RTEL

SEQ ID NO: 248 - protein, 4 aa, Artificial Sequence
  endoplasmic reticulum retention/retrieval signal motif
SDEL

SEQ ID NO: 249 - protein, 4 aa, Artificial Sequence
  endoplasmic reticulum retention/retrieval signal motif
TDEL

SEQ ID NO: 250 - protein, 4 aa, Artificial Sequence
  endoplasmic reticulum retention/retrieval signal motif
SKEL

SEQ ID NO: 251 - protein, 4 aa, Artificial Sequence
  endoplasmic reticulum retention/retrieval signal motif
STEL

SEQ ID NO: 252 - protein, 4 aa, Artificial Sequence
  endoplasmic reticulum retention/retrieval signal motif
EDEL

SEQ ID NO: 253-258 - skipped (6 entries)

SEQ ID NO: 259 - protein, 498 aa, Artificial Sequence
  Binding Molecule 114962
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 260 - protein, 498 aa, Artificial Sequence
  Binding Molecule 114963
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKGGGGSQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSS

SEQ ID NO: 261 - protein, 518 aa, Artificial Sequence
  Binding Molecule 114964
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 262 - protein, 210 aa, Artificial Sequence
  linker
  MISC_FEATURE (2)..(6): may be absent
  MISC_FEATURE (8)..(14): may be absent
  MISC_FEATURE (9)..(14): may be absent
  MISC_FEATURE (15)..(21): may be absent
  MISC_FEATURE (16)..(20): may be absent
  MISC_FEATURE (22)..(28): may be absent
  MISC_FEATURE (23)..(27): may be absent
  MISC_FEATURE (29)..(34): may be absent
  MISC_FEATURE (30)..(33): may be absent
  MISC_FEATURE (35)..(42): may be absent
  MISC_FEATURE (36)..(41): may be absent
  MISC_FEATURE (43)..(49): may be absent
  MISC_FEATURE (44)..(48): may be absent
  MISC_FEATURE (50)..(56): may be absent
  MISC_FEATURE (51)..(55): may be absent
  MISC_FEATURE (57)..(63): may be absent
  MISC_FEATURE (58)..(62): may be absent
  MISC_FEATURE (64)..(70): may be absent
  MISC_FEATURE (65)..(69): may be absent
  MISC_FEATURE (71)..(77): may be absent
  MISC_FEATURE (72)..(76): may be absent
  MISC_FEATURE (78)..(84): may be absent
  MISC_FEATURE (79)..(83): may be absent
  MISC_FEATURE (85)..(91): may be absent
  MISC_FEATURE (86)..(90): may be absent
  MISC_FEATURE (92)..(98): may be absent
  MISC_FEATURE (93)..(97): may be absent
  MISC_FEATURE (99)..(105): may be absent
  MISC_FEATURE (100)..(104): may be absent
  MISC_FEATURE (106)..(112): may be absent
  MISC_FEATURE (107)..(111): may be absent
  MISC_FEATURE (113)..(119): may be absent
  MISC_FEATURE (114)..(118): may be absent
  MISC_FEATURE (120)..(126): may be absent
  MISC_FEATURE (121)..(125): may be absent
  MISC_FEATURE (127)..(133): may be absent
  MISC_FEATURE (128)..(132): may be absent
  MISC_FEATURE (134)..(140): may be absent
  MISC_FEATURE (135)..(139): may be absent
  MISC_FEATURE (141)..(147): may be absent
  MISC_FEATURE (142)..(146): may be absent
  MISC_FEATURE (148)..(154): may be absent
  MISC_FEATURE (149)..(153): may be absent
  MISC_FEATURE (155)..(161): may be absent
  MISC_FEATURE (156)..(160): may be absent
  MISC_FEATURE (162)..(168): may be absent
  MISC_FEATURE (163)..(167): may be absent
  MISC_FEATURE (169)..(175): may be absent
  MISC_FEATURE (170)..(174): may be absent
  MISC_FEATURE (176)..(182): may be absent
  MISC_FEATURE (177)..(181): may be absent
  MISC_FEATURE (183)..(189): may be absent
  MISC_FEATURE (184)..(188): may be absent
  MISC_FEATURE (190)..(196): may be absent
  MISC_FEATURE (191)..(195): may be absent
  MISC_FEATURE (197)..(203): may be absent
  MISC_FEATURE (198)..(202): may be absent
  MISC_FEATURE (204)..(210): may be absent
  MISC_FEATURE (205)..(209): may be absent
GGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGS

SEQ ID NO: 263 - protein, 210 aa, Artificial Sequence
  linker
  MISC_FEATURE (2)..(6): may be absent
  MISC_FEATURE (8)..(14): may be absent
  MISC_FEATURE (9)..(14): may be absent
  MISC_FEATURE (15)..(21): may be absent
  MISC_FEATURE (16)..(20): may be absent
  MISC_FEATURE (22)..(28): may be absent
  MISC_FEATURE (23)..(27): may be absent
  MISC_FEATURE (29)..(34): may be absent
  MISC_FEATURE (30)..(33): may be absent
  MISC_FEATURE (35)..(42): may be absent
  MISC_FEATURE (36)..(41): may be absent
  MISC_FEATURE (43)..(49): may be absent
  MISC_FEATURE (44)..(48): may be absent
  MISC_FEATURE (50)..(56): may be absent
  MISC_FEATURE (51)..(55): may be absent
  MISC_FEATURE (57)..(63): may be absent
  MISC_FEATURE (58)..(62): may be absent
  MISC_FEATURE (64)..(70): may be absent
  MISC_FEATURE (65)..(69): may be absent
  MISC_FEATURE (71)..(77): may be absent
  MISC_FEATURE (72)..(76): may be absent
  MISC_FEATURE (78)..(84): may be absent
  MISC_FEATURE (79)..(83): may be absent
  MISC_FEATURE (85)..(91): may be absent
  MISC_FEATURE (86)..(90): may be absent
  MISC_FEATURE (92)..(98): may be absent
  MISC_FEATURE (93)..(97): may be absent
  MISC_FEATURE (99)..(105): may be absent
  MISC_FEATURE (100)..(104): may be absent
  MISC_FEATURE (106)..(112): may be absent
  MISC_FEATURE (107)..(111): may be absent
  MISC_FEATURE (113)..(119): may be absent
  MISC_FEATURE (114)..(118): may be absent
  MISC_FEATURE (120)..(126): may be absent
  MISC_FEATURE (121)..(125): may be absent
  MISC_FEATURE (127)..(133): may be absent
  MISC_FEATURE (128)..(132): may be absent
  MISC_FEATURE (134)..(140): may be absent
  MISC_FEATURE (135)..(139): may be absent
  MISC_FEATURE (141)..(147): may be absent
  MISC_FEATURE (142)..(146): may be absent
  MISC_FEATURE (148)..(154): may be absent
  MISC_FEATURE (149)..(153): may be absent
  MISC_FEATURE (155)..(161): may be absent
  MISC_FEATURE (156)..(160): may be absent
  MISC_FEATURE (162)..(168): may be absent
  MISC_FEATURE (163)..(167): may be absent
  MISC_FEATURE (169)..(175): may be absent
  MISC_FEATURE (170)..(174): may be absent
  MISC_FEATURE (176)..(182): may be absent
  MISC_FEATURE (177)..(181): may be absent
  MISC_FEATURE (183)..(189): may be absent
  MISC_FEATURE (184)..(188): may be absent
  MISC_FEATURE (190)..(196): may be absent
  MISC_FEATURE (191)..(195): may be absent
  MISC_FEATURE (197)..(203): may be absent
  MISC_FEATURE (198)..(202): may be absent
  MISC_FEATURE (204)..(210): may be absent
  MISC_FEATURE (205)..(209): may be absent
SSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSG

SEQ ID NO: 264 - protein, 30 aa, Artificial Sequence
  linker
  MISC_FEATURE (2)..(30): each residue individually may be absent or present
GGGGGGGGGGGGGGGGGGGGGGGGGGGGGG

SEQ ID NO: 265 - protein, 4 aa, Artificial Sequence
  cleavable spacer
HHAA

SEQ ID NO: 266 - protein, 150 aa, Artificial Sequence
  linker
  MISC_FEATURE (6)..(10): may be absent
  MISC_FEATURE (11)..(15): may be absent
  MISC_FEATURE (16)..(20): may be absent
  MISC_FEATURE (21)..(25): may be absent
  MISC_FEATURE (26)..(30): may be absent
  MISC_FEATURE (31)..(35): may be absent
  MISC_FEATURE (36)..(40): may be absent
  MISC_FEATURE (41)..(45): may be absent
  MISC_FEATURE (46)..(50): may be absent
  MISC_FEATURE (51)..(55): may be absent
  MISC_FEATURE (56)..(60): may be absent
  MISC_FEATURE (61)..(65): may be absent
  MISC_FEATURE (66)..(70): may be absent
  MISC_FEATURE (71)..(75): may be absent
  MISC_FEATURE (76)..(80): may be absent
  MISC_FEATURE (81)..(85): may be absent
  MISC_FEATURE (86)..(90): may be absent
  MISC_FEATURE (91)..(95): may be absent
  MISC_FEATURE (96)..(100): may be absent
  MISC_FEATURE (106)..(110): may be absent
  MISC_FEATURE (111)..(115): may be absent
  MISC_FEATURE (116)..(120): may be absent
  MISC_FEATURE (121)..(125): may be absent
  MISC_FEATURE (126)..(130): may be absent
  MISC_FEATURE (131)..(135): may be absent
  MISC_FEATURE (136)..(140): may be absent
  MISC_FEATURE (141)..(145): may be absent
  MISC_FEATURE (146)..(150): may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 267-299 - skipped (33 entries)

SEQ ID NO: 300 - protein, 11 aa, Artificial Sequence
  CD8+ T-cell epitope
YSEHPTFTSQY

SEQ ID NO: 301 - protein, 9 aa, Artificial Sequence
  CD8+ T-cell epitope
KLGGALQAK

SEQ ID NO: 302 - protein, 9 aa, Artificial Sequence
  CD8+ T-cell epitope
QYDPVAALF

SEQ ID NO: 303 - protein, 516 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAAYSEHPTFTSQY

SEQ ID NO: 304 - protein, 514 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAAVTEHDTLLY

SEQ ID NO: 305 - protein, 514 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAAKLGGALQAK

SEQ ID NO: 306 - protein, 514 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAAQYDPVAALF

SEQ ID NO: 307 - protein, 512 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAYSEHPTFTSQYEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 308 - protein, 510 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAVTEHDTLLYEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 309 - protein, 510 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAKLGGALQAKEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 310 - protein, 510 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQYDPVAALFEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELK

SEQ ID NO: 311 - protein, 536 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAYSEHPTFTSQYEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAANLVPMVATVRRVTEHDTLLY

SEQ ID NO: 312 - protein, 536 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAYSEHPTFTSQYEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAAKLGGALQAKRRQYDPVAALF

SEQ ID NO: 313 - protein, 536 aa, Artificial Sequence
  PD-L1 binding molecule
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAYSEHPTFTSQYEFPKPSTPPGSSGGAPEVQLQQSGPELVKPGASVKISCKTSGYTFTEYTMHWVKQRHGKSLEWIGGINPNNGGTWYNQKFKGKATLTVDKSSSTAYMELRSLTSEDSAVYFCARPYYYGSREDYFDYWGQGTTLTVSSGGGGSDIQMTQSPSSLSASVGDRVTITCSASSSVSYMYWYQQKPRSSPKPWIYLTSNLASGVPARFSGSGSGTSYSLTISSMEAEDAATYYCQQWSSNPPTFGGGTKLELKHHAAQYDPVAALFRRKLGGALQAK

SEQ ID NO: 314 - protein, 9 aa, Artificial Sequence
  CD8+ T-cell epitope
AYAQKIFKI

SEQ ID NO: 315 - protein, 9 aa, Artificial Sequence
  CD8+ T-cell epitope
TVRSHCVSK

SEQ ID NO: 316 - protein, 9 aa, Artificial Sequence
  CD8+ T-cell epitope
TLLNCAVTK

(19) United States
(12) Patent Application Publication
(10) Pub. No.: US 2022/0306700 A1
(43) Pub. Date: Sep. 29, 2022

(54) PD-L1 BINDING PROTEINS COMPRISING SHIGA TOXIN A SUBUNIT SCAFFOLDS AND CD8+ T CELL ANTIGENS

(71) Applicant: Molecular Templates, Inc., Austin, TX (US)

(72) Inventors: Eric Poma, New York, NY (US); Hilario Ramos, Austin, TX (US); Erin Willert, Round Rock, TX (US); Joseph Dekker, Austin, TX (US); Swati Khanna, Austin, TX (US)

(21) Appl. No.: 17/697,564

(22) Filed: Mar. 17, 2022

Related U.S. Application Data
(60) Provisional application No. 63/162,488, filed on Mar. 17, 2021.

(51) Int. Cl. C07K 14/25; A61P 35/00; C07K 14/005

(52) U.S. Cl. CPC C07K 14/25; A61P 35/00; C07K 14/005; C07K 2317/24; C07K 2317/53; C07K 2317/565; C07K 2317/622; C07K 2317/75

(57) ABSTRACT

Provided herein are PD-L1 binding molecules comprising a toxin, e.g. a Shiga toxin A Subunit derived polypeptide. In some embodiments, the PD-L1 binding molecules are cytotoxic. In some embodiments, the PD-L1 binding molecules are capable of delivering a CD8+ T-cell epitope to an MHC class molecule inside a PD-L1 positive cell. The PD-L1 binding molecules described herein have uses for selectively killing specific cells (e.g., PD-L1 positive tumor cells and/or immune cells); for selectively delivering cargos to specific cells (e.g., PD-L1 positive tumor cells or immune cells), and as therapeutic and/or diagnostic molecules for treating and diagnosing a variety of conditions, including cancers and tumors involving PD-L1 expressing cells (e.g., PD-L1 positive tumor cells or immune cells).

### CROSS-REFERENCE TO RELATED APPLICATIONS

This application claims priority to U.S. Provisional Patent Application No. 63/162,488, filed Mar. 17, 2021, which is incorporated by reference herein in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: MTEM_031_01US_SeqList_ST25.txt, date recorded: Mar. 17, 2022, file size ˜548,710 bytes).

### TECHNICAL FIELD

The present application relates to PD-L1 binding molecules comprising toxins, such as, e.g., a catalytic active protein toxin or fragment thereof. In some embodiments, the PD-L1-targeting molecules described herein can kill a PD-L1-expressing cell due to the catalytic activity of a toxin component. In some embodiments, the PD-L1 binding molecules described herein can deliver a CD8+ T-cell epitope to the MHC class I system of a PD-L1-expressing cell. In some embodiments, the PD-L1-targeting molecules described herein comprise a Shiga toxin effector polypeptide derived from the A Subunit of a naturally occurring Shiga toxin. In some embodiments, the PD-L1-targeting molecules described herein comprise Shiga toxin effector polypeptides that comprise multiple amino acid substitution mutations relative to a wild-type Shiga toxin. The PD-L1 binding molecules described herein are useful (1) for selectively killing a specific PD-L1-expressing cell type(s) amongst other cells and (2) as therapeutic molecules for treating a variety of diseases, disorders, and conditions involving PD-L1-expressing cells, including cancers and tumors.

### BACKGROUND

PD-L1, programmed cell death ligand 1 (also known as CD274), is expressed on the cell surface of tumors from a variety of malignancies. PD-L1 can bind to the immune checkpoint receptor PD-1 on T-cells and inhibit T-cell activation signals leading to evasion of immune surveillance by the tumor cell, tumor, and/or other cells in the tumor microenvironment, i.e. T-cell suppression and/or T-cell anergy.

Blockade of the PD-L1/PD-1 signaling axis by therapeutic antibodies can have clinical efficacy for certain diverse indications and may allow for proliferation and/or activation of anti-tumor T-cells beyond normal physiologic conditions. Oncological indications which may benefit from a PD-L1 targeted agent include but are not limited to lung cancer, melanoma, bladder cancer, Hodgkin's lymphoma, breast cancer (including, but not limited to, triple negative breast cancer, i.e., breast cancer that is negative for HER2, estrogen receptor, and progesterone receptor), as well as other neoplasms involving cells which express PD-L1, such as tumor cells with high mutational burdens and/or frequencies of indels. Thus, PD-L1 is a target for delivery of anti-neoplastic agents, including immunotoxins for the alleviation and treatment of certain diseases, disorders, and conditions.

PD-L1 is also expressed on the surface of certain immune cell types. Thus, PD-L1 is a putative target for delivery of immunomodulatory agents (including immunotoxins, immunogens, and vaccines) to such immune cells for the prevention, alleviation, and treatment of certain diseases, disorders, and conditions, such as, e.g., certain immune disorders.

PD-L1 expression may serve a diagnostic marker for the characterization of a cell-type, tissue, disease, disorder, or condition. PD-L1 expression may serve a diagnostic marker for the selection or stratification of patients most likely to respond to certain therapies or therapeutic approaches or to monitor changes in patients during or after receipt of a therapeutic regimen or other intervention. Thus, PD-L1 is a target for diagnostic detection and characterization, such as, e.g., to detect or characterize cells capable of internalizing an immunotoxin-linked diagnostic agent for information-gathering regarding the status of certain diseases, disorders, and conditions, including the progression and effects of treatments thereof.

There is a need in the art to develop molecules comprising PD-L1-targeting immunoglobulin binding domains and toxin components for the creation of PD-L1-targeting molecules which deliver toxins to PD-L1-expressing cells for therapeutic or diagnostic purposes. For example, there is an urgent need for new therapeutics to supplement present day therapies aimed at killing PD-L1-bearing neoplasms.

Thus, it would be desirable to have cytotoxic molecules which bind PD-L1 for use as therapeutic and/or diagnostic molecules to treat and diagnose a variety of diseases, such as, e.g., cancers and tumors, that can be treated by selective killing of or selective delivery of a beneficial agent into a PD-L1 positive cell. In particular, it would be desirable to have PD-L1-binding, cytotoxic, binding molecules comprising toxins that exhibit low immunogenicity, low off-target toxicity, and potent on-target cytotoxicity. Furthermore, it would be desirable to have PD-L1-targeting therapeutic and/or diagnostic molecules exhibiting low immunogenicity, low off-target toxicity, high stability, and/or the ability to deliver peptide-epitope cargos for presentation by the MHC class I system of a target cell. For example, it would be desirable to have PD-L1 binding molecules comprising Shiga toxin A Subunit derived components that maintain potent cytotoxicity while 1) reducing the potential for unwanted antigenicities and/or immunogenicities, 2) reducing the potential for non-specific toxicities, and/or 3) having the ability to deliver peptide-epitope cargos for presentation by the MHC class I system of a target cell, and which also exhibit potent Shiga toxin A Subunit based cytotoxicity to target cells. Certain PD-L1 binding molecules targeting human PD-L1 may offer additional advantages if they do not compete for binding with an already approved anti-PD-L1 therapeutic(s) and/or diagnostic(s). PD-L1 binding molecules comprising toxins (e.g. an immunotoxin) may exhibit unique mechanisms of action if their binding to PD-L1 functions to modulate the PD-L1/PD-1 signaling axis.

### SUMMARY

Provided herein are various embodiments of PD-L1 binding molecules, and compositions thereof. In some embodiments, the present disclosure provides a PD-L1 binding molecule comprising: (i) a Shiga-like toxin A subunit effector polypeptide; (ii) a binding region capable of specifically binding an extracellular part of PD-L1; wherein the binding region comprises: (a) a heavy chain variable region (VH) comprising: (1) a CDR1 comprising the amino acid sequence EYTMH (SEQ ID NO:27), (2) a CDR2 comprising the amino acid sequence GINPNNGGTWYNQKFKG (SEQ ID NO:29), and (3) a CDR3 comprising the amino acid sequence PYYYGSREDYFDY (SEQ ID NO:32); and (b) a light chain variable region (VL) comprising: (1) a CDR1 comprising the amino acid sequence SASSSVSYMY (SEQ ID NO:19). (2) a CDR2 comprising the amino acid sequence LTSNLAS (SEQ ID NO:20), and (3) a CDR3 comprising the amino acid sequence QQWSSNPPT (SEQ ID NO:26); and (iii) at least one CD8+ T-cell epitope that is heterologous to Shiga-like toxin A subunits.

In some embodiments, the CD8+ T-cell epitope comprises the sequence of SEQ ID NO: 300 or 301. In some embodiments, the CD8+ T-cell epitope comprises the sequence of any one of SEQ ID NO: 78-84.

In some embodiments, the at least one CD8+ T-cell epitope is an antigen recognized by HLA subtypes HLA-A, HLA-B, or HLA-C. In some embodiments, the at least one CD8+ T-cell epitope is an HLA:A01 restricted antigen. In some embodiments, the at least one CD8+ T-cell epitope is an HLA:A02 restricted antigen. In some embodiments, the at least one CD8+ T-cell epitope is an HLA:A03 restricted antigen. In some embodiments, the at least one CD8+ T-cell epitope is an HLA:A24 restricted antigen

In some embodiments, the at least one CD8+ T-cell epitope is isolated or derived from Human Cytomegalovirus (HCMV).

In some embodiments, the at least one CD8+ T-cell epitope is embedded or inserted into the Shiga-like toxin A subunit effector polypeptide. In some embodiments, the at least one CD8+ T-cell epitope is located at the C-terminus of the Shiga-like toxin A subunit effector polypeptide.

In some embodiments, the at least one CD8+ T-cell epitope is embedded or inserted into the binding region. In some embodiments, the at least one CD8+ T-cell epitope is located at the C-terminus of the binding region.

In some embodiments, the at least one CD8+ T-cell epitope is located between the Shiga-like toxin A subunit effector polypeptide and the binding region.

In some embodiments, the molecule comprises at least two CD8+ T-cell epitopes that are each heterologous to Shiga-like toxin A subunits.

In some embodiments, the molecule comprises at least three CD8+ T-cell epitopes that are each heterologous to Shiga-like toxin A subunits. In some embodiments, the molecule comprises at least four CD8+ T-cell epitopes that are each heterologous to Shiga-like toxin A subunits.

In some embodiments, the molecule comprises, in order from N-terminus to C-terminus the Shiga-like toxin A subunit effector polypeptide; the binding region; and the at least one CD8+ T-cell epitope. In some embodiments, the molecule comprises, in order from N-terminus to C-terminus, the Shiga-like toxin A subunit effector polypeptide; the binding region; and at least two CD8+ T-cell epitopes. In some embodiments, the molecule comprises, in order from N-terminus to C-terminus the Shiga-like toxin A subunit effector polypeptide; the at least one CD8+ T-cell epitope; and the binding region.

In some embodiments, the molecule comprises, in order from N-terminus to C-terminus the Shiga-like toxin A subunit effector polypeptide; a first CD8+ T-cell epitope; the binding region; and a second CD8+ T-cell epitope. In some embodiments, the first and the second CD8+ T-cell epitopes are different.

In some embodiments, the molecule comprises, in order from N-terminus to C-terminus the Shiga-like toxin A subunit effector polypeptide; a first CD8+ T-cell epitope; the binding region; a second CD8+ T-cell epitope; and a third CD8+ T-cell epitope. In some embodiments, at least one of the first, second, and third CD8+ T-cell epitopes is different from the others.

In some embodiments, the molecule comprises, in order from N-terminus to C-terminus the binding region; the Shiga-like toxin A subunit effector polypeptide; and the at least one CD8+ T-cell epitope.

In some embodiments, the molecule comprises, in order from N-terminus to C-terminus, the binding region; the Shiga-like toxin A subunit effector polypeptide; and at least two CD8+ T-cell epitopes.

In some embodiments, the molecule comprises, in order from N-terminus to C-terminus the binding region; the at least one CD8+ T-cell epitope; and the Shiga-like toxin A subunit effector polypeptide.

In some embodiments, the molecule comprises, in order from N-terminus to C-terminus, the binding region; a first CD8+ T-cell epitope; the Shiga-like toxin A subunit effector polypeptide; and a second CD8+ T-cell epitope. In some embodiments, the first and the second CD8+ T-cell epitopes are different.

In some embodiments, the molecule comprises, in order from N-terminus to C-terminus the binding region; a first CD8+ T-cell epitope; the Shiga-like toxin A subunit effector polypeptide; a second CD8+ T-cell epitope; and a third CD8+ T-cell epitope. In some embodiments, at least one of the first, second, and third CD8+ T-cell epitopes is different from the others.

In some embodiments, the Shiga-like toxin A subunit effector polypeptide comprises the sequence of SEQ ID NO: 41, or a sequence at least 90% or at least 95% identical thereto. In some embodiments, the VH comprises the sequence of SEQ ID NO: 34, or a sequence at least 90% or at least 95% identical thereto. In some embodiments, the VL comprises the sequence of SEQ ID NO: 35, or a sequence at least 90% or at least 95% identical thereto. In some embodiments, the VH comprises the sequence of SEQ ID NO: 34 and the VL comprises the sequence of SEQ ID NO: 35.

In some embodiments, the binding region comprises the sequence of SEQ ID NO: 106, or a sequence at least 90% or at least 95% identical thereto. In some embodiments, the PD-L1 binding molecule comprises the sequence of any one of SEQ ID NOs: 303-313, or a sequence at least 90% or at least 95% identical thereto.

In some embodiments, the PD-L1 binding molecule is a single continuous polypeptide. In some embodiments, the PD-L1 binding molecule comprises two polypeptides. In some embodiments, each of the two polypeptide comprises the sequence of any one of SEQ ID NO: 303-313. In some embodiments, the two polypeptides are non-covalently linked to each other.

In some embodiments, the binding molecule is cytotoxic.

In some embodiments, the present disclosure provides a cell binding molecule comprising: (i) a Shiga-like toxin A subunit effector polypeptide; (ii) a binding region capable of specifically binding an extracellular target on a cell; and (iii) CD8+ T-cell epitope comprising the sequence of SEQ ID NO: 300 or 301.

In some embodiments, the at least one CD8+ T-cell epitope is embedded or inserted into the Shiga-like toxin A subunit effector polypeptide. In some embodiments, the at least one CD8+ T-cell epitope is located at the C-terminus of the Shiga-like toxin A subunit effector polypeptide. In some embodiments, the at least one CD8+ T-cell epitope is embedded or inserted into the binding region. In some embodiments, the at least one CD8+ T-cell epitope is located at the C-terminus of the binding region. In some embodiments, the at least one CD8+ T-cell epitope is located between the Shiga-like toxin A subunit effector polypeptide and the binding region.

In some embodiments, the molecule comprises at least two CD8+ T-cell epitopes that are each heterologous to Shiga-like toxin A subunits. In some embodiments, the molecule comprises at least three CD8+ T-cell epitopes that are each heterologous to Shiga-like toxin A subunits. In some embodiments, the molecule comprises at least four CD8+ T-cell epitopes that are each heterologous to Shiga-like toxin A subunits.

In some embodiments, the molecule comprises, in order from N-terminus to C-terminus the Shiga-like toxin A subunit effector polypeptide; the binding region; and the at least one CD8+ T-cell epitope. In some embodiments, the molecule comprises, in order from N-terminus to C-terminus, the Shiga-like toxin A subunit effector polypeptide; the binding region; and at least two CD8+ T-cell epitopes. In some embodiments, the molecule comprises, in order from N-terminus to C-terminus, the Shiga-like toxin A subunit effector polypeptide; the at least one CD8+ T-cell epitope; and the binding region.

In some embodiments, the molecule comprises, in order from N-terminus to C-terminus the Shiga-like toxin A subunit effector polypeptide; a first CD8+ T-cell epitope; the binding region; and a second CD8+ T-cell epitope. In some embodiments, the first and the second CD8+ T-cell epitopes are different.

In some embodiments, the molecule comprises, in order from N-terminus to C-terminus the Shiga-like toxin A subunit effector polypeptide; a first CD8+ T-cell epitope; the binding region; a second CD8+ T-cell epitope; and a third CD8+ T-cell epitope. In some embodiments, at least one of the first, second, and third CD8+ T-cell epitopes is different from the others.

In some embodiments, the molecule comprises, in order from N-terminus to C-terminus the binding region; the Shiga-like toxin A subunit effector polypeptide; and the at least one CD8+ T-cell epitope.

In some embodiments, the molecule comprises, in order from N-terminus to C-terminus, the binding region; the Shiga-like toxin A subunit effector polypeptide; and at least two CD8+ T-cell epitopes.

In some embodiments, the molecule comprises, in order from N-terminus to C-terminus, the binding region; the at least one CD8+ T-cell epitope; and the Shiga-like toxin A subunit effector polypeptide.

In some embodiments, the molecule comprises, in order from N-terminus to C-terminus, the binding region; a first CD8+ T-cell epitope; the Shiga-like toxin A subunit effector polypeptide; and a second CD8+ T-cell epitope. In some embodiments, the first and the second CD8+ T-cell epitopes are different.

In some embodiments, the molecule comprises, in order from N-terminus to C-terminus the binding region; a first CD8+ T-cell epitope; the Shiga-like toxin A subunit effector polypeptide; a second CD8+ T-cell epitope; and a third CD8+ T-cell epitope. In some embodiments, at least one of the first, second, and third CD8+ T-cell epitopes is different from the others.

In some embodiments, the Shiga-like toxin A subunit effector polypeptide comprises the sequence of SEQ ID NO: 41, or a sequence at least 90% or at least 95% identical thereto. In some embodiments, the Shiga-like toxin A subunit effector polypeptide comprises the amino acids 1-251 of SEQ ID NO: 1, or a sequence at least 90% or at least 95% identical thereto.

In some embodiments, the present disclosure provides a pharmaceutical composition comprising the binding molecule described herein, and at least one pharmaceutically acceptable excipient or carrier.

In some embodiments, the present disclosure provides a polynucleotide encoding the binding molecule described herein, or a complement thereof. In some embodiments, the present disclosure provides an expression vector comprising the polynucleotide. In some embodiments, the present disclosure provides a host cell comprising the polynucleotide or the expression vector.

In some embodiments, the present disclosure provides a method for making the binding molecule described herein, the method comprising (a) expressing the binding molecule and (b) recovering the binding molecule. In some embodiments, expressing the binding molecule comprises culturing the host cell described herein under conditions wherein the binding molecule is expressed.

In some embodiments, the present disclosure provides a method of killing a cell, the method comprising the step of contacting the cell with a binding molecule described herein or a pharmaceutical composition described herein.

In some embodiments, the present disclosure provides a method of treating a disease, disorder, or condition in a subject, the method comprising a step of administering to a subject in need thereof a therapeutically effective amount of a binding molecule described herein or a pharmaceutical composition described herein. In some embodiments, the disease, disorder, or condition is an immune disorder or microbial infection.

In some embodiments, the present disclosure provides a method of treating cancer, the method comprising administering to a subject in need thereof an effective amount of the binding molecule described herein, or a pharmaceutical composition described herein. In some embodiments, the cancer is characterized by a high mutational burden and/or a high frequency of indels. In some embodiments, the cancer is a solid tumor. In some embodiments, the cancer is bladder cancer, breast cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, or urothelial cancer. In some embodiments, the cancer is bladder cancer, and the bladder cancer is urothelial carcinoma. In some embodiments, the cancer is breast cancer, and the breast cancer is HER2 positive breast cancer or triple negative breast cancer. In some embodiments, the cancer is colon cancer, and the colon cancer is colorectal cancer. In some embodiments, the cancer is gastrointestinal cancer, and the gastrointestinal cancer is gastric cancer, biliary tract neoplasm, or gastroesophageal junction cancer. In some embodiments, the cancer is glioma, and the glioma is glioblastoma. In some embodiments, the cancer is head and neck cancer, and the head and neck cancer is squamous cell carcinoma of the head and neck. In some embodiments, the cancer is kidney cancer, and the kidney cancer is renal cell carcinoma. In some embodiments, the cancer is liver cancer, and the liver cancer is hepatocellular carcinoma. In some embodiments, the cancer is lung cancer, and the lung cancer is non-small cell lung cancer or small-cell lung cancer. In some embodiments, the cancer is lymphoma, and the lymphoma is Hodgkin lymphoma, non-Hodgkin lymphoma, primary mediastinal large B-cell lymphoma, or diffuse large B-cell lymphoma. In some embodiments, the cancer is mesothelioma, and the mesothelial carcinoma is pleural mesothelioma. In some embodiments, the cancer is myeloma, and the myeloma is multiple myeloma. In some embodiments, the cancer is skin cancer, and the skin cancer is squamous cell cancer of the skin or melanoma. In some embodiments, the cancer is relapsed or refractory to treatment with one or more checkpoint inhibitors. In some embodiments, the cancer is relapsed or refractory to a treatment involving at least one of ipilimumab, nivolumab, pembrolizumab, atezolizumab, durvalumab, avelumab, tremelimumab and cemiplimab. In some embodiments, the cancer is metastatic.

These and other features, aspects and advantages of the present invention will become better understood with regard to the following description, appended claims, and accompanying figures. The aforementioned elements of the invention may be individually combined or removed freely in order to make other embodiments of the invention, without any statement to object to such synthesis or removal hereinafter.

### BRIEF DESCRIPTION OF THE DRAWINGS

FIG. 1 is a schematic showing the benefits of PD-L1 binding molecules and their mechanisms of action (MOA) to elicit an anti-tumor response.

FIG. 2 is a schematic of the antigen seeding mechanism of the single and multi-antigen PD-L1 binding molecules. A PD-L1 binding molecule delivers an antigen to a PD-L1 expressing cell and the antigen is presented on the surface of the cell and recognized by cytotoxic T cells to induce an anti-tumor response.

FIG. 3A shows the results of a ribosome inhibition assay for the PD-L1 binding molecule MT-6402.

FIG. 3B shows the results of a PD-L1 target binding assay for the PD-L1 binding molecule MT-6402.

FIG. 3C shows PD-L1 cell-surface expression on tumor cell lines.

FIG. 3D shows the results of a cytotoxicity assay for the PD-L1 binding molecule MT-6402.

FIG. 4A shows the results of a co-culture cytotoxicity assay for the PD-L1 binding molecule MT-6402 comprising a CMV-restricted MHC-1 peptide (NLVPMVATV, SEQ ID NO: 78) compared to PD-L1 binding molecules without a CMV-restricted MHC-I peptide.

FIG. 4B shows the results of cytotoxic T cell (CTL) activation for the PD-L1 binding molecule MT-6402 comprising a CMV-restricted MHC-I peptide (NLVPMVATV, SEQ ID NO: 78).

FIG. 5 is a schematic of PD-L1 binding molecules comprising single or multiple HLA:A01-restricted antigens in different locations of the PD-L1 binding molecule.

FIG. 6A shows the results of a PD-L1 target binding assay for exemplary single and multi-antigen PD-L1 binding molecules.

FIG. 6B shows the results of a cytotoxicity assay for exemplary single and multi-antigen PD-L1 binding molecules.

FIG. 7A is a schematic of culture and expansion of human antigen-specific T cells ex vivo. Healthy PBMCs are cultured ex vivo with antigenic peptide, cytokines, and peptide-loaded dendritic cells (DCs) to induce expansion of antigen-specific T cells.

FIG. 7B shows the expansion of antigen-specific T cells from human PBMCs cultured with Antigen #1 and Antigen #2. Antigen-specific T cells were detected by flow cytometry using MHC tetramers.

FIG. 8A shows the results of cytotoxic T cell (CTL) activation for single and multi-antigen PD-L1 binding molecules. PD-L1-high and PD-L1-low HLA:A1 target cells were incubated with single (Molecule F and Molecule B) or multi-antigen (Molecule I) PD-L1 binding molecules and then co-cultured with antigen-restricted T cells. IFN-γ secretion was determined by ELISA.

FIG. 8B shows the results of cytotoxic T cell (CTL) activation for single and multi-antigen PD-L1 binding molecules. PD-L1-high and PD-L1-low HLA:A1 target cells were incubated with single (Molecule E and Molecule A) or multi-antigen (Molecule I) PD-L1 binding molecules and then co-cultured with antigen-restricted T cells. IFN-γ secretion was determined by ELISA.

FIG. 8C shows the results of cytotoxic T cell (CTL) activation for single and multi-antigen PD-L1 binding molecules. PD-L1-high and PD-L1-low HLA:A24 target cells were incubated with single (Molecule D and Molecule H) or multi-antigen (Molecule J) PD-L1 binding molecules and then co-cultured with antigen-restricted T cells. IFN-γ secretion was determined by ELISA.

FIG. 9A shows the results of a cell viability assay for single and multi-antigen PD-L1 binding molecules. PD-L1-high and PD-L1-low HLA:A1 target cells (A375 cell line) were incubated with single (Molecule F and Molecule B) or multi-antigen (Molecule I) PD-L1 binding molecules and then co-cultured with antigen-restricted T cells. IFN-T secretion was determined by ELISA.

FIG. 9B shows the results of a cell viability assay for single and multi-antigen PD-L1 binding molecules. PD-L1-high HLA:A1 target cells (A375 cell line) were incubated with single (Molecule E and Molecule A) or multi-antigen (Molecule I) PD-L1 binding molecules and then co-cultured with antigen-restricted T cells. IFN-γ secretion was determined by ELISA.

FIG. 9C shows the results of a cell viability assay for single and multi-antigen PD-L1 binding molecules. PD-L1-high, HLA:A24 target cells (PC-3 cell line) were incubated with single (Molecule D and Molecule H) or multi-antigen (Molecule J) PD-L1 binding molecules and then co-cultured with antigen-restricted T cells. IFN-γ secretion was determined by ELISA.

FIG. 10A shows the results of an in vitro cytotoxicity assay for single-antigen PD-L1 binding molecules. HCC1954 cells were incubated with various concentrations of single-antigen (Molecule A, Molecule B, Molecule C, or Molecule D) PD-L1 binding molecules and percentage cell viability was measured.

FIG. 10B and FIG. 10C show the results of a study in an in vivo tumor efficacy model. Tumor-bearing mice were administered single or multi-antigen PD-L1 binding molecules and tumor volume was measured over time. A buffer diluted in saline was used as a vehicle control. Arrowheads indicate the days mice were dosed with single- (Molecule A, Molecule B. Molecule E, or Molecule F) or multi-antigen (Molecule J) PD-L1 binding molecules.

FIG. 11A shows a workflow for an AST assay timeline followed by cytokine analysis.

FIG. 11B shows the results of an assay measuring antigen specific T cell driven TNFα cytokine release from HLA-A*01 and HLA-A*02 co-culture assays 48 hours post intoxication with PD-L1 binding molecules carrying matched or mismatched antigens.

FIG. 11C shows a workflow for a PBMC cytokine release assay timeline followed by cytokine analysis.

FIG. 11D shows the results of an assay measuring IP-10 cytokine release from HLA-A*01 and HLA-A*02 donor PBMCs intoxicated with PD-L1 binding molecules carrying matched or mismatched antigens.

FIG. 11E shows the results of an assay measuring IP-10 cytokine release from HLA-A*01 and HLA-A*02 donor PBMCs intoxicated with PD-L1 binding molecules carrying matched or mismatched antigens.

FIG. 11F is a Venn diagram of overlapping cytokines released in HLA matched PBMCs for HLA-A*01, HLA-A*02, and HLA-A*24.

FIG. 11G is a table showing a summary of cytokines from the AST assay, the PBMC assay, and clinical data in an HLA matched setting. Shaded cells indicate HLA matched cytokine release for each respective setting.

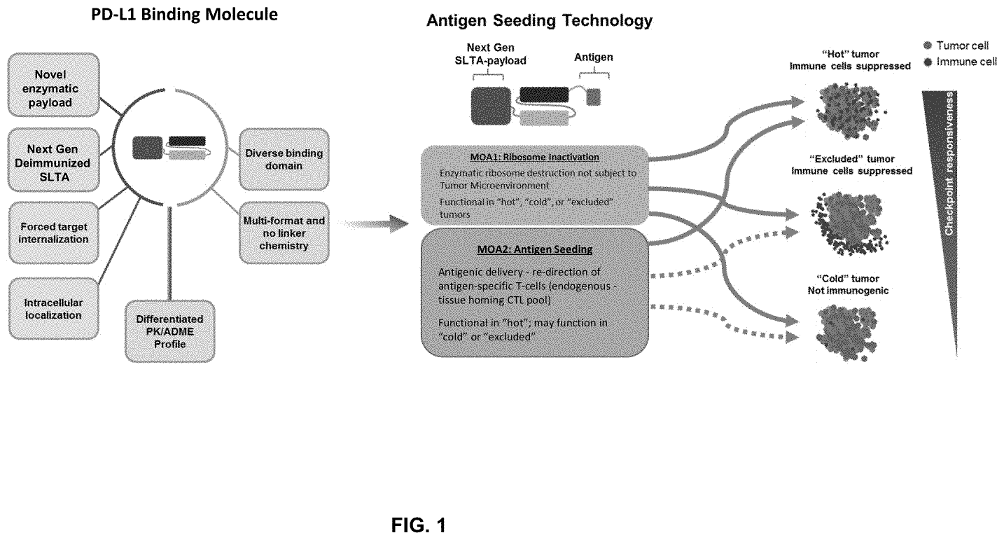

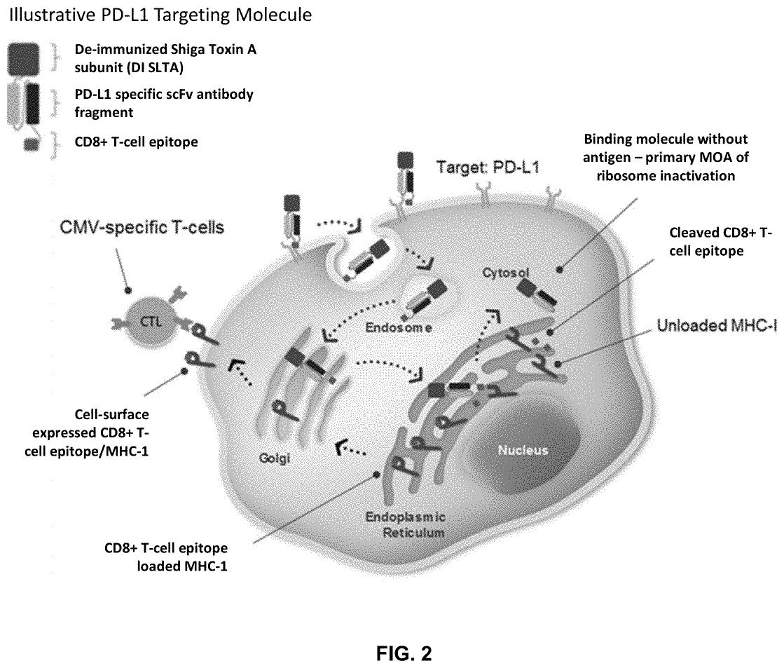

### DETAILED DESCRIPTION

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art.

In order that the present invention may be more readily understood, certain terms are defined below. Additional definitions may be found within the detailed description of the invention.

As used in the specification and the appended claims, the terms “a,” “an” and “the” include both singular and the plural referents unless the context clearly dictates otherwise.

As used in the specification and the appended claims, the term “and/or” when referring to two species, A and B, means at least one of A and B. As used in the specification and the appended claims, the term “and/or” when referring to greater than two species, such as A, B. and C, means at least one of A, B, or C, or at least one of any combination of A, B, or C (with each species in singular or multiple possibility).

The term “amino acid residue” or “amino acid” includes reference to an amino acid that is incorporated into a protein, polypeptide, or peptide. The term “polypeptide” includes any polymer of amino acids or amino acid residues. The term “polypeptide sequence” refers to a series of amino acids or amino acid residues which physically comprise a polypeptide. A “protein” is a macromolecule comprising one or more polypeptides or polypeptide “chains.” A “peptide” is a small polypeptide of sizes less than about a total of 15 to 20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino-terminus to a carboxy-terminus.

The terms “amino acid,” “amino acid residue,” “amino acid sequence,” or polypeptide sequence include naturally occurring amino acids (including L and D isostereomers) and, unless otherwise limited, also include known analogs of natural amino acids that can function in a similar manner as naturally occurring amino acids, such as selenocysteine, pyrrolysine. N-formylmethionine, gamma-carboxyglutamate, hydroxyprolinehypusine, pyroglutamic acid, and selenomethionine. The amino acids referred to herein are described by shorthand designations as follows in Table 1:

TABLE 1
Amino Acid Nomenclature

| Name | 3-letter | 1-letter |
|---|---|---|
| Alanine | Ala | A |
| Arginine | Arg | R |
| Asparagine | Asn | N |
| Aspartic Acid or Aspartate | Asp | D |
| Cysteine | Cys | C |
| Glutamic Acid or Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The phrase “conservative substitution” with regard to an amino acid residue of a peptide, peptide region, polypeptide region, protein, or molecule refers to a change in the amino acid composition of the peptide, peptide region, polypeptide region, protein, or molecule that does not substantially alter the function and structure of the overall peptide, peptide region, polypeptide region, protein, or molecule (see Creighton, Proteins: Structures and Molecular Properties (W. H. Freeman and Company, New York (2nd ed., 1992))).

The phrase “derived from” when referring to a polypeptide or polypeptide region means that the polypeptide or polypeptide region comprises amino acid sequences originally found in a “parental” protein and which may now comprise certain amino acid residue additions, deletions, truncations, rearrangements, or other alterations relative to the original polypeptide or polypeptide region as long as a certain function(s) and a structure(s) of the “parental” molecule are substantially conserved. The skilled worker will be able to identify a parental molecule from which a polypeptide or polypeptide region was derived using techniques known in the art, e.g., protein sequence alignment software.

As used herein, the term “comparable” means similar. When “comparable” refers to a particular value (e.g., a binding affinity), the term may encompass values which are within about 5%, about 10%, about 15%, about 20%, or about 25%, or more, of one another.

As used herein, the term “antibody” refers to immunoglobulin proteins and encompasses the broadest of antibody formats having antigen binding capability, such as, e.g., various protein structures comprising at least one immunoglobulin domain, including but not limited to monoclonal antibodies, polyclonal antibodies, human antibodies, humanized antibodies, chimeric antibodies, camelized antibodies, or antigen-binding antibody fragments (e.g. a Fab, Fv, scFv, sdAb fragment), so long as they exhibit the desired antigen-binding activity.

As used herein, the term “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. Antibody fragments can be made by various techniques, including but not limited to proteolytic digestion of an intact antibody as well as production by recombinant host cells (e.g. E. coli or phage), as described herein.

In some embodiments, an antibody or antibody fragment described herein is a single-domain antibody fragment, single-chain variable fragment, antibody variable fragment, Fd fragment, Fab (antigen-binding fragment), an autonomous VH domain, single domain immunoglobulin-derived region VHH, heavy-chain antibody domain derived from a camelid VHH fragment or VH domain fragment, heavy-chain antibody domain derived from cartilaginous fish VHH fragment or VH domain fragment, immunoglobulin new antigen receptor (IgNAR), VNAR fragment, disulfide stabilized antibody variable (Fv) fragment. Armadillo repeat polypeptide, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold and so forth.

In some embodiments, the antibody or antibody fragment is a multivalent antibody. For example, the antibody or antibody fragment may be a multimerizing scFv fragment such as diabody, triabody, tetrabody, bispecific tandem scFv fragment, bispecific tandem VHH fragment, bispecific minibody or bivalent minibody.

As used herein, the term “chimeric” antibody refers to an antibody in which a portion of the heavy and/or light chain is derived from a particular source or species, while the remainder of the heavy and/or light chain is derived from a different source or species.

As used herein, a “humanized antibody” is one which possesses an amino acid sequence and/or residues involved in antigen-binding (e.g. a CDR) that are derived from a non-human source and wherein one or more other amino acid sequences is derived from a human source (e.g. a framework sequence).

As used herein, a “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues (e.g. CDRs). A human single-domain antibody is one comprising only a human heavy chain or human light chain; however, the CDR sequence may be naturally occurring or synthetic (see e.g. U.S. Pat. No. 6,248,516).

As used herein, a “camelized antibody” is one which possesses an amino acid sequence derived from a non-camelid source and comprises two heavy chains and no light chains and comprises a hinge region derived from a camelid source or species.

The terms “toxin”, “toxin agent”, “toxin component”, or “cytotoxin” as used herein refers to a substance that inhibits or prevents a cellular function and/or causes cell death or destruction, including tissue damage. The toxin component of a binding molecule or antibody toxin conjugate may include, but is not limited to, natural toxins, biotoxins, proteinaceous toxins, venom, cytotoxins, small molecule toxins, and synthetic toxicants derived from any of the aforementioned, such as, e.g. ABx toxin, ribosome inactivating protein toxin, abrin, anthrax toxin, Aspf1, bouganin, bryodin, cholix toxin, claudin, diphtheria toxin, gelonin, heat-labile enterotoxin, mitogillin, pertussis toxin, pokeweed antiviral protein, pulchellin, Pseudomonas exotoxin A, restrictocin, ricin, saporin, sarcin. Shiga toxin, and subtilase cytotoxin, and the various toxin agents described herein or known to the skilled worker.

For purposes of the instant disclosure and with regard to a Shiga toxin polypeptide sequence or Shiga toxin derived polypeptide, the term “wild-type” generally refers to a naturally occurring. Shiga toxin protein sequence(s) found in a living species, such as, e.g., a pathogenic bacterium, wherein that Shiga toxin protein sequence(s) is one of the most frequently occurring variants. This is in contrast to infrequently occurring Shiga toxin protein sequences that, while still naturally occurring, are found in less than one percent of individual organisms of a given species when sampling a statistically powerful number of naturally occurring individual organisms of that species which comprise at least one Shiga toxin protein variant. A clonal expansion of a natural isolate outside its natural environment (regardless of whether the isolate is an organism or molecule comprising biological sequence information) does not alter the naturally occurring requirement as long as the clonal expansion does not introduce new sequence variety not present in naturally occurring populations of that species and/or does not change the relative proportions of sequence variants to each other.

The terms “associated,” “associating,” “linked,” or “linking” refers to the state of two or more components of a molecule being joined, attached, connected, or otherwise coupled to form a single molecule or the act of making two molecules associated with each other to form a single molecule by creating an association, linkage, attachment, and/or any other connection between the two molecules. For example, the term “linked” may refer to two or more components associated by one or more atomic interactions such that a single molecule is formed and wherein the atomic interactions may be covalent and/or non-covalent. Non-limiting examples of covalent associations between two components include peptide bonds and cysteine-cysteine disulfide bonds. Non-limiting examples of non-covalent associations between two molecular components include ionic bonds.

The term “linked” refers to two or more molecular components associated by one or more atomic interactions such that a single molecule is formed and wherein the atomic interactions includes at least one covalent bond. The term “linking” refers to the act of creating a linked molecule as described above.

By “linker” herein is meant a domain linker that joins two protein domains together, such as are used in scFv and/or other protein and protein fusion structures. For example, a “binding region linker” may be used to link a Shiga Toxin A subunit effector polypeptide with a binding region, and a “scFv linker” may be used to link the VH and the VL in an scFv. A “cleavable spacer” is a type of linker that contains a cleavage site for one or more proteases. Generally, there are a number of suitable linkers that can be used, including traditional peptide bonds, generated by recombinant techniques that allows for recombinant attachment of the two domains with sufficient length and flexibility to allow each domain to retain its biological function. In some embodiments, the linker peptide can predominantly include the following amino acid residues: Gly, Ser, Ala, or Thr. The linker peptide should have a length that is adequate to link two molecules in such a way that they assume the correct conformation relative to one another so that they retain the desired activity. In some embodiments, the linker is from about 1 to about 50 amino acids in length. In some embodiments, the linker is from about 1 to about 30 amino acids in length. In one embodiment, linkers of 1 to 20 amino acids in length can be used, with from about 5 to about 10 amino acids finding use in some embodiments. Useful linkers include glycine-serine polymers, including for example (GS)n (SEQ ID NO: 201), (GSGGS)n (SEQ ID NO: 202), (GGGGS)n (SEQ ID NO: 203), and (GGGS)n (SEQ ID NO: 204), where n is an integer of at least one (and generally from 3 to 4), glycine-alanine polymers, alanine-serine polymers, and other flexible linkers. Alternatively, a variety of non-proteinaceous polymers, including but not limited to polyethylene glycol (PEG), polypropylene glycol, polyoxyalkylenes, or copolymers of polyethylene glycol and polypropylene glycol, can find use as linkers. Other linker sequences can include any sequence of any length of CL/CH1 domain but not all residues of CL/CH1 domain; for example, the first 5-12 amino acid residues of the CL/CH1 domains. Linkers can also be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences can also be derived from other proteins such as Ig-like proteins (e.g., TCR, FcR, KIR), hinge region-derived sequences, and other natural sequences from other proteins. While any suitable linker can be used, some embodiments utilize a glycine-serine polymer, including for example (GS)n (SEQ ID NO: 201), (GSGGS)n (SEQ ID NO: 202), (GGGGS)n (SEQ ID NO: 203), and (GGGS)n (SEQ ID NO: 204), where n is an integer of at least one (and generally from 2 to 3 to 4 to 5). “scFv linkers” generally include these glycine-serine polymers.

The term “fused” refers to two or more proteinaceous components associated by at least one covalent bond which is a peptide bond, regardless of whether the peptide bond involves the participation of a carbon atom of a carboxyl acid group or involves another carbon atom, such as, e.g., the α-carbon, β-carbon, γ-carbon, σ-carbon, etc. Non-limiting examples of two proteinaceous components fused together include, e.g., an amino acid, peptide, or polypeptide fused to a polypeptide via a peptide bond such that the resulting molecule is a single, continuous polypeptide. The term “fusing” refers to the act of creating a fused molecule as described above, such as, e.g., a fusion protein generated from the recombinant fusion of genetic regions which when translated produces a single proteinaceous molecule.

The symbol “::” means the polypeptide regions before and after it are physically linked together to form a continuous polypeptide.

As used herein, the terms “expressed,” “expressing,” or “expresses,” and grammatical variants thereof, refer to translation of a polynucleotide or nucleic acid into a protein. The expressed protein may remain intracellular, become a component of the cell surface membrane or be secreted into an extracellular space.

As used herein, cells which express a significant amount of an extracellular target biomolecule at least one cellular surface are “target positive cells”, “target+ cells”, or “+ve cells” and are cells physically coupled to the specified, extracellular target biomolecule.

As used herein, the symbol “α” is shorthand for an immunoglobulin-type binding region capable of binding to the biomolecule following the symbol. The symbol “α” is used to refer to the functional characteristic of an immunoglobulin-type binding region based on its ability to bind to the biomolecule following the symbol with a binding affinity described by a dissociation constant (K_{D}) of 10⁻⁵ or less.

As used herein, the term “heavy chain variable (V_{H}) domain” or “light chain variable (V_{L}) domain” respectively refer to any antibody V_{H} or V_{L} domain (e.g. a human V_{H} or V_{L} domain) as well as any derivative thereof retaining at least qualitative antigen binding ability of the corresponding native antibody (e.g. a humanized V_{H} or V_{L} domain derived from a native murine V_{H} or V_{L} domain). A V_{H} or V_{L} domain consists of a “framework” region interrupted by the three CDRs or ABRs. As used herein, the term “framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in a VH (or VL): FR1-H1(L1)-FR2-H2(L2)-FR3-H3(L3)-FR4. The framework regions serve to align the CDRs or ABRs for specific binding to an epitope of an antigen. From amino-terminus to carboxy-terminus, both V_{H} and V_{L} domains comprise the following framework (FR) and CDR regions or ABR regions: FR1, CDR1, FR2, CDR2, FR3, CDR3, and FR4; or, similarly, FR1, ABR1, FR2, ABR2, FR3, ABR3, and FR4. As used herein, the terms “HCDR1,” “HCDR2,” or “HCDR3” are used to refer to CDRs 1, 2, or 3, respectively, in a V_{H} domain, and the terms “LCDR1,” “LCDR2,” and “LCDR3” are used to refer to CDRs 1, 2, or 3, respectively, in a V_{L} domain. As used herein, the terms “HABR1,” “HABR2,” or “HABR3” are used to refer to ABRs 1, 2, or 3, respectively, in a V_{H} domain, and the terms “LABR1,” “LABR2,” or “LABR3” are used to refer to CDRs 1, 2, or 3, respectively, in a V_{L} domain. For camelid V_{H}H fragments, IgNARs of cartilaginous fish, V_{NAR} fragments, certain single domain antibodies, and derivatives thereof, there is a single, heavy chain variable domain comprising the same basic arrangement: FR1, CDR1, FR2, CDR2, FR3, CDR3, and FR4. As used herein, the terms “HCDR1,” “HCDR2,” or “HCDR3” may be used to refer to CDRs 1.2, or 3, respectively, in a single heavy chain variable domain. A single V_{H} or V_{L} domain may be sufficient to confer antigen-binding specificity.

The term “effector” means providing a biological activity, such as cytotoxicity, biological signaling, enzymatic catalysis, subcellular routing, and/or intermolecular binding resulting in an allosteric effect(s) and/or the recruitment of one or more factors.

The term “Shiga toxin” herein refers to two families of related toxins: Shiga toxin (Stx)/Shiga-like toxin 1 (SLT-1/Stx1) and Shiga-like toxin 2 (SLT-2/Stx2). Six is produced by Shigella dysenteriae, while SLT-1 and SLT-2 are derived from Escherichia coli. Members of the Shiga toxin family share the same overall structure and mechanism of action (Engedal N et al., Microbial Biotech 4: 32-46 (2011)). For example, Stx, SLT-1 and SLT-2 display indistinguishable enzymatic activity in cell free systems (Head S et al., J Biol Chem 266: 3617-21 (1991); Tesh V et al., Infect Immun 61: 3392402 (1993); Brigotti M et al., Toxicon 35:1431-1437 (1997)).

Stx, SLT-1, and SLT-2 are multimeric molecules comprised of two polypeptide subunits, A and B. The B Subunit is a pentamer that binds the toxin to glycolipids on host cell membranes and enters the cell via endocytosis. Once inside the cell, the A Subunit undergoes proteolytic cleavage and the reduction of an internal disulfide bond to generate the A1 Subunit and the A2 Subunit. The Shiga toxin or Shiga-like toxin A1 Subunits (e.g., SLT-1-A1) are N-glycosidases that catalytically inactivate the 28S ribosomal RNA subunit to inhibit protein synthesis.

As described herein, the phrase “Shiga toxin effector region” refers to a polypeptide derived from a Shiga toxin A Subunit or Shiga-like toxin A Subunit of the Shiga toxin family, which exhibits at least one Shiga toxin effector function. For example, SEQ ID NO: 49-61 are derived from StxA and/or SLT-1A. In some embodiments, the Shiga toxin effector region of the PD-L1-binding molecule is a Shiga toxin A Subunit, such as StxA. In some embodiments, the Shiga toxin effector region of the PD-L1-binding molecule is a Shiga-like toxin A Subunit, such as SLT-1A or SLT-2A. In some embodiments, the Shiga toxin effector region of the PD-L1-binding molecule is an A1 Subunit of SLT-1 (e.g., SLT-1-A1). In some embodiments, the Shiga toxin effector region of the PD-L1-binding molecule is an enzymatically active, de-immunized Shiga-like toxin A1 Subunit of SLT-1 (e.g., SLT-1-A1 V1). In some embodiments, the Shiga toxin effector region has a sequence of SEQ ID NO: 41, or a sequence at least 85%, at least 90%, at least 95%, or at least 99% identical thereto. In some embodiments, the Shiga toxin effector region has a sequence of SEQ ID NO: 41 with 1-10, 10-20, 20-30, 30-40, 40-50 or more amino acid substitutions. In some embodiments, the Shiga toxin effector region has a sequence of any one of SEQ ID NO: 259 or 261-284, or a sequence at least 85%, at least 90%, at least 95%, or at least 99% identical thereto. In some embodiments, the Shiga toxin effector region has a sequence of any one of SEQ ID NO: 259 or 264-284 with 1-10, 10-20, 20-30, 30-40, 40-50 or more amino acid substitutions.

As described herein, a Shiga toxin effector function is a biological activity conferred by a polypeptide region derived from a Shiga toxin A Subunit. Non-limiting examples of Shiga toxin effector functions include promoting cell entry; lipid membrane deformation; promoting cellular internalization; stimulating clathrin-mediated endocytosis; directing intracellular routing to various intracellular compartments such as, e.g., the Golgi, endoplasmic reticulum, and cytosol; directing intracellular routing with a cargo; inhibiting a ribosome function(s); catalytic activities, such as, e.g., N-glycosidase activity and catalytically inhibiting ribosomes; reducing protein synthesis, inducing caspase activity, activating effector caspases, effectuating cytostatic effects, and cytotoxicity. Shiga toxin catalytic activities include, for example, ribosome inactivation, protein synthesis inhibition, N-glycosidase activity, polynucleotide:adenosine glycosidase activity, RNAase activity, and DNAase activity. Shiga toxins are ribosome inactivating proteins (RIPs). RIPs can depurinate nucleic acids, polynucleosides, polynucleotides, rRNA, ssDNA, dsDNA, mRNA (and polyA), and viral nucleic acids (see e.g., Barbieri L et al., Biochem J 286: 1-4 (1992); Barbieri L et al., Nature 372: 624 (1994); Ling J et al., FEBS Lett 345: 143-6 (1994); Barbieri L et al., Biochem J 319: 507-13 (1996); Roncuzzi L. Gasperi-Campani A, FEBS Lett 392: 16-20 (19%); Stirpe F et al., FEBS Lett 382: 309-12 (1996); Barbieri L et al., Nucleic Acids Res 25: 518-22 (1997); Wang P, Turner N, Nucleic Acids Res 27: 1900-5 (1999); Barbieri L et al., Biochim Biophys Acta 1480: 258-66 (2000); Barbieri L et al., J Biochem 128: 883-9 (2000); Brigotti M et al., Toxicon 39: 341-8 (2001); Brigotti M et al., FASEB J 16: 365-72 (2002); Bagga S et al., J Biol Chem 278: 4813-20 (2003); Picard D et al., J Biol Chem 280: 20069-75 (2005)). Some RIPs show antiviral activity and superoxide dismutase activity (Erice A et al., Antimicrob Agents Chemother 37: 835-8 (1993); Au T et al., FEBS Lett 471: 169-72 (2000); Parikh B, Turner N, Mini Rev Med Chem 4: 523-43 (2004); Sharma N et al., Plant Physiol 134: 171-81 (2004)). Shiga toxin catalytic activities have been observed both in vitro and in vivo. Non-limiting examples of assays for Shiga toxin effector activity measure various activities, such as, e.g., protein synthesis inhibitory activity, depurination activity, inhibition of cell growth, cytotoxicity, supercoiled DNA relaxation activity, and nuclease activity.

The term “IC50” or “IC₅₀” is used herein to refer to the half-maximal inhibitory concentration as measured using in an in vitro ribosome function assay. The term “CD50” or “CD₅₀” is used herein to refer to the half-maximal cytotoxicity concentration in an in vitro cell killing and/or survival assay. The term “EC50” or “EC₅₀” is used herein to refer to the concentration that gives half-maximal response (e.g., inhibition of signaling). The skilled artisan will readily understand the meaning of each of these terms, when taken in context. Each of IC₅₀, C₅₀, and EC₅₀ may be measured by generating a multiple data points using different molecule concentrations or a concentration series. For some samples, accurate values for either IC₅₀ or CD₅₀ might be unobtainable due to the inability to collect the required data points for an accurate curve fit. For example, theoretically, neither an IC₅₀ nor CD₅₀ can be determined if greater than 50% ribosome inhibition or cell death, respectively, does not occur in a concentration series for a given sample. Data insufficient to accurately fit a curve should not be considered as representative of actual molecule activity.

As used herein, the retention of Shiga toxin effector function refers to being capable of exhibiting a level of Shiga toxin functional activity, as measured by an appropriate quantitative assay with reproducibility, comparable to a wild-type, Shiga toxin effector polypeptide control (e.g. a Shiga toxin A1 fragment) or PD-L1 binding molecule comprising a wild-type Shiga toxin effector polypeptide (e.g. a Shiga toxin A1 fragment) under the same conditions. For the Shiga toxin effector function of ribosome inactivation or ribosome inhibition, retained Shiga toxin effector function is exhibiting an IC₅₀ of 10,000 pM or less in an in vitro setting, such as, e.g., by using an assay known to the skilled worker and/or described herein. For the Shiga toxin effector function of cytotoxicity in a target positive cell-kill assay, retained Shiga toxin effector function is exhibiting a CD₅₀ of 1,000 nM or less, depending on the cell type and its expression of the appropriate extracellular target biomolecule, as shown, e.g., by using an assay known to the skilled worker and/or described herein.

As used herein, the term “equivalent” with regard to ribosome inhibition means an empirically measured level of ribosome inhibitory activity, as measured by an appropriate quantitative assay with reproducibility, which is reproducibly within 10% or less of the activity of the reference molecule (e.g., the second PD-L1 binding molecule or third PD-L1 binding molecule) under the same conditions.

As used herein, the term “equivalent” with regard to cytotoxicity means an empirically measured level of cytotoxicity, as measured by an appropriate quantitative assay with reproducibility, which is reproducibly within 10% or less of the activity of the reference molecule (e.g., the second PD-L1 binding molecule or third binding molecule) under the same conditions.

As used herein, the term “attenuated” with regard to cytotoxicity means a molecule exhibits or exhibited a CD₅₀ between 10-fold to 100-fold of a CD₅₀ exhibited by a reference molecule under the same conditions.

Inaccurate IC₅₀ and CD₅₀ values should not be considered when determining a level of Shiga toxin effector function activity. For some samples, accurate values for either IC₅₀ or CD₅₀ might be unobtainable due to the inability to collect the required data points for an accurate curve fit. For example, theoretically, neither an IC₅₀ nor CD₅₀ can be determined if greater than 50% ribosome inhibition or cell death, respectively, does not occur in a concentration series for a given sample. Data insufficient to accurately fit a curve as described in the analysis of the data from exemplary Shiga toxin effector function assays, such as, e.g., assays described in the Examples below, should not be considered as representative of actual Shiga toxin effector function.

A failure to detect activity in Shiga toxin effector function may be due to improper expression, polypeptide folding, and/or protein stability rather than a lack of cell entry, subcellular routing, and/or enzymatic activity. Assays for Shiga toxin effector functions may not require much polypeptide to measure significant amounts of Shiga toxin effector function activity. To the extent that an underlying cause of low or no effector function is determined empirically to relate to protein expression or stability, one of skill in the art may be able to compensate for such factors using protein chemistry and molecular engineering techniques known in the art, such that a Shiga toxin functional effector activity may be restored and measured. As examples, improper cell-based expression may be compensated for by using different expression control sequences; and improper polypeptide folding and/or stability may benefit from stabilizing terminal sequences, or compensatory mutations in non-effector regions which stabilize the three-dimensional structure of the molecule.

Certain Shiga toxin effector functions are not easily measurable, e.g. subcellular routing functions. For example, there is no routine, quantitative assay to distinguish whether the failure of a Shiga toxin effector polypeptide to be cytotoxic and/or deliver a heterologous epitope is due to improper subcellular routing, but at a time when tests are available, then Shiga toxin effector polypeptides may be analyzed for any significant level of subcellular routing as compared to the appropriate wild-type Shiga toxin effector polypeptide. However, if a Shiga toxin effector polypeptide component of a binding molecule exhibits cytotoxicity comparable or equivalent to a wild-type Shiga toxin A Subunit construct, then the subcellular routing activity level is inferred to be comparable or equivalent, respectively, to the subcellular routing activity level of a wild-type Shiga toxin A Subunit construct at least under the conditions tested.

When new assays for individual Shiga toxin functions become available, Shiga toxin effector polypeptides and/or binding molecules comprising Shiga toxin effector polypeptides may be analyzed for any level of those Shiga toxin effector functions, such as a being within 1000-fold or 100-fold or less the activity of a wild-type Shiga toxin effector polypeptide or exhibiting 3-fold to 30-fold or greater activity as compared to a functional knockout, Shiga toxin effector polypeptide.

Sufficient subcellular routing may be merely deduced by observing a molecule's cytotoxic activity levels in cytotoxicity assays, such as, e.g., cytotoxicity assays based on T-cell epitope presentation or based on a toxin effector function involving a cytosolic and/or endoplasmic reticulum-localized, target substrate.

As used herein, the retention of “significant” Shiga toxin effector function refers to a level of Shiga toxin functional activity, as measured by an appropriate quantitative assay with reproducibility comparable to a wild-type Shiga toxin effector polypeptide control (e.g. a Shiga toxin A1 fragment). For in vitro ribosome inhibition, significant Shiga toxin effector function is exhibiting an IC₅₀ of 300 pM or less depending on the source of the ribosomes used in the assay (e.g. a bacterial, archaeal, or eukaryotic (algal, fungal, plant, or animal) source). This is significantly greater inhibition as compared to the approximate IC₅₀ of 100,000 pM for the catalytically disrupted SLT-1A 1-251 double mutant (Y77S/E167D). For cytotoxicity in a target-positive cell-kill assay in laboratory cell culture, significant Shiga toxin effector function is exhibiting a CD₅₀ of 100, 50, 30 nM, or less, depending on the target biomolecule(s) of the binding region and the cell type, particularly that cell type's expression and/or cell-surface representation of the appropriate extracellular target biomolecule(s) and/or the extracellular epitope(s) targeted by the molecule being evaluated. This is significantly greater cytotoxicity to the appropriate, target-positive cell population as compared to a Shiga toxin A Subunit alone (or a wild-type Shiga toxin A1 fragment), without a cell targeting binding region, which has a CD₅₀ of 100-10,000 nM, depending on the cell line.

For purposes of the present disclosure and with regard to the Shiga toxin effector function of a molecule as described herein, the term “reasonable activity” refers to exhibiting at least a moderate level (e.g. within 11-fold to 1,000-fold) of Shiga toxin effector activity as defined herein in relation to a molecule comprising a naturally occurring Shiga toxin, wherein the Shiga toxin effector activity is selected from the group consisting of: internalization efficiency, subcellular routing efficiency to the cytosol, delivered epitope presentation by a target cell(s), ribosome inhibition, and cytotoxicity. For cytotoxicity, a reasonable level of Shiga toxin effector activity includes being within 1,000-fold of a wild-type, Shiga toxin construct, such as, e.g., exhibiting a CD₅₀ of 500 nM or less when a wild-type Shiga toxin construct exhibits a CD₅₀ of 0.5 nM (e.g. a binding molecule comprising a wild-type Shiga toxin A1 fragment).

For purposes of the present disclosure and with regard to the cytotoxicity of a molecule as described herein, the term “optimal” refers to a level of Shiga toxin catalytic domain mediated cytotoxicity that is within 2, 3, 4, 5, 6, 7, 8, 9, or 10-fold of the cytotoxicity of a molecule comprising wild-type Shiga toxin A1 fragment (e.g. a Shiga toxin A Subunit or certain truncated variants thereof) and/or a naturally occurring Shiga toxin.

It should be noted that even if the cytotoxicity of a Shiga toxin effector polypeptide is reduced relative to a wild-type Shiga toxin A Subunit or fragment thereof, in practice, applications using attenuated, Shiga toxin effector polypeptides might be equally or more effective than using wild-type Shiga toxin effector polypeptides because the highest potency variants might exhibit undesirable effects which are minimized or reduced in reduced cytotoxic-potency variants. Wild-type Shiga toxins are very potent, being able to kill an intoxicated cell after only one toxin molecule has reached the cytosol of the intoxicated cell or perhaps after only forty toxin molecules have been internalized into the intoxicated cell. Shiga toxin effector polypeptides with even considerably reduced Shiga toxin effector functions, such as, e.g., subcellular routing or cytotoxicity, as compared to wild-type Shiga toxin effector polypeptides might still be potent enough for practical applications, such as, e.g., applications involving targeted cell-killing, heterologous epitope delivery, and/or detection of specific cells and their subcellular compartments. In addition, certain reduced-activity Shiga toxin effector polypeptides may be particularly useful for delivering cargos (e.g. an additional exogenous material or T-cell epitope) to certain intracellular locations or subcellular compartments of target cells.

As used herein, the phrase “antibody effector function” refer to those biological activities attributable to a Fc region of an antibody or derivative thereof, which vary with the antibody isotype. Examples of antibody effector functions include: Clq binding and complement dependent cytotoxicity (CDC); Fc receptor binding (including the neonatal Fc receptor (FcRn) or Brambell receptor), antibody-dependent cell-mediated cytotoxicity (ADCC); phagocytosis; down-regulation of cell surface receptors (e.g. PD-L1); T-cell activation, and B-cell activation.

The term “selective cytotoxicity” with regard to the cytotoxic activity of a molecule refers to the relative level of cytotoxicity between a biomolecule target positive cell population (e.g. a targeted cell-type) and a non-targeted bystander cell population (e.g. a biomolecule target negative cell-type), which can be expressed as a ratio of the half-maximal cytotoxic concentration (CD₅₀) for a targeted cell type over the CD₅₀ for an untargeted cell type to provide a metric of cytotoxic selectivity or indication of the preferentiality of killing of a targeted cell versus an untargeted cell.

The cell surface representation and/or density of a given extracellular target biomolecule (or extracellular epitope of a given target biomolecule) may influence the applications for which certain binding molecules may be most suitably used. Differences in cell surface representation and/or density of a given target biomolecule between cells may alter, both quantitatively and qualitatively, the efficiency of cellular internalization and/or cytotoxicity potency of a given binding molecule. The cell surface representation and/or density of a given target biomolecule can vary greatly among target biomolecule positive cells or even on the same cell at different points in the cell cycle or cell differentiation. The total cell surface representation of a given target biomolecule and/or of certain extracellular epitopes of a given target biomolecule on a particular cell or population of cells may be determined using methods known to the skilled worker, such as methods involving fluorescence-activated cell sorting (FACS) flow cytometry.

As used herein, the terms “disrupted,” “disruption,” or “disrupting,” and grammatical variants thereof, with regard to a polypeptide region or feature within a polypeptide refers to an alteration of at least one amino acid within the region or composing the disrupted feature. Amino acid alterations include various mutations, such as, e.g., a deletion, inversion, insertion, or substitution which alter the amino acid sequence of the polypeptide. Amino acid alterations also include chemical changes, such as, e.g., the alteration one or more atoms in an amino acid functional group or the addition of one or more atoms to an amino acid functional group.

As used herein, “de-immunized” means reduced antigenic and/or immunogenic potential after administration to a chordate as compared to a reference molecule, such as, e.g., a wild-type peptide region, polypeptide region, or polypeptide. This includes a reduction in overall antigenic and/or immunogenic potential despite the introduction of one or more, de novo, antigenic and/or immunogenic epitopes as compared to a reference molecule. In some embodiments. “de-immunized” means a molecule exhibited reduced antigenicity and/or immunogenicity after administration to a mammal as compared to a “parental” molecule from which it was derived, such as, e.g., a wild-type Shiga toxin A1 fragment or binding molecule comprising the aforementioned. In some embodiments, the de-immunized, Shiga toxin effector polypeptide is capable of exhibiting a relative antigenicity compared to a reference “parental” molecule which is reduced by 10%, 20%, 30%, 40%, 50%, 60%, 70%, 80%, 90%, or greater than the antigenicity of the reference molecule under the same conditions measured by the same assay, such as, e.g., an assay known to the skilled worker and/or described herein like a quantitative ELISA or Western blot analysis. In some embodiments, the de-immunized, Shiga toxin effector polypeptide is capable of exhibiting a relative immunogenicity compared to a reference “parental” molecule which is reduced by 10%, 20%, 30%, 40%, 50%, 60%, 70%, 80%, 90%, 95%, 97%, 99%, or greater than the immunogenicity of the reference molecule under the same conditions measured by the same assay, such as, e.g., an assay known to the skilled worker and/or described herein like a quantitative measurement of anti-molecule antibodies produced in a mammal(s) after receiving parenteral administration of the molecule at a given time-point.

The relative immunogenicities of exemplary binding molecules were determined using an assay for in vivo antibody responses to the binding molecules after repeat, parenteral administrations over periods of time.

The phrase “B-cell and/or CD4+ T-cell de-immunized” means that the molecule has a reduced antigenic and/or immunogenic potential after administration to a mammal regarding either B-cell antigenicity or immunogenicity and/or CD4+ T-cell antigenicity or immunogenicity. In some embodiments, “B-cell de-immunized” means a molecule exhibited reduced B-cell antigenicity and/or immunogenicity after administration to a mammal as compared to a “parental” molecule from which it was derived, such as, e.g., a wild-type Shiga toxin A1 fragment. In some embodiments, “CD4+ T-cell de-immunized” means a molecule exhibited reduced CD4 T-cell antigenicity and/or immunogenicity after administration to a mammal as compared to a “parental” molecule from which it was derived, such as, e.g., a wild-type Shiga toxin A1 fragment.

The term “endogenous” with regard to a B-cell epitope, CD4+ T-cell epitope, B-cell epitope region, or CD4+ T-cell epitope region in a Shiga toxin effector polypeptide refers to an epitope present in a wild-type Shiga toxin A Subunit.

The phrase “CD8+ T-cell hyper-immunized” means that the molecule, when present inside a nucleated, chordate cell within a living chordate, has an increased antigenic and/or immunogenic potential regarding CD8+ T-cell antigenicity or immunogenicity. Commonly. CD8+ T-cell immunized molecules are capable of cellular internalization to an early endosomal compartment of a nucleated, chordate cell due either to an inherent feature(s) or as a component of a binding molecule.

The term “heterologous” means of a different source than an A Subunit of a naturally occurring Shiga toxin, e.g. a heterologous polypeptide is not naturally found as part of any A Subunit of a native Shiga toxin. The term “heterologous” with regard to T-cell epitope or T-cell epitope-peptide component of a binding molecule refers to an epitope or peptide sequence which did not initially occur in the polypeptide component to be modified, but which has been added to the polypeptide, whether added via the processes of embedding, fusion, insertion, and/or amino acid substitution as described herein, or by any other engineering means. The result is a modified poly peptide comprising a T-cell epitope foreign to the original, unmodified polypeptide, i.e. the T-cell epitope was not present in the original polypeptide.

The term “embedded” and grammatical variants thereof with regard to a T-cell epitope or T-cell epitope-peptide component of a binding molecule refers to the internal replacement of one or more amino acids within a polypeptide region with different amino acids in order to generate a new polypeptide sequence sharing the same total number of amino acid residues with the starting polypeptide region. Thus, the term “embedded” does not include any external, terminal fusion of any additional amino acid, peptide, or polypeptide component to the starting polypeptide nor any additional internal insertion of any additional amino acid residues, but rather includes only substitutions for existing amino acids. The internal replacement may be accomplished merely by amino acid residue substitution or by a series of substitutions, deletions, insertions, and/or inversions. If an insertion of one or more amino acids is used, then the equivalent number of proximal amino acids must be deleted next to the insertion to result in an embedded T-cell epitope. This is in contrast to use of the term “inserted” with regard to a T-cell epitope contained within a polypeptide component of a binding molecule to refer to the insertion of one or more amino acids internally within a polypeptide resulting in a new polypeptide having an increased number of amino acids residues compared to the starting polypeptide.

The term “inserted” and grammatical variants thereof with regard to a T-cell epitope contained within a polypeptide component of a binding molecule refers to the insertion of one or more amino acids within a polypeptide resulting in a new polypeptide sequence having an increased number of amino acids residues compared to the starting polypeptide. The “pure” insertion of a T-cell epitope-peptide is when the resulting polypeptide increased in length by the number of amino acid residues equivalent to the number of amino acid residues in the entire, inserted T-cell epitope-peptide. The phrases “partially inserted.” “embedded and inserted,” and grammatical variants thereof with regard to a T-cell epitope contained within a polypeptide component of a binding molecule, refers to when the resulting polypeptide increased in length, but by less than the number of amino acid residues equivalent to the length of the entire, inserted T-cell epitope-peptide. Insertions, whether “pure” or “partial,” include any of the previously described insertions even if other regions of the polypeptide not proximal to the insertion site within the polypeptide are deleted thereby resulting in a decrease in the total length of the final polypeptide because the final polypeptide still comprises an internal insertion of one or more amino acids of a T-cell epitope-peptide within a polypeptide region.

As used herein, the term “T-cell epitope delivering” when describing a functional activity of a molecule means that a molecule provides the biological activity of localizing within a cell to a subcellular compartment that is competent to result in the proteasomal cleavage of a proteinaceous part of the molecule which comprises a T-cell epitope-peptide. The “T-cell epitope delivering” function of a molecule can be assayed by observing the MHC presentation of a T-cell epitope-peptide cargo of the molecule on a cell surface of a cell exogenously administered the molecule or in which the assay was begun with the cell containing the molecule in one or more of its endosomal compartments. Generally, the ability of a molecule to deliver a T-cell epitope to a proteasome can be determined where the initial location of the “T-cell epitope delivering” molecule is an early endosomal compartment of a cell, and then, the molecule is empirically shown to deliver the epitope-peptide to the proteasome of the cell. However, a “T-cell epitope delivering” ability may also be determined where the molecule starts at an extracellular location and is empirically shown, either directly or indirectly, to deliver the epitope into a cell and to proteasomes of the cell. For example, certain “T-cell epitope delivering” molecules pass through an endosomal compartment of the cell, such as, e.g. after endocytotic entry into that cell. Alternatively, “T-cell epitope delivering” activity may be observed for a molecule starting at an extracellular location whereby the molecule does not enter any endosomal compartment of a cell-instead the “T-cell epitope delivering” molecule enters a cell and delivers a T-cell epitope-peptide to proteasomes of the cell, presumably because the “T-cell epitope delivering” molecule directed its own routing to a subcellular compartment competent to result in proteasomal cleavage of its T-cell epitope-peptide component.

The phrase “proximal to an amino-terminus” with reference to the position of a Shiga toxin effector polypeptide region of a binding molecule refers to a distance wherein at least one amino acid residue of the Shiga toxin effector polypeptide region is within 1, 2, 3, 4, 5, 6, 7, 8, 9, 10, 11, 12 or more, e.g., up to 18-20 amino acid residues, of an amino-terminus of the binding molecule as long as the binding molecule is capable of exhibiting the appropriate level of Shiga toxin effector functional activity noted herein (e.g., a certain level of cytotoxic potency). Thus, in some embodiments, any amino acid residue(s) fused amino-terminal to the Shiga toxin effector polypeptide does not reduce any Shiga toxin effector function (e.g., by sterically hindering a structure(s) near the amino-terminus of the Shiga toxin effector polypeptide region) such that a functional activity of the Shiga toxin effector polypeptide is reduced below the appropriate activity level required herein.

The phrase “more proximal to an amino-terminus” with reference to the position of a Shiga toxin effector polypeptide region within a binding molecule as compared to another component (e.g., a cell-targeting, binding region, molecular moiety, and/or additional exogenous material) refers to a position wherein at least one amino acid residue of the amino-terminus of the Shiga toxin effector polypeptide is closer to the amino-terminus of a linear, polypeptide component of the binding molecule as compared to the other referenced component.

The phrase “active enzymatic domain derived from one A Subunit of a member of the Shiga toxin family” refers to having the ability to inhibit protein synthesis via a catalytic ribosome inactivation mechanism. The enzymatic activities of naturally occurring Shiga toxins may be defined by the ability to inhibit protein translation using assays known to the skilled worker, such as, e.g., in vitro assays involving RNA translation in the absence of living cells or in vivo assays involving RNA translation in a living cell. Using assays known to the skilled worker and/or described herein, the potency of a Shiga toxin enzymatic activity may be assessed directly by observing N-glycosidase activity toward ribosomal RNA (rRNA), such as, e.g., a ribosome nicking assay, and/or indirectly by observing inhibition of ribosome function and/or protein synthesis.

The term “Shiga toxin A1 fragment region” refers to a polypeptide region consisting essentially of a Shiga toxin A1 fragment and/or derived from a Shiga toxin A1 fragment of a Shiga toxin.

The terms “terminus,” “amino-terminus,” or “carboxy-terminus” with regard to a binding molecule refers generally to the last amino acid residue of a polypeptide chain of the binding molecule (e.g., a single, continuous polypeptide chain). A binding molecule may comprise more than one polypeptides or proteins, and, thus, a binding molecule may comprise multiple amino-terminals and carboxy-terminals. For example, the “amino-terminus” of a binding molecule may be defined by the first amino acid residue of a polypeptide chain representing the amino-terminal end of the polypeptide, which is generally characterized by a starting, amino acid residue which does not have a peptide bond with any amino acid residue involving the primary amino group of the starting amino acid residue or involving the equivalent nitrogen for starting amino acid residues which are members of the class of N-alkylated alpha amino acid residues. Similarly, the “carboxy-terminus” of a binding molecule may be defined by the last amino acid residue of a polypeptide chain representing the carboxyl-terminal end of the polypeptide, which is generally characterized by a final, amino acid residue which does not have any amino acid residue linked by a peptide bond to the alpha-carbon of its primary carboxyl group.

The terms “terminus,” “amino-terminus,” or “carboxy-terminus” with regard to a polypeptide region refers to the regional boundaries of that region, regardless of whether additional amino acid residues are linked by peptide bonds outside of that region. In other words, the terminals of the polypeptide region regardless of whether that region is fused to other peptides or polypeptides. For example, a fusion protein comprising two proteinaceous regions, e.g., a binding region comprising a peptide or polypeptide and a Shiga toxin effector polypeptide, may have a Shiga toxin effector polypeptide region with a carboxy-terminus ending at amino acid residue 251 of the Shiga toxin effector polypeptide region despite a peptide bond involving residue 251 to an amino acid residue at position 252 representing the beginning of another proteinaceous region, e.g., the binding region. In this example, the carboxy-terminus of the Shiga toxin effector polypeptide region refers to residue 251, which is not a terminus of the fusion protein but rather represents an internal, regional boundary. Thus, for polypeptide regions, the terms “terminus,” “amino-terminus,” and “carboxy-terminus” are used to refer to the boundaries of polypeptide regions, whether the boundary is a physically terminus or an internal, position embedded within a larger polypeptide chain.

The phrase “carboxy-terminus region of a Shiga toxin A1 fragment” refers to a polypeptide region derived from a naturally occurring Shiga toxin A1 fragment, the region beginning with a hydrophobic residue (e.g., V236 of StxA-A1 and SLT-1A1, and V235 of SLT-2A1) that is followed by a hydrophobic residue and the region ending with the furin-cleavage site conserved among Shiga toxin A1 fragment polypeptides and ending at the junction between the A1 fragment and the A2 fragment in native, Shiga toxin A Subunits. The carboxy-terminal region of a Shiga toxin A1 fragment includes a peptidic region derived from the carboxy-terminus of a Shiga toxin A1 fragment polypeptide, such as, e.g., a peptidic region comprising or consisting essentially of the carboxy-terminus of a Shiga toxin A1 fragment. Non-limiting examples of peptidic regions derived from the carboxy-terminus of a Shiga toxin A1 fragment include the amino acid residue sequences natively positioned from position 236 to position 239, 240, 241, 242, 243, 244, 245, 246, 247, 248, 249, 250, or 251 in Stx1A (SEQ ID NO:2) or SLT-1A (SEQ ID NO:1); and from position 235 to position 239, 240, 241, 242, 243, 244, 245, 246, 247, 248, 249, or 250 in SLT-2A (SEQ ID NO:3).

The phrase “proximal to the carboxy-terminus of an A1 fragment polypeptide” with regard to a linked molecular moiety and/or binding region refers to being within 1, 2, 3, 4, 5, 6, 7, 8, 9, 10, 11, or 12 amino acid residues from the amino acid residue defining the last residue of the Shiga toxin A1 fragment polypeptide.

The phrase “sterically covers the carboxy-terminus of the A1 fragment-derived region” includes any molecular moiety of a size of 4.5 kDa or greater (e.g., an immunoglobulin-type binding region) linked and/or fused to an amino acid residue in the carboxy-terminus of the A1 fragment-derived region, such as, e.g., the amino acid residue derived from the amino acid residue natively positioned at any one of positions 236 to 251 in Stx1A (SEQ ID NO:2) or SLT-1A (SEQ ID NO:1) or from 235 to 250 in SLT-2A (SEQ ID NO:3). The phrase “sterically covers the carboxy-terminus of the A1 fragment-derived region” also includes any molecular moiety of a size of 4.5 kDa or greater (e.g., an immunoglobulin-type binding region) linked and/or fused to an amino acid residue in the carboxy-terminus of the A1 fragment-derived region, such as, e.g., the amino acid residue carboxy-terminal to the last amino acid A1 fragment-derived region and/or the Shiga toxin effector polypeptide. The phrase “sterically covers the carboxy-terminus of the A1 fragment-derived region” also includes any molecular moiety of a size of 4.5 kDa or greater (e.g., an immunoglobulin-type binding region) physically preventing cellular recognition of the carboxy-terminus of the A1 fragment-derived region, such as, e.g. recognition by the ERAD machinery of a eukaryotic cell.

A binding region, such as, e.g., an immunoglobulin-type binding region, that comprises a polypeptide comprising at least forty amino acids and that is linked (e.g., fused) to the carboxy-terminus of the Shiga toxin effector polypeptide region comprising an A1 fragment-derived region is a molecular moiety which is “sterically covering the carboxy-terminus of the A1 fragment-derived region.”

A binding region, such as, e.g., an immunoglobulin-type binding region, that comprises a polypeptide comprising at least forty amino acids and that is linked (e.g., fused) to the carboxy-terminus of the Shiga toxin effector polypeptide region comprising an A1 fragment-derived region is a molecular moiety “encumbering the carboxy-terminus of the A1 fragment-derived region.”

The term “A1 fragment of a member of the Shiga toxin family” refers to the remaining amino-terminal fragment of a Shiga toxin A Subunit after proteolysis by furin at the furin-cleavage site conserved among Shiga toxin A Subunits and positioned between the A1 fragment and the A2 fragment in wild-type Shiga toxin A Subunits.

The phrase “furin-cleavage site at the carboxy-terminus of the A1 fragment region” refers to a specific, furin-cleavage site conserved among Shiga toxin A Subunits and bridging the junction between the A1 fragment and the A2 fragment in naturally occurring, Shiga toxin A Subunits.

The phrase “furin-cleavage site proximal to the carboxy-terminus of the A1 fragment region” refers to any identifiable, furin-cleavage site having an amino acid residue within a distance of less than 1, 2, 3, 4, 5, 6, 7, or more amino acid residues of the amino acid residue defining the last amino acid residue in the A1 fragment region or A1 fragment derived region, including a furin-cleavage site located carboxy-terminal of an A1 fragment region or A1 fragment derived region, such as, e.g., at a position proximal to the linkage of the A1 fragment-derived region to another component of the molecule, such as, e.g., a molecular moiety of a binding molecule.

The phrase “disrupted furin-cleavage site” refers to (i) a specific furin-cleavage site as described herein in Section I-B and (ii) which comprises a mutation and/or truncation that can confer a molecule with a reduction in furin-cleavage as compared to a reference molecule, such as, e.g., a reduction in furin-cleavage reproducibly observed to be 30%, 40%, 50%, 60%, 70%, 80%, 90%, 95%, 97%, 98%, 99%, or less (including 100% for no cleavage) than the furin-cleavage of a reference molecule observed in the same assay under the same conditions. The percentage of furin-cleavage as compared to a reference molecule can be expressed as a ratio of cleaved uncleaved material of the molecule of interest divided by the cleaved:uncleaved material of the reference molecule (see e.g. WO 2015/191764; WO 2016/196344). Non-limiting examples of suitable reference molecules include certain molecules comprising a wild-type Shiga toxin furin-cleavage site as described herein.

The phrase “furin-cleavage resistant” means a molecule or specific polypeptide region thereof exhibits reproducibly less furin cleavage than (i) the carboxy-terminus of a Shiga toxin A1 fragment in a wild-type Shiga toxin A Subunit or (ii) the carboxy-terminus of the Shiga toxin A1 fragment derived region of construct wherein the naturally occurring furin-cleavage site natively positioned at the junction between the A1 and A2 fragments is not disrupted; as assayed by any available means to the skilled worker, including by using a method described herein.

The phrase “active enzymatic domain derived form an A Subunit of a member of the Shiga toxin family” refers to a polypeptide structure having the ability to inhibit protein synthesis via catalytic inactivation of a ribosome based on a Shiga toxin enzymatic activity. The ability of a molecular structure to exhibit inhibitory activity of protein synthesis and/or catalytic inactivation of a ribosome may be observed using various assays known to the skilled worker, such as, e.g., in vitro assays involving RNA translation assays in the absence of living cells or in vivo assays involving the ribosomes of living cells. For example, using assays known to the skilled worker, the enzymatic activity of a molecule based on a Shiga toxin enzymatic activity may be assessed directly by observing N-glycosidase activity toward ribosomal RNA (rRNA), such as, e.g., a ribosome nicking assay, and/or indirectly by observing inhibition of ribosome function, RNA translation, and/or protein synthesis.

As used herein with respect to a Shiga toxin effector polypeptide, a “combination” describes a Shiga toxin effector polypeptide comprising two or more sub-regions wherein each sub-region comprises at least one of the following: (1) a disruption in an endogenous epitope or epitope region; (2) an embedded, heterologous, T-cell epitope-peptide; (3) an inserted, heterologous, T-cell epitope-peptide; and (4) a disrupted furin-cleavage site at the carboxy-terminus of an A1 fragment region.

As used herein, a “binding molecule” is used interchangeably with a “PD-L1 binding molecule”, and “PD-L1 binding molecule”, which encompasses “DI-SLT-1A fusion proteins” and “SLT-1A fusion proteins”. All of the aforementioned molecule types include various “PD-L1-binding proteins”.

### PD-L1 Binding Molecules

Provided herein are various binding molecules which bind PD-L1 and comprise a toxin component (referred to herein as “PD-L1 binding molecules” or “PD-L1 binding molecules”. All of the aforementioned molecule types include various “PD-L1-binding proteins). The PD-L1 binding molecules are useful, for e.g., (1) as cytotoxic molecules for killing PD-L1 expressing cells, (2) for selectively killing specific PD-L1-positive cell type(s) amongst other cells, (3) as delivery vehicles for delivering a CD8+ T-cell epitope to the MHC class I presentation pathway of a PD-L1 expressing cell, (4) as nontoxic delivery vehicles for delivering an atom or molecule to the interior of a PD-L1 expressing cell, (5) as diagnostic molecules for the diagnosis, prognosis, or characterization of diseases and conditions involving PD-L1 expressing cell, and (6) as therapeutic molecules for treating a variety of diseases, disorders, and conditions involving PD-L1-expressing cells, such as various cancers and tumors.

In some embodiments, the binding molecule comprises a PD-L1 binding immunoglobulin domain and a Shiga toxin A Subunit effector polypeptide. Shiga toxin A Subunit effector polypeptides provide robust and powerful scaffolds for engineering novel, binding molecules (see e.g. WO 2014/164680, WO 2014/164693, WO 2015/138435, WO 2015/138452, WO 2015/113005, WO 2015/113007, WO 2015/191764. WO 2016/196344, WO 2017/019623, WO 2018/106895, and WO 2018/140427). The association of PD-L1 binding immunoglobulin-derived fragments as cell-targeting moieties with Shiga toxin A Subunit effector polypeptides enables the engineering of therapeutic and diagnostic molecules that target PD-L1.

### I, The General Structure of the PD-L1 Binding Molecules

The PD-L1 binding molecules described herein each comprise (1) a PD-L1 binding region for cell-targeting and (2) a toxin.

In some embodiments, a binding molecule comprises (1) a binding region capable of specifically binding an extracellular part of PD-L1 associated with a cell surface and (2) a toxin effector polypeptide. In some embodiments, a binding molecule comprises (1) a binding region capable of specifically binding an extracellular part of PD-L1 associated with a cell surface and (2) a Shiga toxin effector polypeptide region comprising a Shiga toxin A Subunit effector polypeptide (referred to herein as a “Shiga toxin effector polypeptide”). In some embodiments, the binding molecule comprises two or more PD-L1 binding regions, whether the same or different, and two or more Shiga toxin effector polypeptide regions, whether the same or different. One non-limiting example of a binding molecule is a Shiga toxin effector polypeptide fused to an immunoglobulin-type binding region comprising a single-chain variable fragment, or a homo- or hetero-dimer of the aforementioned. The PD-L1 binding molecules described herein may optionally comprise a T-cell epitope for delivery to the interior of a target cell and subsequent cell-surface presentation.

In some embodiments, the binding molecule is a homo-dimer or a hetero-dimer. In some embodiments, the binding molecule is a homo-dimer comprising two monomers, wherein each monomer comprises a PD-L1 binding region and a Shiga toxin effector polypeptide. In some embodiments, a dimeric binding molecule exhibits properties which are more favorable than the properties of a monomeric variant comprising identical binding region and toxin region. For example, in some embodiments, a binding molecule in dimeric form may more efficiently deliver an antigenic epitope (i.e., a CD8+ T-cell epitope) to a target cell than a similar molecule in monomeric form.

In some embodiments, the Shiga toxin A Subunit effector polypeptide of the binding molecule combines structural elements resulting in two or more properties in a single molecule, such as, e.g., the ability to 1) exhibit reduced antigenicity and/or immunogenicity as compared to molecular variants lacking that particular structural element(s), 2) exhibit reduced protease-cleavage as compared to molecular variants lacking that particular structural element(s), 3) exhibit reduced non-specific toxicity to a multicellular organism at certain dosages as compared to molecular variants lacking that particular element(s), 4) deliver an embedded or inserted CD8+ T-cell epitope to the MHC class I system a cell for cell-surface presentation, and/or 5) exhibit potent cytotoxicity.

### A. PD-L1 Binding Regions

In some embodiments, the PD-L1 binding molecule comprises a binding region comprising an immunoglobulin-type polypeptide capable of exhibiting specific and high-affinity binding to human PD-L1 and/or PD-L1 present on a cellular surface of a cell, such as, e.g., PD-L1 expressing cell or PD-L1 positive cell.

In some embodiments, a binding region of a binding molecule is a cell-targeting component, such as, e.g., a domain, molecular moiety, or agent, capable of binding specifically to an extracellular part of a PD-L1 target biomolecule on a cell surface (i.e. an extracellular target biomolecule) with high affinity. As used herein, the term “PD-L1 binding region” refers to a molecular moiety (e.g. a proteinaceous molecule) or agent capable of specifically binding an extracellular part of a PD-L1 molecule with high affinity, such as, e.g., having a dissociation constant with regard to PD-L1 of 10⁻⁵ to 10⁻¹² moles per liter. As used herein, PD-L1 binding refers to the ability to bind to an extracellular part of PD-L1, including an isoform or variant of human PD-L1.

An extracellular part of a target biomolecule refers to a portion of its structure exposed to the extracellular environment when the molecule is physically coupled to a cell, such as, e.g., when the target biomolecule is expressed at a cellular surface by the cell. In this context, exposed to the extracellular environment means that part of the target biomolecule is accessible by, e.g., an antibody or at least a binding moiety smaller than an antibody such as a single-domain antibody domain, a nanobody, a heavy-chain antibody domain derived from camelids or cartilaginous fishes, a single-chain variable fragment, or any number of engineered alternative scaffolds to immunoglobulins (see below). The exposure to the extracellular environment of or accessibility to a part of target biomolecule physically coupled to a cell may be empirically determined by the skilled worker using methods well known in the art.

In some embodiments, a binding molecule comprises a binding region comprising one or more polypeptides capable of selectively and specifically binding an extracellular part of PD-L1.

In some embodiments, the PD-L1 binding region is an immunoglobulin-type binding region. In some embodiments, the immunoglobulin-type, PD-L1 binding region is derived from an immunoglobulin, PD-L1 binding region, such as an antibody paratope capable of binding an extracellular part of PD-L1. This engineered polypeptide may optionally include polypeptide scaffolds comprising or consisting essentially of complementary determining regions and/or antigen binding regions from immunoglobulins as described herein.

In some embodiments, the PD-L1 binding region comprises a heavy chain variable region (HVR-H) comprising three CDRs, each having at least 85%, 90%, 91%, 92%, 93%, 94%, 95%, 96%, 97%, 98%, or 99% identity to any one of SEQ ID NOs: 22-24 and 27-32; or consisting essentially of an amino acid sequence show in any one of SEQ ID NOs: 22-24 and 27-32. In some embodiments, the binding region further comprises: (a) a light chain variable region (HVR-L) comprising three CDRs, each having at least 85%, 90%, 91%, 92%, 93%, 94%, 95%, 96%, 97%, 98%, or 99% identity to any one of SEQ ID NO:19, SEQ ID NO:20, SEQ ID NO:21, SEQ ID NO:25, and SEQ ID NO:26; or consisting essentially of an amino acid sequence shown in any one of SEQ ID NO:19, SEQ ID NO:20, SEQ ID NO:21, SEQ ID NO:25, and SEQ ID NO:26. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:21; or consisting essentially of an amino acid sequence shown in any one of SEQ ID NO:19, SEQ ID NO:20, and SEQ ID NO:21. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:25, SEQ ID NO:20, and SEQ ID NO:21; or consisting essentially of an amino acid sequence shown in any one of SEQ ID NO:25, SEQ ID NO:20, and SEQ ID NO:21. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:26; or consisting essentially of an amino acid sequence shown in any one of SEQ ID NO: 19, SEQ ID NO:20, and SEQ ID NO:26.

In some embodiments, the PD-L1 binding region comprises a heavy chain variable region (HVR-H) comprising three CDRs, each having at least 85%, 90%, 91%, 92%, 93%, 94%, 95%, 96%, 97%, 98%, or 99% identity to any one of SEQ ID NOs: 22-24 and 27-32; or consisting essentially of an amino acid sequence show in any one of SEQ ID NOs: 22-24 and 27-32. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, SEQ ID NO:21, SEQ ID NO:25, and SEQ ID NO:26; or consisting essentially of an amino acid sequence shown in any one SEQ ID NO:19, SEQ ID NO:20, SEQ ID NO:21, SEQ ID NO:25, and SEQ ID NO:26. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:26; or consisting essentially of an amino acid sequence shown in any one of SEQ ID NO:19, SEQ ID NO:20, and SEQ ID NO:26. In some embodiments, the binding region comprises: (a) a light chain variable region (HVR-L) comprising three CDRs, each comprising or consisting essentially of an amino acid sequence shown in any one of SEQ ID NO:19, SEQ ID NO:20. SEQ ID NO:21, SEQ ID NO:25, and SEQ ID NO:26; and (b) a heavy chain variable region (HVR-H) comprising three CDRs, each comprising or consisting essentially of an amino acid sequence show in any one of SEQ ID NOs: 22-24 and 27-32. In some embodiments, the binding region comprises: a) a heavy chain variable region (HVR-H) comprising (i) a HCDR1 comprising or consisting essentially, or consisting of the amino acid sequence of SEQ ID NO:27; (ii) a HCDR2 comprising, consisting essentially of, or consisting of the amino acid sequence of SEQ ID NO:29 or 30; and (iii) a HCDR3 comprising, consisting essentially of, or consisting of the amino acid sequence of SEQ ID NO:32; and/or b) a light chain variable region (HVR-L) comprising (i) a LCDR1 comprising, consisting essentially of, or consisting of the amino acid sequence of SEQ ID NO:19; (ii) a LCDR2 comprising, consisting essentially of, or consisting of the amino acid sequence of SEQ ID NO:20; and (iii) a LCDR3 comprising, consisting essentially of or consisting of the amino acid sequence of SEQ ID NO:26. In some embodiments, the binding region comprises: a) a heavy chain variable region (HVR-H) comprising (i) a HCDR1 consisting of the amino acid sequence of SEQ ID NO:27; (ii) a HCDR2 consisting of the amino acid sequence of SEQ ID NO:29 or 30; and (iii) a HCDR3 consisting of the amino acid sequence of SEQ ID NO:32; and b) a light chain variable region (HVR-L) comprising (i) a LCDR1 consisting of the amino acid sequence of SEQ ID NO:19; (ii) a LCDR2 consisting of the amino acid sequence of SEQ ID NO:20; and (iii) a LCDR3 consisting of the amino acid sequence of SEQ ID NO:26. In some embodiments, the binding region comprises: a) a heavy chain variable region (HVR-H) comprising (i) a HCDR1 consisting of the amino acid sequence of SEQ ID NO:27; (ii) a HCDR2 consisting of the amino acid sequence of SEQ ID NO:29; and (iii) a HCDR3 consisting of the amino acid sequence of SEQ ID NO:32; and b) a light chain variable region (HVR-L) comprising (i) a LCDR1 consisting of the amino acid sequence of SEQ ID NO:19; (ii) a LCDR2 consisting of the amino acid sequence of SEQ ID NO:20; and (iii) a LCDR3 consisting of the amino acid sequence of SEQ ID NO:26.

In some embodiments, the binding region comprises: (a) a light chain region having at least 85%, 90%, 91%, 92%, 93%, 94%, 95%, 96%, 97%, 98%, or 99%, identity to any one of SEQ ID NOs: 33, 35-36, and 38, or consisting essentially of the amino acid sequence of any one of SEQ ID NOs: 33, 35-36, and 38; and/or (b) a heavy chain region having at least 85%, 90%, 91%, 92%, 93%, 94%, 95%, 96%, 97%, 98%, or 99% identity to any one of SEQ ID NOs: 34, 37, and 39, or consisting essentially of the amino acid sequence of any one of SEQ ID NOs: 34, 37, and 39. In some embodiments, the binding region comprises a polypeptide having at least 85%, 90%, 91%, 92%, 93%, 94%, 95%, 96%, 97%, 98%, or 99% identity to any one of SEQ ID NOs: 85-107 and 156-157 or consists essentially of the polypeptide shown in any one of SEQ ID NOs: 85-107 and 156-157. In some embodiments, the binding region is a single-chain variable fragment, such as, e.g., consisting of, comprising, or consisting essentially of the polypeptide of any one of SEQ ID NOs: 85-107 and 156-157. In some embodiments, the binding region comprises: (a) a light chain variable region (HVR-L) comprising three CDRs, each comprising, consisting essentially of, or consisting of an amino acid sequence shown in any one of SEQ ID NO:33, SEQ ID NO:35, SEQ ID NO:36, and SEQ ID NO:38; and (b) a heavy chain variable region (HVR-H) comprising three CDRs, each comprising, consisting essentially of, or consisting of an amino acid sequence show in any one of SEQ ID NO:34, SEQ ID NO:37, and SEQ ID NO:39.

In some embodiments, the binding region of the binding molecule may be, e.g., a monoclonal antibody or engineered antibody derivative. In some embodiments, the binding region is an antibody fragment, e.g., a Fv, Fab, Fab′, scFv, diabody, Fab′-SH, or F(ab′)2 fragment. In another embodiment, the binding region is a full-length antibody. e.g., an intact IgG1 antibody or other antibody class or isotype as defined herein and/or known to the skilled worker. The “class” of an antibody refers to the type of constant domain or constant region present in the heavy chain. There are five major classes of antibodies: IgA, IgD, IgE, IgG, and IgM, and several of these may be further divided into isotypes, e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

In some embodiments, the binding region is a synthetically engineered antibody derivate, such as, e.g. an autonomous V_{H} domain (such as, e.g., from camelids, murine, or human sources), single-domain antibody domain (sdAb), heavy-chain antibody domains derived from a camelid (V_{H}H fragment or V_{H} domain fragment), heavy-chain antibody domains derived from a camelid V_{H}H fragments or V_{H} domain fragments, heavy-chain antibody domain derived from a cartilaginous fish, immunoglobulin new antigen receptor (IgNAR), V_{NAR} fragment, single-chain variable (scFv) fragment, nanobody, “camelized” scaffolds comprising a V_{H} domain. Fd fragment consisting of the heavy chain and C_{H}1 domains, single chain Fv-C_{H}3 minibody, Fc antigen binding domain (Fcabs), scFv-Fc fusion, multimerizing scFv fragment (diabodies, triabodies, tetrabodies), disulfide-stabilized antibody variable (Fv) fragment (dsFv), disulfide-stabilized antigen-binding (Fab) fragment consisting of the V_{L}, V_{H}, C_{L} and C_{H}1 domains, single-chain variable-region fragments comprising a disulfide-stabilized heavy and light chain (sc-dsFvs), bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, Fab-FCabs (mAb²'s), and any genetically manipulated counterparts of the foregoing that retain its paratope and binding function, such as, e.g., wherein the relative orientation or order of the heavy and light chains is reversed or “flipped”.

According to one specific, but non-limiting aspect, the binding region may comprise an immunoglobulin-type binding region. The term “immunoglobulin-type binding region” as used herein refers to a polypeptide region capable of binding one or more target biomolecules, such as an antigen or epitope. Binding regions may be functionally defined by their ability to bind to target molecules. Immunoglobulin-type binding regions are commonly derived from antibody or antibody-like structures.

Immunoglobulin (Ig) proteins have a structural domain known as an Ig domain. Ig domains range in length from about 70-110 amino acid residues and possess a characteristic Ig-fold, in which typically 7 to 9 antiparallel beta strands arrange into two beta sheets which form a sandwich-like structure. The Ig fold is stabilized by hydrophobic amino acid interactions on inner surfaces of the sandwich and highly conserved disulfide bonds between cysteine residues in the strands. Ig domains may be variable (IgV or V-set), constant (IgC or C-set) or intermediate (IgI or I-set). Some Ig domains may be associated with a complementarity determining region (CDR), also called a “complementary determining region,” which is important for the specificity of antibodies binding to their epitopes. Ig-like domains are also found in non-immunoglobulin proteins and are classified on that basis as members of the Ig superfamily of proteins. The HUGO Gene Nomenclature Committee (HGNC) provides a list of members of the Ig-like domain containing family.

An immunoglobulin-type binding region may be a polypeptide sequence of an antibody or antigen-binding fragment thereof wherein the amino acid sequence has been varied from that of a native antibody or an Ig-like domain of a non-immunoglobulin protein, for example by molecular engineering or selection by library screening. Because of the relevance of recombinant DNA techniques and in vitro library screening in the generation of immunoglobulin-type binding regions, antibodies can be redesigned to obtain desired characteristics, such as smaller size, cell entry, or other improvements for in vivo and/or therapeutic applications. The possible variations are many and may range from the changing of just one amino acid to the complete redesign of, for example, a variable region. Typically, changes in the variable region will be made in order to improve the antigen-binding characteristics, improve variable region stability, or reduce the potential for immunogenic responses.

There are numerous immunoglobulin-type binding regions contemplated as components of molecules described herein. In some embodiments, the immunoglobulin-type binding region is derived from an immunoglobulin binding region, such as an antibody paratope capable of binding an extracellular part of PD-L1. In certain other embodiments, the immunoglobulin-type binding region comprises an engineered polypeptide not derived from any immunoglobulin domain but which functions like an immunoglobulin binding region by providing high-affinity binding to an extracellular part of PD-L1. This engineered polypeptide may optionally include polypeptide scaffolds comprising or consisting essentially of complementary determining regions from immunoglobulins as described herein.

There are also numerous binding regions in the prior art that are useful for targeting polypeptides to specific cell-types via their high-affinity binding characteristics. In some embodiments, the binding region of the binding molecule is selected from the group which includes autonomous V_{H} domains, single-domain antibody domains (sdAbs), heavy-chain antibody domains derived from camelids (V_{H}H fragments or V_{H} domain fragments), heavy-chain antibody domains derived from camelid V_{H}H fragments or V_{H} domain fragments, heavy-chain antibody domains derived from cartilaginous fishes, immunoglobulin new antigen receptors (IgNARs). V_{NAR} fragments, single-chain variable (scFv) fragments, nanobodies, Fd fragments consisting of the heavy chain and C_{H}1 domains, single chain Fv-C_{H}3 minibodies, dimeric C_{H}2 domain fragments (C_{H}2D), Fc antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the V_{L}, V_{H}, C_{L} and C_{H}1 domains, bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, and any genetically manipulated counterparts of the foregoing that retain its paratope and binding function, such as, e.g., wherein the relative orientation or order of the heavy and light chains is reversed or flipped (see Ward E et al., Nature 341: 544-6 (1989); Davies J, Riechmann L, Biotechnology (NY) 13: 475-9 (1995); Reiter Y et al., Mol Biol 290: 685-98 (1999); Riechmann L, Muyldermans S, J Immunol Methods 231: 25-38 (1999); Tanha J et al., J Immunol Methods 263: 97-109 (2002): Vranken W et al., Biochemistry 41: 8570-9 (2002); Jespers L et al., J Mol Biol 337: 893-903 (2004); Jespers L et al., Nat Biotechnol 22: 1161-5 (2004); To R et al., J Biol Chem 280: 41395-403 (2005); Saerens D et al., Curr Opin Pharmacol 8: 600-8 (2008); Dimitrov D, MAbs 1: 26-8 (2009); Weiner L, Cell 148: 1081-4 (2012); Ahmad Z et al., Clin Dev Immunol 2012: 980250 (2012)).

There are a variety of binding regions comprising polypeptides derived from the constant regions of immunoglobulins, such as, e.g., engineered dimeric Fc domains, monomeric Fcs (mFcs), scFv-Fcs, V_{H}H-Fcs, C_{H}2 domains, monomeric C_{H}3s domains (mC_{H}3s), synthetically reprogrammed immunoglobulin domains, and/or hybrid fusions of immunoglobulin domains with ligands (Hofer T et al., Proc Natl Acad Sci U.S.A 105: 12451-6 (2008); Xiao J et al., J Am Chem Soc 131: 13616-13618 (2009); Xiao X et al., Biochem Biophys Res Commun 387: 387-92 (2009); Wozniak-Knopp G et al., Protein Eng Des Sel 23 289-97 (2010); Gong R et al., PLoS ONE 7: e42288 (2012); Wozniak-Knopp G et al., PLoS ONE 7: e30083 (2012); Ying T et al., J Biol Chem 287: 19399-408 (2012); Ying T et al., J Biol Chem 288: 25154-64 (2013); Chiang M et al., J Am Chem Soc 136: 3370-3 (2014); Rader C, Trends Biotechnol 32: 186-97 (2014); Ying T et al., Biochimica Biophys Acta 1844: 1977-82 (2014)).

In some embodiments, the binding region of the binding molecule is an intact antibody and/or comprises an Fc region. The term “Fc region” refers to part of the fragment crystallizable region, a C-terminal proximal region of certain heavy chains of native immunoglobulins that contains at least a portion of the constant region, such as, e.g., at least the second and third constant (C_{H}) domains and a glycosylation site. However, as used herein, the term “Fc region” includes native sequence Fc regions and variant or mutated Fc regions or fragments thereof. Unless otherwise specified herein, numbering of amino acid residues in the Fc region or constant region is according to the EU numbering system, also called the EU index, as described in Kabat, E. A., Sequences of Proteins of Immunological Interest, 5th Ed. Public Health Service, National Institutes of Health, Bethesda, Md., USA (1991).

In accordance with certain other embodiments, the binding region comprises an engineered, alternative scaffold to immunoglobulin domains. Engineered alternative scaffolds are known in the art which exhibit similar functional characteristics to immunoglobulin-derived structures, such as high-affinity and specific binding of target biomolecules, and might provide improved characteristics to certain immunoglobulin domains, such as, e.g., greater stability or reduced immunogenicity. Generally, alternative scaffolds to immunoglobulins are less than 20 kilodaltons, consist of a single polypeptide chain, lack cysteine residues, and exhibit relatively high thermodynamic stability.

Any of the aforementioned PD-L1 binding molecules may be suitable for use as a PD-L 1 binding region or modified to create one or more PD-L1 binding regions for use in a binding molecule. Any of the above binding region structures may be used as a component of a molecule as long as the binding region component has a dissociation constant of 10⁻⁵ to 10⁻¹² moles per liter, preferably less than 200 nanomolar (nM), towards an extracellular part of a PD-L1 molecule.

### B. Shiga Toxin Effector Polypeptides

The binding molecules comprise at least one toxin component. In some embodiments, the binding molecule comprises the toxin component which is a Shiga toxin effector polypeptide derived from a Shiga toxin A Subunit. A Shiga toxin effector polypeptide is a polypeptide derived from a Shiga toxin A Subunit member of the Shiga toxin family that is capable of exhibiting one or more Shiga toxin functions (see e.g., Cheung M et al., Mol Cancer 9: 28 (2010); WO 2014/164680, WO 2014/164693, WO 2015/138435, WO 2015/138452, WO 2015/113005, WO 2015/113007, WO 2015/191764, WO 2016/196344, WO 2017/019623, WO 2018/106895, and WO 2018/140427). Shiga toxin functions include, e.g., increasing cellular internalization, directing subcellular routing from an endosomal compartment to the cytosol, avoiding intracellular degradation, catalytically inactivating ribosomes, and effectuating cytostatic and/or cytotoxic effects.

In some embodiments, the binding molecules described herein comprise a Shiga toxin effector polypeptide comprising any one of SEQ ID NO: 1-18, 40-68, 169, 170, or 173. In some embodiments, the binding molecules described herein comprise a Shiga toxin effector polypeptide comprising a variant of any one of SEQ ID NO: 1-18, 40-68, 169, 170, or 173. In some embodiments, the binding molecules described herein comprise a Shiga toxin effector polypeptide comprising a sequence with at least 90%, at least 95%, at least 96%, at least 97%, at least 98%, or at least 99% identity to any one of SEQ ID NO: 1-18, 40-68, 169, 170, or 173. In some embodiments, the binding molecules described herein comprise a Shiga toxin effector polypeptide comprising any one of SEQ ID NO: 1-18, 40-68, 169, 170, or 173 with one or more mutations, such as 2, 3, 4, 5, 6, 7, 8, or 10 mutations. In some embodiments, the Shiga toxin effector comprises any one of SEQ ID NO: 1-18, 40-68, 169, 170, or 173 with 1-5, 5-10, 11-5, 15-20, 10-25, 25-30, or more than 30 mutations. In some embodiments, the binding molecules described herein comprise a Shiga toxin effector polypeptide comprising a variant of any one of SEQ ID NO: 1-18, 40-68, 169, 170, or 173, wherein the variant comprises a S45C mutation. In some embodiments, mutations in the Shiga toxin effector polypeptide render the polypeptide catalytically inactive. In some embodiments, mutations in the Shiga toxin effector polypeptide do not affect the catalytic activity of the polypeptide. In some embodiments, mutations in the Shiga toxin effector polypeptide increase the catalytic activity of the polypeptide. In some embodiments, mutations in the Shiga toxin effector polypeptide decrease the catalytic activity of the polypeptide.

In some embodiments, the binding molecules described herein comprise a Shiga toxin effector polypeptide SEQ ID NO: 41. In some embodiments, the binding molecules described herein comprise a Shiga toxin effector polypeptide that is a variant of SEQ ID NO: 41. In some embodiments, the binding molecules described herein comprise a Shiga toxin effector polypeptide comprising a sequence with at least 90%, at least 95%, at least 96%, at least 97%, at least 98%, or at least 99% identity to SEQ ID NO: 41. In some embodiments, the binding molecules described herein comprise a Shiga toxin effector polypeptide comprising SEQ ID NO: 41 with one or more mutations, such as 2, 3, 4, 5, 6, 7, 8, or 10 mutations. In some embodiments, the Shiga toxin effector comprises SEQ ID NO: 41, with 1-5, 5-10, 11-5, 15-20, 10-25, 25-30, or more than 30 mutations. In some embodiments, mutations in the Shiga toxin effector polypeptide render the polypeptide catalytically inactive. In some embodiments, mutations in the Shiga toxin effector polypeptide do not affect the catalytic activity of the polypeptide. In some embodiments, mutations in the Shiga toxin effector polypeptide increase the catalytic activity of the polypeptide. In some embodiments, mutations in the Shiga toxin effector polypeptide decrease the catalytic activity of the polypeptide.

In some embodiments, the Shiga toxin effector polypeptide comprises amino acids 75 to 251 of SEQ ID NO: 1, SEQ ID NO: 2, or SEQ ID NO: 3; amino acids 1 to 241 of SEQ ID NO: 1, SEQ ID NO: 2, or SEQ ID NO: 3; amino acids 1 to 251 of SEQ ID NO: 1, SEQ ID NO: 2, or SEQ ID NO: 3; or amino acids 1 to 261 of SEQ ID NO: 1, or SEQ ID NO: 2, or SEQ ID NO: 3. In some embodiments, the Shiga toxin effector polypeptide comprises a sequence having at least 90% identity to any one of amino acids 75 to 251 of SEQ ID NO: 1, SEQ ID NO: 2, or SEQ ID NO: 3; amino acids 1 to 241 of SEQ ID NO: 1, SEQ ID NO: 2, or SEQ ID NO: 3; amino acids 1 to 251 of SEQ ID NO: 1, SEQ ID NO: 2, or SEQ ID NO: 3; or amino acids 1 to 261 of SEQ ID NO: 1, or SEQ ID NO: 2, or SEQ ID NO: 3. In some embodiments, the Shiga toxin effector polypeptide comprises a sequence having at least 95% identity to any one of amino acids 75 to 251 of SEQ ID NO: 1, SEQ ID NO: 2, or SEQ ID NO: 3; amino acids 1 to 241 of SEQ ID NO: 1, SEQ ID NO: 2, or SEQ ID NO: 3; amino acids 1 to 251 of SEQ ID NO: 1, SEQ ID NO: 2, or SEQ ID NO: 3; or amino acids 1 to 261 of SEQ ID NO: 1, or SEQ ID NO: 2, or SEQ ID NO: 3. In some embodiments, the Shiga toxin effector polypeptide comprises a sequence having at least 99% identity to any one of amino acids 75 to 251 of SEQ ID NO: 1, SEQ ID NO: 2, or SEQ ID NO: 3; amino acids 1 to 241 of SEQ ID NO: 1, SEQ ID NO: 2, or SEQ ID NO: 3; amino acids 1 to 251 of SEQ ID NO: 1, SEQ ID NO: 2, or SEQ ID NO: 3; or amino acids 1 to 261 of SEQ ID NO: 1, or SEQ ID NO: 2, or SEQ ID NO: 3. In some embodiments, the Shiga toxin effector polypeptide comprises a sequence having between 1 and 25 amino acid substitutions relative to any one of amino acids 75 to 251 of SEQ ID NO: 1, SEQ ID NO: 2, or SEQ ID NO: 3; amino acids 1 to 241 of SEQ ID NO: 1, SEQ ID NO: 2, or SEQ ID NO: 3; amino acids 1 to 251 of SEQ ID NO: 1, SEQ ID NO: 2, or SEQ ID NO: 3; or amino acids 1 to 261 of SEQ ID NO: 1, or SEQ ID NO: 2, or SEQ ID NO: 3.

In some embodiments, the Shiga toxin effector comprises SEQ ID NO: 41 plus an E167D mutation, a R170S mutation, or both an E167D and a R170S mutation. In some embodiments, the Shiga toxin effector comprises any one of SEQ ID NO: 167, 170, or 173.

The Shiga toxin family of protein toxins is composed of various naturally occurring toxins which are structurally and functionally related, e.g., Shiga toxin, Shiga-like toxin 1, and Shiga-like toxin 2 (Johannes L, Römer W, Nat Rev Microbiol 8: 105-16 (2010)). Holotoxin members of the Shiga toxin family contain targeting domains that preferentially bind a specific glycosphingolipid present on the surface of some host cells and an enzymatic domain capable of permanently inactivating ribosomes once inside a cell (Johannes L, Römer W, Nat Rev Microbiol 8: 105-16 (2010)). Members of the Shiga toxin family share the same overall structure and mechanism of action (Engedal N et al., Microbial Biotech 4: 32-46 (2011)). For example, Stx, SLT-1 and SLT-2 display indistinguishable enzymatic activity in cell free systems (Head S et al., J Biol Chem 266: 3617-21 (1991); Tesh V et al., Infect Immun 61: 3392-402 (1993); Brigotti M et al., Toxicon 35:1431-1437 (1997)).

The Shiga toxin family encompasses true Shiga toxin (Stx) isolated from S. dysenteriae serotype 1, Shiga-like toxin 1 variants (SLT1 or Stx1 or SLT-1 or Slt-1) isolated from serotypes of enterohemorrhagic E. coli, and Shiga-like toxin 2 variants (SLT2 or Stx2 or SLT-2) isolated from serotypes of enterohemorrhagic E coli. SLT1 differs by only one amino acid residue from Stx, and both have been referred to as Verocytotoxins or Verotoxins (VTs) (O'Brien A, Curr Top Microbiol Immunol 180: 65-94 (1992)). Although SLT1 and SLT2 variants are only about 53-60% similar to each other at the primary amino acid sequence level, they share mechanisms of enzymatic activity and cytotoxicity common to the members of the Shiga toxin family (Johannes L, Römer W, Nat Rev Microbiol 8: 105-16 (2010)). Over 39 different Shiga toxins have been described, such as the defined subtypes Stx1a, Stx1c, Stx1d, and Stx2a-g (Scheutz F et al., J Clin Microbiol 50: 2951-63 (2012)). Members of the Shiga toxin family are not naturally restricted to any bacterial species because Shiga-toxin-encoding genes can spread among bacterial species via horizontal gene transfer (Strauch E et al., Infect Immun 69: 7588-95 (2001); Bielaszewska M et al., Appl Environ Micrbiol 73: 3144-50 (2007); Zhaxybayeva O, Doolittle W, Curr Biol 21: R242-6 (2011)). As an example of interspecies transfer, a Shiga toxin was discovered in a strain of A. haemolyticus isolated from a patient (Grotiuz G et al., J Clin Microbiol 44: 3838-41 (2006)). Once a Shiga toxin encoding polynucleotide enters a new subspecies or species, the Shiga toxin amino acid sequence is presumed to be capable of developing slight sequence variations due to genetic drift and/or selective pressure while still maintaining a mechanism of cytotoxicity common to members of the Shiga toxin family (see Scheutz F et al., J Cin Microbiol 50: 2951-63 (2012)).

In some embodiments of the PD-L1 binding molecules described herein, the Shiga toxin A Subunit effector polypeptide component comprises a combination of two or more of the following Shiga toxin effector polypeptide sub-regions: (1) a de-immunized sub-region, (2) a protease-cleavage resistant sub-region near the carboxy-terminus of a Shiga toxin A1 fragment region, and (3) a T-cell epitope-peptide embedded or inserted sub-region.

### 1. De-Immunized, Shiga Toxin A Subunit Effector Polypeptides

In some embodiments, the Shiga toxin effector polypeptide of the binding molecule is de-immunized, such as, e.g., as compared to a wild-type Shiga toxin, wild-type Shiga toxin polypeptide, and/or Shiga toxin effector polypeptide comprising only wild-type polypeptide sequences. A Shiga toxin effector polypeptide and/or Shiga toxin A Subunit polypeptide, whether naturally occurring or not, can be de-immunized by a method described herein, described in WO 2015/113005, WO 2015/113007, WO 2016/196344, and WO 2018/140427, and/or known to the skilled worker, wherein the resulting molecule retains one or more Shiga toxin A Subunit functions. The de-immunized, Shiga toxin effector polypeptide may comprise a disruption of at least one, putative, endogenous, epitope region in order to reduce the antigenic and/or immunogenic potential of the Shiga toxin effector polypeptide after administration of the polypeptide to a chordate.

In some embodiments, the Shiga toxin effector polypeptide comprises a disruption of an endogenous epitope or epitope region, such as, e.g., a B-cell and/or CD4+ T-cell epitope. In some embodiments, the Shiga toxin effector polypeptide comprises a disruption of at least one, endogenous, epitope region described herein, wherein the disruption reduces the antigenic and/or immunogenic potential of the Shiga toxin effector polypeptide after administration of the polypeptide to a chordate, and wherein the Shiga toxin effector polypeptide is capable of exhibiting one or more Shiga toxin A Subunit functions, such as, e.g., a significant level of Shiga toxin cytotoxicity.

The term “disrupted” or “disruption” as used herein with regard to an epitope region refers to the deletion of at least one amino acid residue in an epitope region, inversion of two or more amino acid residues where at least one of the inverted amino acid residues is in an epitope region, insertion of at least one amino acid into an epitope region, and a substitution of at least one amino acid residue in an epitope region. An epitope region disruption by mutation includes amino acid substitutions with non-standard amino acids and/or non-natural amino acids. Epitope regions may alternatively be disrupted by mutations comprising the modification of an amino acid by the addition of a covalently-linked chemical structure which masks at least one amino acid in an epitope region, see, e.g. PEGylation (see Zhang C et al., BioDrugs 26: 209-15 (2012), small molecule adjuvants (Flower D, Expert Opin Drug Discov 7: 807-17 (2012), and site-specific albumination (Lim S et al., J Control Release 207-93 (2015)).

Certain epitope regions and disruptions are indicated herein by reference to specific amino acid positions of native Shiga toxin A Subunits provided in the Sequence Listing, noting that naturally occurring Shiga toxin A Subunits may comprise precursor forms containing signal sequences of about 22 amino acids at their amino-terminals which are removed to produce mature Shiga toxin A Subunits and are recognizable to the skilled worker. Further, certain epitope region disruptions are indicated herein by reference to specific amino acids (e.g. S for a serine residue) natively present at specific positions within native Shiga toxin A Subunits (e.g. S33 for the serine residue at position 33 from the amino-terminus) followed by the amino acid with which that residue has been substituted in the particular mutation under discussion (e.g. S33I represents the amino acid substitution of isoleucine for serine at amino acid residue 33 from the amino-terminus).

In some embodiments, the de-immunized, Shiga toxin effector polypeptide comprises a disruption of at least one epitope region provided herein. In some embodiments, the de-immunized, Shiga toxin effector polypeptide comprises a disruption of at least one epitope region described in WO 2015/113005 or WO 2015/113007.

In some embodiments, the de-immunized, Shiga toxin effector polypeptide comprises or consists essentially of a full-length Shiga toxin A Subunit (e.g. SLT-1A (SEQ ID NO:1), StxA (SEQ ID NO:2), or SLT-2A (SEQ ID NO:3)) comprising at least one disruption of the amino acid sequence selected from the group of natively positioned amino acids consisting of; 1-15 of SEQ ID NO:1 or SEQ ID NO:2; 3-14 of SEQ ID NO:3; 26-37 of SEQ ID NO:3; 27-37 of SEQ ID NO:1 or SEQ ID NO:2; 39-48 of SEQ ID NO:1 or SEQ ID NO:2; 42-48 of SEQ ID NO:3; 53-66 of SEQ ID NO:1, SEQ ID NO:2, or SEQ ID NO:3; 94-115 of SEQ ID NO:1, SEQ ID NO:2, or SEQ ID NO:3; 141-153 of SEQ ID NO:1 or SEQ ID NO:2; 140-156 of SEQ ID NO:3; 179-190 of SEQ ID NO:1 or SEQ ID NO:2; 179-191 of SEQ ID NO:3; 204 of SEQ ID NO:3; 205 of SEQ ID NO:1 or SEQ ID NO:2; 210-218 of SEQ ID NO:3; 240-258 of SEQ ID NO:3; 243-257 of SEQ ID NO:1 or SEQ ID NO:2; 254-268 of SEQ ID NO:1 or SEQ ID NO:2; 262-278 of SEQ ID NO:3; 281-297 of SEQ ID NO:3; and 285-293 of SEQ ID NO:1 or SEQ ID NO:2, or the equivalent position in a Shiga toxin A Subunit polypeptide, conserved Shiga toxin effector polypeptide sub-region, and/or non-native, Shiga toxin effector polypeptide sequence.

In some embodiments, a Shiga toxin effector polypeptide comprises the sequence of SEQ ID NO: 169. In some embodiments, a Shiga toxin effector polypeptide comprises the sequence of SEQ ID NO: 170. In some embodiments, a Shiga toxin effector polypeptide comprises the sequence of SEQ ID NO: 173.

In some embodiments, the Shiga toxin effector polypeptide comprises or consists essentially of a truncated Shiga toxin A Subunit. Truncations of Shiga toxin A Subunits might result in the deletion of an entire epitope region(s) without affecting Shiga toxin effector function(s). The smallest, Shiga toxin A Subunit fragment shown to exhibit significant enzymatic activity was a polypeptide composed of residues 75-247 of StxA (Al-Jaufy A et al., Infect Immun 62: 956-60 (1994)). Truncating the carboxy-terminus of SLT-1A, StxA, or SLT-2A to amino acids 1-251 removes two predicted B-cell epitope regions, two predicted CD4 positive (CD4+) T-cell epitopes, and a predicted, discontinuous, B-cell epitope. Truncating the amino-terminus of SLT-1A, Stx A, or SLT-2A to 75-293 removes at least three, predicted, B-cell epitope regions and three predicted CD4+ T-cell epitopes. Truncating both amino- and carboxy-terminals of SLT-1A, StxA, or SLT-2A to 75-251 deletes at least five, predicted, B-cell epitope regions; four, putative, CD4+ T-cell epitopes; and one, predicted, discontinuous, B-cell epitope.

In some embodiments, a Shiga toxin effector polypeptide comprises or consists essentially of a full-length or truncated Shiga toxin A Subunit with at least one mutation, e.g. deletion, insertion, inversion, or substitution, in a provided epitope region. In some embodiments, the polypeptides comprise a disruption which comprises a deletion of at least one amino acid within the epitope region. In some embodiments, the polypeptides comprise a disruption which comprises an insertion of at least one amino acid within the epitope region. In some embodiments, the polypeptides comprise a disruption which comprises an inversion of amino acids, wherein at least one inverted amino acid is within the epitope region. In some embodiments, the polypeptides comprise a disruption which comprises a mutation, such as an amino acid substitution to a non-standard amino acid or an amino acid with a chemically modified side chain.

In some embodiments, the Shiga toxin effector polypeptides comprise or consist essentially of a full-length or truncated Shiga toxin A Subunit with one or more mutations as compared to the native sequence which comprises at least one amino acid substitution selected from the group consisting of: A, G, V, L, I, P, C, M, F, S, D, N, Q, H, and K. In some embodiments, the polypeptide comprises or consists essentially of a full-length or truncated Shiga toxin A Subunit with a single mutation as compared to the native sequence wherein the substitution is selected from the group consisting of: D to A, D to G, D to V, D to L, D to I, D to F, D to S, D to Q, E to A, E to G, E to V, E to L, E to I, E to F, E to S, E to Q, E to N, E to D, E to M, E to R, G to A, H to A, H to G, H to V, H to L, H to I, H to F, H to M, K to A, K to G, K to V, K to L, K to I, K to M, K to H, L to A, L to G, N to A, N to G, N to V, N to L, N to I, N to F, P to A, P to G, P to F, R to A, R to G, R to V, R to L, R to I, R to F, R to M, R to Q, R to S, R to K, R to H, S to A, S to G, S to V, S to L, S to I, S to F, S to M, T to A, T to G, T to V, T to L, T to I, T to F, T to M, T to S, Y to A, Y to G, Y to V, Y to L, Y to I, Y to F, and Y to M.

In some embodiments, the Shiga toxin effector polypeptides comprise or consist essentially of a full-length or truncated Shiga toxin A Subunit with one or more mutations as compared to the native amino acid residue sequence which comprises at least one amino acid substitution of an immunogenic residue and/or within an epitope region, wherein at least one substitution occurs at the natively positioned group of amino acids selected from the group consisting of 1 of SEQ ID NO:1 or SEQ ID NO:2; 4 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO:1, SEQ ID NO:2, or SEQ ID NO:3; 59 of SEQ ID NO:1. SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1. SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO: 1. SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2.

In some embodiments, the Shiga toxin effector polypeptides comprise or consist essentially of a full-length or truncated Shiga toxin A Subunit with at least one substitution of an immunogenic residue and/or within an epitope region, wherein at least one amino acid substitution is to a non-conservative amino acid (see, e.g., Table 4, infra) relative to a natively occurring amino acid positioned at one of the following native positions: 1 of SEQ ID NO:1 or SEQ ID NO:2; 4 of SEQ ID NO:1, SEQ ID NO:2, or SEQ ID NO:3; 8 of SEQ ID NO:1. SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2.

In some embodiments, the Shiga toxin effector polypeptides comprise or consist essentially of a full-length or truncated Shiga toxin A Subunit with at least one amino acid substitution selected from the group consisting of: KI to A, G, V, L, I, F, M and H; T4 to A, G, V, L, I, F, M, and S; D6 to A, G, V, L, I, F, S, and Q; S8 to A, G, V, I, L, F, and M; T8 to A, G, V, I, L, F, M, and S; T9 to A, G, V, I, L, F, M, and S; S9 to A, G, V, L, I, F, and M; K11 to A, G, V, L, I, F, M and H; T12 to A, G, V, I, L, F, M, and S; S33 to A, G, V, L, I, F, and M; S43 to A, G, V, L, I, F, and M; G44 to A and L; S45 to A, G, V, L, I, F, and M; T45 to A, G, V, L, I, F, and M; G46 to A and P; D47 to A, G, V, L, I, F, S, and Q; N48 to A, G, V, L, and M; L49 to A or G; F50; A51 to V; D53 to A, G, V, L, I, F, S, and Q; V54 to A, G, and L; R55 to A, G, V, L, I, F, M, Q, S, K, and H; G56 to A and P; 157 to A, G, M, and F; L57 to A, G, M, and F; D58 to A, G, V, L, I, F, S, and Q; P59 to A, G, and F; E60 to A, G, V, L, I, F, S, Q, N, D, M, and R; E61 to A, G, V, L, I, F, S, Q, N, D, M, and R; G62 to A; D94 to A, G, V, L, I, F, S, and Q; R84 to A, G, V, L, I, F, M, Q, S, K, and H; V88 to A and G; 188 to A, G, and V; D94; S96 to A, G, V, I, L, F, and M; T104 to A, G, V, I, L, F, M, and S; A105 to L; T107 to A, G, V, I, L, F, M, and S; S107 to A, G, V, L, I, F, and M; L108 to A, G, and M; S109 to A, G, V, I, L, F, and M; T109 to A, G, V, I, L, F, M, and S; G110 to A; DIII to A, G, V, L, I, F, S, and Q; S112 to A, G, V, L, 1, F, and M; D141 to A, G, V, L, I, F, S, and Q; G147 to A; V154 to A and G; R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, and V; S186 to A, G, V, I, L, F, and M; G187 to A; R188 to A, G, V, L, I, F, M, Q, S, K, and H; S189 to A, G, V, I, L, F, and M; D197 to A, G, V, L, I, F, S, and Q; D198 to A, G, V, L, I, F, S, and Q; R204 to A, G, V, L, I, F, M, Q, S, K, and H; R205 to A, G, V, L, 1, F, M, Q, S, K and H; C242 to A, G, V, and S; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; C262 to A, G, V, and S; D264 to A, G, V, L, I, F, S, and Q; G264 to A; and T286 to A, G, V, L, I, F, M, and S.

In some embodiments, the Shiga toxin effector polypeptides comprise or consist essentially of a full-length or truncated Shiga toxin A Subunit with at least one of the following amino acid substitutions K1A, K1M, T41, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, 157M, D58A, D58V, D58F, P59A, P59F, E60I, E60T, E60R. E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I. These epitope disrupting substitutions may be combined to form a de-immunized, Shiga toxin effector polypeptide with multiple substitutions per epitope region and/or multiple epitope regions disrupted while still retaining Shiga toxin effector function. For example, substitutions at the natively positioned K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I may be combined, where possible, with substitutions at the natively positioned residues K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T1(9V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I to create de-immunized, Shiga toxin effector polypeptides.

Any of the de-immunized. Shiga toxin effector polypeptide sub-regions and/or epitope disrupting mutations described herein may be used alone or in combination with each individual embodiment as described herein, including methods described herein.

### 2. Protease-Cleavage Resistant, Shia Toxin a Subunit Effector Polypeptides

In some embodiments, the Shiga toxin effector polypeptide of the binding molecule comprises (1) a Shiga toxin A1 fragment derived region having a carboxy-terminus and (2) a disrupted furin-cleavage site at the carboxy-terminus of the Shiga toxin A1 fragment region. Improving the stability of connections between the Shiga toxin component and other components of binding molecules, e.g., cell-targeting binding regions, can improve their toxicity profiles after administration to organisms by reducing non-specific toxicities caused by the breakdown of the connection and loss of cell-targeting, such as, e.g., as a result of proteolysis.

Shiga toxin A Subunits of members of the Shiga toxin family comprise a conserved, furin-cleavage site at the carboxy-terminal of their A1 fragment regions important for Shiga toxin function. Furin-cleavage sites can be identified by the skilled worker using standard techniques and/or by using the information herein.

Furin-cleavage sites in Shiga toxin A Subunits and Shiga toxin effector polypeptides can be identified by the skilled worker using standard methods and/or by using the information herein. Furin cleaves the minimal, consensus sequence R-x-x-R (Schalken J et al., J Clin Invest 80: 1545-9 (1987); Bresnahan P et al., J Cell Biol 111: 2851-9 (1990); Hatsuzawa K et al., J Biol Chem 265: 22075-8 (1990); Wise R et al., Proc Natl Acad Sci USA 87: 9378-82 (1990); Molloy S et al., J Biol Chem 267: 16396-402 (1992)). Consistent with this, many furin inhibitors comprise peptides comprising the sequence R-x-x-R. An example of a synthetic inhibitor of furin is a molecule comprising the peptide R-V-K-R (Henrich S et al., Nat Struct Biol 10: 520-6 (2003)). In general, a peptide or protein comprising a surface accessible, dibasic amino acid motif with two positively charged, amino acids separated by two amino acid residues can be predicted to be sensitive to furin-cleavage with cleavage occurring at the carboxy bond of the last basic amino acid in the sequence.

Consensus sequences in substrates cleaved by furin have been identified with some degree of specificity. A furin-cleavage site has been described that comprises a region of twenty, continuous, amino acid residues, which can be labeled P14 through P6′ (Tian S et al., Int J Mol Sci 12: 1060-5 (2011)) using the nomenclature described in Schechter 1, Berger, A, Biochem Biophys Res Commun 32: 898-902 (1968). According to this nomenclature, the furin-cleavage site is at the carboxy bond of the amino acid residue designated P1, and the amino acid residues of the furin-cleavage site are numbered P2, P3, P4, etc., in the direction going toward the amino-terminus from this reference P1 residue. The amino acid residues of the furin-cleavage site going toward the carboxy-terminus from the P1 reference residue are numbered with the prime notation P2′, P3′, P4′, etc. Using this nomenclature, the P6 to P2′ region delineates the core substrate of the furin cleavage site which is bound by the enzymatic domain of furin. The two flanking regions P14 to P7 and P3′ to P6′ are often rich in polar, amino acid residues to increase the accessibility to the core furin cleavage site located between them.

The twenty amino acid residue, furin-cleavage site found in native. Shiga toxin A Subunits at the junction between the Shiga toxin A1 fragment and A2 fragment is well characterized in certain Shiga toxins. For example in StxA (SEQ ID NO:2) and SLT-1A (SEQ ID NO:1), this furin-cleavage site is natively positioned from L238 to F257, and in SLT-2A (SEQ ID NO:3), this furin-cleavage site is natively positioned from V237 to Q256. Based on amino acid homology, experiment, and/or furin-cleavage assays described herein, the skilled worker can identify furin-cleavage sites in other native. Shiga toxin A Subunits or Shiga toxin effector polypeptides, where the sites are actual furin-cleavage sites or are predicted to result in the production of A1 and A2 fragments after furin cleavage of those molecules within a eukaryotic cell.

In some embodiments, the Shiga toxin effector polypeptide comprises (1) a Shiga toxin A1 fragment derived polypeptide having a carboxy-terminus and (2) a disrupted furin-cleavage site at the carboxy-terminus of the Shiga toxin A1 fragment derived polypeptide. The carboxy-terminus of a Shiga toxin A1 fragment derived polypeptide may be identified by the skilled worker by using techniques known in the art, such as, e.g., by using protein sequence alignment software to identify (i) a furin-cleavage site conserved with a naturally occurring Shiga toxin, (ii) a surface exposed, extended loop conserved with a naturally occurring Shiga toxin, and/or (iii) a stretch of amino acid residues which are predominantly hydrophobic (i.e. a hydrophobic “patch”) that may be recognized by the ERAD system.

A protease-cleavage resistant, Shiga toxin effector polypeptide of the binding molecule (1) may be completely lacking any furin-cleavage site at a carboxy-terminus of its Shiga toxin A1 fragment region and/or (2) comprise a disrupted furin-cleavage site at the carboxy-terminus of its Shiga toxin A1 fragment region and/or region derived from the carboxy-terminus of a Shiga toxin A1 fragment. A disruption of a furin-cleavage site include various alterations to an amino acid residue in the furin-cleavage site, such as, e.g., a post-translation modification(s), an alteration of one or more atoms in an amino acid functional group, the addition of one or more atoms to an amino acid functional group, the association to a non-proteinaceous moiety(ies), and/or the linkage to an amino acid residue, peptide, polypeptide such as resulting in a branched proteinaceous structure.

Protease-cleavage resistant, Shiga toxin effector polypeptides may be created from a Shiga toxin effector polypeptide and/or Shiga toxin A Subunit polypeptide, whether naturally occurring or not, using a method described herein, described in WO 2015/191764, and/or known to the skilled worker, wherein the resulting molecule still retains one or more Shiga toxin A Subunit functions.

With regard to a furin-cleavage site or furin-cleavage site, the term “disruption” or “disrupted” refers to an alteration from the naturally occurring furin-cleavage site and/or furin-cleavage site, such as, e.g., a mutation, that results in a reduction in furin-cleavage proximal to the carboxy-terminus of a Shiga toxin A1 fragment region, or identifiable region derived thereof, as compared to the furin-cleavage of a wild-type Shiga toxin A Subunit or a polypeptide derived from a wild-type Shiga toxin A Subunit comprising only wild-type polypeptide sequences. An alteration to an amino acid residue in the furin-cleavage site includes a mutation in the furin-cleavage site, such as, e.g., a deletion, insertion, inversion, substitution, and/or carboxy-terminal truncation of the furin-cleavage site, as well as a post-translation modification, such as, e.g., as a result of glycosylation, albumination, and the like which involve conjugating or linking a molecule to the functional group of an amino acid residue. Because the furin-cleavage site is comprised of about twenty, amino acid residues, in theory, alterations, modifications, mutations, deletions, insertions, and/or truncations involving one or more amino acid residues of any one of these twenty positions might result in a reduction of furin-cleavage sensitivity (Tian S et al., Sci Rep 2: 261 (2012)). The disruption of a furin-cleavage site and/or furin-cleavage site might or might not increase resistance to cleavage by other proteases, such as, e.g., trypsin and extracellular proteases common in the vascular system of mammals. The effects of a given disruption to cleavage sensitivity of a given protease may be tested by the skilled worker using techniques known in the art.

A “disrupted furin-cleavage site” is furin-cleavage site comprising an alteration to one or more amino acid residues derived from the 20 amino acid residue region representing a conserved, furin-cleavage site found in native, Shiga toxin A Subunits at the junction between the Shiga toxin A1 fragment and A2 fragment regions and positioned such that furin cleavage of a Shiga toxin A Subunit results in the production of the A1 and A2 fragments; wherein the disrupted furin-cleavage site exhibits reduced furin cleavage in an experimentally reproducible way as compared to a reference molecule comprising a wild-type, Shiga toxin A1 fragment region fused to a carboxy-terminal polypeptide of a size large enough to monitor furin cleavage using the appropriate assay known to the skilled worker and/or described herein.

In some embodiments, the Shiga toxin effector polypeptide comprises (1) a Shiga toxin A1 fragment derived polypeptide having a carboxy-terminus and (2) a disrupted furin-cleavage site at the carboxy-terminus of the Shiga toxin A1 fragment polypeptide region; wherein the Shiga toxin effector polypeptide (and any binding molecule comprising it) is more furin-cleavage resistant as compared to a reference molecule, such as, e.g., a wild-type Shiga toxin polypeptide comprising the carboxy-terminus of an A1 fragment and/or the conserved, furin-cleavage site between A1 and A2 fragments. For example, a reduction in furin cleavage of one molecule compared to a reference molecule may be determined using an in vitro, furin-cleavage assay described in WO 2015/191764, conducted using the same conditions, and then performing a quantitation of the band density of any fragments resulting from cleavage to quantitatively measure in change in furin cleavage.

In some embodiments, the Shiga toxin effector polypeptide is more resistant to furin-cleavage in vitro and/or in vivo as compared to a wild-type, Shiga toxin A Subunit.

In general, the protease-cleavage sensitivity of a binding molecule is tested by comparing it to the same molecule having its furin-cleavage resistant, Shiga toxin effector polypeptide replaced with a wild-type, Shiga toxin effector polypeptide comprising a Shiga toxin A1 fragment. In some embodiments, the PD-L1 binding molecules comprising a disrupted furin-cleavage site exhibits a reduction in in vitro furin cleavage of 30%, 40%, 50%, 60%, 70%, 80%, 90%, 95%, 97%, 98% or greater compared to a reference molecule comprising a wild-type, Shiga toxin A1 fragment fused at its carboxy-terminus to a peptide or polypeptide.

Several furin-cleavage site disruptions have been described. For example, mutating the two conserved arginines to alanines in the minimal R-x-x-R site completely blocked processing by furin and/or furin-like proteases (see e.g Duda A et al., J Virology 78: 13865-70 (2004)). Because the furin-cleavage site is comprised of about twenty amino acid residues, in theory, certain mutations involving one or more of any one of these twenty, amino acid residue positions might abolish furin cleavage or reduce furin cleavage efficiency (see e.g. Tian S et al., Sci Rep 2: 261 (2012)).

In some embodiments, the molecules described herein comprise a Shiga toxin effector polypeptide derived from at least one A Subunit of a member of the Shiga toxin family wherein the Shiga toxin effector polypeptide comprises a disruption in one or more amino acids derived from the conserved, highly accessible, protease-cleavage sensitive loop of Shiga toxin A Subunits. For example, in StxA and SLT-1A, this highly accessible, protease-sensitive loop is natively positioned from amino acid residues 242 to 261, and in SLT-2A, this conserved loop is natively positioned from amino acid residues 241 to 260. Based on polypeptide sequence homology, the skilled worker can identify this conserved, highly accessible loop structure in other Shiga toxin A Subunits. Certain mutations to the amino acid residues in this loop can reduce the accessibility of certain amino acid residues within the loop to proteolytic cleavage and this might reduce furin-cleavage sensitivity.

In some embodiments, a PD-L1 binding molecule comprises a Shiga toxin effector polypeptide comprising a disrupted furin-cleavage site comprising a mutation in the surface-exposed, protease sensitive loop conserved among Shiga toxin A Subunits. In some embodiments, a PD-L1 binding molecule comprises a Shiga toxin effector polypeptide comprising a disrupted furin-cleavage site comprising a mutation in this protease-sensitive loop of Shiga toxin A Subunits, the mutation which reduce the surface accessibility of certain amino acid residues within the loop such that furin-cleavage sensitivity is reduced.

In some embodiments, the disrupted furin-cleavage site of a Shiga toxin effector polypeptide comprises a disruption in terms of existence, position, or functional group of one or both of the consensus amino acid residues P1 and P4, such as, e.g., the amino acid residues in positions 1 and 4 of the minimal furin-cleavage site R/Y-x-x-R. For example, mutating one or both of the two arginine residues in the minimal, furin consensus site R-x-x-R to alanine will disrupt a furin-cleavage site and prevent furin-cleavage at that site. Similarly, amino acid residue substitutions of one or both of the arginine residues in the minimal furin-cleavage site R-x-x-R to any non-conservative amino acid residue known to the skilled worker will reduced the furin-cleavage sensitivity of the site. In particular, amino acid residue substitutions of arginine to any non-basic amino acid residue which lacks a positive charge, such as, e.g., A, G, P, S, T, D, E, Q, N, C, I, L, M, V, F, W, and Y, will result in a disrupted furin-cleavage site.

In some embodiments, the disrupted furin-cleavage site of a Shiga toxin effector polypeptide comprises a disruption in the spacing between the consensus amino acid residues P4 and P1 in terms of the number of intervening amino acid residues being other than two, and, thus, changing either P4 and/or P1 into a different position and eliminating the P4 and/or P1 designations. For example, deletions within the furin-cleavage site of the minimal furin-cleavage site or the core, furin-cleavage site will reduce the furin-cleavage sensitivity of the furin-cleavage site.

In some embodiments, the disrupted furin-cleavage site comprises one or more amino acid residue substitutions, as compared to a wild-type, Shiga toxin A Subunit. In some embodiments, the disrupted furin-cleavage site comprises one or more amino acid residue substitutions within the minimal furin-cleavage site R/Y-x-x-R, such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises an un-disrupted, minimal furin-cleavage site R/Y-x-x-R but instead comprises a disrupted flanking region, such as, e.g., amino acid residue substitutions in one or more amino acid residues in the furin-cleavage site flanking regions natively positioned at, e.g., 241-247 and/or 252-259. In some embodiments, the disrupted furin cleavage site comprises a substitution of one or more of the amino acid residues located in the P1-P6 region of the furin-cleavage site; mutating P1′ to a bulky amino acid, such as, e.g., R, W. Y. F, and H and mutating P2′ to a polar and hydrophilic amino acid residue; and substituting one or more of the amino acid residues located in the P1′-P6′ region of the furin-cleavage site with one or more bulky and hydrophobic amino acid residues

In some embodiments, the disruption of the furin-cleavage site comprises a deletion, insertion, inversion, and/or mutation of at least one amino acid residue within the furin-cleavage site. In some embodiments, a protease-cleavage resistant, Shiga toxin effector polypeptide comprises a disruption of the amino acid sequence natively positioned at 249-251 of the A Subunit of Shiga-like toxin 1 (SEQ ID NO:1) or Shiga toxin (SEQ ID NO:2), or at 247-250 of the A Subunit of Shiga-like toxin 2 (SEQ ID NO:3) or the equivalent position in a conserved Shiga toxin effector polypeptide and/or non-native Shiga toxin effector polypeptide sequence. In some embodiments, protease-cleavage resistant, Shiga toxin effector polypeptides comprise a disruption which comprises a deletion of at least one amino acid within the furin-cleavage site. In some embodiments, protease-cleavage resistant, Shiga toxin effector polypeptides comprise a disruption which comprises an insertion of at least one amino acid within the protease-cleavage region. In some embodiments, the protease-cleavage resistant. Shiga toxin effector polypeptides comprise a disruption which comprises an inversion of amino acids, wherein at least one inverted amino acid is within the protease cleavage site. In some embodiments, the protease-cleavage resistant, Shiga toxin effector polypeptides comprise a disruption which comprises a mutation, such as an amino acid substitution to a non-standard amino acid or an amino acid with a chemically modified side chain.

In some embodiments, the disrupted furin-cleavage site comprises the deletion of nine, ten, eleven, or more of the carboxy-terminal amino acid residues within the furin-cleavage site. In these embodiments, the disrupted furin-cleavage site will not comprise a furin-cleavage site. In other words, certain embodiments lack a furin-cleavage site at the carboxy-terminus of the A1 fragment region.

In some embodiments, the disrupted furin-cleavage site comprises both an amino acid residue deletion and an amino acid residue substitution as compared to a wild-type, Shiga toxin A Subunit. In some embodiments, the disrupted furin-cleavage site comprises one or more amino acid residue deletions and substitutions within the minimal furin-cleavage site R/Y-x-x-R, such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises an amino acid residue deletion and an amino acid residue substitution as well as a carboxy-terminal truncation as compared to a wild-type, Shiga toxin A Subunit. In some embodiments, the disrupted furin-cleavage site comprises one or more amino acid residue deletions and substitutions within the minimal furin-cleavage site R/Y-x-x-R, such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises both an amino acid substitution within the minimal furin-cleavage site R/Y-x-x-R and a carboxy-terminal truncation as compared to a wild-type, Shiga toxin A Subunit, such as, e.g., for StxA and SLT-1A derived Shiga toxin effector polypeptides, truncations ending at the natively amino acid position 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate.

In some embodiments, the disrupted furin-cleavage site comprises an insertion of one or more amino acid residues as compared to a wild-type, Shiga toxin A Subunit as long as the inserted amino residue(s) does not create a de novo furin-cleavage site. In some embodiments, the insertion of one or more amino acid residues disrupts the natural spacing between the arginine residues in the minimal, furin-cleavage site R/Y-x-x-R, such as, e.g., StxA and SLT-1A derived polypeptides comprising an insertion of one or more amino acid residues at 249 or 250 and thus between R248 and R251; or SLT-2A derived polypeptides comprising an insertion of one or more amino acid residues at 248 or 249 and thus between Y247 and R250.

In some embodiments, the disrupted furin-cleavage site comprises both an amino acid residue insertion and a carboxy-terminal truncation as compared to a wild-type, Shiga toxin A Subunit. In some embodiments, the disrupted furin-cleavage site comprises both an amino acid residue insertion and an amino acid residue substitution as compared to a wild-type, Shiga toxin A Subunit. In some embodiments, the disrupted furin-cleavage site comprises both an amino acid residue insertion and an amino acid residue deletion as compared to a wild-type, Shiga toxin A Subunit.

In some embodiments, the disrupted furin-cleavage site comprises an amino acid residue deletion, an amino acid residue insertion, and an amino acid residue substitution as compared to a wild-type, Shiga toxin A Subunit.

In some embodiments, the disrupted furin-cleavage site comprises an amino acid residue deletion, insertion, substitution, and carboxy-terminal truncation as compared to a wild-type, Shiga toxin A Subunit.

In some embodiments, the Shiga toxin effector polypeptide comprising a disrupted furin-cleavage site is directly fused by a peptide bond to a molecular moiety comprising an amino acid, peptide, and/or polypeptide wherein the fused structure involves a single, continuous polypeptide. In these fusion embodiments, the amino acid sequence following the disrupted furin-cleavage site should not create a de now), furin-cleavage site at the fusion junction.

Any of the above protease-cleavage resistant, Shiga toxin effector polypeptide sub-regions and/or disrupted furin-cleavage sites may be used alone or in combination with each individual embodiment as described herein, including methods described herein.

### 3. T-Cell Hyper-Immunized, Shiga Toxin a Subunit Effector Polypeptides

In some embodiments, the Shiga toxin effector polypeptide of the binding molecule comprises an embedded or inserted epitope-peptide. In some embodiments, the epitope-peptide is a heterologous, T-cell epitope-peptide, such as, e.g., an epitope considered heterologous to Shiga toxin A Subunits. In some embodiments, the epitope-peptide is a CD8+ T-cell epitope. In some embodiments, the CD8+ T-cell epitope-peptide has a binding affinity to a MHC class I molecule characterized by a dissociation constant (K_{D}) of 10⁻⁴ molar or less and/or the resulting MHC class I-epitope-peptide complex has a binding affinity to a T-cell receptor (TCR) characterized by a dissociation constant (K_{D}) of 10⁻⁴ molar or less.

In some embodiments, the Shiga toxin effector polypeptide comprises an embedded or inserted, heterologous, T-cell epitope, such as, e.g., a human CD8+ T-cell epitope. In some embodiments, the heterologous, T-cell epitope is embedded or inserted so as to disrupt an endogenous epitope or epitope region (e.g. a B-cell epitope and/or CD4+ T-cell epitope) identifiable in a naturally occurring Shiga toxin polypeptide or parental Shiga toxin effector polypeptide from which the Shiga toxin effector polypeptide is derived.

In some embodiments, the Shiga toxin effector polypeptide (and any binding molecule comprising it) is CD8+ T-cell hyper-immunized, such as, e.g., as compared to a wild-type Shiga toxin polypeptide. Each CD8+ T-cell hyper-immunized, Shiga toxin effector polypeptide comprises an embedded or inserted T-cell epitope-peptide. Hyper-immunized, Shiga toxin effector polypeptides can be created from Shiga toxin effector polypeptides and/or Shiga toxin A Subunit polypeptides, whether naturally occurring or not, using a method described herein, described in WO 2015/113005, and/or known to the skilled worker, wherein the resulting molecule still retains one or more Shiga toxin A Subunit functions.

A T-cell epitope is a molecular structure which is comprised by an antigenic peptide and can be represented by a linear, amino acid sequence. Commonly, T-cell epitopes are peptides of sizes of eight to eleven amino acid residues (Townsend A, Bodmer H, Annu Rev Immunol 7: 601-24 (1989)); however, certain T-cell epitope-peptides have lengths that are smaller than eight or larger than eleven amino acids long (see e.g. Livingstone A, Fathman C, Annu Rev Immunol 5: 477-501 (1987); Green K et al., Eur J Immunol 34: 2510-9 (2004)). In some embodiments, the embedded or inserted epitope is at least seven amino acid residues in length. In some embodiments, the embedded or inserted epitope is bound by a TCR with a binding affinity characterized by a K_{D} less than 10 mM (e.g. 1-100 pM) as calculated using the formula in Stone J et al., Immunology 126: 165-76 (2009). However, it should be noted that the binding affinity within a given range between the MHC-epitope and TCR may not correlate with antigenicity and/or immunogenicity (see e.g. Al-Ramadi B et al., J Immunol 155: 662-73 (1995)), such as due to factors like MHC-peptide-TCR complex stability, MHC-peptide density and MHC-independent functions of TCR cofactors such as CD8 (Baker B et al., Immunity 13: 475-84 (2000); Hornell T et al., J Immunol 170; 4506-14 (2003); Woolridge L et al., J Immunol 171: 6650-60 (2003)).

In some embodiments, the molecule comprises a CD8+ T-cell epitope. In some further embodiments, the CD8+ T-cell epitope is a CD8+ T-cell epitope with regard to a human immune system. In some embodiments, the CD8+ T-cell epitope is a peptide having at least seven, eight, nine, or ten amino acid residues. In some embodiments, the CD8+ T-cell epitope comprises or consists of nine amino acid residues. In some embodiments, the CD8+ T-cell epitope may bound by a human TCR with a binding affinity characterized by a K_{D} less than 10 mM (e.g. 1-100 μM), e.g. as determined using an in vitro assay known to the skilled worker.

In some embodiments, the molecule comprises a CD8+ T-cell epitope having a sequence of NLVPMVATV (SEQ ID NO: 78). In some embodiments, the molecule comprises a CD8+ T-cell epitope having a sequence VTEHDTLLY (SEQ ID NO: 79). In some embodiments, the molecule comprises a CD8+ T-cell epitope having a sequence SIINFEKYL (SEQ ID NO: 80). In some embodiments, the molecule comprises a CD8+ T-cell epitope having a sequence GLDRNSGNY (SEQ ID NO: 81). In some embodiments, the molecule comprises a CD8+ T-cell epitope having a sequence GVMTRGRLK (SEQ ID NO: 82). In some embodiments, the molecule comprises a CD8+ T-cell epitope having a sequence GILGFVFTL (SEQ ID NO: 83). In some embodiments, the molecule comprises a CD8+ T-cell epitope having a sequence ILRGSVAHK (SEQ ID NO: 84). In some embodiments, the molecule comprises a CD8+ T-cell epitope having a sequence YSEHPTFTSQY (SEQ ID NO: 300). In some embodiments, the molecule comprises a CD8+ T-cell epitope having a sequence KLGGALQAK (SEQ ID NO: 301). In some embodiments, the molecule comprises a CD8+ T-cell epitope having a sequence QYDPVAALF (SEQ ID NO: 302). In some embodiments, the molecule comprises a CD8+ T-cell epitope having a sequence AYAQKIFKI (SEQ ID NO: 314). In some embodiments, the molecule comprises a CD8+ T-cell epitope having a sequence TVRSHCVSK (SEQ ID NO: 315). In some embodiments, the molecule comprises a CD8+ T-cell epitope having a sequence TLLNCAVTK (SEQ ID NO: 316).

In some embodiments, a binding molecule described herein comprises a Shiga toxin effector polypeptide comprising any one of SEQ ID NO: 1-18, 40-68, 169, 170, or 173 and a CD8+ T-cell epitope comprising the sequence of any one of SEQ ID NO: 78-84 and 300-302. In some embodiments, a binding molecule comprises a Shiga toxin effector polypeptide comprising SEQ ID NO: 41 and a CD8+ T-cell epitope comprising the sequence of any one of SEQ ID NO: 78-84 and 300-302. In some embodiments, a binding molecule comprises a Shiga toxin effector polypeptide comprising SEQ ID NO: 41 and a CD8+ T-cell epitope comprising the sequence of SEQ ID NO: 78. In some embodiments, a binding molecule comprises a Shiga toxin effector polypeptide comprising SEQ ID NO: 41 and a CD8+ T-cell epitope comprising the sequence of SEQ ID NO: 79. In some embodiments, a binding molecule comprises a Shiga toxin effector polypeptide comprising SEQ ID NO: 41 and a CD8+ T-cell epitope comprising the sequence of SEQ ID NO: 80. In some embodiments, a binding molecule comprises a Shiga toxin effector polypeptide comprising SEQ ID NO: 41 and a CD8+ T-cell epitope comprising the sequence of SEQ ID NO: 81. In some embodiments, a binding molecule comprises a Shiga toxin effector polypeptide comprising SEQ ID NO: 41 and a CD8+ T-cell epitope comprising the sequence of SEQ ID NO: 82. In some embodiments, a binding molecule comprises a Shiga toxin effector polypeptide comprising SEQ ID NO: 41 and a CD8+ T-cell epitope comprising the sequence of SEQ ID NO: 83. In some embodiments, a binding molecule comprises a Shiga toxin effector polypeptide comprising SEQ ID NO: 41 and a CD8+ T-cell epitope comprising the sequence of SEQ ID NO: 84. In some embodiments, a binding molecule comprises a Shiga toxin effector polypeptide comprising SEQ ID NO: 41 and a CD8+ T-cell epitope comprising the sequence of SEQ ID NO: 300. In some embodiments, a binding molecule comprises a Shiga toxin effector polypeptide comprising SEQ ID NO: 41 and a CD8+ T-cell epitope comprising the sequence of SEQ ID NO: 301. In some embodiments, a binding molecule comprises a Shiga toxin effector polypeptide comprising SEQ ID NO: 41 and a CD8+ T-cell epitope comprising the sequence of SEQ ID NO: 302.

A heterologous, T-cell epitope is an epitope not already present in a wild-type Shiga toxin A Subunit; a naturally occurring Shiga toxin A Subunit; and/or a parental, Shiga toxin effector polypeptide used as a source polypeptide for modification by a method described herein, described in WO 2015/113005, and/or known to the skilled worker.

A heterologous, T-cell epitope-peptide may be incorporated into a source polypeptide via numerous methods known to the skilled worker, including, e.g., the processes of creating one or more amino acid substitutions within the source polypeptide, fusing one or more amino acids to the source polypeptide, inserting one or more amino acids into the source polypeptide, linking a peptide to the source polypeptide, and/or a combination of the aforementioned processes. The result of such a method is the creation of a modified variant of the source polypeptide which comprises one or more embedded or inserted, heterologous, T-cell epitope-peptides.

T-cell epitopes may be chosen or derived from a number of source molecules for use as described herein. T-cell epitopes may be created or derived from various naturally occurring proteins. T-cell epitopes may be created or derived from various naturally occurring proteins foreign to mammals, such as, e.g., proteins of microorganisms. T-cell epitopes may be created or derived from mutated human proteins and/or human proteins aberrantly expressed by malignant human cells. T-cell epitopes may be synthetically created or derived from synthetic molecules (see e.g., Carbone F et al., J Exp Med 167: 1767-9 (1988); Del Val M et al., J Virol 65: 3641-6 (1991); Appella E et al., Biomed Pept Proteins Nucleic Acids 1: 177-84 (1995); Perez S et al., Cancer 116: 2071-80 (2010)).

Although any T-cell epitope-peptide is contemplated as being used as a heterologous, T-cell epitope, certain epitopes may be selected based on desirable properties. One objective described herein is to create CD8+ T-cell hyper-immunized, Shiga toxin effector polypeptides for administration to vertebrates, meaning that the heterologous, T-cell epitope is highly immunogenic and can elicit robust immune responses in vivo when displayed complexed with a MHC class I molecule on the surface of a cell. In some embodiments, the Shiga toxin effector polypeptide comprises one or more, embedded or inserted, heterologous, T-cell epitopes which are CD8+ T-cell epitopes. A Shiga toxin effector polypeptide that comprises a heterologous. CD8+ T-cell epitope is considered a CD8+ T-cell hyper-immunized, Shiga toxin effector polypeptide.

T-cell epitope components may be chosen or derived from a number of source molecules already known to be capable of eliciting a vertebrate immune response. T-cell epitopes may be derived from various naturally occurring proteins foreign to vertebrates, such as, e.g., proteins of pathogenic microorganisms and non-self, cancer antigens. In particular, infectious microorganisms may contain numerous proteins with known antigenic and/or immunogenic properties. Further, infectious microorganisms may contain numerous proteins with known antigenic and/or immunogenic sub-regions or epitopes.

For example, the proteins of intracellular pathogens with mammalian hosts are sources for T-cell epitopes. There are numerous intracellular pathogens, such as viruses, bacteria, fungi, and single-cell eukaryotes, with well-studied antigenic proteins or peptides. T-cell epitopes can be selected or identified from human viruses or other intracellular pathogens, such as, e.g., bacteria like mycobacterium, fungi like toxoplasmae, and protists like trypanosomes.

For example, there are many immunogenic, viral peptide components of viral proteins from viruses that are infectious to humans. Numerous, human T-cell epitopes have been mapped to peptides within proteins from influenza A viruses, such as peptides in the proteins HA glycoproteins FE17, S139/1, CH65, C05, hemagglutinin 1 (HA1), hemagglutinin 2 (HA2), nonstructural protein 1 and 2 (NS1 and NS 2), matrix protein 1 and 2 (M1 and M2), nucleoprotein (NP), neuraminidase (NA)), and many of these peptides have been shown to elicit human immune responses, such as by using ex vivo assay. Similarly, numerous, human T-cell epitopes have been mapped to peptide components of proteins from human cytomegaloviruses (HCMV), such as peptides in the proteins pp65 (UL83), UL128-131, immediate-early 1 (IE-1; UL123), glycoprotein B, tegument proteins, and many of these peptides have been shown to elicit human immune responses, such as by using ex vivo assays.

In some embodiments, the CD8+ T cell epitope is derived from an influenza A virus. In some embodiments, the CD8+ T cell epitope is derived from cytomegalovirus. In some embodiments, the CD8+ T cell epitope is derived from human cytomegalovirus. In some embodiments, the CD8+ T cell epitope is derived from an Epstein-Barr virus. In some embodiments, the CD8+ T cell epitope is derived from a coronavirus, e.g., SARS-CoV-1 or SARS-CoV-2. In some embodiments, the CD8+ T cell epitope is derived from a hepatitis A virus. In some embodiments, the CD8+ T cell epitope is derived from a hepatitis B virus. In some embodiments, the CD8+ T cell epitope is derived from a hepatitis C virus. In some embodiments, the CD8+ T cell epitope is derived from a Rubeola virus.

Another example is there are many immunogenic, cancer antigens in humans. The CD8+ T-cell epitopes of cancer and/or tumor cell antigens can be identified by the skilled worker using techniques known in the art, such as, e.g., differential genomics, differential proteomics, immunoproteomics, prediction then validation, and genetic approaches like reverse-genetic transfection (see e.g., Admon A et al., Mol Cell Proteomics 2: 388-98 (2003); Purcell A, Gorman J, Mol Cell Proteomics 3: 193-208 (2004); Comber J, Philip R, Ther Adv Vaccines 2: 77-89 (2014)). There are many antigenic and/or immunogenic T-cell epitopes already identified or predicted to occur in human cancer and/or tumor cells. For example, T-cell epitopes have been predicted in human proteins commonly mutated or overexpressed in neoplastic cells, such as, e.g., ALK, CEA, N-acetylglucosaminyl-transferase V (GnT-V), HCA587, PD-L1/neu, MAGE, Melan-A/MART-1, MUC-1, p53, and TRAG-3 (see e.g., van der Bruggen P et al., Science 254: 1643-7 (1991); Kawakami Y et al., J Exp Med 180: 347-52 (1994); Fisk B et al., J Exp Med 181: 2109-17 (1995); Guilloux Y et al., J Exp Med 183: 1173 (1996); Skipper J et al., J Exp Med 183: 527 (1996); Brossart P et al., 93: 4309-17 (1999); Kawashima I et al., Cancer Res 59: 431-5 (1999); Papadopoulos K et al., Clin Cancer Res 5: 2089-93 (1999); Zhu B et al., Clin Cancer Res 9: 1850-7 (2003); Li B et al., Clin Exp Immunol 140: 310-9 (2005); Ait-Tahar K et al., Int J Cancer 118: 688-95 (2006); Akiyama Y et al., Cancer Immunol Immunother 61: 2311-9 (2012)). In addition, synthetic variants of T-cell epitopes from human cancer cells have been created (see e.g., Lazoura E, Apostolopoulos V, Curr Med Chem 12: 629-39 (2005); Douat-Casassus C et al., J Med Chem 50: 1598-609 (2007)).

In some embodiments, the PD-L1 binding molecule comprises at least one CD8+ T-cell epitope. In some embodiments, the at least one CD8+ T-cell epitope is an HLA:A restricted antigen. In some embodiments, the at least one CD8+ T-cell epitope is an HLA:A01 restricted antigen. In some embodiments, the at least one CD8+ T-cell epitope is an HLA:A02 restricted antigen. In some embodiments, the at least one CD8+ T-cell epitope is an HLA:A03 restricted antigen. In some embodiments, the at least one CD8+ T-cell epitope is an HLA:A11 restricted antigen. In some embodiments, the at least one CD8+ T-cell epitope is an HLA:A24 restricted antigen. In some embodiments, the at least one CD8+ T-cell epitope is an HLA:A26 restricted antigen. In some embodiments, the at least one CD8+ T-cell epitope is an HLA:A29 restricted antigen. In some embodiments, the at least one CD8+ T-cell epitope is an HLA:A30 restricted antigen. In some embodiments, the at least one CD8+ T-cell epitope is an HLA:A31 restricted antigen. In some embodiments, the at least one CD8+ T-cell epitope is an HLA:A33 restricted antigen. In some embodiments, the at least one CD8+ T-cell epitope is an HLA:A68 restricted antigen.

In some embodiments, the PD-L1 binding molecule comprises at least one CD8+ T-cell epitope. In some embodiments, the at least one CD8+ T-cell epitope is an HLA:B restricted antigen. In some embodiments, the at least one CD8+ T-cell epitope is an HLA:B07 restricted antigen. In some embodiments, the at least one CD8+ T-cell epitope is an HLA:B08 restricted antigen. In some embodiments, the at least one CD8+ T-cell epitope is an HLA:B15 restricted antigen. In some embodiments, the at least one CD8+ T-cell epitope is an HLA:B35 restricted antigen. In some embodiments, the at least one CD8+ T-cell epitope is an HLA:B40 restricted antigen. In some embodiments, the at least one CD8+ T-cell epitope is an HLA:B44 restricted antigen. In some embodiments, the at least one CD8+ T-cell epitope is an HLA:B51 restricted antigen. In some embodiments, the at least one CD8+ T-cell epitope is an HLA:B52 restricted antigen. In some embodiments, the at least one CD8+ T-cell epitope is an HLA:B60 restricted antigen. In some embodiments, the at least one CD8+ T-cell epitope is an HLA:B65 restricted antigen.

In addition, multiple, immunogenic, T-cell epitopes for MHC class I presentation may be embedded in the same Shiga toxin effector polypeptide for use, such as, e.g., in the targeted delivery of a plurality of T-cell epitopes simultaneously.

In some embodiments, the PD-L1-binding molecule comprises at least one CD8+ T cell epitope that is embedded or inserted into the Shiga-like toxin A subunit effector polypeptide. In some embodiments, the at least one CD8+ T cell epitope is located on the C-terminus of the Shiga-like toxin A subunit effector polypeptide. In some embodiments, the at least one CD8+ T cell epitope is located on the N-terminus of the Shiga-like toxin A subunit effector polypeptide.

In some embodiments, the PD-L1-binding molecule comprises at least one CD8+ T cell epitope that is embedded or inserted into the binding region. In some embodiments, the at least one CD8+ T cell epitope is located on the C-terminus of the binding region. In some embodiments, the at least one CD8+ T cell epitope is located on the N-terminus of the binding region.

In some embodiments, the PD-L1-binding molecule comprises at least one CD8+ T cell epitope that is embedded or inserted in between the Shiga-like toxin A subunit effector polypeptide and binding region.

In some embodiments, the PD-L1-binding molecule comprises, in order from N-terminus to C-terminus the Shiga-like toxin A subunit effector polypeptide; the binding region; and the at least one CD8+ T-cell epitope. In some embodiments, the PD-L1-binding molecule comprises, in order from N-terminus to C-terminus, the Shiga-like toxin A subunit effector polypeptide; the binding region; and at least two CD8+ T-cell epitopes.

In some embodiments, the PD-L1-binding molecule comprises, in order from N-terminus to C-terminus the Shiga-like toxin A subunit effector polypeptide; the at least one CD8+ T-cell epitope; and the binding region.

In some embodiments, the PD-L1-binding molecule comprises, in order from N-terminus to C-terminus the Shiga-like toxin A subunit effector polypeptide; a first CD8+ T-cell epitope; the binding region; and a second CD8+ T-cell epitope. In some embodiments, the first and the second CD8+ T-cell epitopes are different.

In some embodiments, the PD-L1-binding molecule comprises, in order from N-terminus to C-terminus the Shiga-like toxin A subunit effector polypeptide; a first CD8+ T-cell epitope; the binding region; a second CD8+ T-cell epitope; and a third CD8+ T-cell epitope.

In some embodiments, at least one of the first, second, and third CD8+ T-cell epitopes is different from the others.

In some embodiments, the PD-L1-binding molecule comprises, in order from N-terminus to C-terminus, the binding region; the Shiga-like toxin A subunit effector polypeptide; and at least two CD8+ T-cell epitopes.

In some embodiments, the PD-L1-binding molecule comprises, in order from N-terminus to C-terminus the binding region; the at least one CD8+ T-cell epitope; and the Shiga-like toxin A subunit effector polypeptide.

In some embodiments, the PD-L1-binding molecule comprises, in order from N-terminus to C-terminus, the binding region; a first CD8+ T-cell epitope; the Shiga-like toxin A subunit effector polypeptide; and a second CD8+ T-cell epitope. In some embodiments, the first and second CD8+ T-cell epitopes are different.

In some embodiments, the PD-L1-binding molecule comprises, in order from N-terminus to C-terminus the binding region; a first CD8+ T-cell epitope; the Shiga-like toxin A subunit effector polypeptide; a second CD8+ T-cell epitope; and a third CD8+ T-cell epitope. In some embodiments, at least one of the first, second, and third CD8+ T-cell epitopes is different from the others.

In some embodiments, the PD-L1 binding molecule comprises at least one CD8+ T-cell epitopes. In some embodiments, the PD-L1 binding molecule comprises at least two CD8+ T-cell epitopes. In some embodiments, the PD-L1 binding molecule comprises at least three CD8+ T-cell epitopes. In some embodiments, the PD-L1 binding molecule comprises at least four CD8+ T-cell epitopes. In some embodiments, the PD-L1 binding molecule comprises at least five CD8+ T-cell epitopes. In some embodiments, the PD-L1 binding molecule comprises at least six CD8+ T-cell epitopes. In some embodiments, the PD-L1 binding molecule comprises at least seven CD8+ T-cell epitopes. In some embodiments, the PD-L1 binding molecule comprises at least eight CD8+ T-cell epitopes. In some embodiments, the PD-L1 binding molecule comprises at least nine CD8+ T-cell epitopes. In some embodiments, the PD-L1 binding molecule comprises at least ten CD8+ T-cell epitopes. In some embodiments, the PD-L1 binding molecule comprises at least one CD8+ T-cell epitopes that are each heterologous to Shiga-like toxin A subunits. In some embodiments, the PD-L1 binding molecule comprises at least two CD8+ T-cell epitopes that are each heterologous to Shiga-like toxin A subunits. In some embodiments, the PD-L1 binding molecule comprises at least three CD8+ T-cell epitopes that are each heterologous to Shiga-like toxin A subunits.

In some embodiments, the PD-L1 binding molecule comprises one to ten CD8+ T-cell epitopes. In some embodiments, the PD-L1 binding molecule comprises one to eight CD8+ T-cell epitopes. In some embodiments, the PD-L1 binding molecule comprises one to six CD8+ T-cell epitopes. In some embodiments, the PD-L1 binding molecule comprises one to four CD8+ T-cell epitopes. In some embodiments, the PD-L1 binding molecule comprises one to three CD8+ T-cell epitopes. In some embodiments, the PD-L1 binding molecule comprises one to two CD8+ T-cell epitopes.

In some embodiments, the PD-L1 binding molecule comprises one CD8+ T-cell epitope. In some embodiments, the PD-L1 binding molecule comprises two CD8+ T-cell epitopes. In some embodiments, the PD-L1 binding molecule comprises three CD8+ T-cell epitopes. In some embodiments, the PD-L1 binding molecule comprises four CD8+ T-cell epitopes. In some embodiments, the PD-L1 binding molecule comprises five CD8+ T-cell epitopes. In some embodiments, the PD-L1 binding molecule comprises six CD8+ T-cell epitopes. In some embodiments, the PD-L1 binding molecule comprises seven CD8+ T-cell epitopes. In some embodiments, the PD-L1 binding molecule comprises eight CD8+ T-cell epitopes. In some embodiments, the PD-L1 binding molecule comprises nine CD8+ T-cell epitopes. In some embodiments, the PD-L1 binding molecule comprises ten CD8+ T-cell epitopes. In some embodiments, the one, two, three, four, five, six, seven, eight, nine, or ten CD8+ T-cell epitopes are heterologous to Shiga-like A toxin A subunits.

In some embodiments, the PD-L1-binding molecule comprises multiple, immunogenic. T-cell epitopes for MHC class I presentation. In some embodiments, the Shiga toxin effector region of the PD-L1-binding molecule comprises multiple, immunogenic, T-cell epitopes for MHC class I presentation. In some embodiments, the PD-L1-binding molecule comprises at least one, at least two, at least three, at least four, at least five, or at least six T-cell epitopes for MHC class I presentation.

Any of the protease-cleavage resistant, Shiga toxin effector polypeptide sub-regions and/or disrupted furin-cleavage sites described herein may be used alone or in combination with each individual embodiment described herein, including methods described herein.

### C. Additional Exogenous Materials

In some embodiments, the binding molecules comprises an additional exogenous material. An “additional exogenous material” as used herein refers to one or more atoms or molecules that can be transported to the interior of a cell by a binding molecule. In some embodiments, an additional exogenous material is any material transported into the interior of a cell by a binding molecule, whether or not it is typically present in the native target cell or in a native Shiga toxin. In some embodiments, an additional exogenous material is a material that is not generally present in Shiga toxins and/or native target cells. In one sense, the entire binding molecule is an exogenous material which will enter the cell; thus, the “additional” exogenous materials are heterologous materials linked to but other than the core binding molecule itself. Non-limiting examples of additional exogenous materials are radionucleides, peptides, detection promoting agents, proteins, small molecule chemotherapeutic agents, and polynucleotides.

In some embodiments of the binding molecules, the additional exogenous material is one or more radionucleides, such as, e.g., ²¹¹At, ¹³¹I, ¹²⁵I, ⁹⁰Y, ¹¹¹In, ¹⁸⁶Re, ¹⁸⁸Re, ¹⁵³Sm, ²¹²Bi, ³²P, ⁶⁰C, and/or radioactive isotopes of lutetium.

In some embodiments, the additional exogenous material comprises a proapoptotic peptide, polypeptide, or protein, such as, e.g., BCL-2, caspases (e.g. fragments of caspase-3 or caspase-6), cytochromes, granzyme B, apoptosis-inducing factor (AIF), BAX, tBid (truncated Bid), and proapoptotic fragments or derivatives thereof (see e.g., Ellerby H et al.. Nat Med 5: 1032-8 (1999); Mai J et al., Cancer Res 61: 7709-12 (2001); Jia L et al., Cancer Res 63: 3257-62 (2003); Liu Y et al., Mol Cancer Ther 2: 1341-50 (2003); Perea S et al., Cancer Res 64: 7127-9 (2004); Xu Y et al., J Immunol 173: 61-7 (2004); Dálken B et al., Cell Death Differ 13: 576-85 (2006); Wang T et al., Cancer Res 67: 11830-9 (2007); Kwon M et al., Mol Cancer Ther 7: 1514-22 (2008); Qiu X et al., Mol Cancer Ther 7: 1890-9 (2008); Shan L et al., Cancer Biol Ther 11: 1717-22 (2008); Wang F et al., Clin Cancer Res 16: 2284-94 (2010); Kim J et al., J Virol 85: 1507-16 (2011)).

In some embodiments, the additional exogenous material comprises a protein or polypeptide comprising an enzyme. In certain other embodiments, the additional exogenous material is a nucleic acid, such as, e.g. a ribonucleic acid that functions as a small inhibiting RNA (siRNA) or microRNA (miRNA). In some embodiments, the additional exogenous material is an antigen, such as antigens derived from pathogens, bacterial proteins, viral proteins, proteins mutated in cancer, proteins aberrantly expressed in cancer, or T-cell complementary determining regions. For example, exogenous materials include antigens, such as those characteristic of antigen-presenting cells infected by bacteria, and T-cell complementary determining regions capable of functioning as exogenous antigens. Exogenous materials comprising polypeptides or proteins may optionally comprise one or more antigens whether known or unknown to the skilled worker.

In some embodiments of the binding molecules, all heterologous antigens and/or epitopes associated with the Shiga toxin effector polypeptide are arranged in the binding molecule amino-terminal to the carboxy-terminus of the Shiga toxin A1 fragment region of the Shiga toxin effector polypeptide. In some embodiments, all heterologous antigens and/or epitopes associated with the Shiga toxin effector polypeptide are associated, either directly or indirectly, with the Shiga toxin effector polypeptide at a position amino-terminal to the carboxy-terminus of the Shiga toxin A1 fragment region of the Shiga toxin effector poly peptide. In some embodiments, all additional exogenous material(s) which is an antigen is arranged amino-terminal to the Shiga toxin effector polypeptide, such as, e.g., fused directly or indirectly to the amino terminus of the Shiga toxin effector polypeptide.

In some embodiments of the binding molecules, the additional exogenous material is a cytotoxic agent, such as, e.g., a small molecule chemotherapeutic agent, anti-neoplastic agent, cytotoxic antibiotic, alkylating agent, antimetabolite, topoisomerase inhibitor, and/or tubulin inhibitor. Non-limiting examples of cytotoxic agents suitable for use with as described herein include aziridines, cisplatins, tetrazines, procarbahne, hexamethylmelamine, vinca alkaloids, taxanes, camptothecins, etoposide, doxorubicin, mitoxantrone, teniposide, novobiocin, aclarubicin, anthracyclines, actinomycin, amanitin, amatoxins, bleomycin, centanamycin (indolecarboxamide), plicamycin, mitomycin, daunorubicin, epirubicin, idarubicins, dolastatins, maytansines, maytansionoids, duromycin, docetaxel, duocarmycins, adriamycin, calicheamicin, auristatins, pyrrolobenzodiazepines, pyrrolobenzodiazepine dimers (PBDs), carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, gemcitabine, aceglatone, acetogenins (e.g. bullatacin and bullatacinone), aclacinomysins, AG1478, AG1571, aldophosphamide glycoside, alkyl sulfonates (e.g., busulfan, improsulfan, and piposulfan), alkylating agents (e.g. thiotepa and cyclosphosphamide), aminolevulinic acid, aminopterin, amsacrine, ancitabine, anthramycin, arabinoside, azacitidine, azaserine, aziridines (e.g., benzodopa, carboquone, meturedopa, and uredopa), azauridine, bestrabucil, bisantrene, bisphosphonates (e.g. clodronate), bleomycins, bortezomib, bryostatin, cactinomycin, callystatin, carabicin, carminomycin, carmofur, carmustine, carzinophilin. CC-1065, chlorambucil, chloranbucil, chlomaphazine, chlorozotocin, chromomycinis, chromoprotein enediyne antibiotic chromophores. CPT-11, cryptophycins (e.g. cryptophycin 1 and cryptophycin 8), cyclophosphamide, cytarabine, dacarbazine, dactinomycin, daunomycin, defofamine, demecolcine, detorubicin, diaziquone, 6-diazo-5-oxo-L-norleucine, dideoxyuridine, difluoromethylomithine (DMFO), doxifluridine, doxorubicins (e.g., morpholinodoxorubicin, cyanomorpholino-doxorubicin, 2-pyrrolinodoxorubicin, and deoxydoxorubicin), dynemicins, edatraxate, edatrexate, eleutherobins, elformithine, elliptinium acetate, enediyne antibiotics (e.g. calicheamicins), eniluracil, enocitabine, epirubicins, epothilone, esorubicins, esperamicins, estramustine, ethylenimines, 2-ethylhydrazide, etoglucid, fludarabine, folic acid analogues (e.g., denopterin, methotrexate, pteropterin, and trimetrexate), folic acid replenishers (e.g. frolinic acid), fotemustine, fulvestrant, gacytosine, gallium nitrate, gefitinib, gemcitabine, hydroxyurea, ibandronate, ifosfamide, imatinib mesylate, erlotinib, fulvestrant, letrozole. PTK787/ZK 222584 (Novartis. Basel, CH), oxaliplatin, leucovorin, rapamycin, lapatinib, lonafamib, sorafenib, methylamelamines (e.g., altretamine, triethylenemelamine, triethylenephosphoramide, triethylenethiophosphoramide and trimethylomelamine), pancratistatins, sarcodictyins, spongistatins, nitrogen mustards (e.g., chlorambucil, chlomaphazine, cyclophosphamide, mechlorethamine, mechlorethamine oxide hydrochloride, melphalan, novembichin, phenesterine, prednimustine, trofosfamide, and uracil mustard), nitrosureas (e.g., carmustine, fotemustine, lomustine, nimustine, and ranimnustine), dynemicins, neocarzinostatin chromophores, anthramycin, detorubicin, epirubicins, marcellomycins, mitomycins (e.g. mitomycin C), mycophenolic acid, nogalamycins, olivomycins, peplomycins, potfiromycins, puromycins, quelamycins, rodorubicins, ubenimex, zinostatins, zorubicins, purine analogs (e.g., fludarabine, 6-mercaptopurine, thiamiprine, and thioguanine), pyrimidine analogs (e.g., ancitabine, azacitidine, 6-azauridine, dideoxyuridine, doxifluridine, enocitabine, and floxuridine), aceglatone, lentinan, lonidainine, maytansinoids (e.g. maytansins and ansamitocins), mitoguazone, mitoxantrone, mopidanmol, nitraerine, pentostatin, phenamet, pirarubicin, podophyllinic acid, 2-ethylhydrazide, rhizoxin, sizofuran, spirogermanium, tenuazonic acid, triaziquone, 2,2′,2″trichlorotriethylamine, trichothecenes (e.g., T-2 toxin, verracurin A, roridin A, and anguidine), urethan, vindesine, mannomustine, mitobronitol, mitolactol, pipobroman, arabinoside, cyclophosphamide, toxoids (e.g. paclitaxel and doxetaxel), 6-thioguanine, mercaptopurine, platinum, platinum analogs (e.g. cisplatin and carboplatin), etoposide (VP-16), mitoxantrone, vinorelbine, novantrone, daunomycin, xeloda, topoisomerase inhibitor RFS 2000, retinoids (e.g. retinoic acid), capecitabine, lomustine, losoxantrone, mercaptopurines, nimustine, nitraerine, rapamycin, razoxane, roridin A, spongistatins, streptonigrins, streptozocins, sutent, T-2 toxin, thiamiprine, thiotepa, toxoids (e.g. paclitaxel and doxetaxel), tubercidins, verracurin A, vinblastine, vincristine, and structural analogs of any of the aforementioned (e.g. synthetic analogs), and/or derivatives of any of the aforementioned (see e.g., Lindell T et al., Science 170: 447-9 (1970); Remillard S et al., Science 189: 1002-5 (1975); Ravry M et al., Am J Clin Oncol 8: 148-50 (1985); Ravry M et al., Cancer Treat Rep 69: 1457-8 (1985); Stemberg C et al., Cancer 64: 2448-58 (1989); Bai R et al., Biochem Pharmacol 39: 1941-9 (1990); Boger D, Johnson D, Proc Natl Acad Sci USA 92: 3642-9 (1995); Beck J et al., Leuk Lymphoma 41: 117-24 (2001); Cassady J et al., Chem Pharm Bull (Tokyo) 52: 1-26 (2004); Sapra P et al., Clin Cancer Res 11: 5257-64 (2005); Okeley N et al., Clinc Cancer Res 16: 888-97 (2010); Oroudjev E et al., Mol Cancer Ther 9: 2700-13 (2010); Ellestad G, Chirality 23: 660-71 (2011); Kantarjian H et al., Lancet Oncol 13: 403-11 (2012); Moldenhauer G et al., J Natl Cancer Inst 104: 622-34 (2012); Meulendijks D et al., Invest New Drugs 34: 119-28 (2016)).

A non-limiting list of illustrative carboxy-terminal exogenous materials are provided below in Table 2. These carboxy-terminal exogenous materials may, for example, be delivered into a target cell by a binding molecule.

TABLE 2
Illustrative Carboxy-Terminal Moieties

| Sequence | SEQ ID NO |
|---|---|
| HHAANLVPMVATV | 176 |
| HHAANLVPMVATVRRNLVPMVATVRRNLVP | 177 |
| NLVPMVATVRRNLVPMVATVRRNLVPMVATV | 175 |
| NLVPMVATVRRNLVPMVATVRRNLVP | 174 |
| NLVPMVATVRRMLVPMVATV | 178 |
| NLVPMVATVHHAANLVPMVATV | 179 |
| RRNLVPMVATV | 180 |
| RRNLVPMVATVRRNLVPMVATVRRNLVP | 181 |
| NLVPMVATVRRNLVPMVATVHHAANLVPMVATV | 182 |
| NLVPMVATVRRAANLVPMVATVHHAANLVP | 183 |
| NLVPMVATVHHAANLVPMVATVRRNLVPMVATV | 184 |
| NLVPMVATVHHAANLVPMVATVRRNLVP | 185 |
| NLVPMVATVHHAANLVPMVATVHHAANLVPMVATV | 186 |
| NLVPMVATVHHAANLVPMVATVHHAANLVP | 187 |

In some embodiments, a binding molecule comprises a Shiga toxin effector polypeptide and a carboxy-terminal moiety, such as a carboxy terminal moiety comprising the sequence of any one of SEQ ID NOs: 174-187. In some embodiments, a binding molecule comprises a Shiga toxin effector polypeptide and a carboxy terminal moiety, wherein the Shiga toxin effector polypeptide comprises the sequence of any one of SEQ ID NOs: 1-18, 40-68, 169, 170, or 173. In some embodiments, a binding molecule comprises a Shiga toxin effector polypeptide and a carboxy terminal moiety, wherein the Shiga toxin effector polypeptide comprises the sequence of SEQ ID NO: 41. In some embodiments, a binding molecule comprises a Shiga toxin effector polypeptide and a carboxy terminal moiety, wherein the Shiga toxin effector polypeptide comprises the sequence of SEQ ID NO: 41, and the carboxy terminal moiety comprises the sequence of any one of SEQ ID NOs: 174-178.

II. Linkages Connecting Components and/or their Subcomponents

Individual PD-L1 binding regions, toxin components, and/or other components of the binding molecules described herein may be suitably linked to each, such as, e.g., fused directly or indirectly linked to each other via one or more linkers well known in the art and/or described herein. Individual polypeptide subcomponents of the binding regions, e.g. heavy chain variable regions (V_{H}), light chain variable regions (V_{L}), CDR, and/or ABR regions, may be suitably linked to each other via one or more linkers (e.g., scFv linkers) well known m the art and/or described herein, including via chemical conjugation. Proteinaceous components, e.g., multi-chain binding regions, may be suitably linked to each other or other polypeptide components directly via peptide bonds and/or indirectly via one or more linkers well known in the art. Peptide components, e.g., KDEL family endoplasmic reticulum retention/retrieval signal motifs (see SEQ ID NOs: 205-252), may be suitably linked to another component directly via peptide bonds or indirectly via one or more linkers, such as a proteinaceous linker, which are well known in the art. For example, in some embodiments of the binding molecule, an individual PD-L1 binding region and a Shiga toxin effector polypeptide (and/or additional components of the binding molecule, such as, e.g., a T-cell epitope, additional exogenous material, and/or KDEL motif) are suitably linked and/or conjugated to each other via one or more binding region linkers well known in the art and/or described herein.

Suitable linkers are generally those which allow each polypeptide component to fold with a three-dimensional structure very similar to the polypeptide components produced individually without any linker or other component. Suitable linkers include single amino acids, peptides, polypeptides, and linkers lacking any of the aforementioned, such as various non-proteinaceous carbon chains, whether branched or cyclic.

Suitable linkers may be proteinaceous and comprise one or more amino acids, peptides, and/or polypeptides. Proteinaceous linkers are suitable for both recombinant fusion proteins and chemically linked conjugates. A proteinaceous linker typically has from about 2 to about 50 amino acid residues, such as, e.g., from about 5 to about 30 or from about 6 to about 25 amino acid residues. The length of the linker selected will depend upon a variety of factors, such as, e.g., the desired property or properties for which the linker is being selected. In some embodiments, the linker is proteinaceous and is linked near the terminus of a protein component, typically within about 20 amino acids of the terminus.

Suitable linkers may be non-proteinaceous, such as, e.g. chemical linkers. Various non-proteinaceous linkers known in the art may be used to link cell-targeting binding regions to the Shiga toxin effector polypeptide components of the binding molecules, such as linkers commonly used to conjugate immunoglobulin polypeptides to heterologous polypeptides. For example, polypeptide regions may be linked using the functional side chains of their amino acid residues and carbohydrate moieties such as, e.g., a carboxy, amine, sulfhydryl, carboxylic acid, carbonyl, hydroxyl, and/or cyclic ring group. For example, disulfide bonds and thioether bonds may be used to link two or more polypeptides. In addition, non-natural amino acid residues may be used with other functional side chains, such as ketone groups (see e.g. Axup J et al., Proc Natl Acad Sci U.S.A. 109:16101-6 (2012); Sun S et al., Chembiochem Jul. 18 (2014); Tian F et al., Proc Natl Acad Sci USA 111: 1766-71 (2014)). In addition, non-natural amino acid residues may be used with other functional side chains, such as ketone groups, alkyne groups, or azides (see e.g. the use of antibodies engineered to comprise p-acetyl-L-phenylalanine or p-azidomethyl-N-phenylalanine residues for conjugation to cargos U.S. patent application Publication Ser. No. 14/786,402 US 2017/0362334)). Examples of non-proteinaceous chemical linkers include but are not limited to N-hydroxysuccinimide esters (NHS esters) such as sulfo-NHS esters, isothiocyanates, isocyanates, acyl azides, sulfonyl chlorides, aldehydes, glyoxals, epoxides, oxiranes, carbonates, aryl halides, imidoesters, carbodiimides, anhydrides, and fluorophenyl esters. Further examples of non-proteinaceous chemical linkers include but are not limited to N-succinimidyl (4-iodoacetyl)-aminobenzoate, S—(N-succinimidyl) thioacetate (SATA), N-succinimidyl-oxycarbonyl-cu-methyl-a-(2-pyridyldithio) toluene (SMPT), N-succinimidyl 4-(2-pyridyldithio)-pentanoate (SPP), succinimidyl 4-(N-maleimidomethyl) cyclohexane carboxylate (SMCC or MCC), sulfosuccinimidyl (4-iodoacetyl)-aminobenzoate, 4-succinimidyl-oxycarbonyl-a-(2-pyridyldithio) toluene, sulfosuccinimidyl-6-(α-methyl-α-(pyridyldithiol)-toluamido) hexanoate, N-succinimidyl-3-(-2-pyridyldithio)-proprionate (SPDP), succinimidyl 6(3(-(-2-pyridyldithio)-proprionamido) hexanoate, sulfosuccinimidyl 6(3(-(-2-pyridyldithio)-propionamido) hexanoate, maleimidocaproyl (MC), maleimidocaproyl-valine-citrulline-p-aminobenzyloxycarbonyl (MC-vc-PAB), 3-maleimidobenzoic acid N-hydroxysuccinimide ester (MBS), alpha-alkyl derivatives, sulfoNHS-ATMBA (sulfosuccinimidyl N-[3-(acetylthio)-3-methylbutyryl-beta-alanine]), sulfodichlorophenol, 2-iminothiolane, 3-(2-pyridyldithio)-propionyl hydrazide, Ellman's reagent, dichlorotriazinic acid, and S-(2-thiopyridyl)-L-cysteine.

Suitable linkers, whether proteinaceous or non-proteinaceous, may include, e.g., protease sensitive, environmental redox potential sensitive, pH sensitive, acid cleavable, photocleavable, and/or heat sensitive linkers.

Proteinaceous linkers may be chosen for incorporation into recombinant fusion binding molecules. For recombinant fusion cell-targeting proteins, linkers typically comprise about 2 to 50 amino acid residues, preferably about 5 to 30 amino acid residues. Commonly, proteinaceous linkers comprise a majority of amino acid residues with polar, uncharged, and/or charged residues, such as, e.g., threonine, proline, glutamine, glycine, and alanine. Non-limiting examples of proteinaceous linkers include alanine-serine-glycine-glycine-proline-glutamate (ASGGPE), valine-methionine (VM), alanine-methionine (AM), AM(G_{2 to 4}S)ₓAM where G is glycine. S is serine, and x is an integer from 1 to 10.

Proteinaceous linkers may be selected based upon the properties desired. Proteinaceous linkers may be chosen by the skilled worker with specific features in mind, such as to optimize one or more of the fusion molecule's folding, stability, expression, solubility, pharmacokinetic properties, pharmacodynamic properties, and/or the activity of the fused domains in the context of a fusion construct as compared to the activity of the same domain by itself. For example, proteinaceous linkers may be selected based on flexibility, rigidity, and/or cleavability. The skilled worker may use databases and linker design software tools when choosing linkers. In certain linkers may be chosen to optimize expression. In certain linkers may be chosen to promote intermolecular interactions between identical polypeptides or proteins to form homomultimers or different polypeptides or proteins to form heteromultimers. For example, proteinaceous linkers may be selected which allow for desired non-covalent interactions between polypeptide components of the binding molecules, such as, e.g., interactions related to the formation dimers and other higher order multimers.

Flexible proteinaceous linkers are often greater than 12 amino acid residues long and rich in small, non-polar amino acid residues, polar amino acid residues, and/or hydrophilic amino acid residues, such as, e.g., glycines, serines, and threonines. Flexible proteinaceous linkers may be chosen to increase the spatial separation between components and/or to allow for intramolecular interactions between components. For example, various “GS” linkers are known to the skilled worker and are composed of multiple glycines and/or one or more serines, sometimes in repeating units, such as, e.g., (GₓS)ₙ, (SₓG)ₙ, (GGGGS)ₙ, and (G)ₙ, in which x is 1 to 6 and n is 1 to 30 (SEQ ID NOs. 262-264, 266). Non-limiting examples of flexible proteinaceous linkers include GKSSGSGSESKS (SEQ ID NO: 188), EGKSSGSGSESKEF (SEQ ID NO: 189), GSTSGSGKSSEGKG (SEQ ID NO: 190), GSTSGSGKSSEGSGSTKG (SEQ ID NO: 191), GSTSGSGKPGSGEGSTKG (SEQ ID NO: 192), SRSSG (SEQ ID NO: 193), and SGSSC (SEQ ID NO: 194).

Rigid proteinaceous linkers are often stiff alpha-helical structures and rich in proline residues and/or one or more strategically placed prolines. Rigid linkers may be chosen to prevent intramolecular interactions between linked components.

Suitable linkers may be chosen to allow for in vivo separation of components, such as, e.g., due to cleavage and/or environment-specific instability. In vivo cleavable proteinaceous linkers are capable of unlinking by proteolytic processing and/or reducing environments often at a specific site within an organism or inside a certain cell type. In vivo cleavable proteinaceous linkers often comprise protease sensitive motifs and/or disulfide bonds formed by one or more cysteine pairs. In vivo cleavable proteinaceous linkers may be designed to be sensitive to proteases that exist only at certain locations in an organism, compartments within a cell, and/or become active only under certain physiological or pathological conditions (such as, e.g., involving proteases with abnormally high levels, proteases overexpressed at certain disease sites, and proteases specifically expressed by a pathogenic microorganism). For example, there are proteinaceous linkers known in the art which are cleaved by proteases present only intracellularly, proteases present only within specific cell types, and proteases present only under pathological conditions like cancer or inflammation, such as, e.g., R-x-x-R and AMGRSGGGCAGNRVGSSLSCGGLNLQAM (SEQ ID NO: 195).

In some embodiments of the binding molecules, a linker may be used which comprises one or more protease sensitive sites to provide for cleavage by a protease present within a target cell. In some embodiments, a linker may be used which is not cleavable to reduce unwanted toxicity after administration to a vertebrate organism.

Suitable linkers may include, e.g., protease sensitive, environmental redox potential sensitive, pH sensitive, acid cleavable, photocleavable, and/or heat sensitive linkers, whether proteinaceous or non-proteinaceous (see e.g., Doronina S et al., Bioconjug Chem 17: 114-24 (2003); Saito G et al., Adv Drug Deliv Rev 55: 199-215 (2003); Jeffrey S et al., J Med Chem 48: 1344-58 (2005); Sanderson R et al., Clin Cancer Res 11: 843-52 (2005); Erickson H et al., Cancer Res 66: 4426-33 (2006); Chen X et al., Adv Drug Deliv Rev 65: 1357-69 (2013)). Suitable cleavable linkers may include linkers comprising cleavable groups which are known in the art.

Suitable linkers may include pH sensitive linkers. For example, certain suitable linkers may be chosen for their instability in lower pH environments to provide for dissociation inside a subcellular compartment of a target cell (see e.g., van Der Velden V et al., Blood 97: 3197-204 (2001); Ulbrich K, Subr V, Adv Drug Deliv Rev 56: 1023-50 (2004)). For example, linkers that comprise one or more trityl groups, derivatized trityl groups, bismaleimideothoxy propane groups, adipic acid dihydrazide groups, and/or acid labile transferrin groups, may provide for release of components of the binding molecules, e.g. a polypeptide component, in environments with specific pH ranges. In certain linkers may be chosen which are cleaved in pH ranges corresponding to physiological pH differences between tissues, such as, e.g., the pH of tumor tissue is lower than in healthy tissues.

Photocleavable linkers are linkers that are cleaved upon exposure to electromagnetic radiation of certain wavelength ranges, such as light in the visible range. Photocleavable linkers may be used to release a component of a binding molecule, e.g. a polypeptide component, upon exposure to light of certain wavelengths. Non-limiting examples of photocleavable linkers include a nitrobenzyl group as a photocleavable protective group for cysteine, nitrobenzyloxycarbonyl chloride cross-linkers, hydroxypropylmethacrylamide copolymer, glycine copolymer, fluorescein copolymer, and methylrhodamine copolymer. Photocleavable linkers may have particular uses in linking components to form binding molecules designed for treating diseases, disorders, and conditions that can be exposed to light using fiber optics.

In some embodiments of the binding molecules, a PD-L1 binding region is linked to a Shiga toxin effector polypeptide using any number of means known to the skilled worker, including both covalent and noncovalent linkages.

In some embodiments of the binding molecules, the molecule comprises a binding region which is a scFv with a linker (i.e., a scFv linker) connecting a heavy chain variable (V_{II}) domain and a light chain variable (V_{L}) domain. There are numerous linkers known in the art suitable for this purpose, such as, e.g., the 15-residue (Gly₄Ser)₃ peptide. Suitable scFv linkers which may be used in forming non-covalent multivalent structures include GGS, GGGS (SEQ ID NO: 196), GGGGS (SEQ ID NO: 72), GGGGSGGG (SEQ ID NO: 197), GGSGGGG (SEQ ID NO: 198), GSTSGGGSGGGSGGGGSS (SEQ ID NO: 199), and GSTSGSGKPGSSEGSTKG (SEQ ID NO: 200).

Suitable methods for linkage of the components of the binding molecules may be by any method presently known in the art for accomplishing such, so long as the attachment does not substantially impede the binding capability of the cell-targeting binding region, the cellular internalization of the Shiga toxin effector polypeptide component, and/or when appropriate the desired Shiga toxin effector function(s) as measured by an appropriate assay, including assays described herein.

The components of the binding molecule, e.g. a Shiga toxin A Subunit effector polypeptide and/or immunoglobulin-type PD-L1-binding region, may be linked via a binding region linker. In some embodiments, the components may be engineered to provide a suitable attachment moiety for the linkage of additional components, e.g. an additional exogenous material (see WO 2018/106895).

For the purposes of the binding molecules, the specific order or orientation is not fixed for the components: the Shiga toxin effector polypeptide(s), the binding region(s), and any optional linker(s), in relation to each other or the entire binding molecule unless specifically noted. The components of the binding molecules may be arranged in any order provided that the desired activity(ies) of the binding region and Shiga toxin effector polypeptide are not eliminated.

### III. Examples of Structural Variations of the Binding Molecules

In some embodiments, a Shiga toxin effector polypeptide of the binding molecule comprises or consists essentially of a truncated Shiga toxin A Subunit. Truncations of Shiga toxin A Subunits might result in the deletion of an entire epitope(s) and/or epitope region(s), B-cell epitopes, CD4+ T-cell epitopes, and/or furin-cleavage sites without affecting Shiga toxin effector functions, such as, e.g., catalytic activity and cytotoxicity. The smallest Shiga toxin A Subunit fragment shown to exhibit full enzymatic activity was a polypeptide composed of residues 1-239 of Slt1A (LaPointe P et al., J Biol Chem 280: 23310-18 (2005)). The smallest Shiga toxin A Subunit fragment shown to exhibit significant enzymatic activity was a polypeptide composed of residues 75-247 of StxA (Al-Jaufy A et al., Infect Immun 62: 956-60 (1994)).

Although Shiga toxin effector polypeptides may commonly be smaller than the full-length Shiga toxin A Subunit, it is preferred that the Shiga toxin effector polypeptide region of a binding molecule maintain the polypeptide region from amino acid position 77 to 239 (SLT-1A (SEQ ID NO:1) or StxA (SEQ ID NO:2)) or the equivalent in other A Subunits of members of the Shiga toxin family (e.g. 77 to 238 of (SEQ ID NO:3)). For example, in some embodiments, the Shiga toxin effector polypeptide derived from SLT-1A may comprise or consist essentially of amino acids 75 to 251 of SEQ ID NO:1, 1 to 241 of SEQ ID NO:1, 1 to 251 of SEQ ID NO:1, or amino acids 1 to 261 of SEQ ID NO:1, wherein relative to a wild-type Shiga toxin A Subunit at least one amino acid residue is mutated or has been deleted in an endogenous epitope and/or epitope region, and/or wherein there is a disrupted, furin-cleavage site region at the carboxy-terminus of a Shiga toxin A1 fragment derived region. Similarly, Shiga toxin effector polypeptide regions derived from StxA may comprise or consist essentially of amino acids 75 to 251 of SEQ ID NO:2, 1 to 241 of SEQ ID NO:2, 1 to 251 of SEQ ID NO:2, or amino acids 1 to 261 of SEQ ID NO:2, wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region. Additionally, Shiga toxin effector polypeptide regions derived from SLT-2 may comprise or consist essentially of amino acids 75 to 251 of SEQ ID NO:3, 1 to 241 of SEQ ID NO:3, 1 to 251 of SEQ ID NO:3, or amino acids 1 to 261 of SEQ ID NO:3, wherein relative to a wild-type Shiga toxin A Subunit at least one amino acid residue is mutated or has been deleted in an endogenous epitope and/or epitope region, and/or wherein there is a disrupted, furin-cleavage site region at the carboxy-terminus of a Shiga toxin A1 fragment derived region.

Also provided herein are variants of Shiga toxin effector polypeptides and binding molecules, wherein the Shiga toxin effector polypeptide differs from a naturally occurring Shiga toxin A Subunit by only or up to 1, 2, 3, 4, 5, 6, 7, 8, 9, 10, 15, 20, 25, 30, 35, 40 or more amino acid residues (but by no more than that which retains at least 85%, 90%, 95%, 99% or more amino acid sequence identity). Thus, a molecule derived from an A Subunit of a member of the Shiga toxin family may comprise additions, deletions, truncations, or other alterations from the original sequence as long as at least 85%, 90%, 95%, 99% or more amino acid sequence identity is maintained to a naturally occurring Shiga toxin A Subunit and wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region.

Accordingly, in some embodiments, the Shiga toxin effector polypeptide of a molecule described herein comprises or consists essentially of amino acid sequences having at least 55%, 60%, 65%, 70%, 75%, 80%, 85%, 90%, 95%, 97%, 98%, 99%, 99.5% or 99.7% overall sequence identity to a naturally occurring Shiga toxin A Subunit, such as SLT-1A (SEQ ID NO:1), StxA (SEQ ID NO:2), and/or SLT-2A (SEQ ID NO:3) wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region.

Optionally, either a full-length or a truncated version of the Shiga toxin A Subunit may comprise the Shiga toxin effector polypeptide region of a molecule of the present, wherein the Shiga toxin derived polypeptide comprises one or more mutations (e.g. substitutions, deletions, insertions, or inversions) as compared to a naturally occurring Shiga toxin. It is preferred in some embodiments that the Shiga toxin effector polypeptides have sufficient sequence identity to a naturally occurring Shiga toxin A Subunit to retain cytotoxicity after entry into a cell, either by well-known methods of host cell transformation, transfection, infection or induction, or by internalization mediated by a cell-targeting binding region linked with the Shiga toxin effector polypeptide. The most critical residues for enzymatic activity and/or cytotoxicity in the Shiga toxin A Subunits have been mapped to the following residue-positions: asparagine-75, tyrosine-77, glutamate-167, arginine-170, and arginine-176 among others (Di R et al., Toxicon 57: 525-39 (2011)). In any one of the embodiments described herein, the Shiga toxin effector polypeptides may preferably but not necessarily maintain one or more conserved amino acids at positions, such as those found at positions 77, 167, 170, and 176 in StxA. SLT-1A, or the equivalent conserved position in other members of the Shiga toxin family which are typically required for cytotoxic activity. The capacity of a cytotoxic molecule to cause cell death, e.g. its cytotoxicity, may be measured using any one or more of a number of assays well known in the art.

### A. Examples of De-Immunized, Shiga Toxin Effector Polypeptides

In some embodiments, the de-immunized, Shiga toxin effector polypeptide of the binding molecule may consist essentially of a truncated Shiga toxin A Subunit having two or more mutations. Truncations of Shiga toxin A Subunits might result in the deletion of an entire epitope(s) and/or epitope region(s), B-cell epitopes, CD4+ T-cell epitopes, and/or furin-cleavage sites without affecting Shiga toxin effector functions, such as, e.g., catalytic activity and cytotoxicity. Truncating the carboxy-terminus of SLT-1A, StxA, or SLT-2A to amino acids 1-251 removes two predicted B-cell epitope regions, two predicted CD4 positive (CD4+) T-cell epitopes, and a predicted discontinuous B-cell epitope. Truncating the amino-terminus of SLT-1A, StxA, or SLT-2A to 75-293 removes at least three predicted B-cell epitope regions and three predicted CD4+ T-cell epitopes. Truncating both amino- and carboxy-terminals of SLT-1A, StxA, or SLT-2A to 75-251 deletes at least five predicted B-cell epitope regions, four putative CD4+ T-cell epitopes and one predicted discontinuous B-cell epitope.

In some embodiments, a de-immunized, Shiga toxin effector polypeptide may comprise or consist essentially of a full-length or truncated Shiga toxin A Subunit with at least one mutation (relative to a wild-type Shiga toxin polypeptide), e.g. deletion, insertion, inversion, or substitution, in a provided, endogenous, B-cell and/or CD4+ T-cell epitope region. In some embodiments, the Shiga toxin effector polypeptide comprises a disruption which comprises a mutation (relative to a wild-type Shiga toxin polypeptide) which includes a deletion of at least one amino acid residue within the endogenous, B-cell and/or CD4+ T-cell epitope region. In some embodiments, the Shiga toxin effector polypeptide comprises a disruption which comprises an insertion of at least one amino acid residue within the endogenous, B-cell and/or CD4+ T-cell epitope region. In some embodiments, the Shiga toxin effector polypeptide comprises a disruption which comprises an inversion of amino acid residues, wherein at least one inverted amino acid residue is within the endogenous, B-cell and/or CD4+ T-cell epitope region. In some embodiments, the Shiga toxin effector polypeptide comprises a disruption which comprises a mutation (relative to a wild-type Shiga toxin polypeptide), such as, e.g., an amino acid substitution, an amino acid substitution to a non-standard amino acid, and/or an amino acid residue with a chemically modified side chain. Non-limiting examples of de-immunized, Shiga toxin effector sub-regions suitable for use as described herein are described in WO 2015/113005, WO 2015/113007, WO 2015/191764. WO 2016/196344, and WO 2018/140427.

In other embodiments, the de-immunized, Shiga toxin effector polypeptide comprises a truncated Shiga toxin A Subunit which is shorter than a full-length Shiga toxin A Subunit wherein at least one amino acid residue is disrupted in a natively positioned, B-cell and/or CD4+ T-cell epitope region.

To create a de-immunized, Shiga toxin effector poly peptide, in principle modifying any amino acid residue in a provided epitope region by various means can result in a disruption of an epitope, such as, e.g., a modification which represents a deletion, insertion, inversion, rearrangement, substitution, and chemical modification of a side chain relative to a wild-type Shiga toxin polypeptide. However, modifying certain amino acid residues and using certain amino acid modifications are more likely to successfully reduce antigenicity and/or immunogenicity while maintaining a certain level of a Shiga toxin effector function(s). For example, terminal truncations and internal amino acid substitutions are preferred because these types of modifications maintain the overall spacing of the amino acid residues in a Shiga toxin effector polypeptide and thus are more likely to maintain Shiga toxin effector polypeptide structure and function.

In some embodiments, the de-immunized, Shiga toxin effector polypeptide comprising or consisting essentially of amino acids 75 to 251 of SLT-1A (SEQ ID NO:1), StxA (SEQ ID NO:2), and/or SLT-2A (SEQ ID NO:3) wherein at least one amino acid residue is disrupted in a natively positioned, epitope region. Among certain other embodiments are de-immunized, Shiga toxin effector polypeptides which comprise or consist essentially of amino acids 1 to 241 of SLT-1A (SEQ ID NO:1), StxA (SEQ ID NO:2), and/or SLT-2A (SEQ ID NO:3) wherein at least one amino acid residue is disrupted in a natively positioned, epitope region. Further embodiments are de-immunized, Shiga toxin effector polypeptides which comprise or consist essentially of amino acids 1 to 251 of SLT-1A (SEQ ID NO:1), StxA (SEQ ID NO:2), and/or SLT-2A (SEQ ID NO:3) wherein at least one amino acid residue is disrupted in a natively positioned, epitope region provided. Further embodiments are Shiga toxin effector polypeptides comprising amino acids 1 to 261 of SLT-1A (SEQ ID NO:1), StxA (SEQ ID NO:2), and/or SLT-2A (SEQ ID NO:3) wherein at least one amino acid residue is disrupted in a natively positioned, epitope region.

There are numerous, diverse, internal amino acid substitutions that can be used to create de-immunized, Shiga toxin effector polypeptides. Of the possible substitute amino acids to use within an epitope region, the following substitute amino acid residues are predicted to be the most likely to reduce the antigenicity and/or immunogenicity of an epitope—G, D, E, S, T, R, K, and H. Except for glycine, these amino acid residues may all be classified as polar and/or charged residues. Of the possible amino acids to substitute with, the following amino acids A, G, V, L, I, P, C, M, F, S, D, N, Q, H, and K are predicted to be the most likely to reduce antigenicity and/or immunogenicity while providing the retention of a significant level of a Shiga toxin effector function(s), depending on the amino acid substituted for. Generally, the substitution should change a polar and/or charged amino acid residue to a non-polar and uncharged residue (see e.g. WO 2015/113007). In addition, it may be beneficial to epitope disruption to reduce the overall size and/or length of the amino acid residue's R-group functional side chain (see e.g. WO 2015/113007). However despite these generalities of substitutions most likely to confer epitope disruption, because the aim is to preserve significant Shiga toxin effector function(s), the substitute amino acid might be more likely to preserve Shiga toxin effector function(s) if it resembles the amino acid substituted for, such as, e.g., a nonpolar and/or uncharged residue of similar size substituted for a polar and/or charged residue.

WO 2015/113007 and WO 2016/196344 reported the results from the empirically testing of many different mutations and combinations of mutations for effect(s) on the Shiga toxin effector functions of various Shiga toxin effector polypeptides and binding molecules. Table 3 summarizes the results described in WO 2015/113007 and WO 2016/196344 where an amino acid substitution, alone or in combination with one or more other substitutions, did not prevent the exhibition of a potent level of a Shiga toxin effector function(s). Table 3 uses the epitope region numbering scheme described in WO 2016/196344.

TABLE 3
Amino Acid Substitutions in Shiga Toxin Effector Polypeptides

| Epitope Region | natively positioned amino acid positions | | |
|---|---|---|---|
| Disrupted | Substitution | B-Cell Epitope Region | T-Cell Epitope |
| 1 | K1A | 1-15 | |
| 1 | K1M | 1-15 | |
| 1 | T4I | 1-15 | 4-33 |
| 1 | D6R | 1-15 | 4-33 |
| 1 | S8I | 1-15 | 4-33 |
| 1 | T9V | 1-15 | 4-33 |
| 1 | T9I | 1-15 | 4-33 |
| 1 | K11A | 1-15 | 4-33 |
| 1 | K11H | 1-15 | 4-33 |
| 1 | T12K | 1-15 | 4-33 |
| 2 | S33I | 27-37 | 4-33 |
| 2 | S33C | 27-37 | 4-33 |
| 3 | S43N | 39-48 | 34-78 |
| 3 | G44L | 39-48 | 34-78 |
| 3 | T45V | 39-48 | 34-78 |
| 3 | T45I | 39-48 | 34-78 |
| 3 | S45V | 39-48 | 34-78 |
| 3 | S45I | 39-48 | 34-78 |
| 3 | G46P | 39-48 | 34-78 |
| 3 | D47G | 39-48 | 34-78 |
| 3 | D47M | 39-48 | 34-78 |
| 3 | N48V | 39-48 | 34-78 |
| 3 | N48F | 39-48 | 34-78 |
| — | L49A | immunogenic residue | 34-78 |
| — | F50T | | 34-78 |
| — | A51V | | 34-78 |
| 4 | D53A | 53-66 | 34-78 |
| 4 | D53G | 53-66 | 34-78 |
| 4 | D53N | 53-66 | 34-78 |
| 4 | V54L | 53-66 | 34-78 |
| 4 | V54I | 53-66 | 34-78 |
| 4 | R55A | 53-66 | 34-78 |
| 4 | R55V | 53-66 | 34-78 |
| 4 | R55L | 53-66 | 34-78 |
| 4 | G56P | 53-66 | 34-78 |
| 4 | I57M | 53-66 | 34-78 |
| 4 | I57F | 53-66 | 34-78 |
| 4 | D58A | 53-66 | 34-78 |
| 4 | D58V | 53-66 | 34-78 |
| 4 | D58F | 53-66 | 34-78 |
| 4 | P59A | 53-66 | 34-78 |
| 4 | P59F | 53-66 | 34-78 |
| 4 | E60I | 53-66 | 34-78 |
| 4 | E60T | 53-66 | 34-78 |
| 4 | E60R | 53-66 | 34-78 |
| 4 | E61A | 53-66 | 34-78 |
| 4 | E61V | 53-66 | 34-78 |
| 4 | E61L | 53-66 | 34-78 |
| 4 | G62A | 53-66 | 34-78 |
| — | R84A | | 77-103 |
| — | V88A | | 77-103 |
| 5 | D94A | 94-115 | 77-103 |
| 5 | S96I | 94-115 | 77-103 |
| 5 | T104N | 94-115 | |
| 5 | A105L | 94-115 | |
| 5 | T107P | 94-115 | |
| 5 | L108M | 94-115 | |
| 5 | S109V | 94-115 | |
| 5 | G110A | 94-115 | |
| 5 | D111T | 94-115 | |
| 5 | S112V | 94-115 | |
| 6 | D141A | 141-153 | 128-168 |
| 6 | G147A | 141-153 | 128-168 |
| — | VI54A | | 128-168 |
| 7 | R179A | 179-190 | 160-183 |
| 7 | T180G | 179-190 | 160-183 |
| 7 | T181I | 179-190 | 160-183 |
| 7 | D183A | 179-190 | 160-183 |
| 7 | D183G | 179-190 | 160-183 |
| 7 | D184A | 179-190 | |
| 7 | D184F | 179-190 | |
| 7 | L185V | 179-190 | |
| 7 | S186A | 179-190 | |
| 7 | S186F | 179-190 | |
| 7 | G187A | 179-190 | |
| 7 | G187T | 179-190 | |
| 7 | R188A | 179-190 | |
| 7 | R188L | 179-190 | |
| — | S189A | 179-190 | |
| — | D198A | immunogenic residue | |
| — | R205A | immunogenic residue | |
| — | C242S | | 236-258 |
| 8 | R248A | 243-257 | 236-258 |
| 8 | R251A | 243-257 | 236-258 |

Based on the empirical evidence in WO 2015/11307 and WO 2016/190344, certain amino acid positions in the A Subunits of Shiga toxins are predicted to tolerate epitope disruptions while still retaining significant Shiga toxin effector functions. For example, the following natively occurring positions tolerate amino acid substitutions, either alone or in combination, while retaining a Shiga toxin effector function(s) such as cytotoxicity—1 of SEQ ID NO:1 or SEQ ID NO:2; 4 of SEQ ID NO: 1, SEQ ID NO:2, or SEQ ID NO:3; 8 of SEQ ID NO: 1, SEQ ID NO:2, or SEQ ID NO:3; 9 of SEQ ID NO: 1, SEQ ID NO:2, or SEQ ID NO:3: 11 of SEQ ID NO: 1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO: 1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO: 1, SEQ ID NO:2, or SEQ ID NO:3; 59 of SEQ ID NO: 1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO 1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1. SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2.

The empirical data in WO 2015/113007 and WO 2016/196344 point towards other epitope disrupting substitutions and combinations of epitope disrupting substitutions that can reduce antigenicity and/or immunogenicity of a Shiga toxin effector polypeptide while retaining the ability of the Shiga toxin effector polypeptide to exhibit a significant Shiga toxin effector function such as, e.g., new combinations of the aforementioned truncations and positions tolerating substitutions as well as new substitutions at identical positions or conserved positions in related Shiga toxin A Subunits.

It is predictable that other amino acid substitutions to amino acid residues of a conservative functional group of a substitution tested herein may reduce antigenicity and/or immunogenicity while preserving a significant Shiga toxin effector function. For example, other substitutions known to the skilled worker to be similar to any of K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R. E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I may disrupt an endogenous epitope while maintaining at least one Shiga toxin effector function. In particular, amino acid substitutions to conservative amino acid residues similar to K1A, K1M, T4I, S8I, T8V, T9I, S9I, K11A, K11H, S33I, S33C, S43N, G44L, S45V, S45I, T45V, T45I, G46P, D47M, N48V, N48F, L49A, A51V, D53A, D53N, V54L, V54I, R55A, R55V, R55L, G56P, I57F, I57M, D58A, D58V, D58F, P59A, E60I, E60T, E61A, E61V, E61L, G62A, R84A, V88A, D94A, S96I, T104N, T107P, L108M, S109V, T109V, G110A, D111T, S112V, D141A, G147A, V154A, R179A, T180G, T181I, D183A, D183G, D184A, D184F, L185V, S186A, S186F, G187A, R188A, R188L, S189A, D198A, R204A, R205A, C242S, S247I, Y247A, R248A, R250A, R251A, D264A, G264A, T286A, and T286I may have the same or similar effects. In some embodiments, a Shiga toxin effector polypeptide may comprise similar conservative amino acid substitutions to empirically tested ones, such as, e.g., K1 to G, V, L, I, F, and H; T4 to A, G, V, L, F, M, and S; S8 to A, G, V, L, F, and M; T9 to A, G, L, F, M, and S; S9 to A, G, L, I, F. and M; K11 to G, V, L, I, F, and M; S33 to A, G, V, L, F, and M; S43 to A, G, V, L, I, F, and M; S45 to A, G, L, F, and M; T45 to A, G, L, F, and M; D47 to A, V, L, I, F, S, and Q; N48 to A, G, L, and M; L49 to G; Y49 to A; D53 to V, L, I, F, S, and Q; R55 to G, I, F, M, Q, S, K, and H; D58 to G, L, I, S, and Q; P59 to G; E60 to A, G, V, L, F, S, Q, N, D, and M; E61 to G, I, F, S, Q, N, D, M, and R; R84 to G, V, L, I, F, M, Q, S, K, and H; V88 to G; I88 to G; D94 to G, V, L, I, F, S, and Q; S96 to A, G, V, L, F, and M; T107 to A, G, V, L, I, F, M, and S; S107 to A, G, V, L, I, F, and M; S109 to A, G, I, L, F, and M; T109 to A, G, I, L, F, M, and S; S112 to A, G, L, I, F, and M; D141 to V, L, I, F, S, and Q; V154 to G; R179 to G, V, L, I, F, M, Q, S, K, and H; T180 to A, V, L, I, F, M, and S; T181 to A, G, V, L, F, M, and S; D183 to V, L, I, F, S, and Q; D184 to G, V, L, I, S, and Q; S186 to G, V, I, L, and M; R188 to G, V, I, F, M, Q, S, K, and H; S189 to G, V, I, L, F, and M; D197 to V, L, I, F, S, and Q; D198 to A, V, L, I, F, S, and Q; R204 to G, V, L, I, F, M, Q, S, K, and H; R205 to G, V, L, I, F, M, Q, S, K and H; S247 to A, G, V, I, L, F, and M; Y247 to A, G, V, L, I, F, and M; R248 to G, V, L, I, F, M, Q, S, K, and H; R250 to G, V, L, I, F, M, Q, S, K, and H; R251 to G, V, L, I, F, M, Q, S, K, and H; D264 to A, G, V, L, I, F, S, and Q; and T286 to A, G, V, L, I, F, M, and S.

Similarly, amino acid substitutions which remove charge, polarity, and/or reduce side chain length can disrupt an epitope while maintaining at least one Shiga toxin effector function. In some embodiments, a Shiga toxin effector polypeptide may comprise one or more epitopes disrupted by substitutions such that side chain charge is removed, polarity is removed, and/or side chain length is reduced such as, e.g., substituting the appropriate amino acid selected from the following group A, G, V, L, I, P, C, M, F, S, D, N, Q. H. or K for the amino acid residue at position 1 of SEQ ID NO:1 or SEQ ID NO:2; 4 of SEQ ID NO:1, SEQ ID NO:2, or SEQ ID NO:3; 6 of SEQ ID NO:1 or SEQ ID NO:2; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 12 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO: 1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1, SEQ ID NO:2, or SEQ ID NO:3; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1, SEQ ID NO:2, or SEQ ID NO:3; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1, SEQ ID NO:2, or SEQ ID NO:3; 108 of SEQ ID NO:1 or SEQ ID NO:2; 109 of SEQ ID NO: 1. SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO: or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 197 of SEQ ID NO:3; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2. In some embodiments, a Shiga toxin effector polypeptide may comprise one or more of the following amino acid substitutions: K1 to A, G, V, L, I, F, M and H; T4 to A, G, V, L, I, F, M, and S; D6 to A, G, V, L, I, F, S, and Q; S8 to A, G, V, I, L, F, and M; T8 to A, G, V, I, L, F, M, and S; T9 to A, G, V, I, L, F, M, and S; S9 to A, G, V, L, I, F, and M; K11 to A, G, V, L, I, F, M and H; T12 to A, G, V, I, L, F, M, and S; S33 to A, G, V, L, I, F, and M; S43 to A, G, V, L, I, F, and M; G44 to A and L; S45 to A, G, V, L, I, F, and M; T45 to A, G, V, L, I, F, and M; G46 to A and P; D47 to A, G, V, L, I, F, S, and Q; N48 to A, G, V, L, and M; L49 to A or G; F50; A51 to V; D53 to A, G, V, L, I, F, S, and Q; V54 to A, G, and L; R55 to A, G, V, L, I, F, M, Q, S, K, and H; G56 to A and P; 157 to A, G, M, and F; L57 to A, G, M, and F; D58 to A, G, V, L, I, F, S, and Q; P59 to A, G, and F; E60 to A, G, V, L, I, F, S, Q, N, D, M, and R; E61 to A, G, V, L, I, F, S, Q, N, D, M, and R; G62 to A; D94 to A, G, V, L, I, F, S, and Q; R84 to A, G, V, L, I, F, M, Q, S, K, and H; V88 to A and G; I88 to A, G, and V; D94; S96 to A, G, V, I, L, F, and M; T104 to A, G, V, I, L, F, M, and S; A105 to L; T107 to A, G, V, I, L, F, M, and S; S107 to A, G, V, L, 1, F, and M; L108 to A, G, and M; S109 to A, G, V, I, L, F, and M; T109 to A, G, V, I, L, F, M, and S; G110 to A; D111 to A, G, V, L, I, F, S, and Q; S112 to A, G, V, L, I, F, and M; D141 to A, G, V, L, I, F, S, and Q; G147 to A; V154 to A and G; R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, and V; S186 to A, G, V, I, L, F, and M; G187 to A; R188 to A, G, V, L, I, F, M, Q, S, K, and H; S189 to A, G, V, I, L, F, and M; D197 to A, G, V, L, I, F, S, and Q; D198 to A, G, V, L, I, F, S, and Q; R204 to A, G, V, L, I, F, M, Q, S, K, and H; R205 to A, G, V, L, I, F, M, Q, S, K and H; C242 to A, G, V, and S; S247 to A, G, V, I, L, F, and M; Y247 to A, G, V, L, I, F, and M; R248 to A, G, V, L, 1. F, M, Q, S, K, and H; R250 to A, G, V, L, I, F, M, Q, S, K, and H; R251 to A, G, V, L, I, F, M, Q, S, K, and H; C262 to A, G, V, and S; D264 to A, G, V, L, I, F, S, and Q; G264 to A; and T286 to A, G, V, L, I, F, M, and S.

In addition, any amino acid substitution in one epitope region of a Shiga toxin effector polypeptide which disrupts an epitope while retaining significant Shiga toxin effector function is combinable with any other amino acid substitution in the same or a different epitope region which disrupts an epitope while retaining significant Shiga toxin effector function to form a de-immunized, Shiga toxin effector polypeptide with multiple epitope regions disrupted while still retaining a significant level of Shiga toxin effector function. In some embodiments, a Shiga toxin effector polypeptide may comprise a combination of two or more of the aforementioned substitutions and/or the combinations of substitutions described in WO 2015/113007, WO 2016/196344, and/or WO 2018/140427.

Based on work described in WO 2015/113007, WO 2016/196344, and WO 2018/140427, certain amino acid regions in the A Subunits of Shiga toxins are predicted to tolerate epitope disruptions while still retaining significant Shiga toxin effector functions. For example, the epitope regions natively positioned at 1-15, 39-48, 53-66, 55-66, 94-115, 180-190, 179-190, and 243-257 tolerated multiple amino acid substitution combinations simultaneously without compromising Shiga toxin enzymatic activity and cytotoxicity.

### B. Examples of Furin-Cleavage Resistant, Shiga Toxin Effector Polypeptides

In some embodiments, the Shiga toxin effector polypeptide may comprise a disrupted, furin cleavage site at the carboxy-terminus of a Shiga toxin A1 fragment derived region. In some embodiments, the Shiga toxin effector polypeptide does not comprise any known compensatory structure which may provide furin cleavage proximal to the carboxy-terminus of the Shiga toxin A1 fragment derived region. Non-limiting examples of disrupted furin cleavage sites and furin cleave sites are described in WO 2015/191764.

Certain furin-cleavage site disruptions are indicated herein by reference to specific amino acid positions of native Shiga toxin A Subunits provided in the Sequence Listing, noting that naturally occurring Shiga toxin A Subunits includes precursor forms containing signal sequences of about 22 amino acids at their amino-terminals which are removed to produce mature Shiga toxin A Subunits and are recognizable to the skilled worker. Further, certain furin-cleavage site disruptions comprising mutations are indicated herein by reference to specific amino acids (e.g. R for an arginine residue) natively present at specific positions within native Shiga toxin A Subunits (e.g. R251 for the arginine residue at position 251 from the amino-terminus) followed by the amino acid with which that residue has been substituted in the particular mutation under discussion (e.g. R251A represents the amino acid substitution of alanine for arginine at amino acid residue 251 from the amino-terminus).

In some embodiments, the Shiga toxin effector polypeptide comprises a disrupted furin-cleavage site at the carboxy-terminus of a Shiga toxin A1 fragment derived region, and such embodiments are referred to herein as “furin-cleavage resistant” or “protease-cleavage resistant,” Shiga toxin effector polypeptides to describe their property(ies) relative to wild-type, Shiga toxin A Subunits and/or wild-type. Shiga toxin A1 fragment fusion proteins.

In some embodiments, the protease-cleavage resistant, Shiga toxin effector polypeptide consists essentially of a truncated Shiga toxin A Subunit having two or more mutations.

In some embodiments, the protease-cleavage resistant, Shiga toxin effector poly peptide comprises the disrupted furin-cleavage site comprising the amino acid residue substitution (relative to a wild-type Shiga toxin polypeptide) of one or both of the arginine residues in the minimal, furin-cleavage site consensus sequence with A, G, or H. In some embodiments, the protease-cleavage resistant, Shiga toxin effector polypeptide comprises a disruption which comprises an amino acid substitution within a furin-cleavage site, where in the substitution occurs at the natively positioned amino acid selected from the group consisting of: 247 of SEQ ID NO:3, 248 of SEQ ID NO:1 or SEQ ID NO:2, 250 of SEQ ID NO:3, 251 of SEQ ID NO:1 or SEQ ID NO:2, or the equivalent position in a conserved Shiga toxin effector polypeptide and/or non-native Shiga toxin effector polypeptide sequence. In some embodiments, the substitution is to any non-conservative amino acid and the substitution occurs at the natively positioned amino acid residue position. In some embodiments, the mutation comprises an amino acid substitution selected from the group consisting of: R247A, R248A, R250A R251A, or the equivalent position in a conserved Shiga toxin effector polypeptide and/or non-native Shiga toxin effector polypeptide sequence.

In some embodiments, the protease-cleavage resistant Shiga toxin effector poly peptide comprises the disrupted furin-cleavage site comprising the mutation which is a deletion. In some embodiments, the disrupted furin-cleavage site comprises a mutation which is a deletion of the region natively positioned at 247-252 in StxA (SEQ ID NO:2) and SLT-1A (SEQ ID NO:3), or the region natively positioned at 246-251 in SLT-2A (SEQ ID NO:3); a deletion of the region natively positioned at 244-246 in StxA (SEQ ID NO:2) and SLT-1A (SEQ ID NO:3), or the region natively positioned at 243-245 in SLT-2A (SEQ ID NO:3); or a deletion of the region natively positioned at 253-259 in StxA (SEQ ID NO:2) and SLT-1A (SEQ ID NO:3), or the region natively positioned at 252-258 in SLT-2A (SEQ ID NO:3).

In some embodiments, the protease-cleavage resistant Shiga toxin effector polypeptide comprises the disrupted furin-cleavage site comprising the mutation which is a carboxy-terminal truncation as compared to a wild-type Shiga toxin A Subunit, the truncation which results in the deletion of one or more amino acid residues within the furin-cleavage site. In some embodiments, the disrupted furin-cleavage site comprises the carboxy-terminal truncation which deletes one or more amino acid residues within the minimal cleavage site Y/R-x-x-R, such as, e.g., for StxA and SLT-1A derived Shiga toxin effector polypeptides, truncations ending at the natively amino acid residue position 250, 249, 248, 247, 246, 245, 244, 243, 242, 241, 240, or less; and for SLT-2A derived Shiga toxin effector polypeptides, truncations ending at the natively amino acid residue position 249, 248, 247, 246, 245, 244, 243, 242, 241, or less. Some embodiments comprise the disrupted furin-cleavage site comprising a combination of any of the aforementioned mutations, where possible.

In some embodiments, the disrupted furin-cleavage site comprises the mutation(s) that is a partial, carboxy-terminal truncation of the furin-cleavage site; however, some molecules described herein do not comprise the disrupted furin-cleavage site which is a complete, carboxy-terminal truncation of the entire 20 amino acid residue, furin-cleavage site. For example, certain Shiga toxin effector polypeptides comprise the disrupted furin-cleavage site comprising a partial, carboxy-terminal truncation of the Shiga toxin A1 fragment region up to native position 240 in StxA (SEQ ID NO:2) or SLT-1A (SEQ ID NO:1) but not a carboxy-terminal truncation at position 239 or less. Similarly, certain Shiga toxin effector polypeptides comprise the disrupted furin-cleavage site comprising a partial, carboxy-terminal truncation of the Shiga toxin A1 fragment region up to native position 239 in SLT-2A (SEQ ID NO:3) but not a carboxy-terminal truncation at position 238 or less. In the largest carboxy-terminal truncation of the furin-cleavage resistant, Shiga toxin effector polypeptide, mutations comprising the disrupted furin-cleavage site, positions P14 and P13 of the furin-cleavage site are still present.

In some embodiments, the disrupted furin-cleavage site comprises both an amino acid residue substitution within the furin-cleavage site and a carboxy-terminal truncation as compared to a wild-type, Shiga toxin A Subunit. In some embodiments, the disrupted furin-cleavage site comprises both an amino acid residue substitution within the minimal furin-cleavage site R/Y-x-x-R and a carboxy-terminal truncation as compared to a wild-type, Shiga toxin A Subunit, such as, e.g., for StxA and SLT-1A derived Shiga toxin effector polypeptides, truncations ending at the natively amino acid residue position 249, 250, 251, 252, 253, 254, 255, 256, 257, 258, 259, 260, 261, 262, 263, 264, 265, 266), 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid residue position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate. In some embodiments, the truncated Shiga toxin effector polypeptide comprising a disrupted furin-cleavage site also comprises the furin-cleavage site, amino acid residues at positions P9, P8, and/or P7 in order to maintain optimal cytotoxicity.

In some embodiments, the disrupted furin-cleavage site comprises a mutation(s) which is one or more internal, amino acid residue deletions, as compared to a wild-type, Shiga toxin A Subunit. In some embodiments, the disrupted furin-cleavage site comprises a mutation(s) which has one or more amino acid residue deletions within the minimal furin-cleavage site R/Y-x-x-R. For example, StxA and SLT-1A derived Shiga toxin effector polypeptides comprising internal deletions of the natively positioned amino acid residues R248 and/or R251, which may be combined with deletions of surrounding residues such as, e.g., 249, 250, 247, 252, etc.; and SLT-2A derived Shiga toxin effector polypeptides comprising internal deletions of the natively positioned amino acid residues Y247 and/or R250, which may be combined with deletions of surrounding residues such as, e.g., 248, 249, 246, 251, etc. In some embodiments, the disrupted furin-cleavage site comprises a mutation which is a deletion of four, consecutive, amino acid residues which deletes the minimal furin-cleavage site R/Y-x-x-R, such as, e.g., StxA and SLT-1A derived Shiga toxin effector polypeptides lacking R248-R251 and SLT-2A derived Shiga toxin effector polypeptides lacking Y247-R250. In some embodiments, the disrupted furin-cleavage site comprises a mutation(s) having one or more amino acid residue deletions in the amino acid residues flanking the core furin-cleavage site, such as, e.g., a deletion of 244-247 and/or 252-255 in SLT-1A or StxA. In some embodiments, the disrupted furin-cleavage site comprises a mutation which is an internal deletion of the entire surface-exposed, protease-cleavage sensitive loop as compared to a wild-type, Shiga toxin A Subunit, such as, e.g., for StxA and SLT-1A derived Shiga toxin effector polypeptides, a deletion of natively positioned amino acid residues 241-262; and for SLT-2A derived Shiga toxin effector polypeptides, a deletion of natively positioned amino acid residues 240-261.

In some embodiments, the disrupted furin-cleavage site comprises both a mutation which is an internal, amino acid residue deletion within the furin-cleavage site and a mutation which is carboxy-terminal truncation as compared to a wild-type, Shiga toxin A Subunit. In some embodiments, the disrupted furin-cleavage site comprises both a mutation which is an amino acid residue deletion within the minimal furin-cleavage site R/Y-x-x-R and a mutation which is a carboxy-terminal truncation as compared to a wild-type, Shiga toxin A Subunit. For example, protease-cleavage resistant, Shiga toxin effector polypeptides may comprise a disrupted furin-cleavage site comprising mutation(s) which are deletions of the natively positioned amino acid residues 248-249 and/or 250-251 in a truncated StxA or SLT-1A polypeptide which still has amino acid residue 247 and/or 252, or the amino acid residues 247-248 and/or 249-250 in a truncated SLT-2A which still has amino acid residue 246 and/or 251. In some embodiments, the disrupted furin-cleavage site comprises a mutation having a deletion of four, consecutive, amino acid residues which deletes the minimal furin-cleavage site R/Y-x-x-R and a carboxy-terminal truncation as compared to a wild-type, Shiga toxin A Subunit, such as, e.g., for StxA and SLT-1A derived Shiga toxin effector polypeptides, truncations ending at the natively amino acid residue position 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and lacking R248-R251; and for SLT-2A derived Shiga toxin effector polypeptides, truncations ending at the natively amino acid residue position 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and lacking Y247-R250.

### C. Examples of Shiga Toxin Effector Polypeptides Having an Embedded Epitope

In some embodiments, the Shiga toxin effector polypeptide may comprise one or more embedded or inserted, heterologous, T-cell epitopes for purposes of de-immunization and/or delivery to a MHC class I presentation pathway of a target cell. In some embodiments and/or certain Shiga toxin effector polypeptide sub-regions, embedding or partial embedding a T-cell epitope may be preferred over inserting a T-cell epitope because, e.g., embedding-type modifications are more likely to be successful in diverse sub-regions of a Shiga toxin effector polypeptide whereas successful insertions may be more limited to a smaller subset of Shiga toxin effector polypeptide sub-regions. The term “successful” is used here to mean the modification to the Shiga toxin effector polypeptide (e.g. introduction of a heterologous, T-cell epitope) results in a modified Shiga toxin effector polypeptide which retains one or more Shiga toxin effector functions at the requisite level of activity either alone or as a component of a binding molecule.

Any of the Shiga toxin effector polypeptide sub-regions described in WO 2015/113007 may be suitable. In some embodiments, and any of the Shiga toxin effector polypeptides described in WO 2015/113007 may be modified into a Shiga toxin effector polypeptide of a binding molecule, e.g., by the addition of one or more new epitope region disruptions for de-immunization (such one as described herein) and/or a furin-cleavage site disruption (such as one described herein).

In some embodiments, the Shiga toxin effector polypeptide consists essentially of a truncated Shiga toxin A Subunit comprising an embedded or inserted, heterologous, T-cell epitope and one or more other mutations. In some embodiments, the Shiga toxin effector polypeptide comprises an embedded or inserted, heterologous. T-cell epitope and is smaller than a full-length, Shiga toxin A Subunit, such as, e.g., consisting of the polypeptide represent by amino acids 77 to 239 of SLT-1A (SEQ ID NO:1) or StxA (SEQ ID NO:2) or the equivalent in other A Subunits of members of the Shiga toxin family (e.g. amino acids 77 to 238 of SLT-2A (SEQ ID NO:3)). For example, in some embodiments, the Shiga toxin effector polypeptides is derived from amino acids 75 to 251 of SEQ ID NO:1, 1 to 241 of SEQ ID NO:1, 1 to 251 of SEQ ID NO: 1, or amino acids 1 to 261 of SEQ ID NO:1, wherein the Shiga toxin effector polypeptide comprises at least one embedded or inserted, heterologous T-cell epitope and at least one amino acid is disrupted in an endogenous, B-cell and/or CD4+ T-cell epitope region and wherein the disrupted amino acid does not overlap with the embedded or inserted epitope. Similarly in other embodiments, the Shiga toxin effector polypeptide is derived from amino acids 75 to 251 of SEQ ID NO:2, 1 to 241 of SEQ ID NO:2, 1 to 251 of SEQ ID NO:2, or amino acids 1 to 261 of SEQ ID NO:2, wherein the Shiga toxin effector polypeptide comprises at least one embedded or inserted, heterologous T-cell epitope and at least one amino acid is disrupted in an endogenous. B-cell and/or CD4+ T-cell epitope region and wherein the disrupted amino acid does not overlap with the embedded or inserted epitope. Additionally, the Shiga toxin effector polypeptide may be derived from amino acids 75 to 251 of SEQ ID NO:3, 1 to 241 of SEQ ID NO:3, 1 to 251 of SEQ ID NO:3, or amino acids 1 to 261 of SEQ ID NO:3, wherein the Shiga toxin effector polypeptide comprises at least one embedded or inserted, heterologous T-cell epitope and at least one amino acid is disrupted in an endogenous, B-cell and/or CD4+ T-cell epitope region and wherein the disrupted amino acid does not overlap with the embedded or inserted epitope. In some embodiments, the Shiga toxin effector polypeptide comprises an embedded or inserted, heterologous, T-cell epitope and a disrupted furin-cleavage site at the carboxy-terminus of a Shiga toxin A1 fragment derived region. For example in some embodiments, the Shiga toxin effector polypeptide is derived from amino acids 75 to 251 of SEQ ID NO:1, 1 to 241 of SEQ ID NO:1, 1 to 251 of SEQ ID NO:1, or amino acids 1 to 261 of SEQ ID NO:1, wherein the Shiga toxin effector polypeptide comprises at least one embedded or inserted, heterologous T-cell epitope and a disrupted furin-cleavage site at the carboxy-terminus of a Shiga toxin A1 fragment derived region. Similarly in other embodiments, the Shiga toxin effector polypeptide is derived from amino acids 75 to 251 of SEQ ID NO:2, 1 to 241 of SEQ ID NO:2, 1 to 251 of SEQ ID NO:2, or amino acids 1 to 261 of SEQ ID NO:2, wherein the Shiga toxin effector polypeptide comprises at least one embedded or inserted, heterologous T-cell epitope and a disrupted furin-cleavage site at the carboxy-terminus of a Shiga toxin A1 fragment derived region. Additionally, the Shiga toxin effector polypeptide may be derived from amino acids 75 to 251 of SEQ ID NO:3, 1 to 241 of SEQ ID NO:3, 1 to 251 of SEQ ID NO:3, or amino acids 1 to 261 of SEQ ID NO:3, wherein the Shiga toxin effector polypeptide comprises at least one embedded or inserted, heterologous T-cell epitope and a disrupted furin-cleavage site at the carboxy-terminus of a Shiga toxin A1 fragment derived region.

### D. Examples of Combination Shiga Toxin Effector Polypeptides

A combination Shiga toxin effector polypeptide comprises two or more sub-regions (i.e. non-overlapping sub-regions) wherein each sub-region comprises at least one of the following: (1) a disruption in an endogenous epitope or epitope region; (2) an embedded, heterologous, T-cell epitope-peptide; (3) an inserted, heterologous, T-cell epitope-peptide; and (4) a disrupted furin-cleavage site at the carboxy-terminus of an A1 fragment derived region.

Certain embodiments of the combination Shiga toxin effector polypeptides comprise both (1) a disruption in an endogenous epitope or epitope region and (2) a disrupted furin-cleavage site at the carboxy-terminus of an A1 fragment derived region. It is predicted that any of the individual, de-immunized, Shiga toxin effector sub-regions described in WO 2015/113007, WO 2016/196344, and WO 2018/140427 (see e.g. Table 3, supra) may generally be combined with any Shiga toxin effector sub-region comprising a disrupted furin-cleavage site described herein, described in WO 2015/191764, and/or known in the art in order to create a Shiga toxin effector polypeptide for use as a component of a binding molecule.

In some embodiments, the Shiga toxin effector polypeptide comprises a disruption of at least one, endogenous, B-cell and/or T-cell epitope region which does not overlap with an embedded or inserted, heterologous, CD8+ T-cell epitope; wherein the disruption comprises one or more amino acid residue substitutions relative to a wild-type Shiga toxin. In some embodiments the substitution is selected from the group consisting of: K1 to A, G, V, L, I, F, M and H; T4 to A, G, V, L, I, F, M, and S; D6 to A, G, V, L, I, F, S, Q and R; S8 to A, G, V, I, L, F, and M; T9 to A, G, V, I, L, F, M, and S; S9 to A, G, V, L, I, F, and M; K11 to A, G, V, L, I, F, M and H; T12 to A, G, V, I, L, F, M, S, and K; S12 to A, G, V, I, L, F, and M; S33 to A, G, V, L, I, F, M, and C; S43 to A, G, V, L, I, F, and M; G44 to A or L; S45 to A, G, V, L, I, F, and M; T45 to A, G, V, L, I, F, and M; G46 to A and P; D47 to A, G, V, L, I, F, S, M, and Q; N48 to A, G, V, L, M and F; L49 to A, V, C, and G; Y49 to A, G, V, L, 1. F, M, and T; F50 to A, G, V, L, I, and T; A51; D53 to A, G, V, L, I, F, S, and Q; V54 to A, G, I, and L; R55 to A, G, V, L, I, F, M, Q, S, K, and H; G56 to A and P; 157 to A, G, V, and M; L57 to A, V, C, G, M, and F; D58 to A, G, V, L, I, F, S, and Q; P59 to A, G, and F; E60 to A, G, V, L, I, F, S, Q, N, D, M, T, and R; E61 to A, G, V, L, I, F, S, Q, N, D, M, and R; G62 to A; R84 to A, G, V, L, I, F, M, Q, S, K, and H; V88 to A and G; 188 to A, V, C, and G; D94 to A, G, V, L, I, F, S, and Q; S96 to A, G, V, I, L, F, and M; T104 to A, G, V, L, I, F, M; and N; A105 to L; T107 to A, G, V, L, I, F, M, and P; S107 to A, G, V, L, I, F, M, and P; L108 to A, V, C, and G; S109 to A, G, V, I, L, F, and M; T109 to A, G, V, I, L, F, M, and S; G110 to A; S112 to A, G, V, L, I, F, and M; D111 to A, G, V, L, I, F, S, Q, and T; S112 to A, G, V, L, I, F, and M; D141 to A, G, V, L, I, F, S, and Q; G147 to A; V154 to A and G, R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, V and C; S186 to A, G, V, I, L, F, and M; G187 to A; R188 to A, G, V, L, I, F, M, Q, S, K, and H; S189 to A, G, V, I, L, F, and M; D198 to A, G, V, L, I, F, S, and Q; R204 to A, G, V, L, I, F, M, Q, S, K, and H; R205 to A, G, V, L, I, F, M, Q, S, K and H; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; D264 to A, G, V, L, 1. F, S, and Q; G264 to A; and T286 to A, G, V, L, 1. F, M, and S. In some embodiments, there are multiple disruptions of multiple, endogenous B-cell and/or CD8+ T-cell epitope regions wherein each disruption involves at least one amino acid residue substitution selected from the group consisting of: K1 to A, G, V, L, I, F, M and H; T4 to A, G, V, L, I, F, M, and S; D6 to A, G, V, L, I, F, S, Q and R; S8 to A, G, V, I, L, F, and M; T9 to A, G, V, I, L, F, M, and S; S9 to A, G, V, L, I. F, and M; K11 to A, G, V, L, I, F, M and H; T12 to A, G, V, I, L, F, M, S, and K; S12 to A, G, V, I, L, F, and M; S33 to A, G, V, L, I, F, M, and C; S43 to A, G, V, L, I, F, and M; G44 to A or L; S45 to A, G, V, L, I, F, and M; T45 to A, G, V, L, I, F, and M; G46 to A and P; D47 to A, G, V, L, I, F, S, M, and Q; N48 to A, G, V, L, M and F; L49 to A, V, C, and G; Y49 to A, G, V, L, I, F, M, and T; F50 to A, G, V, L, I, and T; A51; D53 to A, G, V, L, I, F. S, and Q; V54 to A, G, I, and L; R55 to A, G, V, L, I, F, M, Q, S, K, and H; G56 to A and P; I57 to A, G, V, and M; L57 to A, V, C, G, M, and F; D58 to A, G, V, L, I, F, S, and Q; P59 to A, G, and F; E60 to A, G, V, L, I, F, S, Q, N, D, M, T, and R; E61 to A, G, V, L, I, F, S, Q, N, D, M, and R; G62 to A; R84 to A, G, V, L, I, F, M, Q, S, K, and H; V88 to A and G; 188 to A, V, C. and G; D94 to A, G, V, L, I, F, S, and Q; S96 to A, G, V, I, L, F, and M; T104 to A, G, V, L, I, F, M; and N; A105 to L; T107 to A, G, V, L, I, F, M, and P; S107 to A, G, V, L, I, F, M, and P; L108 to A, V, C, and G; S109 to A, G, V, I, L, F, and M; T109 to A, G, V, I, L, F, M, and S; G110 to A; S112 to A, G, V, L, I, F, and M; D111 to A, G, V, L, I, F, S, Q, and T; S112 to A, G, V, L, I, F, and M D141 to A, G, V, L, I, F, S, and Q; G147 to A; V154 to A and G. R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, V and C; S186 to A, G, V, I, L, F, and M; G187 to A; R188 to A, G, V, L, I, F, M, Q, S, K, and H; S189 to A, G, V, I, L, F, and M; D198 to A, G, V, L, I, F, S, and Q; R204 to A, G, V, L, I, F, M, Q, S, K, and H; R205 to A, G, V, L, I, F, M, Q, S, K and H; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; D264 to A, G, V, L, I, F, S, and Q; G264 to A; and T286 to A, G, V, L, I, F, M, and S.

Certain embodiments, the Shiga toxin effector polypeptide comprises both (1) an embedded or inserted, heterologous, T-cell epitope-peptide and (2) a disrupted furin-cleavage site at the carboxy-terminus of an A1 fragment derived region. Any of the Shiga toxin effector polypeptide sub-regions comprising an embedded or inserted, heterologous, T-cell epitope described in WO 2015/113007 may generally be combined with any protease-cleavage resistant, Shiga toxin effector polypeptide sub-region (e.g., modified. Shiga toxin A Subunit sub-regions described herein, described in WO 2015/191764, and/or known in the art) in order to create a combination, Shiga toxin effector polypeptide which, as a component of a binding molecule, is both protease-cleavage resistant and capable of delivering a heterologous, T-cell epitope to the MHC class 1 presentation pathway of a target cell. Non-limiting examples of this type of combination Shiga toxin effector polypeptide are shown in SEQ ID NOs: 19-21.

Certain embodiments of the combination Shiga toxin effector polypeptides comprise both (1) a disruption in an endogenous epitope or epitope region and (2) an embedded, heterologous, T-cell epitope-peptide. However, the Shiga toxin effector sub-regions comprising inserted or embedded, heterologous, T-cell epitopes described herein or in WO 2015/191764 are generally not combinable with every de-immunized, Shiga toxin effector sub-regions described herein, except where empirically shown to be successfully combined such that the resulting combination molecule retained a sufficient level of a Shiga toxin effector function(s). The disclosure herein shows how such embodiments may be made and tested to empirically demonstrate success.

The term “successful” is used here to mean two or more amino acid residue substitutions in a Shiga toxin effector polypeptide results in a functional feature, such as, e.g., de-immunization, reduced furin-cleavage, and/or ability to deliver an embedded or inserted epitope, while the modified Shiga toxin effector polypeptide retains one or more Shiga toxin effector functions. The approaches and assays described herein show how to design, make and empirically test embodiments described herein, which represent combination, Shiga toxin effector polypeptides and binding molecules comprising the same.

The combination, Shiga toxin effector polypeptide may combine the features of their respective sub-regions, such as, e.g., a furin-cleavage site disruption, individual epitope disruptions, and/or a heterologous T-cell epitope cargo, and these combinations sometimes result in Shiga toxin effector polypeptides with synergistic reductions in immunogenicity as compared to the sum of their partially de-immunized sub-regions.

De-immunized, Shiga toxin effector polypeptides which exhibit no cytotoxicity or reduced cytotoxicity at certain concentrations, e.g. Shiga toxin effector polypeptides comprising R179A, may still be useful as de-immunized, Shiga toxin effector polypeptides for delivering exogenous materials into cells. Similarly, CD8+ T-cell hyper-immunized, Shiga toxin effector polypeptides of the which exhibit no cytotoxicity or reduced cytotoxicity at certain concentrations, e.g. a Shiga toxin effector polypeptide comprising an epitope embedded into its catalytic domain (see e.g. WO 2015/113005: Example 1-F), may still be useful for delivering a T-cell epitope(s) to a desired subcellular compartment of a cell in which the Shiga toxin effector polypeptide is present or as a component of a binding molecule for delivery of a T-cell epitope(s) into a target cell.

### E. Examples of Binding Molecules

The following embodiments describe in more detail certain structures of exemplary binding molecules which target cells physically coupled to PD-L1 at a cellular surface, e.g. cells which express PD-L1 and/or PD-L1 positive cells.

Provided herein are various embodiments of PD-L1 binding molecules, and compositions thereof, wherein each PD-L1 binding molecule comprises (1) at least one toxin component and (2) at least one PD-L1 binding region capable of specifically binding an extracellular part of a PD-L1 molecule. For each PD-L1 binding molecule described herein, the at least one binding region is heterologous to the toxin from which the toxin effector polypeptide is derived, such as, e.g., a PD-L1 binding region comprising an immunoglobulin domain unrelated to the toxin. In some embodiments, the at least one toxin component comprises a toxin effector polypeptide. In some embodiments, the toxin effector polypeptide is a Shiga toxin A Subunit effector polypeptide derived from the A Subunit of a Shiga toxin.

In some embodiments, the PD-L1 binding molecule comprises (1) at least one Shiga toxin A Subunit effector polypeptide derived from the A Subunit of at least one member of the Shiga toxin family and (2) at least one PD-L1 binding region capable of specifically binding an extracellular part of a PD-L1 molecule.

In some embodiments, the PD-L1 binding region comprises a heavy chain variable region (HVR-H) comprising three CDRs, each having at least 85%, 90%, 91%, 92%, 93%, 94%, 95%, 96%, 97%, 98%, or 99% identity to any one of SEQ ID NOs: 22-24 and 27-32; or consisting essentially of an amino acid sequence show in any one of SEQ ID NOs: 22-24 and 27-32. In some embodiments, the binding region further comprises: (a) a light chain variable region (HVR-L) comprising three CDRs, each having at least 85%, 90%, 91%, 92%, 93%, 94%, 95%, 96%, 97%, 98%, or 99% identity to any one of SEQ ID NO:19, SEQ ID NO:20. SEQ ID NO:21, SEQ ID NO:25, and SEQ ID NO:26; or consisting essentially of an amino acid sequence shown in any one of SEQ ID NO:19, SEQ ID NO:20, SEQ ID NO:21, SEQ ID NO:25, and SEQ ID NO:26. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:21; or consisting essentially of an amino acid sequence shown in any one of SEQ ID NO:19, SEQ ID NO:20, and SEQ ID NO:21. In certain other further embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:25, SEQ ID NO:20, and SEQ ID NO:21; or consisting essentially of an amino acid sequence shown in any one of SEQ ID NO:25, SEQ ID NO:20, and SEQ ID NO:21. In certain other further embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:26; or consisting essentially of an amino acid sequence shown in any one of SEQ ID NO:19, SEQ ID NO:20, and SEQ ID NO:26.

In some embodiments, the binding region comprises: (a) a light chain variable region (HVR-L) comprising three CDRs, each comprising or consisting essentially of an amino acid sequence shown in any one of SEQ ID NO:19, SEQ ID NO:20, SEQ ID NO:21, SEQ ID NO:25, and SEQ ID NO:26; and (b) a heavy chain variable region (HVR-H) comprising three CDRs, each comprising or consisting essentially of an amino acid sequence show in any one of SEQ ID NOs: 22-24 and 27-32.

In some embodiments, the binding region comprises: (a) a light chain region having at least 85%, 90%, 91%, 92%, 93%, 94%, 95%, 96%, 97%, 98%, or 99%, identity to any one of SEQ ID NOs: 33, 35-36, and 38, or consisting essentially of the amino acid sequence of any one of SEQ ID NOs: 33, 35-36, and 38, and/or (b) a heavy chain region having at least 85%, 90%, 91%, 92%, 93%, 94%, 95%, 96%, 97%, 98%, or 99% identity to any one of SEQ ID NOs: 34, 37, and 39, or consisting essentially of the amino acid sequence of any one of SEQ ID NOs: 34, 37, and 39. In some embodiments, the binding region comprises a polypeptide having at least 85%, 90%, 91%, 92%, 93%, 94%, 95%, 96%, 97%, 98%, or 99% identity to any one of SEQ ID NOs: 85-107 and 156-157 or consists essentially of the polypeptide shown in any one of SEQ ID NOs: 85-107 and 156-157. In some embodiments, the binding region is a single-chain variable fragment, such as, e.g., consisting of, comprising, or consisting essentially of the polypeptide of any one of SEQ ID NOs: 85-107 and 156-157.

In some embodiments, a PD-L1 binding molecule comprising a Shiga toxin A subunit effector polypeptide and a binding region capable of specifically binding an extracellular part of PD-L1; wherein the binding region comprises (a) a heavy chain variable region (VH) comprising (i) a CDR1 comprising the amino acid sequence EYTMH (SEQ ID NO:27), (ii) a CDR2 comprising the amino acid sequence GINPNNGGTWYNQKFKG (SEQ ID NO:29), and (iii) a CDR3 comprising the amino acid sequence PYYYGSREDYFDY (SEQ ID NO:32); and (b) a light chain variable region (VL) comprising (i) a CDR1 comprising the amino acid sequence SASSSVSYMY (SEQ ID NO:19), (ii) a CDR2 comprising the amino acid sequence LTSNLAS (SEQ ID NO:20), and (iii) a CDR3 comprising the amino acid sequence QQWSSNPPT (SEQ ID NO:26). In some embodiments, a PD-L1 binding molecule comprising a Shiga toxin A subunit effector polypeptide and a binding region capable of specifically binding an extracellular part of PD-L1; wherein the binding region comprises (a) a heavy chain variable region (VH) comprising (i) a CDR1 consisting of the amino acid sequence EYTMH (SEQ ID NO:27), (ii) a CDR2 consisting of the amino acid sequence GINPNNGGTWYNQKFKG (SEQ ID NO:29), and (iii) a CDR3 consisting of the amino acid sequence PYYYGSREDYFDY (SEQ ID NO:32); and (b) a light chain variable region (VL) comprising (i) a CDR1 consisting of the amino acid sequence SASSSVSYMY (SEQ ID NO:19), (ii) a CDR2 consisting of the amino acid sequence LTSNLAS (SEQ ID NO:20), and (iii) a CDR3 consisting of the amino acid sequence QQWSSNPPT (SEQ ID NO:26). In some embodiments, a PD-L1 binding molecule comprising a Shiga toxin A subunit effector polypeptide and a binding region capable of specifically binding an extracellular part of PD-L1; wherein the binding region comprises (a) a heavy chain variable region (VH) comprising (i) a CDR1 having the amino acid sequence EYTMH (SEQ ID NO:27), (ii) a CDR2 having the amino acid sequence GINPNNGGTWYNQKFKG (SEQ ID NO:29), and (iii) a CDR3 having the amino acid sequence PYYYGSREDYFDY (SEQ ID NO:32); and (b) a light chain variable region (VL) comprising (i) a CDR1 having the amino acid sequence SASSSVSYMY (SEQ ID NO:19), (ii) a CDR2 having the amino acid sequence LTSNLAS (SEQ ID NO:20), and (iii) a CDR3 having the amino acid sequence QQWSSNPPT (SEQ ID NO:26).

In some embodiments, the Shiga toxin A subunit effector polypeptide comprises the sequence of SEQ ID NO: 41, or a sequence at least 90% or at least 95% identical thereto.

In some embodiments, the VH comprises the sequence of SEQ ID NO: 34, or a sequence at least 90% or at least 95% identical thereto. In some embodiments, the VL comprises the sequence of SEQ ID NO: 35, or a sequence at least 90% or at least 95% identical thereto. In some embodiments, the VH comprises the sequence of SEQ ID NO: 34 and the VL comprises the sequence of SEQ ID NO: 35.

In some embodiments, the PD-L1-binding molecule comprises an amino acid sequence with at least 90%, at least 95%, at least 96%, at least 97%, at least 98%, or at least 99% identity to SEQ ID NO: 303-313. In some embodiments, the PD-L1-binding molecule comprises an amino acid sequence of any of one SEQ ID NOs: 303-313. In some embodiments, the PD-L1-binding molecule comprises an amino acid sequence of any of one SEQ ID NOs: 303-313 with one or more mutations, such as 2, 3, 4, 5, 6, 7, 8, or 10, or more mutations. In some embodiments, the PD-L1-binding molecule comprises an amino acid sequence of any of one SEQ ID NOs: 303-313 with 1-5, 5-10, 11-5, 15-20, 10-25, 25-30, or more than 30 mutations.

In some embodiments, the PD-L1 binding molecule comprises: (i) a Shiga-like toxin A subunit effector polypeptide; (ii) a binding region capable of specifically binding an extracellular part of PD-L1; wherein the binding region comprises: (a) a heavy chain variable region (VH) comprising: (1) a CDR1 comprising the amino acid sequence EYTMH (SEQ ID NO:27), (2) a CDR2 comprising the amino acid sequence GINPNNGGTWYNQKFKG (SEQ ID NO:29), and (3) a CDR3 comprising the amino acid sequence PYYYGSREDYFDY (SEQ ID NO:32); and (b) a light chain variable region (VL) comprising: (1) a CDR1 comprising the amino acid sequence SASSSVSYMY (SEQ ID NO:19), (2) a CDR2 comprising the amino acid sequence LTSNLAS (SEQ ID NO:20), and (3) a CDR3 comprising the amino acid sequence QQWSSNPPT (SEQ ID NO:26); and (iii) at least one CD8+ T-cell epitope that is heterologous to Shiga-like toxin A subunits.

In some embodiments, the cell binding molecule comprises: (i) a Shiga-like toxin A subunit effector polypeptide; (ii) a binding region capable of specifically binding an extracellular target on a cell; and (iii) CD8+ T-cell epitope comprising the sequence of SEQ ID NO: 300 or 301.

In some embodiments, the binding region comprises a scFv linker that links the VH and the VL, In some embodiments, the scFv linker is 3 to 12 amino acids in length. In some embodiments, the scFv linker is 3 to about 12 amino acids in length. In some embodiments, the scFv linker is about 3 to about 12 amino acids in length. In some embodiments, the scFv linker is about 10-20 amino acids in length. In some embodiments, the scFv linker is greater than 20 amino acids in length. In some embodiments, the scFv linker is a flexible linker. In some embodiments, the scFv linker comprises the sequence of SEQ ID NO: 72, or a sequence at least 900% or at least 95% identical thereto. In some embodiments, the binding region is a single chain variable fragment (scFv). In some embodiments, the binding region comprises the sequence of SEQ ID NO: 106, or a sequence at least 90% or at least 95% identical thereto.

As used herein, the term “binding domain linker” refers to a linker which links the Shiga toxin A subunit effector polypeptide and the binding region (e.g., the scFv). In some embodiments, the PD-L1 binding molecule comprises a binding domain linker. In some embodiments, the binding domain linker comprises the sequence of SEQ ID NO: 73, or a sequence at least 90% or at least 95% identical thereto. In some embodiments, the binding domain linker comprises the sequence of any one of SEQ ID NO: 74-77, or a sequence at least 90% or at least 95% identical thereto.

In some embodiments, a binding molecule comprises a CD8+ T-cell epitope that is heterologous to Shiga toxin A subunits. In some embodiments, the CD8+ T-cell epitope comprises the sequence NLVPMVATV (SEQ ID NO: 78), or a sequence at least 90% or at least 95% identical thereto. In some embodiments, the CD8+ T-cell epitope is linked to the binding region via a cleavable spacer. In some embodiments, a binding molecule has a spacer having the sequence HHAA (SEQ ID NO: 265). In some embodiments, a binding molecule has a spacer having the sequence RR.

In some embodiments, the binding molecule comprises, from N-terminus to C-terminus, a Shiga toxin A subunit effector polypeptide, a binding domain linker, and a binding region. In some embodiments, the binding molecule comprises, from N-terminus to C-terminus, a Shiga toxin A subunit effector polypeptide, a binding domain linker, a VH and a VL, In some embodiments, the binding molecule comprises, from N-terminus to C-terminus, a Shiga toxin A subunit effector polypeptide, a binding domain linker, a VH, a scFv linker, and a VL.

In some embodiments, a binding molecule comprises, from N-terminus to C-terminus, a Shiga toxin A subunit effector polypeptide, a binding domain linker, a binding region, and a CD8+ T-cell epitope. In some embodiments, the binding molecule comprises, from N-terminus to C-terminus, a Shiga toxin A subunit effector polypeptide, a binding domain linker, a VH, a scFv linker, a VL, and a CD8+ T-cell epitope. In some embodiments, a binding molecule comprises, from N-terminus to C-terminus, a Shiga toxin A subunit effector polypeptide, a binding domain linker, a binding region, a cleavable spacer and a CD8+ T-cell epitope.

In some embodiments, a binding molecule comprises the sequence of any one of SEQ ID NO: 108-127, 129-155, 158-159, or 160-168, or a sequence at least 90% or at least 95% identical thereto.

In some embodiments, a binding molecule comprises two or more (e.g., three, four, five, six, seven, or eight) polypeptides. In some embodiments, the two polypeptides are non-covalently linked to each other, for example via the binding region.

In some embodiments, the binding molecule is cytotoxic. In some embodiments, the PD-L1 binding molecule is non-cytotoxic. For example, the PD-L1 binding molecule may be non-cytotoxic if the Shiga toxin subunit effector polypeptide is truncated or comprises one or more mutations which eliminate its cytotoxic activity.

In some embodiments of the PD-L1 binding molecule, upon administration of the PD-L1 binding molecule to a PD-L1-expressing cell results in (i) the internalization of the PD-L1 binding molecule by the cell and (ii) the death of the cell. In some embodiments of the PD-L1 binding molecule, upon administration of the PD-L1 binding molecule to a PD-L1-expressing cell results in (i) the internalization of the PD-L1 binding molecule by the cell and (ii) the death of the cell due to a catalytically active Shiga toxin A subunit effector polypeptide. In some embodiments of the PD-L1 binding molecule, upon administration of the PD-L1 binding molecule to a PD-L1-expressing cell results in (i) the internalization of the PD-L1 binding molecule by the cell and (ii) the death of the cell due to delivery and presentation of T-cell epitope cargo. In some embodiments, the PD-L1 binding molecule is capable, when introduced to cells, of exhibiting a cytotoxicity with a half-maximal inhibitory concentration (CD₅₀) value of 300 nM or less and/or capable of exhibiting a significant level of Shiga toxin cytotoxicity.

In some embodiments of the PD-L1 binding molecule, the Shiga toxin A Subunit effector polypeptide is capable of exhibiting a ribosome inhibition activity with a half-maximal inhibitory concentration (IC₅₀) value of less than 10.000, 5,000, 1,000, 500, or 200 picomolar.

In some embodiments of the PD-L1 binding molecule, the at least one Shiga toxin A Subunit derived polypeptide comprises a combination of features (e.g., de-immunized sub-region(s), heterologous epitope comprising sub-region(s), a protease-cleavage resistant sub-region, and/or a carboxy-terminal, endoplasmic reticulum retention/retrieval signal motif). Certain PD-L1 binding molecules described herein provide a combination of several properties in a single molecule, such as, e.g., efficient cellular internalization, potent cell-targeted cytotoxicity, selective cytotoxicity, de-immunization, low non-specific toxicity at high dosages, high stability, CD8+ T-cell hyper-immunization, and/or the ability to deliver a heterologous, T-cell epitope(s) to the MHC I class pathway of a target cell.

In some embodiments, the PD-L1 binding molecules are useful for administration to chordates, such as, e.g., when it is desirable to (1) reduce or eliminate a certain immune response(s) resulting from the administered molecule, (2) reduce or eliminate non-specific toxicities resulting from the administered molecule, (3) specifically kill a PD-L1-expressing target cell(s) in vivo, and/or (4) target a beneficial immune response(s) to a target cell-type, a tumor mass comprising a target cell-type, and/or a tissue locus comprising such a target cell-type, such as via stimulating intercellular engagement of a CD8+ T-cell(s) of the chordate with a specific MHC class I-epitope complex displaying target cell-type.

In some embodiments, the PD-L1 binding molecule comprises or consists essentially of the polypeptide shown in any one of SEQ ID NOs: 85-107 and 156-157, and optionally the PD-L1 binding molecule comprises an amino-terminal methionine residue.

As used herein, the term “Cmax” refers to the peak serum concentration that a binding molecule achieves after it has been administered to a subject. In some embodiments, the PD-L1 binding molecules described herein have a Cmax in the range of about 1000 to about 50,000 ng/mL. For example, the PD-L1 binding molecules may have a Cmax in the range of about 1 to about 1,000 ng/mL, about 1.000 to about 3,000 ng/mL, about 2,000 to about 5,000 ng/mL, about 5000 to about 10,000 ng/mL, about 10,000 ng/mL to about 15,000 ng/mL, about 15,000 ng/mL to about 20,000 ng/mL, about 20,000 ng/mL to about 25,000 ng/mL, about 25,000 ng/mL to about 30,000 ng/mL, or about 30,000 ng/mL to about 35,000 ng/mL, or about 35,000 ng/mL to about 50,000 ng/mL. In some embodiments the Cmax is about 1,000, about 2,000, about 3,000, about 4,000, about 5,000, about 6,00, about 7,000, about 8,000, about 9,000, or about 10,000 ng/mL. In some embodiments, the Cmax is about 21,000, about 22,000, about 23,000, about 24,000, about 25,000, about 26,000, about 27,000, about 28,000, about 29,000, or about 30,000 ng/mL. In some embodiments, the Cmax is 2,096, 27,063, or 22,375 ng/mL.

As used herein the term “half-life” or “T_{1/2}” refers to the time taken for half the initial dose of PD-L1 binding molecule administered to be eliminated from the body. In some embodiments, the half-life of a PD-L1 molecule described herein is about 1 minute to about 1 hour, about 1 hour to about 3 hours, about 3 hours to about 5 hours, or about 5 hours to about 10 hours. In some embodiments, the half-life of a PD-L1 binding molecule is about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments the half-life of a PD-L1 binding molecule is about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.6 hours, or about 10 hours. In some embodiments, the half-life of a PD-L1 binding molecule is about 2.8 hours, about 3.7 hours, or about 5.6 hours.

In some embodiments of the PD-L1 binding molecule, upon administration of the PD-L1 binding molecule to a PD-L1-expressing cell results in (i) the internalization of the PD-L1 binding molecule by the cell and (ii) the cell presenting on a cellular surface a heterologous, CD8+ T-cell epitope-peptide cargo delivered by the PD-L1 binding molecule complexed With a MHC class I molecule.

Illustrative PD-L1 binding molecules are provided in Table 7 below.

TABLE 7
Illustrative PD-L1 binding molecules

| PD-L1 | | | | |
|---|---|---|---|---|
| binding | | HLA | | SEQ ID |
| molecule | AST Feature | Restriction | SEQUENCE | NO |
| Molecule A | Single, | HLA:A*01 | MKEFTLDFSTAKTYVDSLNVIRSAIGTPLQTISSGGTSLLMI | 303 |
| | C-terminal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAEFPKPSTPPGSSGGAPEVQLQQSGPELVKPG | |
| | | | ASVKISCKTSGYTFTEYTMHWVKQRHGKSLEWIGGINPN | |
| | | | NGGTWYNQKFKGKATLTVTDKSSSTAYMELRSLTSEDSAV | |
| | | | YFCARPYYYGSREDYFDYWGQGTTLTVSSGGGGSDIQMT | |
| | | | QSPSSLSASVGDRVTITCSASSSVSYMYWYQQKPRSSPKP | |
| | | | WIYLTSNLASGVPARFSGSGSGTSYSLTISSMEAEDAATY | |
| | | | YCQQWSSNPPTFGGGTKLELKHHAAYSEHPTFTSQY | |
| Molecule B | Single, | HLA:A*01 | MKEFTLDFSTAKTYVDSLNVIRSAIGTPLQTISSGGTSLLMI | 304 |
| | C-terminal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAEFPKPSTPPGSSGGAPEVQLQQSGPELVKPG | |
| | | | ASVKISCKTSGYTFTEYTMHWVKQRHGKSLEWIGGINPN | |
| | | | NGGTWYNQKFKGKATLTVDKSSSTAYMELRSLTSEDSAV | |
| | | | YFCARPYYYGSREDYFDYWGQGTTLTVSSGGGGSDIQMT | |
| | | | QSPSSLSASVGDRVTITCSASSSVSYMYWYQQKPRSSPKP | |
| | | | WIYLTSNLASGVPARFSGSGSGTSYSLTISSMEAEDAATY | |
| | | | YCQQWSSNPPTFGGGTKLELKHHAAVTEHDTLLY | |
| Molecule C | Single, | HLA:A*03 | MKEFTLDFSTAKTYVDSLNVIRSAIGTPLQTISSGGFSLLMI | 305 |
| | C-terminal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINADLGSVALILNS | |
| | | | HHHASAVAAEFPKPSTPPGSSGGAPEVQLQQSGPELVKPG | |
| | | | ASVKISCKTSGYTFTEYTMHWVKQRHGKSLEWIGGINPN | |
| | | | NGGTWYNQKFKGKATLTVDKSSSTAYMELRSLTSEDSAV | |
| | | | YFCARPYYYGSREDYFDYWGQGTTLTVSSGGGGSDIQMT | |
| | | | QSPSSLSASVGDRVTITCSASSSVSYMYWYQQKPRSSPKP | |
| | | | WIYLTSNLASGVPARFSGSGSGTSYSLTISSMEAEDAATY | |
| | | | YCQQWSSNPPTFGGGTKLELKHHAAKLGGALQAK | |
| Molecule D | Single, | HLA:A*24 | MKEFTLDFSTAKTYVDSLNVIRSAIGTPLQTISSGGTSLLMI | 306 |
| | C-terminal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAEFPKPSTPPGSSGGAPEVQLQQSGPELVKPG | |
| | | | ASVKISCKTSGYTFTEYTMHWVKQRHGKSLEWIGGINPN | |
| | | | NGGTWYNQKFKGKATLTVDKSSSTAYMELRSLTSEDSAV | |
| | | | YFCARPYYYGSREDYFDYWGQGTTLTVSSGGGGSDIQMT | |
| | | | QSPSSLSASVGDRVTITCSASSSVSYMYWYQQKPRSSPKP | |
| | | | WIYLTSNLASGVPARFSGSGSGTSYSLTISSMEAEDAATY | |
| | | | YCQQWSSNPPTFGGGTKLELKHHAAOYDPVAALF | |
| Molecule E | Single, | HLA:A*01 | MKEFTLDFSTAKTYVDSLNVIRSAIGTPLQTISSGGTSLLMI | 307 |
| | internal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAYSEHPTFTSQYEFPKPSTPPGSSGGAPEVQL | |
| | | | QQSGPELVKPGASVKISCKTSGYTFTEYTMHWVKQRHGK | |
| | | | SLEWIGGINPNNGGTWYNQKPKGKATLTVDKSSSTAYME | |
| | | | LRSLTSEDSAVYFCARPYYYGSREDYFDYWGQGTTLTVS | |
| | | | SGGGGSDIQMTQSPSSLSASVGDRVTITCSASSSVSYMYW | |
| | | | YQQKPRSSPKPWIYLTSNLASGVPARFSGSGSGTSYSLTIS | |
| | | | SMEAEDAATYYCQQWSSNPPTFGGGTKLELK | |
| Molecule F | Single, | HLA:A*01 | MKEFTLDFSTAKTYVDSLNVIRSAIGTPLQTISSGGTSLLMI | 308 |
| | internal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAVTEHDTLLYEFPKPSTPPGSSGGAPEVQLQQ | |
| | | | SGPELVKPGASVKISCKTSGYTFTEYTMHWVKQRHGKSL | |
| | | | EWIGGINPNNGGTWYNQKFKGKATLTVDKSSSTAYMELR | |
| | | | SLTSEDSAVYFCARPYYYGSREDYFDYWGQGTTLTVSSG | |
| | | | GGGSDIQMTQSPSSLSASVGDRVTITCSASSSVSYMYWYQ | |
| | | | QKPRSSPKPWIYLTSNLASGVPARFSGSGSGTSYSLTISSM | |
| | | | EAEDAATYYCQQWSSNPPTFGGGTKLELK | |
| Molecule G | Single, | HLA:A*03 | MKEFTLDFSTAKTYVDSLNVIRSAIGTPLQTISSGGTSLLMI | 309 |
| | internal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAKLGGALQAKEFPKPSTPPGSSGGAPEVQLQ | |
| | | | QSGPELVKPGASVKISCKTSGYTFTEYTMHWVKQRHGKS | |
| | | | LEWIGGINPNNGGTWYNQKFKGKATLTVDKSSSTAYMEL | |
| | | | RSLTSEDSAVYFCARPYYYGSREDYFDYWGQGTTLTVSS | |
| | | | GGGGSDIQMTQSPSSLSASVGDRVTITCSASSSVSYMYWY | |
| | | | QQKPRSSPKPWIYLTSNLASGVPARFSGSGSGTSYSLTISS | |
| | | | MEAEDAATYYCQQWSSNPPTFGGGTKLELK | |
| Molecule H | Single, | HLA:A*24 | MKEFTLDFSTAKTYVDSLNVIRSAIGTPLOTISSGGTSLLMI | 310 |
| | internal | | DSGIGDNLFAVDILGFDFTLGRFNNLRLIVERNNLYVTGFV | |
| | antigen | | NRTNNVFYRFADFSHVTFPGTTAVTLSADSSYTTLQRVAG | |
| | | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAQYDPVAALFEFPKPSTPPGSSGGAPEVQLQ | |
| | | | QSGPELVKPGASVKISCKTSGYTFTEYTMHWVKQRHGKS | |
| | | | LEWIGGINPNNGGTWYNOKFKGKATLTVDKSSSTAYMEL | |
| | | | RSLTSEDSAVYFCARPYYYGSREDYFDYWGQGTTLTVSS | |
| | | | GGGGSDIQMTQSPSSLSASVGDRVTITCSASSSVSYMYWY | |
| | | | QQKPRSSPKPWIYLTSNLASGVPARFSGSGSGTSYSLTISS | |
| Molecule I | Multiple | HLA-A*01 | MKEFTLDFSTAKTYVDSLNVIRSAIGTPLQTISSGGTSLLMI | 311 |
| | antigens (1 | HLA-A*02 | DSGIGDNLFAVDILGFDFTLGRFNNLRLIVERNNLYVTGFV | |
| | internal, 2 | | NRTNNVFYRFADFSHVTFPGTTAVTLSADSSYTTLQRVAG | |
| | C-terminal) | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAYSEHPTFTSQYEFPKPSTPPGSSGGAPEVQL | |
| | | | QQSGPELVKPGASVKISCKTSGYTFTEYTMHWVKQRHGK | |
| | | | SLEWIGGINPNNGGTWYNQKFKGKATLTVDKSSSTAYME | |
| | | | LRSLTSEDSAVYFCARPYYYGSREDYFDYWGQGTTLTVS | |
| | | | SGGGGSDIQMTQSPSSLSASVGDRVTITCSASSSVSYMYW | |
| | | | YQQKPRSSPKPWIYLTSNLASGVPARFSGSGSGTSYSLTIS | |
| | | | SMEAEDAATYYCQQWSSNPPTFGGGTKLELKHHAANLV | |
| | | | PMVATVRRVTEHDTLLY | |
| Molecule J | Multiple | HLA-A*01 | MKEFTLDFSTAKTYVDSLNVIRSAIGTPLQTISSGGTSLLMI | 312 |
| | antigens (1 | HLA-A*03 | DSGIGDNLFAVDILGFDFTLGRFNNLRLIVERNNLYVTGFV | |
| | internal, 2 | HLA-A*24 | NRTNNVFYRFADFSHVTFPGTTAVTLSADSSYTTLQRVAG | |
| | C-terminal) | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAYSEHPTFTSQYEFPKPSTPPGSSGGAPEVQL | |
| | | | QQSGPELVKPGASVKISCKTSGYTFTEYTMHWVKQRHGK | |
| | | | SLEWIGGINPNNGGTWYNQKFKGKATLTVDKSSSTAYME | |
| | | | LRSLTSEDSAVYFCARPYYYGSREDYFDYWGQGTTLTVS | |
| | | | SGGGGSDIQMTQSPSSLSASVGDRVTITCSASSSVSYMYW | |
| | | | YQQKPRSSPKPWIYLTSNLASGVPARFSGSGSGTSYSLTIS | |
| | | | SMEAEDAATYYCQQWSSNPPTFGGGTKLELKHHAAKLG | |
| | | | GALQAKRRQYDPVAALF | |
| Molecule K | Multiple | HLA-A*01 | MKEFTLDFSTAKTYVDSLNVIRSAIGTPLQTISSGGTSLLMI | 313 |
| | antigens (1 | HLA-A*24 | DSGIGDNLFAVDILGFDFTLGRFNNLRLIVERNNLYVTGFV | |
| | internal, 2 | HLA-A*03 | NRTNNVFYRFADFSHVTFPGTTAVTLSADSSYTTLQRVAG | |
| | C-terminal) | | ISRTGMQINRHSLTTSYLDLMSHSGTSLTQSVARAMLRFV | |
| | | | TVTAEALRFRQIQRGFRTTLDDLSGASYVMTAEDVDLTL | |
| | | | NWGRLSSVLPDYHGQDSVRVGRISFGSINAILGSVALILNS | |
| | | | HHHASAVAAYSEHPTFTSQYEFPKPSTPPGSSGGAPEVQL | |
| | | | QQSGPELVKPGASVKISCKTSGYTFTEYTMHWVKQRHGK | |
| | | | SLEWIGGINPNNGGTWYNQKFKGKATLTVDKSSSTAYME | |
| | | | LRSLTSEDSAVYFCARPYYYGSREDYFDYWGQGTTLTVS | |
| | | | SGGGGSDIQMTQSPSSLSASVGDRVTITCSASSSVSYMYW | |
| | | | YQQKPRSSPKPWIYLTSNLASGVPARFSGSGSGTSYSLTIS | |
| | | | SMEAEDAATYYCQQWSSNPPTFGGGTKLELKHHAAQYD | |
| | | | PVAALFRRKLGGALQAK | |

### Other Structural Variations

In some embodiments, fragments, variants, and/or derivatives of the binding molecules are used, which contain a functional binding site to any extracellular part of a PD-L1 target biomolecule, and even more preferably capable of binding a target biomolecule with high affinity (e.g. as shown by K_{D}). For example, any binding region which binds an extracellular part of a target biomolecule with a dissociation constant (K_{D}) of 10⁻⁵ to 10⁻¹² moles/liter, preferably less than 200 nM, may be substituted for use in making binding molecules and methods as described herein.

The skilled worker will recognize that variations may be made to the Shiga toxin effector polypeptides, antibodies, and binding molecules, and polynucleotides encoding any of the former, without diminishing their biological activities, e.g., by maintaining the overall structure and function of the Shiga toxin effector polypeptide, such as in conjunction with one or more 1) endogenous epitope disruptions which reduce antigenic and/or immunogenic potential, 2) furin-cleavage site disruptions which reduce proteolytic cleavage, and/or 3) embedded or inserted epitopes which reduce antigenic and/or immunogenic potential or are capable of being delivered to a MHC I molecule for presentation on a cell surface. For example, some modifications may facilitate expression, facilitate purification, improve pharmacokinetic properties, and/or improve immunogenicity. Such modifications are well known to the skilled worker and include, for example, a methionine added at the amino-terminus to provide an initiation site, additional amino acids placed on either terminus to create conveniently located restriction sites or termination codons, and biochemical affinity tags fused to either terminus to provide for convenient detection and/or purification. A common modification to improve the immunogenicity of a polypeptide produced using a non-chordate system (e.g. a prokaryotic cell) is to remove, after the production of the polypeptide, the starting methionine residue, which may be formylated during production, such as, e.g., in a bacterial host system, because. e.g., the presence of N-formylmethionine (fMet) might induce undesirable immune responses in chordates.

Also contemplated herein is the inclusion of additional amino acid residues at the amino and/or carboxy termini of a binding molecule, or a proteinaceous component of a binding molecule, such as sequences for epitope tags or other moieties. The additional amino acid residues may be used for various purposes including, e.g., facilitating cloning, facilitating expression, post-translational modification, facilitating synthesis, purification, facilitating detection, and administration. Non-limiting examples of epitope tags and moieties are chitin binding protein domains, enteropeptidase cleavage sites, Factor Xa cleavage sites, FIAsH tags, FLAG tags, green fluorescent proteins (GFP), glutathione-S-transferase moieties, HA tags, maltose binding protein domains, myc tags, polyhistidine tags, ReAsH tags, strep-tags, strep-tag II, TEV protease sites, thioredoxin domains, thrombin cleavage site, and V5 epitope tags.

In certain of the above embodiments, the polypeptide sequence of the Shiga toxin effector polypeptides and/or binding molecules are varied by one or more conservative amino acid substitutions introduced into the polypeptide region(s) as long as all required structural features are still present and the Shiga toxin effector polypeptide is capable of exhibiting any required function(s), either alone or as a component of a binding molecule. As used herein, the term “conservative substitution” denotes that one or more amino acids are replaced by another, biologically similar amino acid residue. Examples include substitution of amino acid residues with similar characteristics. e.g. small amino acids, acidic amino acids, polar amino acids, basic amino acids, hydrophobic amino acids and aromatic amino acids (see, for example, Table 4). An example of a conservative substitution with a residue normally not found in endogenous, mammalian peptides and proteins is the conservative substitution of an arginine or lysine residue with, for example, omithine, canavanine, aminoethylcysteine, or another basic amino acid. For further information concerning phenotypically silent substitutions in peptides and proteins see, e.g., Bowie J et al., Science 247: 1306-10 (1990).

TABLE 4
Examples of Conservative Amino Acid Substitutions

| I | II | III | IV | V | VI | VII | VIII | IX | X | XI | XII | XIII | XIV |
|---|---|---|---|---|---|---|---|---|---|---|---|---|---|
| A | D | H | C | F | N | A | C | F | A | C | A | A | D |
| G | E | K | I | W | Q | G | M | H | C | D | C | C | E |
| P | Q | R | L | Y | S | I | P | W | F | E | D | D | G |
| S | N | | M | | T | L | | Y | G | H | G | E | K |
| T | | | V | | | V | | | H | K | N | G | P |
| | | | | | | | | | I | N | P | H | Q |
| | | | | | | | | | L | Q | S | K | R |
| | | | | | | | | | M | R | T | N | S |
| | | | | | | | | | R | S | V | Q | T |
| | | | | | | | | | T | T | | R | |
| | | | | | | | | | V | | | S | |
| | | | | | | | | | W | | | P | |
| | | | | | | | | | Y | | | T | |

In the conservative substitution scheme in Table 4, exemplary conservative substitutions of amino acids are grouped by physicochemical properties—I: neutral, hydrophilic; II: acids and amides; III: basic; IV: hydrophobic; V: aromatic, bulky amino acids. VI hydrophilic uncharged, VII aliphatic uncharged, VIII non-polar uncharged, IX cycloalkenyl-associated, X hydrophobic, XI polar, XII small, XIII turn-permitting, and XIV flexible. For example, conservative amino acid substitutions include the following: 1) S may be substituted for C; 2) M or L may be substituted for F; 3) Y may be substituted for M; 4) Q or E may be substituted for K; 5) N or Q may be substituted for H; and 6) H may be substituted for N.

Additional conservative amino acid substitutions include the following: 1) S may be substituted for C; 2) M or L may be substituted for F; 3) Y may be substituted for M; 4) Q or E may be substituted for K; 5) N or Q may be substituted for H; and 6) H may be substituted for N.

Variants of the Shiga toxin effector polypeptides and binding molecules may be prepared by changing a polypeptide described herein by altering one or more amino acid residues or deleting or inserting one or more amino acid residues, such as within the binding region or Shiga toxin effector polypeptide region, in order to achieve desired properties, such as changed cytotoxicity, changed cytostatic effects, changed immunogenicity, and/or changed serum half-life. The Shiga toxin effector polypeptides and binding molecules may further be with or without a signal sequence. In some embodiments, the binding molecules may comprise functional fragments or variants of a polypeptide region described herein that have, at most, 20, 15, 10, 9, 8, 7, 6, 5, 4, 3, 2, or 1 amino acid substitutions compared to a polypeptide sequence recited herein.

In some embodiments, the Shiga toxin effector polypeptides and binding molecules may comprise functional fragments or variants of a polypeptide region described herein that have, at most, 20, 15, 10, 9, 8, 7, 6, 5, 4, 3, 2, or 1 amino acid substitutions compared to a polypeptide sequence recited herein, as long as it (1) comprises at least one embedded or inserted, heterologous T-cell epitope and at least one amino acid is disrupted in an endogenous, B-cell and/or CD4+ T-cell epitope region, wherein the disrupted amino acid does not overlap with the embedded or inserted epitope; (2) comprises at least one embedded or inserted, heterologous T-cell epitope and a disrupted furin-cleavage site at the carboxy-terminus of a Shiga toxin A1 fragment derived region; or (3) comprises a disrupted furin-cleavage site at the carboxy-terminus of a Shiga toxin A1 fragment derived region and comprises at least one amino acid is disrupted in an endogenous, B-cell and/or CD4+ T-cell epitope region, wherein the disrupted amino acid does not overlap with the disrupted furin-cleavage motif.

Accordingly, in some embodiments, the Shiga toxin effector polypeptide comprises or consists essentially of amino acid sequences having at least 55%, 60%, 65%, 70%, 75%, 80%, 85%, 90%, 95%, 97%, 98%, or 99%, overall sequence identity to a naturally occurring Shiga toxin A Subunit or fragment thereof, such as, e.g., Shiga toxin A Subunit, such as SLT-1A (SEQ ID NO:1), StxA (SEQ ID NO:2), and/or SLT-2A (SEQ ID NO:3), wherein the Shiga toxin effector polypeptide (1) comprises at least one embedded or inserted, heterologous T-cell epitope and at least one amino acid is disrupted in an endogenous, B-cell and/or CD4+ T-cell epitope region, and wherein the disrupted amino acid does not overlap with the embedded or inserted epitope; (2) comprises at least one embedded or inserted, heterologous T-cell epitope and a disrupted furin-cleavage site at the carboxy-terminus of a Shiga toxin A1 fragment derived region; or (3) comprises a disrupted furin-cleavage site at the carboxy-terminus of a Shiga toxin A1 fragment derived region and comprises at least one amino acid is disrupted in an endogenous, B-cell and/or CD4+ T-cell epitope region, and wherein the disrupted amino acid does not overlap with the disrupted furin-cleavage site.

In some embodiments, the Shiga toxin effector polypeptide has one or more amino acid residues may be mutated, inserted, or deleted in order to increase the enzymatic activity of the Shiga toxin effector polypeptide. In some embodiments, the Shiga toxin effector polypeptide has one or more amino acid residues may be mutated or deleted in order to reduce or eliminate catalytic and/or cytotoxic activity of the Shiga toxin effector polypeptide. For example, the catalytic and/or cytotoxic activity of the A Subunits of members of the Shiga toxin family may be diminished or eliminated by mutation or truncation.

The cytotoxicity of the A Subunits of members of the Shiga toxin family may be altered, reduced, or eliminated by mutation and/or truncation. The positions labeled tyrosine-77, glutamate-167, arginine-170, tyrosine-114, and tryptophan-203 have been shown to be important for the catalytic activity of Stx, Stx1, and Stx2 (Hovde C et al., Proc Natl Acad Sci USA 85: 2568-72 (1988); Deresiewicz R et al., Biochemistry 31: 3272-80 (1992); Deresiewicz R et al., Mol Gen Genet 241: 467-73 (1993); Ohmura M et al., Microb Pathog 15: 169-76 (1993); Cao C et al., Microbiol Immunol 38: 441-7 (1994); Suhan M, Hovde C, Infect Immun 66: 5252-9 (1998)). Mutating both glutamate-167 and arginine-170 eliminated the enzymatic activity of Slt-I A1 in a cell-free ribosome inactivation assay (LaPointe P et al., J Biol Chem 280: 23310-18 (2005)). In another approach using de novo expression of Sit-I A1 in the endoplasmic reticulum, mutating both glutamate-167 and arginine-170 eliminated Slt-I A1 fragment cytotoxicity at that expression level (LaPointe P et al., J Biol Chem 280: 23310-18 (2005)). A truncation analysis demonstrated that a fragment of StxA from residues 75 to 268 still retains significant enzymatic activity in vitro (Haddad J et al., J Bacteriol 175: 4970-8 (1993)). A truncated fragment of Slt-I A1 containing residues 1-239 displayed significant enzymatic activity in vitro and cytotoxicity by de novo expression in the cytosol (LaPointe P et al., J Biol Chem 280: 23310-18 (2005)). Expression of a Slt-I A1 fragment truncated to residues 1-239 in the endoplasmic reticulum was not cytotoxic because it could not retrotranslocate to the cytosol (LaPointe P et al., J Biol Chem 280: 23310-18 (2005)).

The most critical residues for enzymatic activity and/or cytotoxicity in the Shiga toxin A Subunits were mapped to the following residue-positions: asparagine-75, tyrosine-77, tyrosine-114, glutamate-167, arginine-170, arginine-176, and tryptophan-203 among others (Di R et al., Toxicon 57: 525-39 (2011)). In particular, a double-mutant construct of Stx2A containing glutamate-E167-to-lysine and arginine-176-to-lysine mutations was completely inactivated; whereas, many single mutations in Stx1 and Stx2 showed a 10-fold reduction in cytotoxicity. Further, truncation of Stx1A to 1-239 or 1-240 reduced its cytotoxicity, and similarly, truncation of Stx2A to a conserved hydrophobic residue reduced its cytotoxicity. The most critical residues for binding eukaryotic ribosomes and/or eukaryotic ribosome inhibition in the Shiga toxin A Subunit have been mapped to the following residue-positions arginine-172, arginine-176, arginine-179, arginine-188, tyrosine-189, valine-191, and leucine-233 among others (McCluskey A et al., PLoS One 7: e31191 (2012). However, certain modification may increase a Shiga toxin functional activity exhibited by a Shiga toxin effector polypeptide. For example, mutating residue-position alanine-231 in Stx1A to glutamate increased Stx1A's enzymatic activity in vitro (Suhan M, Hovde C, Infect Immun 66: 5252-9 (1998)).

In some embodiments, the Shiga toxin effector polypeptide derived from SLT-1A (SEQ ID NO:1) or StxA (SEQ ID NO:2) has one or more amino acid residues mutated include substitution of the asparagine at position 75, tyrosine at position 77, tyrosine at position 114, glutamate at position 167, arginine at position 170, arginine at position 176, and/or substitution of the tryptophan at position 203. Examples of such substitutions will be known to the skilled worker based on the prior art, such as asparagine at position 75 to alanine, tyrosine at position 77 to serine, substitution of the tyrosine at position 114 to serine, substitution of the glutamate position 167 to glutamate, substitution of the arginine at position 170 to alanine, substitution of the arginine at position 176 to lysine, substitution of the tryptophan at position 203 to alanine, and/or substitution of the alanine at 231 with glutamate. Other mutations which either enhance or reduce Shiga toxin enzymatic activity and/or cytotoxicity are within the scope of the disclosure and may be determined using well known techniques and assays disclosed herein.

The Shiga toxin effector polypeptides and binding molecules may optionally be conjugated to one or more additional agents, which may include therapeutic agents, diagnostic agents, and/or other additional exogenous materials known in the art, including such agents as described herein. In some embodiments, the Shiga toxin effector polypeptide or binding molecule is PEGylated or albuminated, such as, e.g., to provide de-immunization, disrupt furin-cleavage by masking the extended loop and/or the furin-cleavage site at the carboxy-terminus of a Shiga toxin A1 fragment derived region, improve pharmacokinetic properties, and/or improve immunogenicity (see e.g., Wang Q et al., Cancer Res 53: 4588-94 (1993); Tsutsumi Y et al., Proc Natl Acad Sci USA 97; 8548-53 (2000); Buse J, El-Aneed A, Nanomed 5: 1237-60 (2010); Lim S et al.. J Control Release 207-93 (2015)).

### 1. Antibody Component Variants

In some embodiments, amino acid sequence variants of the antibody component of the binding molecules (e.g. an antibody-toxin conjugate) described herein are contemplated. For example, it may be desirable to improve the binding affinity and/or other biological properties of the antibody-toxin conjugate. Amino acid sequence variants of an antibody may be prepared by introducing appropriate modifications into the nucleotide sequence encoding the antibody component, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding and/or toxin delivery.

### a) Substitution, Insertion, and Deletion Variants

In some embodiments, antibody variants having one or more amino acid substitutions are provided. Sites of interest for substitutional mutagenesis include the HVRs and FRs. Amino acid substitutions may be introduced into an antibody of interest and the antibody-toxin conjugate products screened for a desired activity, e.g., retained/improved antigen binding, decreased immunogenicity, or improved ADCC or CDC.

One type of substitutional variant involves substituting one or more hypervariable region residues of a parent antibody (e.g. to create a humanized antibody). Generally, the resulting variant(s) selected for further study will have modifications (e.g. improvements) in certain biological properties (e.g. increased affinity or reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more HVR residues are mutated and the variant antibodies displayed and screened for a particular biological activity (e.g. binding affinity) (see e.g. WO 2015/120058).

Alterations (e.g. substitutions) may be made in HVRs, e.g., to improve antibody affinity using methods known to the skilled worker. For example, alterations may be made in HVR “hotspots” or residues encoded by codons that undergo mutation at high frequency during the somatic maturation process (Chowdhury P, Methods Mol Biol 207: 179-196 (2008)), and/or SDRs (a-CDRs), with the resulting variant heavy and/or light chains being tested for binding affinity. In some embodiments, substitutions, insertions, or deletions may occur within one or more HVRs so long as such alterations do not substantially reduce the ability of the antibody to bind antigen. For example, conservative alterations that do not substantially reduce binding affinity may be made in HVRs, including outside of HVR “hotspots” or SDRs. In some embodiments of the variant VH and VL sequences provided above, each HVR either is unaltered, or contains no more than one, two, or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an amino-terminal methionyl residue. Other insertional variants of the antibody molecule include the fusion to the amino- and/or carboxyl-terminus of the antibody to an enzyme (e.g. for antibody-directed enzyme prodrug therapy) or a polypeptide which increases the serum half-life of the antibody.

b) De-Immunized and/or Chimeric Variants

In some embodiments, the antibody component of the binding molecule (e.g. an antibody-toxin conjugate) is chimeric. For example, the chimeric antibody comprises a non-human variable region (e.g., a variable region derived from a mouse, rat, hamster, rabbit, or non-human primate) and a human constant region. In a further example, a chimeric antibody is a “class switched” antibody in which the class or isotype has been changed from that of the parent antibody from which it was derived. In some embodiments, the chimeric antibody is a humanized antibody. Chimeric antibodies include antigen-binding fragments thereof.

In some embodiments, the antibody component of the binding molecule (e.g. an antibody-toxin conjugate) is humanized. Typically, a non-human antibody is humanized to reduce immunogenicity in humans, while retaining the specificity and affinity of the parental non-human antibody. Typically, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a constant region from a human antibody. In some embodiments, some FR residues in a humanized antibody have been substituted with corresponding residues from a non-human antibody (e.g. the antibody from which the HVR residues are derived), e.g., to restore or improve antibody specificity and/or affinity.

### c) Fc Region Variants

In some embodiments, the antibody component of the binding molecule (e.g. an antibody-toxin conjugate) comprises an Fc region. For example, the Fc region variant may comprise a human Fc region sequence (e.g., a Fc region from a human IgG1, IgG2, IgG3, or IgG4) and may optionally comprise one or more amino acid alterations (e.g. a substitution at one or more amino acid positions). In some embodiments, the antibody component comprises an Fc region that has ADCC and/or CDC activity. Such antibodies are particularly useful for mediating killing of target expressing cells. Antibodies with improved Fc effector functions can be generated, for example, through changes in amino acid residues involved in the interaction between the Fc domain and an Fc receptor (FcR) (e.g. FcγRI, FcγRIIA, FcγRIIB, or FcγRIII with FcRn), which may lead to increased cytotoxicity and/or altered pharmacokinetics, such as increased serum half-life. Certain antibody variants with improved or diminished binding to FcRs are known to skilled worker and/or described in Shields R et al., J Biol Chem 9: 6591-6604 (2001).

In some embodiments, the antibody component comprises an Fc region that lacks one or more effector functions (e.g. lacks ADCC and/or CDC activity). Fc regions lacking or having substantially reduced effector function may be obtained, for example, by introducing one or more amino acid substitutions into a native Fc region sequence, such that the Fc region does not bind, or has substantially reduced binding, to cytolytic Fc receptors (e.g. DANA mutant) and/or the C1q complement protein (see e.g. Wilson N et al., Cancer Cell 19: 101-113 (2011); Idusogie E et al. J Immunol 164: 4178-4184 (2000)). In some embodiments, the antibody component is varied in that it possesses some but not all antibody effector functions, which make it a desirable candidate for applications in which the half-life of the binding molecule in vivo is important yet certain effector functions (e.g. CDC or ADCC) are undesirable or deleterious.

In some embodiments, the antibody component comprises an Fc region with one or more amino acid substitutions which improve ADCC, e.g., substitutions at positions 298, 333, and/or 334 of the Fc region (EU numbering of residues).

In some embodiments, the antibody component comprises an Fc region with one or more amino acid substitutions resulting in altered Clq binding and/or CDC effector function (e.g. either improved or diminished) (see e.g. WO 1999/051642; U.S. Pat. No. 6,194,551).

### d) Glycosylation Variants

In some embodiments, the antibody component of the binding molecule (e.g. an antibody-toxin conjugate) is altered to increase or decrease the extent to which the antibody is glycosylated. Addition or deletion of glycosylation sites to an antibody may be conveniently accomplished by altering the amino acid sequence such that one or more glycosylation sites is created or removed. For example, an antibody component comprising a glycosylated Fc region may be altered such that the carbohydrate attached thereto is altered. In another example, the carbohydrate attached to an antibody component may be altered using methods known to the skilled worker.

### e) Cysteine Engineered Antibody Variants

In some embodiments, the antibody component of the binding molecule (e.g. an antibody-toxin conjugate) possesses one or more engineered cysteine residues. In some embodiments of the antibody, it may be desirable to create cysteine engineered antibodies, such as, e.g, in which one or more residues of an antibody are substituted with cysteine residues (e.g. a ThioFab). In some embodiments, the substituted residues occur at sites of the antibody that are readily available for conjugation (see e.g. Junutula J et al., Nature Biotech 26: 925-32 (2008); Dornan D et al, Blood 114: 2721-29 (2009)). By substituting those residues with cysteine, reactive thiol groups are thereby positioned at accessible sites of the antibody and may be used to conjugate the antibody to other moieties, such as drug moieties or linker-drug moieties, to create an immunoconjugates as described further herein. In some embodiments of the antibody, it may be desirable to create cysteine engineered antibodies via one or more cysteine residue substitutions that do not significantly perturb antibody folding and assembly nor significantly alter antigen binding and/or antibody effector functions.

### 2. Immunoconjugates

Also provided herein are various embodiments of PD-L1 binding molecules, wherein each PD-L1 binding molecule comprises (1) at least one toxin component and (2) at least one PD-L1 binding region capable of specifically binding an extracellular part of a PD-L1 molecule, including immunoconjugates comprising an anti-PD-L1 antibody conjugated to one or more toxins components (e.g. protein toxins, enzymatically active toxins of bacterial, fungal, plant, or animal origin, or fragments thereof). An “immunoconjugate” is an antibody (including an antigen-binding antibody fragment) conjugated to one or more heterologous molecule(s), including but not limited to a cytotoxic agent.

In some embodiments of the binding molecule, the immunoconjugate is an antibody-toxin conjugate, which is an antibody conjugated to a toxin, such as, e.g., diphtheria A chain, exotoxin A chain (from P, aeruginosa), ricin A chain, abrin A chain, modeccin A chain, alpha-sarcin, Aleurites fordii protein, dianthin protein, Phytolaca americana protein (e.g. PAPI, PAPII, or PAP-S), Momordica charantia inhibitor, curcin, crotin, Sapaonaria officinalis inhibitor, gelonin, mitogellin, restrictocin, phenomycin, enomycin, Shiga toxin A Subunit, and tricothecenes. Biological immunoconjugates comprising a toxin (e.g. a Shiga toxin A subunit fragment) linked to a PD-L1 binding region (e.g. an antibody or antibody fragment) are useful as therapeutic or diagnostic biological molecules. In addition, such therapeutic or diagnostic molecules may be improved by having a Shiga toxin effector polypeptide conjugated to an additional agent such as, e.g., a solubility-altering agent, pharmacokinetic-altering agent, immunogenicity-altering agent, and/or a pharmacodynamic-altering agent (see e.g. WO 2018/106895). Typically, biopharmaceutical immunoconjugates are created by conjugating an antibody to other agents or cargos using chemical reactions involving a functional group(s) of the biological molecule and a functional group of the agent or cargo, or alternatively of a linker designed to bridge between the biological molecule and the agent or cargo (see section II. Linkages Connecting Components and/or Their Subcomponents, supra).

In some embodiments, the binding molecule is an immunoconjugate utilizing a cysteine engineered into the PD-L1 binding region, such as, e.g., wherein the binding molecule comprises a cysteine engineered antibody. In some embodiments, the binding molecule is an immunoconjugate utilizing a cysteine engineered into the framework region (e.g. FRI) of an immunoglobulin variable region for conjugation (see e.g. WO 2011/00054).

In some embodiments, the binding molecule is an immunoconjugate utilizing a carbohydrate moiety attached to a Fc region, such as, e.g., wherein the binding molecule comprises a glycosylated antibody or antibody fragment.

In some embodiments, the binding molecule is an immunoconjugate comprising an antibody or antibody fragment and a Shiga toxin A subunit effector polypeptide.

The toxin component of a binding molecule or antibody toxin conjugate as described herein may include, but is not limited to, natural toxins, biotoxins, proteinaceous toxins, venom, cytotoxins, small molecule toxins, and synthetic toxicants derived from any of the aforementioned, such as, e.g., aconitine, adriamycin, amanitin, amatoxin, anthracycline, aroin, apitoxin, atropine, bufotoxin, cardiac glycoside, calicheamicin, celandine, cicutoxin, colchicine, coniine, convallatoxin, crotamine, curare, curcin, dauricine, digitalis, dolastatin, duocarmycin, evomonoside, grayanotoxin, gelsemine, gelseminine, hellebrin, helleborin, hyoscyamine, ligatoxin, ligustrin, maytansine, mitomycin C, muscarine, phallotoxin, phoratoxin, phytotoxin, picrotoxin, sea nettle toxin, taxine alkaloid, thionin, vinca alkaloid, viscotoxin, and various toxin agents described herein. Pharmaceutically active cytotoxins suitable for use as a toxin component also include, but are not limited to ABx toxins, ribosome inactivating protein toxin, anthrax toxin, cholix toxin, claudin, diphtheria toxin, heat-labile enterotoxin, pertussis toxin, Pseudomonas exotoxin A, ricin, Shiga toxin, and subtilase cytotoxin; alkylating agents (such as, e.g. bendamustine, busulfan, carmustine, chlorambucil, cyclophosphamide, etramustine, ifosfamide, lomustine, mechlorethamine, melphalan, mustine, thiotepa, and treosulfan), antibiotics (such as, e.g. anthracyclines), anti-microtubule agens (such as, e.g. vinca alkaloids like vincristine, vinblastine, and etoposide or toxoids like paclitaxel and docetaxel), intercalating agents (such as, e.g. daunorubicin, bleomycin, dactinomycin, doxorubicin, epirubicin, mitoxatrone, idarubicin, plicamycin, mitomycin, and steptozotocin), anti-metabolites (such as, e.g. methotrexate, pyrimidine antagonists, and purine antagonists), growth inhibitory agents (such as topoisomerase inhibitors and spindle poisons like camptothecin, colchicine, daunorubicin, fisetin, genistein, irinotecan, lamellarins, myricetin, paclitaxel, thaspine, tricitrinol B, topotecan, vinca alkaloids); enzymes and fragments thereof such as nucleolytic enzymes like asparaginase and certain RNAses such as, e.g., bacterial RNases, fungal ribotoxins, argonaute polypeptides, binase, amphibian RNases, ranpimase, OnconaseV, and mammalian RNases, such as, e.g., bovine semen RNase and the human RNases; toxins such as small molecule toxins or enzymatically active toxins of bacterial, fungal, plant or animal origin, including fragments and/or variants thereof, such as, e.g., abrins, agrostin, amarandins, amaranthin, Amaranthus antiviral/RIP, angiogenin, A. patens RIPs, Articulatin D, asparins, aspergillin, Aspf1, balsamin, B. hispida RIP, bouganin. Bougainvillea×buttiana antiviral protein1, benincasins, bouganin, B. rubra RIPs, bryodins (e.g. bryodin 1, bryodin 2), B. spectabilis RIPs, B. vulgaris RIPs. C. album RIPs, camphorin, C. aculeatum-systemic resistance inducing protein, C. cristata RIPs, C. figarei RIPs, charantin, charybdin, cinnamomin, clavin, C. moschata RIP, cochinin B, colocins, crotins, cucurmosin, curcins, Dianthus spp. RIPs, Corynebacterium spp. diphtheria toxins (diphtheria toxins in C. ulcerans, C. omega, C. pseudotuberculosis), dodecandrins, ebulins, ebulitins, E. hyemalis RIPs, euserratins, eutirucallin, flammin, flammulin, foetidissimin, gelonin, gigantin, gypsophilin, H. crepitans RIPs, Heterotepalin, hispin, hirsutellin A, H. orientalis RIPs, H. vulgare RIPs, hypsin, insularin, I. hollandica RIPs, lagenin, lamjapin, lanceolin, L. cylindrical RIPs, luffacylin, luffaculin, luffagulin, luffins, L. usitatissimum RIPs, lychnin, lyophyllin, manutins, marmorin, mapalmin, M. charantia lectin, M. crystallinum RIPs, melonin, mexin, Mirabilis spp. RIPs, mitogillin, modeccins, MORs, Mormordica spp. RIPs, momorsgrovin, moschatin, musarmins, N. tabacum RIPs, nigrins, nigritins, ocymoidin, pachyerosin, P. californicum lectin, pepocin, petroglaucin, petrograndin, Phytolacca spp. RIPs, pisavin, pleuturegin, Pluturegin, A. thaliana pectin methyl transferase (PME), P, multiforum RIPs, pokeweed antiviral protein (PAP), porrectin, Aeromonas spp. Pseudomonas toxins (A. hydrophila pseudomonas-like toxin), pulchellin, quinqueginsin. R. communis agglutinins, restrictocin, ricins, riproximin, saporins, sarcins, sativin, S. cereale RIPs, sechiumin, Shiga toxin, Shiga-like toxins, sieboldin b, S. nigra RIPs (e.g. S. nigra agglutinins I-V), S. ocymoides RIPs, Spinacia oleracea protein, stellarin, stenodactylin, texanin, tricholin, Trichosanthes spp. RIPs (e.g. karasurins, kirilowins, trichoanguin, trichokirins, trichosanthins, TYchi), Triticum spp. RIPs, V album RIPs, velin, velutin, verotoxins, V. hispanica RIPs, vircumin, volkensin, V. volvacea RIPs, Volvarin, Yucca leaf protein, Z. diploperennis RIPs, Z. mays RIPs, and any ribotoxic fragment of any of the foregoing, and the various antitumor or anticancer agents described herein.

There are numerous proteinaceous toxins suitable for use as a toxin component as described herein. For example, argonaute enzymatic domains or hybrid enzymatic domains composed of fungal ribotoxins and argonaute sequences may be engineered for ribosome inactivation (see Pichinuk E, Wreschner D, Protein Sci 19: 1272-8 (2010)). Examples of RNases with enzymatic domains useful as ribotoxic regions include bacterial RNases, such as, e.g., binase, amphibian RNases, such as e.g., ranpimase and Onconase®, and mammalian RNases, such as, e.g., bovine semen RNase and the human RNases: RNase2, RNase3, and RNase5 (Newton D et al., J Biol Chem 269: 739-45 (1994); Netwon D et al., J Immunol Meth 231: 159-67 (1999); Yoon J et al., Life Sci 64: 1435-45 (1999); Hugh M et al., Cancer Res 61: 8737-42 (2001); Makarov A, Ilinskaya N, FEBS Lett 540: 15-20 (2003)).

TABLE 5
Exemplary Protein Toxins and Sources
of Toxin Effector Polypeptides

| Protein Toxin | Substrate - Subcellular Location |
|---|---|
| Abrins | sarcin-ricin loop - cytosol |
| Anthrax lethal factor | MAPKK - cytosol |
| Aspfl | sarcin-ricin loop - cytosol |
| Bouganin | sarcin-ricin loop - cytosol |
| Bryodins | sarcin-ricin loop - cytosol |
| Cholix toxin | heterotrimeric G protein - cytosol |
| Cinnamomin | sarcin-ricin loop - cytosol |
| Claudin | sarcin-ricin loop - cytosol |
| Clavin | sarcin-ricin loop - cytosol |
| C. difficile TcdA | Ras GTPases - cytosol |
| C. difficile TcdA | Rho GTPases - cytosol |
| C. perfringens iota | Rho GTPases - cytosol |
| cytolethal distending | DNA - nucleus |
| Dianthins | sarcin-ricin loop - cytosol |
| Diphtheria toxins | elongation factor-2 (EF2) - cytosol |
| Ebulins | sarcin-ricin loop - cytosol |
| Gelonin | sarcin-ricin loop - cytosol |
| Gigantin | sarcin-ricin loop - cytosol |
| heat-labile enterotoxins | heterotrimeric G protein - cytosol |
| Maize RIPs | sarcin-ricin loop - cytosol |
| Mitogillin | sarcin-ricin loop - cytosol |
| Nigrins | sarcin-ricin loop - cytosol |
| Pertussis toxins | heterotrimeric G protein - cytosol |
| PD-Ls | sarcin-ricin loop - cytosol |
| PAPs | sarcin-ricin loop - cytosol |
| Pseudomonas toxins | elongation factor-2 (EF2) - cytosol |
| Pulchellin | sarcin-ricin loop - cytosol |
| Restrictocin | sarcin-ricin loop - cytosol |
| Ricins | sarcin-ricin loop - cytosol |
| Saporins | sarcin-ricin loop - cytosol |
| Sarcins | sarcin-ricin loop - cytosol |
| Shiga toxins | sarcin-ricin loop - cytosol |
| Subtilase cytotoxins | endoplasmic chaperon - ER |
| Trichosanthins | sarcin-ricin loop - cytosol |

### IV. General Functions of the Binding Molecules

The binding molecules are useful in diverse applications involving. e.g., cell-killing; cell growth inhibition; intracellular, cargo delivery, biological information gathering; immune response stimulation, and/or remediation of a health condition. The binding molecules are useful as therapeutic and/or diagnostic molecules, such as, e.g., as cell-targeting, cytotoxic, therapeutic molecules, cell-targeting, nontoxic, delivery vehicles; and/or cell-targeting, diagnostic molecules; for examples in applications involving the in vivo targeting of specific cell types for the diagnosis or treatment of a variety of diseases, including cancers, immune disorders, and microbial infections.

In some embodiments, the binding molecules are capable of binding an extracellular part of PD-L1 molecules associated with cell surfaces of particular cell types and entering those cells. Once internalized within a targeted cell type, certain embodiments of the binding molecules are capable of killing the cell via the action(s) of the toxin component. For example, once internalized within a targeted cell type, certain embodiments of the binding molecules are capable of routing an enzymatically active, cytotoxic, Shiga toxin effector polypeptide fragment into the cytosol of the target cell and eventually killing the cell. In another example, once internalized within a targeted cell type, certain embodiments of the binding molecules are capable of delivering a CD8+ T-cell epitope cargo to the MHC class I presentation pathway of the target cell due to the action of the toxin component, leading to cell-surface presentation of that epitope complexed with a MHC class I molecule, and eventually resulting in the death of the cell. In another example, once internalized within a targeted cell type, certain embodiments of the binding molecules are capable of delivering a cytotoxic cargo to the target cell due to the action of the toxin component thereby resulting in the death of the cell.

Alternatively, nontoxic or reduced-toxicity variants of the binding molecules may be used to deliver additional exogenous materials into target cells, such as epitopes, peptides, proteins, polynucleotides, and detection-promoting agents. This system is modular, in that any number of diverse toxin components may be associated with a PD-L1 binding region(s) to produce variants of the binding molecule with different functional characteristics, such as, e.g. de-immunized toxin effectors for applications involving administration of the binding molecule to a chordate, reduced protease-cleavage sensitive toxin effectors to improve stability particularly in vivo, and toxin effectors comprising a CD8+ T-cell epitope for immunotherapy applications.

### A. Cell-Kill via Toxin Component Cytotoxicity

Some embodiments of the binding molecules are cytotoxic. Some embodiments of the binding molecules are cytotoxic only due to the presence of one or more Shiga toxin effector polypeptide components. The A Subunits of members of the Shiga toxin family each comprise an enzymatically active polypeptide region capable of killing a eukaryotic cell once in the cell's cytosol. Because members of the Shiga toxin family are adapted to killing eukaryotic cells, molecules derived from Shiga toxins, such as, e.g., PD-L1 binding molecules comprising certain embodiments of the Shiga toxin effector polypeptides can exhibit potent cell-kill activities.

In some embodiments, upon contacting a cell physically coupled with PD-L1 bound by the binding region of the binding molecule (e.g. a PD-L1 positive cell), the binding molecule is capable of causing death of the cell. For some embodiments, the CD₅₀ value of the binding molecule is less than 5, 2.5, 1, 0.5, or 0.25 nM, which is vastly more potent than an untargeted, wild-type, Shiga toxin effector polypeptide (e.g, SEQ ID NOs: 1-18).

Cell-kill may be accomplished using a molecule described herein under varied conditions of target cells, such as, e.g., an ex vivo manipulated target cell, a target cell cultured in vitro, a target cell within a tissue sample cultured in vitro, or a target cell in an in vivo setting like within a multicellular organism.

In some embodiments, the Shiga toxin effector polypeptides and binding molecules comprise (1) a de-immunized, Shiga toxin effector sub-region, (2) a protease-cleavage resistant region near the carboxy-terminus of a Shiga toxin A1 fragment derived region, (3) a carboxy-terminal, endoplasmic reticulum retention/retrieval signal site; and/or (4) a heterologous, T-cell epitope embedded or inserted region; however, for some embodiments, these structural modifications do not significantly alter the potency of Shiga toxin cytotoxicity as compared to reference molecules comprising a wild-type Shiga toxin A Subunit polypeptide, such as, e.g., a wild-type Shiga toxin A1 fragment. Thus, Shiga toxin effector polypeptides and binding molecules which are de-immunized, protease cleavage resistant, and/or carrying embedded or inserted, heterologous, epitopes can maintain potent cytotoxicity while providing one or more various other functionalities or properties.

Already cytotoxic binding molecules comprising Shiga toxin effector polypeptides may be engineered by the skilled worker using the information and methods provided herein to be more cytotoxic and/or to have redundant, backup cytotoxicities operating via completely different mechanisms. These multiple cytotoxic mechanisms may complement each other by their diversity of functions (such as by providing potent killing via two mechanisms of cell-killing, direct and indirect, as well as mechanisms of immuno-stimulation to the local area), redundantly backup each other (such as by providing one cell-killing mechanism in the absence of the other mechanisms-like if a target cell is resistant to or acquires some immunity to a subset of previously active mechanisms), and/or protect against developed resistance (by limiting resistance to the less probable situation of the malignant or infected cell blocking multiple, different cell-killing mechanisms simultaneously).

### B. Delivery of a T-Cell Epitope for MHC Class I Presentation on a Cell Surface

In some embodiments, the binding molecules comprise a T-cell epitope, which enables the engineering of “T-cell epitope delivering” molecules with virtually unlimited choices of epitope-peptide cargos for delivery and cell-surface presentation by a nucleated, chordate cell. In some embodiments, the binding molecules comprises a toxin effector comprising a T-cell epitope. In some embodiments, the binding molecules are capable via their toxin component of delivering one or more T-cell epitopes to the proteasome of a cell. The delivered T-cell epitope are then proteolytic processed and presented by the MHC class I pathway on the surface of the cell. By engineering MHC class I epitopes into binding molecules, the targeted delivery and presentation of immuno-stimulatory antigens may be accomplished in order to harness and direct a beneficial function(s) of a chordate immune system.

In some embodiments, the Shiga toxin effector polypeptide or binding molecule is capable of delivering a T-cell epitope to a MHC class I molecule of a cell for cell-surface presentation. In some embodiments, the Shiga toxin effector polypeptide or binding molecule comprises a heterologous, T-cell epitope, whether as an additional exogenous material or embedded or inserted within a Shiga toxin effector polypeptide. For some embodiments, the Shiga toxin effector polypeptide or binding molecule is capable of delivering an embedded or inserted T-cell epitope to a MHC class I molecule for cell-surface presentation.

In some embodiments, the Shiga toxin effector polypeptide is capable of delivering a T-cell epitope, which is embedded or inserted in the Shiga toxin effector polypeptide, to a MHC class I molecule of a cell in which the Shiga toxin effector polypeptide is present for presentation of the T-cell epitope by the MHC class I molecule on a surface of the cell. For some embodiments, the T-cell epitope is a heterologous, T-cell epitope. For some embodiments, the T-cell epitope functions as CD8+ T-cell epitope, whether already known or identified in the future using methods which are routine to the skilled worker.

In some embodiments, the binding molecule is capable of delivering a T-cell epitope, which is associated with the binding molecule, to a MHC class I molecule of a cell for presentation of the T-cell epitope by the MHC class I molecule on a surface of the cell. For some embodiments, the T-cell epitope is a heterologous, T-cell epitope which is embedded or inserted in the Shiga toxin effector polypeptide. For some embodiments, the T-cell epitope functions as CD8+ T-cell epitope, whether already known or identified in the future using methods which are routine to the skilled worker.

In some embodiments, upon contacting a cell with the binding molecule, the binding molecule is capable of delivering a T-cell epitope-peptide, which is associated with the binding molecule, to a MHC class I molecule of the cell for presentation of the T-cell epitope-peptide by the MHC class I molecule on a surface of the cell. For some embodiments, the T-cell epitope-peptide is a heterologous epitope which is embedded or inserted in a Shiga toxin effector polypeptide. For some embodiments, the T-cell epitope-peptide functions as CD8+ T-cell epitope, whether already known or identified in the future using methods which are routine to the skilled worker.

The addition of a heterologous epitope into or presence of a heterologous epitope in a binding molecule, whether as an additional exogenous material or embedded or inserted within a Shiga toxin effector polypeptide, enables methods of using such binding molecules for the cell-targeted delivery of a chosen epitope for cell-surface presentation by a nucleated, target cell within a chordate.

One function of certain, CD8+ T-cell hyper-immunized, Shiga toxin effector polypeptides and binding molecules is the delivery of one or more T-cell epitope-peptides to a MHC class I molecule for MHC class I presentation by a cell. Delivery of exogenous, T-cell epitope-peptides to the MHC class I system of a target cell can be used to induce the target cell to present the T-cell epitope-peptide in association with MHC class I molecules on the cell surface, which subsequently leads to the activation of CD8+ effector T-cells to attack the target cell.

The skilled worker, using techniques known in the art, can associate, couple, and/or link certain, Shiga toxin effector polypeptides to various other PD-L1-targeting binding regions to create binding molecules which target specific, extracellular, target biomolecules physically coupled to cells and promote target-cell internalization of these binding molecules. All nucleated vertebrate cells are believed to be capable of presenting intracellular epitopes using the MHC class I system. Thus, extracellular target biomolecules of the binding molecules may in principle target any nucleated vertebrate cell for T-cell epitope delivery to a MHC class I presentation pathway of such a cell.

The epitope-delivering functions of the Shiga toxin effector polypeptides and binding molecules can be detected and monitored by a variety of standard methods known in the art to the skilled worker and/or described herein. For example, the ability of binding molecules to deliver a T-cell epitope-peptide and drive presentation of the epitope-peptide by the MHC class I system of target cells may be investigated using various in vitro and in vivo assays, including, e.g., the direct detection/visualization of MHC class U/peptide complexes, measurement of binding affinities for the heterologous, T-cell epitope-peptide to MHC class I molecules, and/or measurement of functional consequences of MHC class I-peptide complex presentation on target cells by monitoring cytotoxic T-lymphocyte (CTL) responses (see e.g. Examples, infra).

Certain assays to monitor this function of the polypeptides and molecules involve the direct detection of a specific MHC class I/peptide antigen complex in vitro or ex vivo. Common methods for direct visualization and quantitation of peptide-MHC class I complexes involve various immuno-detection reagents known to the skilled worker. For example, specific monoclonal antibodies can be developed to recognize a particular MHC/class I/peptide antigen complex. Similarly, soluble, multimeric T cell receptors, such as the TCR-STAR reagents (Altor Bioscience Corp., Mirmar, Fla., U.S.A.) can be used to directly visualize or quantitate specific MHC I/antigen complexes (Zhu X et al., J Immunol 176: 3223-32 (2006)). These specific mAbs or soluble, multimeric T-cell receptors may be used with various detection methods, including, e.g. immunohistochemistry, flow cytometry, and enzyme-linked immuno assay (ELISA).

An alternative method for direct identification and quantification of MHC I/peptide complexes involves mass spectrometry analyses, such as, e.g., the ProPresent Antigen Presentation Assay (ProImmune, Inc., Sarasota, Fla., U.S.A.) in which peptide-MCH class I complexes are extracted from the surfaces of cells, then the peptides are purified and identified by sequencing mass spectrometry (Falk K et al., Nature 351: 290-6 (1991)).

In certain assays to monitor the T-cell epitope delivery and MHC class I presentation function of the polypeptides and molecules described herein involve computational and/or experimental methods to monitor MHC class I and peptide binding and stability. Several software programs are available for use by the skilled worker for predicting the binding responses of peptides to MHC class I alleles, such as, e.g., The Immune Epitope Database and Analysis Resource (IEDB) Analysis Resource MHC-I binding prediction Consensus tool (Kim Y et al., Nucleic Acid Res 40: W525-30 (2012). Several experimental assays have been routinely applied, such as, e.g., cell surface binding assays and/or surface plasmon resonance assays to quantify and/or compare binding kinetics (Miles K et al., Mol Immunol 48: 728-32 (2011)). Additionally, other MHC-peptide binding assays based on a measure of the ability of a peptide to stabilize the ternary MHC-peptide complex for a given MHC class I allele, as a comparison to known controls, have been developed (e.g., MHC-peptide binding assay from ProImmmune, Inc.).

Alternatively, measurements of the consequence of MHC class I/peptide antigen complex presentation on the cell surface can be performed by monitoring the cytotoxic T-cell (CTL) response to the specific complex. These measurements by include direct labeling of the CTLs with MHC class I tetramer or pentamer reagents. Tetramers or pentamers bind directly to T cell receptors of a particular specificity, determined by the Major Histocompatibility Complex (MHC) allele and peptide complex. Additionally, the quantification of released cytokines, such as interferon gamma or interleukins by ELISA or enzyme-linked immunospot (ELIspot) is commonly assayed to identify specific CTL responses. The cytotoxic capacity of CTL can be measured using a number of assays, including the classical 51 Chromium (Cr) release assay or alternative non-radioactive cytotoxicity assays (e.g., CytoTox96® non-radioactive kits and CellTox™ CellTiter-GLO® kits available from Promega Corp., Madison, Wis., U.S.A.). Granzyme B ELISpot, Caspase Activity Assays or LAMP-1 translocation flow cytometric assays. To specifically monitor the killing of target cells, carboxyfluorescein diacetate succinimidyl ester (CFSE) can be used to easily and quickly label a cell population of interest for in vitro or in vivo investigation to monitor killing of epitope specific CSFE labeled target cells (Durward M et al., J Vis Exp 45 pii 2250 (2010)).

In vivo responses to MHC class I presentation can be followed by administering a MHC class I/antigen promoting agent (e.g., a peptide, protein or inactivated/attenuated virus vaccine) followed by challenge with an active agent (e.g. a virus) and monitoring responses to that agent, typically in comparison with unvaccinated controls. Er vivo samples can be monitored for CTL activity with methods similar to those described previously (e.g. CTL cytotoxicity assays and quantification of cytokine release).

HLA-A, HLA-B, and/or HLA-C molecules are isolated from the intoxicated cells after lysis using immune affinity (e.g., an anti-MHC antibody “pulldown” purification) and the associated peptides (i.e., the peptides presented by the isolated MHC molecules) are recovered from the purified complexes. The recovered peptides are analyzed by sequencing mass spectrometry. The mass spectrometry data is compared against a protein database library consisting of the sequence of the exogenous (non-self) peptide (T-cell epitope X) and the international protein index for humans (representing “self” or non-immunogenic peptides). The peptides are ranked by significance according to a probability database. All detected antigenic (non-self) peptide sequences are listed. The data is verified by searching against a scrambled decoy database to reduce false hits (see e.g. Ma B, Johnson R. Mol Cell Proteomics 11: O111.014902 (2012)). The results will demonstrate that peptides from the T-cell epitope X are presented in MHC complexes on the surface of intoxicated target cells.

The set of presented peptide-antigen-MHC complexes can vary between cells due to the antigen-specific HLA molecules expressed. T-cells can then recognize specific peptide-antigen-MHC complexes displayed on a cell surface using different TCR molecules with different antigen-specificities.

Because multiple T-cell epitopes may be delivered by a binding molecule, such as, e.g., by embedding two or more different T-cell epitopes in a single proteasome delivering effector polypeptide, a single binding molecule may be effective chordates of the same species with different MHC class variants, such as, e.g., in humans with different HLA alleles. This may allow for the combining within a single molecule of different T-cell epitopes with different effectiveness in different sub-populations of subjects based on MHC complex protein diversity and polymorphisms. For example, human MHC complex proteins, HLA proteins, vary among humans based on genetic ancestry, e.g. African (sub-Saharan). Amerindian, Caucasoid. Mongoloid, New Guinean and Australian, or Pacific islander.

The applications involving the T-cell epitope delivering polypeptides and molecules are vast. Every nucleated cell in a mammalian organism may be capable of MHC class I pathway presentation of immunogenic, T-cell epitope-peptides on their cell outer surfaces complexed to MHC class I molecules. In addition, the sensitivity of T-cell epitope recognition is so exquisite that only a few MHC-I peptide complexes are required to be presented to result in an immune response, e.g., even presentation of a single complex can be sufficient for recognition by an effector T-cell (Sykulev Y et al., Immunity 4: 565-71 (1996)).

The activation of T-cell responses are desired characteristics of certain anti-cancer, anti-neoplastic, anti-tumor, and/or anti-microbial biologic drugs to stimulate the patient's own immune system toward targeted cells. Activation of a robust and strong T-cell response is also a desired characteristic of many vaccines. The presentation of a T-cell epitope by a target cell within an organism can lead to the activation of robust immune responses to a target cell and/or its general locale within an organism. Thus, the targeted delivery of a T-cell epitope for presentation may be utilized for as a mechanism for activating T-cell responses during a therapeutic regime.

The presentation of a T-cell immunogenic epitope-peptide by the MHC class I system targets the presenting cell for killing by CTL-mediated lysis and also triggers immune stimulation in the local microenvironment. By engineering immunogenic epitope sequences within Shiga toxin effector polypeptide components of target-cell-internalizing therapeutic molecules, the targeted delivery and presentation of immuno-stimulatory antigens may be accomplished. The presentation of immuno-stimulatory non-self antigens, such as e.g. known viral antigens with high immunogenicity, by target cells signals to other immune cells to destroy the target cells as well as to recruit more immune cells to the area.

The presentation of an immunogenic, T-cell epitope-peptide by the MHC class I complex targets the presenting cell for killing by CTL-mediated cytolysis. The presentation by targeted cells of immuno-stimulatory non-self antigens, such as, e.g., known viral epitope-peptides with high immunogenicity, can signal to other immune cells to destroy the target cells and recruit more immune cells to the target cell site within a chordate.

Thus, already cytotoxic molecules, such as e.g. therapeutic or potentially therapeutic molecules comprising Shiga toxin effector polypeptides, may be engineered using methods as described herein into more cytotoxic molecules and/or to have an additional cytotoxic mechanism operating via delivery of a T-cell epitope, presentation, and stimulation of effector T-cells. These multiple cytotoxic mechanisms may complement each other (such as by providing both direct target-cell-killing and indirect (CTL-mediated) cell-killing, redundantly backup each other (such as by providing one mechanism of cell-killing in the absence of the other), and/or protect against the development of therapeutic resistance (by limiting resistance to the less probable situation of the malignant or infected cell evolving to block two different cell-killing mechanisms simultaneously).

In addition, a cytotoxic molecule comprising a Shiga toxin effector polypeptide region that exhibits catalytic-based cytotoxicity may be engineered by the skilled worker using routine methods into enzymatically inactive variants. For example, the cytotoxic Shiga toxin effector polypeptide component of a cytotoxic molecule may be conferred with reduced activity and/or rendered inactive by the introduction of one or mutations and/or truncations such that the resulting molecule can still be cytotoxic via its ability to deliver a T-cell epitope to the MHC class I system of a target cell and subsequent presentation to the surface of the target cell. In another example, a T-cell epitope may be inserted or embedded into a Shiga toxin effector polypeptide such that the Shiga toxin effector polypeptide is inactivated by the added epitope (see e.g. WO 2015/113005: Example 1-F). This approach removes one cytotoxic mechanism while retaining or adding another and may also provide a molecule capable of exhibiting immuno-stimulation to the local area of a target cell(s) within an organism via delivered T-cell epitope presentation or “antigen seeding.” Furthermore, non-cytotoxic variants of the binding molecules which comprise embedded or inserted, heterologous, T-cell epitopes may be useful in applications involving immune-stimulation within a chordate and/or labeling of target cells within a chordate with MHC class I molecule displayed epitopes.

The ability to deliver a T-cell epitope of certain Shiga toxin effector polypeptides and binding molecules may be accomplished under varied conditions and in the presence of non-targeted bystander cells, such as, e.g., an ex vivo manipulated target cell, a target cell cultured in vitro, a target cell within a tissue sample cultured in vitro, or a target cell in an in vivo setting like within a multicellular organism.

C. Cell-Kill Via Targeted Cytotoxicity and/or Engagement of Cytotoxic T-Cells

In some embodiments, the binding molecule can provide 1) delivery of a T-cell epitope for MHC class I presentation by a target cell and/or 2) potent cytotoxicity. In some embodiments of the binding molecules, upon contacting a cell physically coupled with an extracellular PD-L1 bound by the cell-targeting binding region, the binding molecule is capable of causing death of the cell. The mechanism of cell-kill may be direct, e.g. via the enzymatic activity of a toxin effector polypeptide region, or indirect via CTL-mediated cytolysis.

### 1. Indirect Cell-Kill Via T-Cell Epitope Delivery and MHC Class I Presentation

Certain embodiments of the binding molecules are cytotoxic because they comprise a CD8+ T-cell epitope capable of being delivered to the MHC class I presentation pathway of a target cell and presented on a cellular surface of the target cell. For example, T-cell epitope delivering. CD8+ T-cell hyper-immunized, Shiga toxin effector polypeptides, with or without endogenous epitope de-immunization, may be used as components of binding molecules for applications involving indirect cell-killing.

In certain embodiments of the binding molecules, upon contacting a cell physically coupled with extracellular PD-L1 bound by the cell-targeting binding region, the binding molecule is capable of indirectly causing the death of the cell, such as, e.g., via the presentation of one or more T-cell epitopes by the target cell and the subsequent recruitment of CTLs which kill the target cell. In some embodiments, the recruitment involves an endogenous CTL specific to an antigen cargo of the binding molecule.

The presentation of an antigenic peptide complexed with a MHC class I molecule by a cell sensitizes the presenting cell to targeted killing by cytotoxic T-cells (CTLs) via the induction of apoptosis, lysis, and/or necrosis. In addition, the CTLs which recognize the target cell may release immuno-stimulatory cytokines, such as, e.g., interferon gamma (IFN-gamma), tumor necrosis factor alpha (TNF), macrophage inflammatory protein-1 beta (MIP-1beta), and interleukins such as IL-17, IL-4, and IL-22. Furthermore, CTLs activated by recognition of a presented epitope may indiscriminately kill other cells proximal to the presenting cell regardless of the peptide-MHC class I complex repertoire presented by those proximal cells (Wiedemann A et al., Proc Natl Acad Sci USA 103: 10985-90 (2006)).

Because of MHC allele diversity within different species, a binding molecule comprising only a single epitope may exhibit varied effectiveness to different patients or subjects of the same species. However, certain embodiments of the binding molecules may each comprise multiple, T-cell epitopes that are capable of being delivered to the MHC class I system of a target cell simultaneously. Thus, in some embodiments of the binding molecules, a binding molecule is used to treat different subjects with considerable differences in their MHC molecules' epitope-peptide binding affinities (i.e. considerable differences in their MHC alleles and/or MHC genotypes). In addition, certain embodiments of the binding molecules reduce or prevent target cell adaptations to escape killing (e.g. a target cancer cell mutating to escape therapeutic effectiveness or “mutant escape”) by using multiple cell-killing mechanisms simultaneously (e.g. direct killing and indirect killing via multiple different T-cell epitopes simultaneously).

In some embodiments, the binding molecules induce target cell-killing via at least two distinct mechanisms of action, Shiga toxin A Subunit effector activity and antigenic peptide delivery to promote immune activation, which may function cooperatively to induce more target cell death in the presence of certain MHC class I epitope-specific restricted CD8+ T-cells. In some embodiments of the binding molecules which induce target cell-killing via two distinct mechanisms of action, Shiga toxin A Subunit effector activity and antigenic peptide delivery to promote immune activation, the resulting target cell killing is additive or synergistic as compared to either killing mechanism in isolation.

2. Direct Cell-Kill Via Cell-Targeted. Shiga Toxin Cytotoxicity

Certain embodiments of the binding molecules are cytotoxic because they comprise a catalytically active toxin component and regardless of the presence of an immunogenic, CD8+ T-cell epitope in the molecule. For example. CD8+ T-cell hyper-immunized, Shiga toxin effector or Diphtheria toxin effector polypeptides, with or without endogenous epitope de-immunization, may be used as components of binding molecules for applications involving direct cell-killing, such as, e.g., via the ribotoxic, enzymatic activity of a Shiga toxin effector polypeptide or ribosome binding and interference with ribosome function due to a non-catalytic mechanism(s) (see e.g. WO 2015/113005). Certain binding molecules can permanently inactivate ribosomes within target cells.

In some embodiments of the CD8+ T-cell hyper-immunized, binding molecules, upon contacting a cell physically coupled with extracellular PD-L1 bound by the cell-targeting binding region, the binding molecule is capable of directly causing the death of the cell, such as, e.g., without the involvement of a untargeted, cytotoxic T-cell (see Section V-D, supra).

In some embodiments, the binding molecules is capable, upon contacting a PD-L1 positive peripheral blood mononuclear cell, the binding molecule, of causing the death of the PD-L1 positive peripheral blood mononuclear cell, such as, e.g., in vivo.

### C. Selective Cytotoxicity Among Cell Types

Certain binding molecules have uses in the selective killing of specific target cells in the presence of untargeted, bystander cells. By targeting the delivery of a toxin component to specific cells via a cell-targeting binding region(s), the binding molecules can exhibit cell-type specific, restricted cell-kill activities resulting in the exclusive or preferential killing selected cell types in the presence of untargeted cells. Similarly, by targeting the delivery of immunogenic T-cell epitopes to the MHC class I pathway of target cells, the subsequent presentation of T-cell epitopes and CTL-mediated cytolysis of target cells induced by the binding molecules can be restricted to exclusively or preferentially killing selected cell types in the presence of untargeted cells. In addition, both the cell-targeted delivery of a cytotoxic, toxin component and an immunogenic, T-cell epitope can be accomplished by a single binding molecule such that deliver of both potentially cytotoxic components is restricted exclusively or preferentially to target cells in the presence of untargeted cells.

In some embodiments, the binding molecule is cytotoxic at certain concentrations. In some embodiments, upon administration of the binding molecule to a mixture of cell types, the cytotoxic binding molecule is capable of selectively killing those cells which are physically coupled with extracellular PD-L1 bound by the binding region compared to cell types not physically coupled with any extracellular PD-L1. In some embodiments, the cytotoxic binding molecule is capable of selectively or preferentially causing the death of a specific cell type within a mixture of two or more different cell types. This enables targeting cytotoxic activity to specific cell types with a high preferentiality, such as a 3-fold cytotoxic effect, over “bystander” cell types that do not express the target biomolecule. Alternatively, the expression of the target biomolecule of the binding region may be non-exclusive to one cell type if the target biomolecule is expressed in low enough amounts and/or physically coupled in low amounts with cell types that are not to be targeted. This enables the targeted cell-killing of specific cell types with a high preferentiality, such as a 3-fold cytotoxic effect, over “bystander” cell types that do not express significant amounts of the target biomolecule or are not physically coupled to significant amounts of the target biomolecule.

For some embodiments, upon administration of the cytotoxic binding molecule to two different populations of cell types, the cytotoxic binding molecule is capable of causing cell death as defined by the half-maximal cytotoxic concentration (CD₅₀) on a population of target cells, whose members express an extracellular target biomolecule of the binding region of the cytotoxic binding molecule, at a dose at least three-times lower than the CD₅₀ dose of the same cytotoxic binding molecule to a population of cells whose members do not express an extracellular target biomolecule of the binding region of the cytotoxic binding molecule.

In some embodiments, the cytotoxic activity of a binding molecule toward populations of cell types physically coupled with an extracellular PD-L1 bound by the binding region is at least 3-fold higher than the cytotoxic activity toward populations of cell types not physically coupled with any extracellular PD-L1 bound by the binding region. As described herein, selective cytotoxicity may be quantified in terms of the ratio (a/b) of (a) cytotoxicity towards a population of cells of a specific cell type physically coupled with extracellular PD-L1 bound by the binding region to (b) cytotoxicity towards a population of cells of a cell type not physically coupled with any extracellular PD-L1 bound by binding region. In some embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, or 1000-fold higher for populations of cells or cell types physically coupled with a target biomolecule of the binding region compared to populations of cells or cell types not physically coupled with a target biomolecule of the binding region.

In some embodiments, the preferential cell-killing function or selective cytotoxicity of a binding molecule is due to an additional exogenous material (e.g. a cytotoxic material) and/or heterologous, T-cell epitope present in a Shiga toxin effector polypeptide and not necessarily a result of the catalytic activity of a Shiga toxin effector polypeptide region.

This preferential cell-killing function allows a targeted cell to be killed by certain cytotoxic, binding molecules under varied conditions and in the presence of non-targeted bystander cells, such as ex vivo manipulated mixtures of cell types, in vitro cultured tissues with mixtures of cell types, or in vivo in the presence of multiple cell types (e.g. in situ or in a native location within a multicellular organism).

Although PD-L1-expressing cells may be selectively targeted, certain binding molecules may selectively kill PD-L1-expressing tumor cells in the presence of PD-L1-expressing peripheral blood mononuclear cell types.

### D. PD-L1/PD-1 Signaling Interference

In addition to cytotoxic, cytostatic, and immune stimulation applications, binding molecules optionally may be used for inhibiting PD-1 signaling, such as, e.g., in applications involving immune checkpoint inhibition and anti-cancer immunotherapy. In some embodiments, the PD-L1 binding molecules can block the PD-1/PD-L1 interaction when exogenously administered to cells. Although some embodiments of the binding molecules exhibit half-maximal inhibitory concentrations for PD-L1 signaling inhibition (EC₅₀) that are much less potent (e.g. greater than 500 nM or 1 μM) than their cytotoxic CD₅₀ (e.g. 0.1 to 50 nM), for a given target cell type, this is not always the case. Some embodiments of the binding molecules can exhibit EC₅₀ values equivalent to their CD₅₀ values, indicating potent levels of both PD-1 signaling inhibition and cytotoxicity could occur concurrently. In some embodiments, the binding molecules exhibit EC₅₀ values (e.g. 1 to 200 nM) that are greater than their cytotoxic CD₅₀ values (e.g. greater than 1,000 or 10,000 nM), such as, e.g., binding molecules comprising inactivated toxins like PD-L1 binding molecules comprising an inactive or reduced-activity Shiga toxin effector polypeptide such as 116296 (SEQ ID NO:127)). Certain binding molecules exhibiting EC₅₀ values greater than their cytotoxic CD₅₀ value may be used at certain concentrations for effectuating PD-1 signaling inhibition in the absence of any significant cytotoxic activity.

E. Delivery of Additional Exogenous Material into the Interior of Targeted Cells

In addition to cytotoxic, cytostatic, immune stimulation, and anti-cancer immunotherapy applications, binding molecules optionally may be used for targeted intracellular delivery functions, such as, e.g., in applications involving information gathering and diagnostic functions.

Because the binding molecules, including reduced cytotoxicity and/or nontoxic forms thereof, are capable of entering cells physically coupled with an extracellular PD-L1 molecule recognized by the binding molecule's binding region, certain embodiments of the binding molecules may be used to deliver additional exogenous materials into the interior of targeted cell types. For example, non-toxic variants of the cytotoxic, binding molecules, or optionally cytotoxic variants, may be used to deliver additional exogenous materials to and/or label the interiors of cells physically coupled with an extracellular PD-L1 bound by the binding region of the binding molecule. Various types of cells and/or cell populations which express target biomolecules to at least one cellular surface may be targeted by the binding molecules for receiving exogenous materials. The functional components are modular, in that various toxin components, additional exogenous materials, and binding regions may be associated with each other to provide binding molecules suitable for diverse applications involving cargo delivery, such as, e.g., non-invasive, in vivo imaging of tumor cells.

This delivery of exogenous material function of certain binding molecules may be accomplished under varied conditions and in the presence of non-targeted bystander cells, such as, e.g., an ex vivo manipulated target cell, a target cell cultured in vitro, a target cell within a tissue sample cultured in vitro, or a target cell in an in vivo setting like within a multicellular organism. Furthermore, the selective delivery of exogenous material to certain cells by certain binding molecules may be accomplished under varied conditions and in the presence of non-targeted bystander cells, such as ex vivo manipulated mixtures of cell types, in vitro cultured tissues with mixtures of cell types, or in vivo in the presence of multiple cell types (e.g., in situ or in a native location within a multicellular organism).

Toxin effector polypeptides and binding molecules which are not capable, such as a certain concentration ranges, of killing a target cell and/or delivering an embedded or inserted epitope for cell-surface presentation by a MHC molecule of a target cell may still be useful for delivering exogenous materials into cells, such as, e.g., detection promoting agents.

In some embodiments, the Shiga toxin effector exhibits low to zero cytotoxicity and thus are referred to herein as “noncytotoxic and/or reduced cytotoxic.” In some embodiments, the binding molecule exhibits low to zero cytotoxicity and may be referred to as “noncytotoxic” and/or “reduced cytotoxic variants.” For example, some molecules do not exhibit a significant level of Shiga toxin based cytotoxicity wherein at doses of less than 1000 nM, 500 nM, 100 nM, 75 nM, 50 nM, there is no significant amount of cell death as compared to the appropriate reference molecule, such as, e.g., as measured by an assay known to the skilled worker and/or described herein. In some embodiments, the molecules do not exhibit any toxicity at dosages of 1-100 μg per kg of a mammalian recipient. Reduced-cytotoxic variants may still be cytotoxic at certain concentrations or dosages but exhibit reduced cytotoxicity, such as, e.g., are not capable of exhibiting a significant level of Shiga toxin cytotoxicity in certain situations.

Certain binding molecules comprising the same, can be rendered non-cytotoxic, such as, e.g., via the addition of one or more amino acid substitutions known to the skilled worker to inactivate a toxin effector polypeptide, including exemplary substitutions described herein. The non-cytotoxic and reduced cytotoxic variants of the binding molecules may be in certain situations more suitable for delivery of additional exogenous materials than more cytotoxic variants.

### Diagnostic Functions

In certain binding molecules have uses in the in vitro and/or in vivo detection of specific cells, cell types, and/or cell populations, as well as specific subcellular compartments of any of the aforementioned. Reduced-cytotoxicity and/or nontoxic forms of the cytotoxic, binding molecules that are conjugated to detection promoting agents optionally may be used for diagnostic functions, such as for companion diagnostics used in conjunction with a therapeutic regimen comprising the same or a related binding region, such as, e.g., a binding region with high-affinity binding to the same target biomolecule, an overlapping epitope, and/or the same epitope.

In some embodiments, the binding molecules described herein are used for both diagnosis and treatment, or for diagnosis alone. When the same cytotoxic binding molecule is used for both diagnosis and treatment, in some embodiments the binding molecule variant which incorporates a detection promoting agent for diagnosis may have its cytotoxicity reduced or may be rendered nontoxic by catalytic inactivation of its Shiga toxin effector polypeptide region(s) via one or more amino acid substitutions, including exemplary substitutions described herein. For example, certain nontoxic variants of the binding molecules exhibit less than 5%, 4%, 3%, 2%, or 1% death of target cells after administration of a dose less than 1 mg/kg. Reduced-cytotoxicity variants may still be cytotoxic at certain concentrations or dosages but exhibit reduced cytotoxicity, such as, e.g., are not capable of exhibiting a significant level of Shiga toxin cytotoxicity as described herein.

The ability to conjugate detection promoting agents known in the art to various binding molecules provides useful compositions for the detection of certain cells, such as, e.g., cancer, tumor, immune, and/or infected cells. These diagnostic embodiments of the binding molecules may be used for information gathering via various imaging techniques and assays known in the art. For example, diagnostic embodiments of the binding molecules may be used for information gathering via imaging of intracellular organelles (e.g. endocytotic, Golgi, endoplasmic reticulum, and cytosolic compartments) of individual cancer cells, immune cells, and/or infected cells in a patient or biopsy sample.

Various types of information may be gathered using the diagnostic embodiments of the binding molecules whether for diagnostic uses or other uses. This information may be useful, for example, in diagnosing neoplastic cell types, determining therapeutic susceptibilities of a patient's disease, assaying the progression of anti-neoplastic therapies over time, assaying the progression of immunomodulatory therapies over time, assaying the progression of antimicrobial therapies over time, evaluating the presence of infected cells in transplantation materials, evaluating the presence of unwanted cell types in transplantation materials, and/or evaluating the presence of residual tumor cells after surgical excision of a tumor mass.

For example, subpopulations of patients might be ascertained using information gathered using the diagnostic variants of the binding molecules, and then individual patients could be further categorized into subpopulations based on their unique characteristic(s) revealed using those diagnostic embodiments. For example, the effectiveness of specific pharmaceuticals or therapies might be a criterion used to define a patient subpopulation. For example, a nontoxic diagnostic variant of a particular cytotoxic, binding molecule may be used to differentiate which patients are in a class or subpopulation of patients predicted to respond positively to a cytotoxic variant of that binding molecule. Accordingly, associated methods for patient identification, patient stratification, and diagnosis using binding molecules, including non-toxic variants of cytotoxic, binding molecules, are also provided herein.

The expression of the target biomolecule by a cell need not be native in order for cell-targeting by a binding molecule, such as, e.g., for direct cell-kill, indirect cell-kill, delivery of exogenous materials like T-cell epitopes, and/or information gathering. Cell surface expression of the target biomolecule could be the result of an infection, the presence of a pathogen, and/or the presence of an intracellular microbial pathogen. Expression of a target biomolecule could be artificial such as, for example, by forced or induced expression after infection with a viral expression vector, see e.g. adenoviral, adeno-associated viral, and retroviral systems. Expression of PD-L1 can be induced by exposing a cell to ionizing radiation (Wattenberg M et al., Br J Cancer 110: 1472-80 (2014)).

### Targeting Immunosuppressive Immune Cells

In some embodiments, the PD-L1 binding molecules described herein are capable of specifically binding PD-L1 on the surface of a cell, such as an immunosuppressive immune cell (IIC). Upon binding to PD-L1 on the cell, the binding molecules may be internalized and the activity of the Shiga toxin A subunit effector polypeptide effectively and specifically kills the cell. In some embodiments, this direct cell kill activity depletes immunosuppressive immune cells, such as Tregs in the tumor microenvironment (TME). Once immunosuppression in the TME is lifted, non-suppressive immune cells (e.g., cytotoxic T cells) can attack the tumor.

In some embodiments, the binding molecules described herein bind to PD-L1 that is on an IIC and on a tumor cell. Thus, in some embodiments, in addition to depleting immunosuppressive immune cells in the TME, the binding molecules also bind to and directly kill tumor cells. This dual mechanism of action can enhance effectiveness of the disclosed binding molecules in cancer therapy.

In some embodiments, the binding molecules described herein cause expansion of one or more types of cells, such as immune cells. For example, in some embodiments, the PD-L1 binding molecules cause expansion of B-cells, T-cells, or eosinophils.

In some embodiments, the binding molecules described herein comprise an antigenic epitope, such as a CD8+ T-cell epitope. In some embodiments, after the binding molecule binds to PD-L1 and is internalized into the cell, the antigenic epitope is delivered to the MHC class I system of the cell, targeting the cell for immune-mediated destruction. Therefore, in addition to depleting immunosuppressive immune cells in the TME, and in some embodiments directly killing tumor cells, the binding molecules also enhance recognition of the tumor by the immune system.

In some embodiments, the binding molecules modulate expression of PD-L1 to which the binding molecules' binding region binds. In some embodiments, the binding molecules reduce or downregulate expression of PD-L1. In some embodiments, the binding molecules reduce cell-surface density of PD-L1. In some embodiments, modulation of expression of PD-L1 reduces immunosuppression. In some embodiments, modulation of expression of PD-L1 leads to cell death.

Thus, the disclosed binding molecules are useful (1) for selectively killing a cell type(s) expressing a PD-1 amongst other cells, and (2) as therapeutic molecules for treating a variety of diseases, disorders, and conditions, including cancers.

The binding molecules described herein comprise a binding region capable of specifically binding PD-L1 on the surface of a cell, e.g., a PD-L1 positive cell. In some embodiments, the PD-L1 positive cell is a tumor cell. In some embodiments, the PD-L1 positive cell is an immunosuppressive immune cell, such as an immunosuppressive T cell, an immunosuppressive B cell, an immunosuppressive plasma cell, or an immunosuppressive myeloid cell. In some embodiments, the immunosuppressive immune cell is a Treg, an MDSC, or a TAM. In some embodiments, the immunosuppressive immune cell is a TAN or a CAF. In some embodiments, the binding region does not specifically bind to a resident memory T cell, a tumor-excluded dendritic cell, and/or a CD14+ monocyte.

As used herein, the terms “does not directly kill” or “indirectly kills” refers to a process wherein a binding molecule comprising a Shiga toxin A subunit effector polypeptide and a binding region binds to a target cell (e.g., a ICC), which leads to the downstream killing of a second cell (e.g., a cancer cell). For example, a binding molecule can indirectly kill a tumor cell by binding to and killing an immunosuppressive immune cell in the tumor microenvironment (TME). Once immunosuppression is lifted in the TME, the cancer cell can be killed by non-suppressive immune cells (e.g., cytotoxic T cells, etc).

In some embodiments, methods for reducing the immunosuppressive activity of an immune cell in a subject in need thereof comprise administering to the subject an effective amount of (i) a binding molecule, (ii) a nucleic acid encoding the binding molecule (e.g., an expression vector), or (iii) a composition comprising the binding molecule or the nucleic acid encoding the same.

In some embodiments, the binding molecule binds to PD-L1 that is present on the surface of an immunosuppressive immune cell in the subject, but is not present on the surface of the subject's cancer cells. In some embodiments, the binding molecule directly kills the immunosuppressive immune cell, but does not directly kill the subject's cancer cells.

In some embodiments, the binding molecule binds to PD-L1 that is present on the surface of an immunosuppressive cell in the subject, and the subject's cancer cells. In some embodiments, the binding molecule directly kills the immunosuppressive immune cell and the subject's cancer cells.

In some embodiments, the subject has cancer. In some embodiments, the cancer is characterized by the presence of at least one immunosuppressive cell, for example in the tumor microenvironment. In some embodiments, the cancer is characterized by a high mutational burden (TMB) and/or a high frequency of indels. Mutational burden can be analyzed by various methods, including hybrid-based next-generation sequencing, and is reported as the total number of sequence variants or mutations per tumor genomic region analyzed (e.g., mutations per megabase). Cancers can be classified as having a “high” mutational burden if they have greater than or equal to 20 mutations per magabase. High mutational burden is typical of cancers developed as a consequence of exposure to powerful carcinogens, such as tobacco smoke and polycyclic aromatic hydrocarbons (e.g., in lung and bladder cancers), as well as exposure to mutagens (e.g., UV light in melanoma). Indels (insertions and deletions) are one type of mutation commonly seen in cancer cells. Indels that produce frameshift mutations can generate highly immunogenic tumor neoantigens. Therefore, the presence of a high frequency of indels can lead to a better response to the therapeutic approaches described herein. Cancers are classified as having a “high” frequency of indels if they have greater than or equal to 0.1, 0.5, 1, 2, 3, 4, 5, 6, 7, 8 9, or 10 indels per magabase. In some embodiments, a cancer is classified as having a high frequency of indels if it has 0.1-1, 1-10, 10-50, 50-100, or greater than 100 indels per megabase.

V. Production. Manufacture. and Purification of Shiga Toxin Effector Polypeptides and Binding Molecules

The Shiga toxin effector polypeptides and certain binding molecules may be produced using techniques well known to those of skill in the art. For example, Shiga toxin effector polypeptides and binding molecules may be manufactured by standard synthetic methods, by use of recombinant expression systems, or by any other suitable method. Thus, Shiga toxin effector polypeptides and binding molecules may be synthesized in a number of ways, including, e.g. methods comprising: (1) synthesizing a polypeptide or polypeptide component of a binding molecule using standard solid-phase or liquid-phase methodology, either stepwise or by fragment assembly, and isolating and purifying the final polypeptide compound product; (2) expressing a polynucleotide that encodes a protein or protein component of a binding molecule in a host cell and recovering the expression product from the host cell or host cell culture; or (3) cell-free, in vitro expression of a polynucleotide encoding a polypeptide or polypeptide component of a binding molecule, and recovering the expression product; or by any combination of the methods of (1), (2) or (3) to obtain fragments of the protein component, subsequently joining (e.g. ligating) the peptide or polypeptide fragments to obtain a polypeptide component, and recovering the polypeptide component.

It may be preferable to synthesize a binding molecule, or a protein component of a binding molecule, by means of solid-phase or liquid-phase peptide synthesis. Polypeptides and binding molecules may suitably be manufactured by standard synthetic methods. Thus, peptides may be synthesized by, e.g. methods comprising synthesizing the peptide by standard solid-phase or liquid-phase methodology, either stepwise or by fragment assembly, and isolating and purifying the final peptide product. In this context, reference may be made to WO 1998/011125 or, inter alia, Fields G et al., Principles and Practice of Solid-Phase Peptide Synthesis (Synthetic Peptides, Grant G, ed., Oxford University Press, U.K., 2nd ed., 2002) and the synthesis examples therein.

Shiga toxin effector polypeptides and binding molecules may be prepared (produced and purified) using recombinant techniques well known in the art. In general, methods for preparing proteins by culturing host cells transformed or transfected with a vector comprising the encoding polynucleotide and purifying or recovering the protein from cell culture are described in, e.g., Sambrook J et al., Molecular Cloning: A Laboratory Manual (Cold Spring Harbor Laboratory Press, NY, U.S., 1989); Dieffenbach C et al., PCR Primer: A Laboratory Manual (Cold Spring Harbor Laboratory Press. N.Y., U.S., 1995). Any suitable host cell may be used to produce a polypeptide and/or cell-targeting protein. Host cells may be cells stably or transiently transfected, transformed, transduced or infected with one or more expression vectors which drive expression of a polypeptide. In addition, a Shiga toxin effector polypeptide and/or binding molecule may be produced by modifying the polynucleotide encoding a polypeptide or cell-targeting protein that result in altering one or more amino acids or deleting or inserting one or more amino acids in order to achieve desired properties, such as changed cytotoxicity, changed cytostatic effects, and/or changed serum half-life.

There are a wide variety of expression systems which may be chosen to produce a polypeptide or cell-targeting protein as described herein. For example, host organisms for expression of cell-targeting proteins include prokaryotes, such as E. coli and B. subtilis, eukaryotic cells, such as yeast and filamentous fungi (like S cerevisiae, P, pastoris, A. awamori, and K lactis), algae (like C. reinhardtii), insect cell lines, mammalian cells (like CHO cells), plant cell lines, and eukaryotic organisms such as transgenic plants (like A. thaliana and N. benthamiana).

Accordingly, also provided are methods for producing a Shiga toxin effector polypeptide and/or binding molecule according to above recited methods and using a polynucleotide encoding part or all of a polypeptide or a protein component of a cell-targeting protein, an expression vector comprising at least one polynucleotide capable of encoding part or all of a polypeptide or cell-targeting protein when introduced into a host cell, and/or a host cell comprising a polynucleotide or expression vector.

When a protein is expressed using recombinant techniques in a host cell or cell-free system, it is advantageous to separate (or purify) the desired protein away from other components, such as host cell factors, in order to obtain preparations that are of high purity or are substantially homogeneous. Purification can be accomplished by methods well known in the art, such as centrifugation techniques, extraction techniques, chromatographic and fractionation techniques (e.g. size separation by gel filtration, charge separation by ion-exchange column, hydrophobic interaction chromatography, reverse phase chromatography, chromatography on silica or cation-exchange resins such as DEAE and the like, chromatofocusing, and Protein A Sepharose chromatography to remove contaminants), and precipitation techniques (e.g. ethanol precipitation or ammonium sulfate precipitation). Any number of biochemical purification techniques may be used to increase the purity of a polypeptide and/or binding molecule. In some embodiments, the polypeptides and binding molecules may optionally be purified in homo-multimeric forms (e.g. a molecular complex comprising two or more polypeptides or binding molecules).

Antibodies may be produced using recombinant methods and compositions (see e.g. U.S. Pat. No. 4,816,567). In some embodiments, isolated nucleic acid encoding an antibody or antibody fragment described herein is provided. Such nucleic acid may encode an amino acid sequence comprising the VL and/or an amino acid sequence comprising the VH of the antibody (e.g. a light and/or heavy chain of an antibody). A method of making an antibody as described herein comprises culturing a host cell comprising a nucleic acid encoding the antibody, as provided above, under conditions suitable for expression of the antibody, and optionally recovering the antibody from the host cell (or host cell culture medium). For recombinant production of an antibody, nucleic acid encoding an antibody, e.g. as described above, is isolated and inserted into one or more vectors for further cloning and/or expression in a host cell. Such nucleic acid may be readily isolated and sequenced using routine methods known to the skilled worker.

Suitable host cells for cloning or expression of antibody-encoding vectors include prokaryotic or eukaryotic cells described herein and/or known to the skilled worker. For example, antibodies may be produced in bacteria, in particular when glycosylation and/or Fc effector function are not required (see e.g. U.S. Pat. Nos. 5,648,237, 5,789,199, and 5,840,523). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern (see e.g. Gerngross T, Nat Biotech 22: 1409-14 (2004); Li H et al., Nat Biotech 24: 210-15 (2006)).

Suitable host cells for the expression of glycosylated antibody are also derived from multicellular organisms (invertebrates and vertebrates). Examples of invertebrate cells include plant and insect cells. Plant cells may be utilized as hosts (see e.g. U.S. Pat. Nos. 5,959,177, 6,040,498, 6,420,548, 7,125,978, and 6,417,429). Numerous baculoviral strains have been identified which may be used in conjunction with insect cells, particularly for transfection of Spodoptera frugiperda cells. Vertebrate cells may be used as hosts. For example, mammalian cell lines that are adapted to grow in suspension may be useful. Other examples of useful mammalian host cell lines are monkey kidney CV1 line transformed by SV40 (COS-7); human embryonic kidney line 293 cells; baby hamster kidney cells (BHK); mouse sertoli cells (e.g. TM4 cells); monkey kidney cells (CV); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells; MRC 5 cells; and FS4 cells (Graham F et al., J Gen Virol 36: 59-74 (1977); Mather J et al., Biol Reprod 23: 243-52 (1980); Mather J et al., Ann NY Acad Sci 383: 44-68 (1992)). Other useful mammalian host cell lines include Chinese hamster ovary (CHO) cells, including DHFR-CHO, and myeloma cell lines such as Y0, NS0 and Sp210 cells (see e.g. Urlaub G et al., Proc Natl Acad Sci U.S.A. 77: 4216-20 (1980)). For a review of certain mammalian host cell lines suitable for antibody production, see Yazaki and Wu, Methods in Molecular Biology, Vol. 248 (B. K. C. Lo, ed., Humana Press, Totowa, N.J.), pp. 255-268 (2003).

Antibodies provided herein may be identified, screened for, or characterized for their physical/chemical properties and/or biological activities by various assays known in the art.

Methods of immuno-conjugation include but are not limited to reactive thiols, aldehyde-tagged, sortase-mediated conjugation, MTGase-mediated conjugation, transglutaminase conjugation, bis-linkages, and using a spacer or multifunctional linker (see e.g. WO 2009/052249. WO 2012/097333, WO2013/173391, WO 2014/140317. WO 2014/159835, WO 2015/155753, WO 2015/191883, WO 2016/102632, WO 2018/185526).

An antibody-toxin conjugate or immunoconjugate may be prepared by several routes employing organic chemistry reactions, conditions, and reagents known to those skilled in the art, including reaction of a nucleophilic group of an antibody with a bivalent linker reagent to form a covalent bond between the linker and the antibody, followed by reaction with a toxin component; and reaction of a nucleophilic group of a toxin component with a bivalent linker reagent, to form a covalent bond between the linker and the toxin, followed by reaction with a nucleophilic group of an antibody.

Nucleophilic groups on antibodies include but are not limited to: (i) amino-terminal amine groups, (ii) side chain amine groups. e.g. of a lysine residue. (iii) side chain thiol groups, e.g. of a cysteine residue, and (iv) sugar hydroxyl or amino groups of a carbohydrate moiety when the antibody is glycosylated. Amine, hydroxyl, and thiol groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, e.g. cysteine disulfide bridges. Antibodies may be made reactive for conjugation with linker reagents by treatment with a reducing agent such as dithiothreitol (DTT) or tricarbonylethylphosphine (TCEP), such that the antibody is fully or partially reduced (see e.g. WO 2013/173391, WO 2013/173392, WO 2013/173393, WO 2013/190272, WO 2014/064424, WO 2014/114207, WO 2015/155753, WO 2018/185526). Additional nucleophilic groups can be introduced into antibodies through modification of lysine residues, e.g., by reacting lysine residues with 2-iminothiolane (Traut's reagent), resulting in conversion of an amine into a thiol. Reactive thiol groups may also be introduced into an antibody by introducing one, two, three, four, or more cysteine residues (e.g. by preparing variant antibodies comprising one or more non-native cysteine amino acid residues).

Antibody-toxin conjugates or immunoconjugates may also be produced by reaction between an electrophilic group on an antibody, such as an aldehyde or ketone carbonyl group, with a nucleophilic group on a linker reagent or toxin component. Useful nucleophilic groups on a linker reagent include, but are not limited to, hydrazide, oxime, amino, hydrazine, thiosemicarbazone, hydrazine carboxylate, and arylhydrazide. In one embodiment, an antibody is modified to introduce electrophilic moieties that are capable of reacting with nucleophilic substituents on the linker reagent or toxin component. In another embodiment, the sugars of glycosylated antibodies may be oxidized, e.g. with periodate oxidizing reagents, to form aldehyde or ketone groups which may react with the amine group of linker reagents or toxin components. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody that can react with appropriate groups on the toxin component. In another embodiment, antibodies containing amino-terminal serine or threonine residues can react with sodium meta-periodate, resulting in production of an aldehyde in place of the first amino acid (see e.g. U.S. Pat. No. 5,362,852). Such an aldehyde can be reacted with a toxin component or linker nucleophile.

Carbohydrate(s) on the Fc region of an antibody is a natural site for attaching compounds. Generally, the carbohydrate is modified by periodate oxidation to generate reactive aldehydes, which can then be used to attach reactive amine containing compounds by Schiff base formation. As the aldehydes can react with amine groups, reactions are carried out at low pH so that lysine residues in the antibody or antigen binding domain are protonated and unreactive. Hydrazide groups are most suitable for attachment to the aldehydes generated since they are reactive at low pH to form a hydrazone linkage. The linkage can then be further stabilized by reduction with sodium cyanoborohydride to form a hydrazine linkage.

Exemplary nucleophilic groups on a toxin component include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Conjugate loading may be expressed as an average number of conjugate moieties per antibody (x). Conjugate loading may range from 1 to 20 conjugate moieties per antibody. The average number of conjugate moieties per antibody in preparations of antibody-toxin conjugates or immunoconjugates from conjugation reactions may be characterized by conventional means such as mass spectroscopy, ELISA assay, and high-performance liquid chromatography (HPLC). The quantitative distribution of immunoconjugate in terms of x may also be determined, such as, e.g., by separation, purification, and characterization of homogeneous immunoconjugate where p is a certain value from immunoconjugate with other conjugate loadings may be achieved by means such as reverse phase HPLC or electrophoresis.

In the Examples below are descriptions of non-limiting examples of methods for producing exemplary binding molecules, as well as specific but non-limiting aspects of production methods.

### VI. Pharmaceutical and Diagnostic Compositions Comprising Binding Molecules

Also provided are Shiga toxin effector polypeptides and binding molecules for use, alone or in combination with one or more additional therapeutic agents, in a pharmaceutical composition, for treatment or prophylaxis of conditions, diseases, disorders, or symptoms described in further detail below (e.g. cancers, malignant tumors, non-malignant tumors, growth abnormalities, immune disorders, and microbial infections). Also provided herein are pharmaceutical compositions comprising a binding molecule, or a pharmaceutically acceptable salt or solvate thereof, together with at least one pharmaceutically acceptable carrier, excipient, or vehicle. In some embodiments, the pharmaceutical composition may comprise homo-multimeric and/or hetero-multimeric forms of a binding molecule. The pharmaceutical compositions are useful in methods of treating, ameliorating, or preventing a disease, condition, disorder, or symptom described in further detail below. Each such disease, condition, disorder, or symptom is envisioned to be a separate embodiment with respect to uses of a pharmaceutical composition according as described herein. Also provided are pharmaceutical compositions for use in at least one method of treatment, as described in more detail below.

As used herein, the terms “patient” and “subject” are used interchangeably to refer to any organism, commonly vertebrates such as humans and animals, which presents symptoms, signs, and/or indications of at least one disease, disorder, or condition. These terms include mammals such as the non-limiting examples of primates, livestock animals (e.g. cattle, horses, pigs, sheep, goats, etc.), companion animals (e.g. cats, dogs, etc.) and laboratory animals (e.g. mice, rabbits, rats, etc.).

As used herein, “treat,” “treating,” or “treatment” and grammatical variants thereof refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. In some embodiments, beneficial or desired clinical results include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g. a human) in need of treatment may thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease, disorder, or condition. With regard to tumors and/or cancers, treatment includes reduction in overall tumor burden and/or individual tumor size.

As used herein, the terms “prevent,” “preventing,” “prevention” and grammatical variants thereof refer to an approach for preventing the development of, or altering the pathology of, a condition, disease, or disorder. Accordingly, “prevention” may refer to prophylactic or preventive measures. In some embodiments, beneficial or desired clinical results include, but are not limited to, prevention or slowing of symptoms, progression or development of a disease, whether detectable or undetectable. A subject (e.g. a human) in need of prevention may thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition may in certain contexts refer to reducing the risk of developing the condition or preventing or delaying the development of symptoms associated with the condition.

As used herein, an “effective amount” or “therapeutically effective amount” is an amount or dose of a composition (e.g. a therapeutic composition, compound, or agent) that produces at least one desired therapeutic effect in a subject, such as preventing or treating a target condition or beneficially alleviating a symptom associated with the condition. The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic composition (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a composition and adjusting the dosage accordingly (see e.g. Remington: The Science and Practice of Pharmacy (Gennaro A, ed., Mack Publishing Co., Easton, Pa., U.S., 19th ed., 1995)).

An effective amount of an agent, e.g., a pharmaceutical formulation of a binding molecule, refers to an amount effective, at dosages and for periods of time necessary, to achieve the desired therapeutic or prophylactic result. The effective amount of the drug for treating cancer may reduce the number of cancer cells; reduce the tumor size; inhibit (e.g. slow to some extent and preferably stop) cancer cell infiltration into peripheral organs; inhibit (e.g. slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

Diagnostic compositions comprise a binding molecule and one or more detection promoting agents. When producing or manufacturing a diagnostic composition, a binding molecule may be directly or indirectly linked to one or more detection promoting agents. There are numerous standard techniques known to the skilled worker for incorporating, affixing, and/or conjugating various detection promoting agents to proteins or proteinaceous components of molecules, especially to immunoglobulins and immunoglobulin-derived domains.

There are numerous detection promoting agents known to the skilled worker, such as isotopes, dyes, colorimetric agents, contrast enhancing agents, fluorescent agents, bioluminescent agents, and magnetic agents, which can be operably linked to the polypeptides or binding molecules for information gathering methods, such as for diagnostic and/or prognostic applications to diseases, disorders, or conditions of an organism (see e.g. Cai W et al., J Nucl Med 48: 304-10 (2007); Nayak T. Brechbiel M, Bioconjug Chem 20: 825-41 (2009); Paudyal P et al., Oncol Rep 22: 115-9 (2009); Qiao J et al., PLoS ONE 6: e18103 (2011); Sano K et al., Breast Cancer Res 14: R61 (2012)). These agents may be associated with, linked to, and/or incorporated within the polypeptide or binding molecule at any suitable position. For example, the linkage or incorporation of the detection promoting agent may be via an amino acid residue(s) of a molecule or via some type of linkage known in the art, including via linkers and/or chelators. The incorporation of the agent is in such a way to enable the detection of the presence of the diagnostic composition in a screen, assay, diagnostic procedure, and/or imaging technique.

Similarly, there are numerous imaging approaches known to the skilled worker, such as non-invasive in vivo imaging techniques commonly used in the medical arena, for example: computed tomography imaging (CT scanning), optical imaging (including direct, fluorescent, and bioluminescent imaging), magnetic resonance imaging (MRI), positron emission tomography (PET), single-photon emission computed tomography (SPECT), ultrasound, and x-ray computed tomography imaging.

VII. Production or Manufacture of Pharmaceutical and/or Diagnostic Compositions Comprising Binding Molecules

Pharmaceutically acceptable salts or solvates of any of the Shiga toxin effector polypeptides and binding molecules are also provided herein.

The term “solvate” refers to a complex of defined stoichiometry formed between a solute (in casu, a proteinaceous compound or pharmaceutically acceptable salt thereof) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

Polypeptides and proteins, or salts thereof, may be formulated as pharmaceutical compositions prepared for storage or administration, which typically comprise a therapeutically effective amount of a molecule, or a salt thereof, in a pharmaceutically acceptable carrier. The term “pharmaceutically acceptable carrier” includes any of the standard pharmaceutical carriers. Pharmaceutically acceptable carriers for therapeutic molecule use are well known in the pharmaceutical art, and are described, for example, in Remington's Pharmaceutical Sciences (Mack Publishing Co. (A. Gennaro, ed., 1985). As used herein, “pharmaceutically acceptable carrier” includes any and all physiologically acceptable, i.e. compatible, solvents, dispersion media, coatings, antimicrobial agents, isotonic, and absorption delaying agents, and the like. Pharmaceutically acceptable carriers or diluents include those used in formulations suitable for oral, rectal, nasal or parenteral (including subcutaneous, intramuscular, intravenous, intradermal, and transdermal) administration. Exemplary pharmaceutically acceptable carriers include sterile aqueous solutions or dispersions and sterile powders for the extemporaneous preparation of sterile injectable solutions or dispersions. Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of the required particle size in the case of dispersions, and by the use of surfactants. In some embodiments, the carrier is suitable for intravenous, intramuscular, subcutaneous, parenteral, spinal or epidermal administration (e.g. by injection or infusion). Depending on selected route of administration, the protein or other pharmaceutical component may be coated in a material intended to protect the compound from the action of low pH and other natural inactivating conditions to which the active protein may encounter when administered to a patient by a particular route of administration.

The formulations of the pharmaceutical compositions may conveniently be presented in unit dosage form and may be prepared by any of the methods well known in the art of pharmacy. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration. Subcutaneous or transdermal modes of administration may be particularly suitable for therapeutic proteins described herein.

The pharmaceutical compositions may also contain adjuvants such as preservatives, wetting agents, emulsifying agents and dispersing agents. Preventing the presence of microorganisms may be ensured both by sterilization procedures, and by the inclusion of various antibacterial and antifungal agents, for example, paraben, chlorobutanol, phenol sorbic acid, and the like. Isotonic agents, such as sugars, sodium chloride, and the like into the compositions, may also be desirable. In addition, prolonged absorption of the injectable pharmaceutical form may be brought about by the inclusion of agents which delay absorption such as, aluminum monostearate and gelatin.

A pharmaceutical composition also optionally includes a pharmaceutically acceptable antioxidant. Exemplary pharmaceutically acceptable antioxidants are water soluble antioxidants such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propylgallate, alpha-tocopherol, and the like; and metal chelating agents, such as citric acid, ethylenediamine tetraacetic acid (EDTA), sorbitol, tartaric acid, phosphoric acid, and the like.

Also provided herein are pharmaceutical compositions comprising one or a combination of different binding molecules, or an ester, salt or amide of any of the foregoing, and at least one pharmaceutically acceptable carrier.

Therapeutic compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g., glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In some embodiments, isotonic agents, e.g., sugars and polyalcohols such as mannitol, sorbitol, or sodium chloride, may be desirable in the composition. Prolonged absorption of injectable compositions may be brought about by including in the composition an agent that delays absorption for example, monostearate salts and gelatin.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating a polypeptide or binding molecule in the required amount in an appropriate solvent with one or a combination of ingredients described above, as required, followed by sterilization microfiltration. Dispersions may be prepared by incorporating the active compound into a sterile vehicle that contains dispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of a polypeptide and/or binding molecule is designed to be administered by, e.g. intravenous, cutaneous or subcutaneous injection, the binding agent will be in the form of a pyrogen-free, parenterally acceptable aqueous solution. Methods for preparing parenterally acceptable protein solutions, taking into consideration appropriate pH, isotonicity, stability, and the like, are within the skill in the art. A preferred pharmaceutical composition for intravenous, cutaneous, or subcutaneous injection will contain, in addition to binding agents, an isotonic vehicle such as sodium chloride injection, Ringer's injection, dextrose injection, dextrose and sodium chloride injection, lactated Ringer's injection, or other vehicle as known in the art. A pharmaceutical composition may also contain stabilizers, preservatives, buffers, antioxidants, or other additives well known to those of skill in the art.

As described elsewhere herein, a polypeptide and/or binding molecule may be prepared with carriers that will protect the active therapeutic agent against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see e.g. Sustained and Controlled Release Drug Delivery Systems (Robinson J, ed., Marcel Dekker, Inc., NY, U.S., 1978)).

In some embodiments, the composition (e.g. a pharmaceutical and/or diagnostic composition) may be formulated to ensure a desired in vivo distribution of a binding molecule. For example, the blood-brain barrier excludes many large and/or hydrophilic compounds. To target a therapeutic molecule or composition to a particular in vivo location, they can be formulated, for example, in liposomes which may comprise one or more moieties that are selectively transported into specific cells or organs, thus enhancing targeted drug delivery. Exemplary targeting moieties include folate or biotin; mannosides; antibodies; surfactant protein A receptor; p120 catenin and the like.

Pharmaceutical compositions include parenteral formulations designed to be used as implants or particulate systems. Examples of implants are depot formulations composed of polymeric or hydrophobic components such as emulsions, ion exchange resins, and soluble salt solutions. Examples of particulate systems are microspheres, microparticles, nanocapsules, nanospheres, and nanoparticles (see e.g. Honda M et al., Int J Nanomedicine 8: 495-503 (2013); Sharma A et al., Biomed Res Int 2013: 960821 (2013); Ramishetti S, Huang L, Ther Deliv 3: 1429-45 (2012)). Controlled release formulations may be prepared using polymers sensitive to ions, such as, e.g. liposomes, polaxamer 407, and hydroxyapatite.

### VIII. Polynucleotides, Expression Vectors, and Host Cells

Beyond the polypeptides and binding molecules, the polynucleotides that encode the polypeptide components and binding molecules, or functional portions thereof, are also provided herein. The term “polynucleotide” is equivalent to the term “nucleic acid,” each of which includes one or more of: polymers of deoxyribonucleic acids (DNAs), polymers of ribonucleic acids (RNAs), analogs of these DNAs or RNAs generated using nucleotide analogs, and derivatives, fragments and homologs thereof. The polynucleotide may be single-, double-, or triple-stranded. Such polynucleotides are specifically disclosed to include all polynucleotides capable of encoding an exemplary protein, for example, taking into account the wobble known to be tolerated in the third position of RNA codons, yet encoding for the same amino acid as a different RNA codon (see Stothard P, Biotechniques 28: 1102-4 (2000)).

In some embodiments, provided herein are polynucleotides which encode a Shiga toxin effector polypeptide and/or binding molecule, or a fragment or derivative thereof. The polynucleotides may include, e.g., a nucleic acid sequence encoding a polypeptide at least 50%, 55%, 60%, 65%, 70%, 75%, 80%, 85%, 90%, 95%, 99% or more, identical to a polypeptide comprising one of the amino acid sequences of a polypeptide or binding molecule. Also provided herein are polynucleotides comprising nucleotide sequences that hybridize under stringent conditions to a polynucleotide which encodes a Shiga toxin effector polypeptide component and/or binding molecule, or a fragment or derivative thereof, or the antisense or complement of any such sequence.

Derivatives or analogs of the molecules described herein (e.g., PD-L1 binding molecules) include, inter alia, polynucleotide (or polypeptide) molecules having regions that are substantially homologous to the polynucleotides (or binding molecules), e.g. by at least about 45%, 50%, 70%, 80%, 95%, 98%, or even 99% identity (with a preferred identity of 80-99%) over a polynucleotide (or polypeptide) sequence of the same size or when compared to an aligned sequence in which the alignment is done by a computer homology program known in the art. An exemplary program is the GAP program (Wisconsin Sequence Analysis Package. Version 8 for UNIX, Genetics Computer Group, University Research Park, Madison, Wis., U.S.) using the default settings, which uses the algorithm of Smith T, Waterman M, Adv Appl Math 2: 482-9 (1981). Also included are polynucleotides capable of hybridizing to the complement of a sequence encoding the cell-targeting proteins under stringent conditions (see e.g. Ausubel F et al., Current Protocols in Molecular Biology (John Wiley & Sons, New York, N.Y., U.S., 1993)), and below. Stringent conditions are known to those skilled in the art and may be found, e.g., in Current Protocols in Molecular Biology (John Wiley & Sons, NY, U.S., Ch. Sec. 6.3.1-6.3.6 (1989)).

Also provided herein are expression vectors that comprise the polynucleotides described herein. The polynucleotides capable of encoding the Shiga toxin effector polypeptide components and/or binding molecules may be inserted into known vectors, including bacterial plasmids, viral vectors and phage vectors, using material and methods well known in the art to produce expression vectors. Such expression vectors will include the polynucleotides necessary to support production of contemplated Shiga toxin effector polypeptides and/or binding molecules within any host cell of choice or cell-free expression systems (e.g. pTxb1 and pIVEX2.3). The specific polynucleotides comprising expression vectors for use with specific types of host cells or cell-free expression systems are well known to one of ordinary skill in the art, can be determined using routine experimentation, and/or may be purchased.

The term “expression vector,” as used herein, refers to a polynucleotide, linear or circular, comprising one or more expression units. The term “expression unit” denotes a polynucleotide segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. An expression vector contains one or more expression units. Thus, in some embodiments, an expression vector encoding a Shiga toxin effector polypeptide and/or binding molecule comprising a single polypeptide chain includes at least an expression unit for the single polypeptide chain, whereas a protein comprising, e.g. two or more polypeptide chains (e.g. one chain comprising a V_{L} domain and a second chain comprising a V_{H} domain linked to a toxin effector polypeptide) includes at least two expression units, one for each of the two polypeptide chains of the protein. For expression of multi-chain cell-targeting proteins, an expression unit for each polypeptide chain may also be separately contained on different expression vectors (e.g. expression may be achieved with a single host cell into which expression vectors for each polypeptide chain has been introduced).

Expression vectors capable of directing transient or stable expression of polypeptides and proteins are well known in the art. The expression vectors generally include, but are not limited to, one or more of the following: a heterologous signal sequence or peptide, an origin of replication, one or more marker genes, an enhancer element, a promoter, and a transcription termination sequence, each of which is well known in the art. Optional regulatory control sequences, integration sequences, and useful markers that can be employed are known in the art.

The term “host cell” refers to a cell which can support the replication or expression of the expression vector. Host cells may be prokaryotic cells, such as E. coli or eukaryotic cells (e.g. yeast, insect, amphibian, bird, or mammalian cells). Creation and isolation of host cell lines comprising a polynucleotide or capable of producing a polypeptide and/or binding molecule can be accomplished using standard techniques known in the art.

Shiga toxin effector polypeptides and/or proteins described herein may be variants or derivatives of the poly peptides and molecules described herein that are produced by modifying the polynucleotide encoding a polypeptide and/or proteinaceous component of a binding molecule by altering one or more amino acids or deleting or inserting one or more amino acids that may render it more suitable to achieve desired properties, such as more optimal expression by a host cell.

### IX. PD-L1 Binding Molecules Immobilized on Solid Substrates

In some embodiments, a molecule described herein (e.g. a binding molecule, fusion protein, or polynucleotide), or any effector fragment thereof, is immobilized on a solid substrate. Solid substrates contemplated herein include, but are not limited to, microbeads, nanoparticles, polymers, matrix materials, microarrays, microtiter plates, or any solid surface known in the art (see e.g. U.S. Pat. No. 7,771,955). In accordance with these embodiments, a molecule may be covalently or non-covalently linked to a solid substrate, such as, e.g., a bead, particle, or plate, using techniques known to the skilled worker (see e.g. Jung Y et al., Analyst 133: 697-701 (2008)). Immobilized molecules may be used for screening applications using techniques known in the art (see e.g. Bradbury A et al., Nat Biotechnol 29: 245-54 (2011); Sutton C, Br J Pharmacol 166: 457-75 (2012); Diamante L et al., Protein Eng Des Sel 26: 713-24 (2013); Houlihan G et al., J Immunol Methods 405: 47-56 (2014)).

Non-limiting examples of solid substrates to which a molecule may be immobilized on include: microbeads, nanoparticles, polymers, nanopolymers, nanotubes, magnetic beads, paramagnetic beads, superparamagnetic beads, streptavidin coated beads, reverse-phase magnetic beads, carboxy terminated beads, hydrazine terminated beads, silica (sodium silica) beads and iminodiacetic acid (IDA) -modified beads, aldehyde-modified beads, epoxy-activated beads, diaminodipropylamine (DADPA) -modified beads (beads with primary amine surface group), biodegradable polymeric beads, polystyrene substrates, amino-polystyrene particles, carboxyl-polystyrene particles, epoxy-polystyrene particles, dimethylamino-polystyrene particles, hydroxy-polystyrene particles, colored particles, flow cytometry particles, sulfonate-polystyrene particles, nitrocellulose surfaces, reinforced nitrocellulose membranes, nylon membranes, glass surfaces, activated glass surfaces, activated quartz surfaces, polyvinylidene difluoride (PVDF) membranes, polyacrylamide-based substrates, poly-vinyl chloride substrates, poly-methyl methacrylate substrates, poly(dimethyl siloxane) substrates, and photopolymers which contain photoreactive species (such as nitrenes, carbenes, and ketyl radicals) capable of forming covalent linkages. Other examples of solid substrates to which a molecule may be immobilized on are commonly used in molecular display systems, such as, e.g., cellular surfaces, phages, and virus particles.

### X. Delivery Devices and Kits

In some embodiments, the disclosure relates to a device comprising one or more compositions of matter, such as a pharmaceutical composition or diagnostic composition, for delivery to a subject in need thereof. Thus, a delivery device comprising one or more compositions can be used to administer to a patient a composition of matter by various delivery methods, including: intravenous, subcutaneous, intramuscular or intraperitoneal injection; oral administration; transdermal administration; pulmonary or transmucosal administration; administration by implant, osmotic pump, cartridge or micro pump; or by other means recognized by a person of skill in the art.

Also provided herein are kits comprising at least one composition as described herein, and optionally, packaging and instructions for use. Kits may be useful for drug administration and/or diagnostic information gathering. A kit may optionally comprise at least one additional reagent (e.g., standards, markers and the like). Kits typically include a label indicating the intended use of the contents of the kit. The kit may further comprise reagents and other tools for detecting a cell type (e.g. a tumor cell) in a sample or in a subject, or for diagnosing whether a patient belongs to a group that responds to a therapeutic strategy which makes use of a compound, composition, or related method. e.g., such as a method described herein.

XI. Methods for Using Binding Molecules and/or Pharmaceutical and/or Diagnostic Compositions Thereof

Generally, it is an object of the present disclosure to provide pharmacologically active agents, as well as compositions comprising the same, that can be used in the prevention and/or treatment of diseases, disorders, and conditions, such as certain cancers, tumors, growth abnormalities, immune disorders, or further pathological conditions mentioned herein. Accordingly, provided herein are methods of using the polypeptides, binding molecules, and pharmaceutical compositions for the targeted killing of cells, for delivering additional exogenous materials into targeted cells, for labeling of the interiors of targeted cells, for collecting diagnostic information, for the delivering of T-cell epitopes to the MHC class 1 presentation pathway of target cells, and for treating diseases, disorders, and conditions as described herein. For example, the methods described herein may be used to prevent or treat cancers, cancer initiation, tumor initiation, metastasis, and/or disease reoccurrence.

In particular, it is an object of the disclosure to provide such pharmacologically active agents, compositions, and/or methods that have certain advantages compared to the agents, compositions, and/or methods that are known in the art. Accordingly, the present disclosure provides methods of using Shiga toxin effector polypeptides and binding molecules with specified protein sequences and pharmaceutical compositions thereof. For example, any of the amino acid sequences described herein may be specifically utilized as a component of the binding molecule used in the following methods or any method for using a binding molecule known to the skilled worker, such as, e.g., various methods described in WO 2014/164680, WO 2014/164693, WO 2015/138435, WO 2015/138452, WO 2015/113005, WO 2015/113007, WO 2015/191764, US20150259428, WO 2016/196344, WO 2017/019623, WO 2018/106895, and WO 2018/140427.

Provided herein are methods of killing a cell comprising the step of contacting the cell, either in vitro or in vivo, with a Shiga toxin effector polypeptide, binding molecule, or pharmaceutical composition as described herein. The Shiga toxin effector polypeptides, binding molecules, and pharmaceutical compositions described herein can be used to kill a specific cell type upon contacting a cell or cells with one of the claimed compositions of matter. In some embodiments, a binding molecule or pharmaceutical composition can be used to kill specific cell types in a mixture of different cell types, such as mixtures comprising cancer cells, infected cells, and/or hematological cells. In some embodiments, a binding molecule, or pharmaceutical composition can be used to kill cancer cells in a mixture of different cell types. In some embodiments, a cytotoxic Shiga binding molecule, or pharmaceutical composition can be used to kill specific cell types in a mixture of different cell types, such as pre-transplantation tissues. In some embodiments, a Shiga toxin effector polypeptide, binding molecule, or pharmaceutical composition can be used to kill specific cell types in a mixture of cell types, such as pre-administration tissue material for therapeutic purposes. In some embodiments, a binding molecule or pharmaceutical composition can be used to selectively kill cells infected by viruses or microorganisms, or otherwise selectively kill cells expressing a particular extracellular target biomolecule, such as a cell surface biomolecule. The Shiga toxin effector polypeptides, binding molecules, and pharmaceutical compositions have varied applications, including, e.g., uses in depleting unwanted cell types from tissues either in vitro or in vivo, uses in modulating immune responses to treat graft versus host, uses as antiviral agents, uses as anti-parasitic agents, and uses in purging transplantation tissues of unwanted cell types.

In some embodiments, certain Shiga toxin effector polypeptides, binding molecules, and pharmaceutical compositions, alone or in combination with other compounds or pharmaceutical compositions, can show potent cell-kill activity when administered to a population of cells, in vitro or in vivo in a subject such as in a patient in need of treatment. By targeting the delivery of enzymatically active Shiga toxin A Subunit effector polypeptides and/or T-cell epitopes using high-affinity binding regions to specific cell types, cell-kill activities can be restricted to specifically and selectively killing certain cell types within an organism, such as certain cancer cells, neoplastic cells, malignant cells, non-malignant tumor cells, and/or infected cells.

In some embodiments, a method of killing a cell in a patient in need thereof comprises the step of administering to the patient at least one binding molecule or a pharmaceutical composition thereof.

In some embodiments, the binding molecule or pharmaceutical compositions thereof can be used to kill a cancer cell in a patient by targeting an extracellular PD-L1 found physically coupled with a cancer or tumor cell. The terms “cancer cell” or “cancerous cell” refers to various neoplastic cells which grow and divide in an abnormally accelerated and/or unregulated fashion and will be clear to the skilled person. The term “tumor cell” includes both malignant and non-malignant cells. Generally, cancers and/or tumors can be defined as diseases, disorders, or conditions that are amenable to treatment and/or prevention. The cancers and tumors (either malignant or non-malignant) which are comprised of cancer cells and/or tumor cells which may benefit from methods and compositions will be clear to the skilled person. Neoplastic cells are often associated with one or more of the following: unregulated growth, lack of differentiation, local tissue invasion, angiogenesis, and metastasis. The diseases, disorders, and conditions resulting from cancers and/or tumors (either malignant or non-malignant) which may benefit from the methods and compositions described herein for targeting certain cancer cells and/or tumor cells will be clear to the skilled person.

In some embodiments, the binding molecules and compositions described herein may be used to kill cancer stem cells, tumor stem cells, pre-malignant cancer-initiating cells, and tumor-initiating cells, which commonly are slow dividing and resistant to cancer therapies like chemotherapy and radiation. For example, acute myeloid leukemias (AMLs) may be treated by killing AML stem cells and/or dormant AML progenitor cells (see e.g. Shlush L et al., Blood 120: 603-12 (2012)).

Because of the Shiga toxin A Subunit based mechanism of action, compositions of matter described herein may be more effectively used in methods involving their combination with, or in complementary fashion with other therapies, such as, e.g., chemotherapies, immunotherapies, radiation, stem cell transplantation, and immune checkpoint inhibitors, and/or effective against chemoresistant/radiation-resistant and/or resting tumor cells/tumor initiating cells/stem cells. Similarly, compositions of matter may be more effectively used in methods involving in combination with other cell-targeted therapies targeting other than the same epitope on, non-overlapping, or different targets for the same disease disorder or condition.

Certain embodiments of the binding molecules, or pharmaceutical compositions thereof, can be used to kill an immune cell (whether healthy or malignant) in a patient by targeting an extracellular PD-L1 found physically coupled with an immune cell.

It is within the scope of the present disclosure to utilize a binding molecule, or pharmaceutical composition thereof, for the purposes of purging patient cell populations (e.g. bone marrow) of malignant, neoplastic, or otherwise unwanted T-cells and/or B-cells and then reinfusing the T-cell and/or B-cells depleted material into the patient (see e.g. van Heeckeren W et al., Br J Haematol 132: 42-55 (2006); (see e.g. Alpdogan O, van den Brink M, Semin Oncol 39: 62942 (2012)).

It is within the scope of the present disclosure to utilize the binding molecule, or pharmaceutical composition thereof, for the purposes of ex vivo depletion of T cells and/or B-cells from isolated cell populations removed from a patient. In one non-limiting example, the binding molecule can be used in a method for prophylaxis of organ and/or tissue transplant rejection wherein the donor organ or tissue is perfused prior to transplant with a cytotoxic, binding molecule or a pharmaceutical composition thereof in order to purge the organ of donor T-cells and/or B-cells (see e.g. Alpdogan O, van den Brink M, Semin Oncol 39: 629-42(2012)).

It is also within the scope of the present disclosure to utilize the binding molecule, or pharmaceutical composition thereof, for the purposes of depleting T-cells and/or B-cells from a donor cell population as a prophylaxis against graft-versus-host disease, and induction of tolerance, in a patient to undergo a bone marrow and or stem cell transplant (see e.g. van Heeckeren W et al., Br J Haematol 132: 42-55 (2006); (see e.g. Alpdogan O, van den Brink M, Semin Oncol 39: 629-42 (2012)).

In some embodiments of the Shiga toxin effector polypeptide or binding molecule, or pharmaceutical compositions thereof, can be used to kill an infected cell in a patient by targeting an extracellular PD-L1 found physically coupled with an infected cell.

In some embodiments of the binding molecules, or pharmaceutical compositions thereof, can be used to “seed” a locus within a chordate with non-self, T-cell epitope-peptide presenting cells in order to activate the immune system to enhance policing of the locus. In some embodiments of this “seeding” method, the locus is a tumor mass or infected tissue site. In preferred embodiments of this “seeding” method, the non-self, T-cell epitope-peptide is selected from the group consisting of: peptides not already presented by the target cells of the binding molecule, peptides not present within any protein expressed by the target cell, peptides not present within the proteome or transcriptome of the target cell, peptides not present in the extracellular microenvironment of the site to be seeded, and peptides not present in the tumor mass or infect tissue site to be targeting.

This “seeding” method functions to label one or more target cells within a chordate with one or more MHC class I presented T-cell epitopes for recognition by effector T-cells and activation of downstream immune responses. By exploiting the cell internalizing, intracellularly routing, and T-cell epitope delivering functions of the binding molecules, the target cells which display the delivered T-cell epitope are harnessed to induce recognition of the presenting target cell by host T-cells and induction of further immune responses including target-cell-killing by CTLs. This “seeding” method of using a binding molecule can provide a temporary vaccination-effect by inducing adaptive immune responses to attack the cells within the seeded microenvironment, such as, e.g. a tumor mass or infected tissue site, whether presenting a binding molecule-delivered T-cell epitope(s) or not. This “seeding” method may also induce the breaking of immuno-tolerance to a target cell population, a tumor mass, and/or infected tissue site within a chordate.

In some embodiments, methods involving the seeding of a locus within a chordate with one or more antigenic and/or immunogenic epitopes may be combined with the administration of immunologic adjuvants, whether administered locally or systemically, to stimulate the immune response to certain antigens, such as, e.g., the co-administration of a composition described herein with one or more immunologic adjuvants like a cytokine, bacterial product, or plant saponin. Other examples of immunologic adjuvants which may be suitable for use in the methods described herein include aluminum salts and oils, such as, e.g., alums, aluminum hydroxide, mineral oils, squalene, paraffin oils, peanut oils, and thimerosal.

Additionally, provided herein is a method of treating a disease, disorder, or condition in a patient comprising the step of administering to a patient in need thereof an effective amount of at least one of the binding molecules, or a pharmaceutical composition thereof. In some embodiments, the disease, disorder, or condition involves a PD-L1 expressing cell. Contemplated diseases, disorders, and conditions that can be treated using this method include cancers, malignant tumors, non-malignant tumors, growth abnormalities, immune disorders, and microbial infections. Administration of a “therapeutically effective dosage” of a composition described herein can result in a decrease in severity of disease symptoms, an increase in frequency and duration of disease symptom-free periods, or a prevention of impairment or disability due to the disease affliction.

The therapeutically effective amount of a composition will depend on the route of administration, the type of organism being treated, and the physical characteristics of the specific patient under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by herein and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

An acceptable route of administration may refer to any administration pathway known in the art, including but not limited to aerosol, enteral, nasal, ophthalmic, oral, parenteral, rectal, vaginal, or transdermal (e.g. topical administration of a cream, gel or ointment, or by means of a transdermal patch). “Parenteral administration” is typically associated with injection at or in communication with the intended site of action, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

For administration of a pharmaceutical composition, the dosage range will generally be from about 0.001 to 10 milligrams per kilogram (mg/kg), and more, usually 0.001 to 0.5 mg/kg, of the subject's body weight. Exemplary dosages may be 0.01 mg/kg body weight, 0.03 mg/kg body weight, 0.07 mg/kg body weight, 0.09 mg/kg body weight or 0.1 mg/kg body weight or within the range of 0.01 to 0.1 mg/kg. An exemplary treatment regime is a once or twice daily administration, or a once or twice weekly administration, once every two weeks, once every three weeks, once every four weeks, once a month, once every two or three months or once every three to 6 months. Dosages may be selected and readjusted by the skilled health care professional as required to maximize therapeutic benefit for a particular patient.

Pharmaceutical compositions will typically be administered to the same patient on multiple occasions. Intervals between single dosages can be, for example, two to five days, weekly, monthly, every two or three months, every six months, or yearly. Intervals between administrations can also be irregular, based on regulating blood levels or other markers in the subject or patient. Dosage regimens for a composition include, for example, intravenous administration of 0.01 mg/kg body weight or 0.03 mg/kg body weight with the composition administered every two to four weeks for six dosages, then every three months at 0.03 mg/kg body weight or 0.01 mg/kg body weight.

A pharmaceutical composition may be administered via one or more routes of administration, using one or more of a variety of methods known in the art. As will be appreciated by the skilled worker, the route and/or mode of administration will vary depending upon the desired results. Routes of administration for binding molecules and pharmaceutical compositions include, e.g. intravenous, intramuscular, intradermal, intraperitoneal, subcutaneous, spinal, or other parenteral routes of administration, for example by injection or infusion. For other embodiments, a binding molecule or pharmaceutical composition may be administered by a non-parenteral route, such as a topical, epidermal or mucosal route of administration, for example, intranasally, orally, vaginally, rectally, sublingually, or topically.

Therapeutic binding molecules or pharmaceutical compositions may be administered with one or more of a variety of medical devices known in the art. For example, in one embodiment, a pharmaceutical composition may be administered with a needleless hypodermic injection device. Examples of well-known implants and modules useful are known in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

The binding molecule or pharmaceutical composition may be administered alone or in combination with one or more other therapeutic or diagnostic agents. A combination therapy may include a binding molecule, or pharmaceutical composition thereof, combined with at least one other therapeutic agent selected based on the particular patient, disease or condition to be treated. Examples of other such agents include, inter alia, a cytotoxic, anti-cancer or chemotherapeutic agent, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates one or more signaling pathways, and similar modulating therapeutic molecules which may complement or otherwise be beneficial in a therapeutic or prophylactic treatment regimen.

Treatment of a patient with binding molecule or pharmaceutical composition may, in some embodiments, lead to cell death of targeted cells and/or the inhibition of growth of targeted cells. As such, cytotoxic, binding molecules, and pharmaceutical compositions comprising them, will be useful in methods for treating a variety of pathological disorders in which killing or depleting target cells may be beneficial, such as, inter alia, cancer, tumors, other growth abnormalities, immune disorders, and infected cells. Also provided herein are methods for suppressing cell proliferation, and treating cell disorders, including neoplasia, overactive B-cells, and overactive T-cells.

In some embodiments, the binding molecules and pharmaceutical compositions described herein can be used to treat or prevent cancers, tumors (malignant and non-malignant), growth abnormalities, immune disorders, and microbial infections. In some embodiments, the above ex vivo method can be combined with the above in vivo method to provide methods of treating or preventing rejection in bone marrow transplant recipients, and for achieving immunological tolerance.

In some embodiments, methods for treating malignancies or neoplasms and other blood cell associated cancers in a mammalian subject, such as a human, comprise the step of administering to a subject in need thereof a therapeutically effective amount of a cytotoxic binding molecule or pharmaceutical composition.

The binding molecules and pharmaceutical compositions have varied applications, including, e.g., uses in removing unwanted T-cells, uses in modulating immune responses to treat graft versus host, uses as antiviral agents, uses as antimicrobial agents, and uses in purging transplantation tissues of unwanted cell types. The binding molecules and pharmaceutical compositions described herein are commonly anti-neoplastic agents—meaning they are capable of treating and/or preventing the development, maturation, or spread of neoplastic or malignant cells by inhibiting the growth and/or causing the death of cancer or tumor cells.

In some embodiments, the binding molecule or pharmaceutical composition is used to treat a B-cell-, plasma cell- or antibody-mediated disease or disorder, such as for example leukemia, lymphoma (e.g., primary mediastinal B cell lymphoma. Hodgkin's lymphoma, or non-Hodgkin's lymphoma), myeloma, rheumatic disease, spondylitis, Human Immunodeficiency Virus-related diseases, amyloidosis, hemolytic uremic syndrome, polyarteritis, septic shock, Crohn's Disease, rheumatoid arthritis, ankylosing spondylitis, psoriatic arthritis, ulcerative colitis, psoriasis, asthma, Sjögren's syndrome, graft-versus-host disease, graft rejection, diabetes, vasculitis, scleroderma, and systemic lupus erythematosus.

In some embodiments, certain embodiments of the binding molecules and pharmaceutical compositions described herein are antimicrobial agents—meaning they are capable of treating and/or preventing the acquisition, development, or consequences of microbiological pathogenic infections, such as caused by viruses, bacteria, fungi, prions, or protozoans.

It is within the scope of the present disclosure to provide a prophylaxis or treatment for diseases or conditions mediated by T-cells or B-cells by administering a binding molecule described herein, or a pharmaceutical composition thereof, to a patient for the purpose of killing T-cells or B-cells in the patient. This usage is compatible with preparing or conditioning a patient for bone marrow transplantation, stem cell transplantation, tissue transplantation, or organ transplantation, regardless of the source of the transplanted material, e.g. human or non-human sources.

It is within the scope of the present disclosure to provide a bone marrow recipient for prophylaxis or treatment of host-versus-graft disease via the targeted cell-killing of host T-cells using a cytotoxic binding molecule or pharmaceutical composition as described herein.

In some embodiments, a method of treating cancer comprises administering to a subject in need thereof an effective amount of a PDL-1 binding molecule or a pharmaceutical composition comprising the same. In some embodiments, a method of treating cancer comprises administering to a subject in need thereof an effective amount of a nucleic acid (e.g., an expression vector) encoding a PD-L1 binding molecule. In some embodiments, the cancer is any one of the following: bladder cancer (e.g., urothelial carcinoma), breast cancer (e.g., HER2 positive breast cancer, triple negative breast cancer), colon cancer (e.g., colorectal cancer such as metastatic microsatellite instability-high or mismatch repair deficient colorectal cancer), endometrial cancer, esophageal cancer, fallopian tube cancer, gastrointestinal cancer (e.g., gastric cancer, biliary tract neoplasm, gastroesophageal junction cancer), glioblastoma, glioma, head and neck cancer (e.g., squamous cell carcinoma of the head and neck), kidney cancer (e.g., renal cell carcinoma), liver cancer (e.g., hepatocellular carcinoma), lung cancer (e.g., non-small cell lung cancer, small-cell lung cancer), lymphoma (e.g., diffuse large B-cell lymphoma, Hodgkin lymphoma, non-Hodgkin lymphoma, primary mediastinal large B-cell lymphoma). Merkel cell carcinoma, mesothelioma (e.g., pleural mesothelioma), myeloma (e.g., multiple myeloma), nasopharyngeal neoplasm, ovarian cancer, pancreatic cancer, peritoneal neoplasm, prostate cancer, skin cancer (e.g., squamous cell cancer of the skin, melanoma, transitional cell carcinoma, or urothelial cancer.

Some embodiments of the binding molecules and pharmaceutical compositions can be utilized in a method of treating cancer comprising administering to a patient, in need thereof, a therapeutically effective amount of a binding molecule and/or pharmaceutical composition. In some embodiments, the cancer being treated is selected from the group consisting of: bone cancer (such as multiple myeloma or Ewing's sarcoma), breast cancer (such as HER2 positive breast cancer or triple negative breast cancer), central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer (such as hepatocellular carcinoma), lung/pleura cancer (such as mesothelioma, small cell lung cancer, or non-small cell lung cancer), prostate cancer, sarcoma (such as angiosarcoma, fibrosarcoma. Kaposi's sarcoma, or synovial sarcoma), skin cancer (such as basal cell carcinoma, squamous cell carcinoma, or melanoma), urothelial cancer, gastric cancer, esophageal cancer, head and neck squamous cell cancer, cervical cancer, Merkel cell carcinoma, endometrial cancer, and uterine cancer.

In some embodiments of the methods of treating cancer described herein, the subject received at least one line or regimen of prior treatment, before administration with a binding molecule. In some embodiments, subject has cancer, and the cancer is relapsed or refractory to at least one prior treatment, such as checkpoint inhibitor therapy. In some embodiments, the cancer is relapsed or refractory to ipilimumab, nivolumab, pembrolizumab, atezolizumab, durvalumab, avelumab, tremelimumab or cemiplimab. In some embodiments, the cancer is one of the cancers listed in Table 6, below, and is relapsed or refractory to at least one prior treatment marked with an “X” in the table.

TABLE 6
Cancers treatable with a binding molecule of the disclosure that can be relapsed or refractory to prior treatments

| Cancer | Ipilimumab | Nivolumab | Pembroizumab | Atezolizumab | Durvalumab | Avelumab | Cemiplimab |
|---|---|---|---|---|---|---|---|
| Melanoma | X | X | X | | | | |
| Merkel Cell | | | X | | | X | |
| Cutaneous | | | | | | | X |
| Squamous Cell | | | | | | | |
| Carcinoma | | | | | | | |
| Non-small cell | | X | X | X | X | | |
| lung cancer | | | | | | | |
| Small cell | | X | X | X | | | |
| lung cancer | | | | | | | |
| Squamous cell | | X | X | | X | | |
| carcinoma | | | | | | | |
| of the head | | | | | | | |
| and neck | | | | | | | |
| Esophageal | | | X | | | | |
| cancer | | | | | | | |
| Gastric cancer | | X | X | | | | |
| Colorectal | | X | X | | | | |
| cancer | | | | | | | |
| Hepatocellular | | X | | | | | |
| carcinoma | | | | | | | |
| Bladder cancer | | X | X | X | X | X | |
| Renal Cell | X | X | X | | | X | |
| Carcinoma | | | | | | | |

In some embodiments, a method of treating cancer comprises administering to a subject in need thereof an effective amount of a PDL-1 binding molecule or a pharmaceutical composition comprising the same, wherein the cancer is metastatic.

Some embodiments of the binding molecules and pharmaceutical compositions can be utilized in a method of treating an immune disorder comprising administering to a patient, in need thereof, a therapeutically effective amount of the binding molecules and/or pharmaceutical composition. In some embodiments, the immune disorder is related to an inflammation associated with a disease selected from the group consisting of: rheumatic disease, spondylitis, amyloidosis, ankylosing spondylitis, asthma, Crohn's disease, diabetes, graft rejection, graft-vs.-host disease, Hashimoto's thyroiditis, hemolytic uremic syndrome, HIV-related diseases, lupus erythematosus, multiple sclerosis, polyarteritis, psoriasis, psoriatic arthritis, rheumatoid arthritis, scleroderma, septic shock, Sjögren's syndrome, ulcerative colitis, and vasculitis.

In some embodiments, the Shiga toxin effector polypeptide or binding molecule is used as a component of a pharmaceutical composition or medicament for the treatment or prevention of a cancer, tumor, other growth abnormality, immune disorder, and/or microbial infection. For example, immune disorders presenting on the skin of a patient may be treated with such a medicament in efforts to reduce inflammation. In another example, skin tumors may be treated with such a medicament in efforts to reduce tumor size or eliminate the tumor completely.

Certain cytotoxic binding molecules, and compositions thereof, may be used in molecular neurosurgery applications such as immunolesioning and neuronal tracing (see, Wiley R. Lappi D, Adv Drug Deliv Rev 55: 1043-54 (2003), for review). For example, the targeting domain may be selected or derived from various ligands, such as neurotransmitters and neuropeptides, which target specific neuronal cell types by binding neuronal surface receptors, such as a neuronal circuit specific G-protein coupled receptor. Similarly, the targeting domain may be selected from or derived from antibodies that bind neuronal surface receptors. Because certain Shiga toxin effector polypeptides robustly direct their own retrograde axonal transport, certain binding molecules may be used to kill a neuron(s) which expresses the extracellular target at a site of cytotoxic protein injection distant from the cell body (see Llewellyn-Smith I et al., J Neurosci Methods 103: 83-90 (2000)). These targeted cytotoxic molecules that specifically target neuronal cell types have uses in neuroscience research, such as for elucidating mechanisms of sensations (see e.g. Mishra S, Hoon M, Science 340: 968-71 (2013), and creating model systems of neurodegenerative diseases, such as Parkinson's and Alzheimer's (see e.g. Hamlin A et al., PLoS One e53472 (2013)).

In some embodiments, a method of using a Shiga toxin effector polypeptide, binding molecule, pharmaceutical composition, and/or diagnostic composition as described herein to label or detect the interiors of neoplastic cells and/or immune cell types is provided. This method may be based on the ability of certain binding molecules to enter specific cell types and route within cells via retrograde intracellular transport, to the interior compartments of specific cell types are labeled for detection. This can be performed on cells in situ within a patient or on cells and tissues removed from an organism, e.g. biopsy material.

In some embodiments, a method of using a Shiga toxin effector polypeptide, binding molecule, pharmaceutical composition, and/or diagnostic composition to detect the presence of a cell type for the purpose of information gathering regarding diseases, conditions and/or disorders is provided. The method comprises contacting a cell with a diagnostically effective amount of a binding molecule in order to detect the molecule by an assay or diagnostic technique. The phrase “diagnostically effective amount” refers to an amount that provides adequate detection and accurate measurement for information gathering purposes by the particular assay or diagnostic technique utilized. Generally, the diagnostically effective amount for whole organism in vivo diagnostic use will be a non-cumulative dose of between 0.001 to 10 milligrams of the detection promoting agent linked binding molecule per kg of subject per subject. Typically, the amount of Shiga toxin effector polypeptide or binding molecule used in these information gathering methods will be as low as possible, provided that it is still a diagnostically effective amount. For example, for in vivo detection in an organism, the amount of Shiga toxin effector polypeptide, binding molecule, or pharmaceutical composition administered to a subject will be as low as feasibly possible.

The cell-type specific targeting of binding molecules combined with detection promoting agents provides a way to detect and image cells physically coupled with an extracellular PD-L1 bound by the binding region of the molecule. Imaging of cells using the binding molecules may be performed in vitro or in vivo by any suitable technique known in the art. Diagnostic information may be collected using various methods known in the art, including whole body imaging of an organism or using ex vivo samples taken from an organism. The term “sample” used herein refers to any number of things, but not limited to, fluids such as blood, urine, serum, lymph, saliva, anal secretions, vaginal secretions, and semen, and tissues obtained by biopsy procedures. For example, various detection promoting agents may be utilized for non-invasive in vivo tumor imaging by techniques such as magnetic resonance imaging (MRI), optical methods (such as direct, fluorescent, and bioluminescent imaging), positron emission tomography (PET), single-photon emission computed tomography (SPECT), ultrasound, x-ray computed tomography, and combinations of the aforementioned (see, Kaur S et al., Cancer Lett 315: 97-111 (2012), for review).

Also provided is a method of using a Shiga toxin effector polypeptide, binding molecule, or pharmaceutical composition in a diagnostic composition to label or detect the interiors of a hematologic cell, cancer cell, tumor cell, infected cell, and/or immune cell (see e.g., Koyama Y et al., Clin Cancer Res 13: 2936-45 (2007); Ogawa M et al., Cancer Res 69: 1268-72 (2009); Yang L et al., Small 5: 235-43 (2009)). Based on the ability of certain binding molecules to enter specific cell types and route within cells via retrograde intracellular transport, the interior compartments of specific cell types are labeled for detection. This can be performed on cells in situ within a patient or on cells and tissues removed from an organism, e.g. biopsy material.

Diagnostic compositions may be used to characterize a disease, disorder, or condition as potentially treatable by a related pharmaceutical composition. Some compositions of matter may described herein be used to determine whether a patient belongs to a group that responds to a therapeutic strategy which makes use of a compound, composition or related method as described herein or is well suited for using a delivery device as described herein.

Diagnostic compositions may be used after a disease, e.g. a cancer, is detected in order to better characterize it, such as to monitor distant metastases, heterogeneity, and stage of cancer progression. The phenotypic assessment of disease disorder or infection can help prognostic and prediction during therapeutic decision making. In disease reoccurrence, certain methods may be used to determine if local or systemic problem.

Diagnostic compositions may be used to assess responses to therapies regardless of the type of the type of therapy, e.g. small molecule drug, biological drug, or cell-based therapy. For example, certain embodiments of the diagnostics may be used to measure changes in tumor size, changes in antigen positive cell populations including number and distribution, or monitoring a different marker than the antigen targeted by a therapy already being administered to a patient (see Smith-Jones P et al., Nat. Biotechnol 22: 701-6 (2004); Evans M et al, Proc. Natl. Acad Sci. USA 108: 9578-82 (2011)).

In some embodiments of the method used to detect the presence of a cell type may be used to gather information regarding diseases, disorders, and conditions, such as, for example bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), uterine cancer, AIDS, rheumatic disease, spondylitis, amyloidosis, ankylosing spondylitis, asthma, autism, cardiorheumatic disease, Crohn's disease, diabetes, erythematosus, gastritis, graft rejection, graft-versus-host disease, Grave's disease, Hashimoto's thyroiditis, hemolytic uremic syndrome, HIV-related diseases, lupus erythematosus, lymphoproliferative disorders (including post-transplant lymphoproliferative disorders), multiple sclerosis, myasthenia gravis, neuroinflammation, polyarteritis, psoriasis, psoriatic arthritis, rheumatoid arthritis, scleroderma, septic shock, Sjögren's syndrome, systemic lupus erythematosus, ulcerative colitis, vasculitis, cell proliferation, inflammation, leukocyte activation, leukocyte adhesion, leukocyte chemotaxis, leukocyte maturation, leukocyte migration, neuronal differentiation, acute lymphoblastic leukemia (ALL), T acute lymphocytic leukemia/lymphoma (ALL), acute myelogenous leukemia, acute myeloid leukemia (AML), B-cell chronic lymphocytic leukemia (B-CLL), B-cell prolymphocytic lymphoma, Burkitt's lymphoma (BL), chronic lymphocytic leukemia (CLL), chronic myelogenous leukemia (CML-BP), chronic myeloid leukemia (CML), diffuse large B-cell lymphoma, follicular lymphoma, hairy cell leukemia (HCL), Hodgkin's Lymphoma (HL), intravascular large B-cell lymphoma, lymphomatoid granulomatosis, lymphoplasmacytic lymphoma, MALT lymphoma, mantle cell lymphoma, multiple myeloma (MM), natural killer cell leukemia, nodal marginal B-cell lymphoma, Non-Hodgkin's lymphoma (NHL), plasma cell leukemia, plasmacytoma, primary effusion lymphoma, prolymphocytic leukemia, promyelocytic leukemia, small lymphocytic lymphoma, splenic marginal zone lymphoma, T-cell lymphoma (TCL), heavy chain disease, monoclonal gammopathy, monoclonal immunoglobulin deposition disease, myelodysplastic syndromes (MDS), smoldering multiple myeloma, and Waldenstrom macroglobulinemia.

In some embodiments, the Shiga toxin effector polypeptides and binding molecules, or pharmaceutical compositions thereof, are used for both diagnosis and treatment, or for diagnosis alone. In some situations, it would be desirable to determine or verify the HLA variant(s) and/or HLA alleles expressed in the subject and/or diseased tissue from the subject, such as, e.g., a patient in need of treatment, before selecting a Shiga toxin effector polypeptide or binding molecule for use in treatment(s).

### NUMBERED EMBODIMENTS

The present invention is further illustrated by the following numbered embodiments, and the non-limiting examples of binding molecules capable of specifically targeting PD-L1 and comprising one or more proteinaceous toxin components.

Embodiment 1. A PD-L1 binding molecule comprising:

(i) a Shiga-like toxin A subunit effector polypeptide;

(ii) a binding region capable of specifically binding an extracellular part of PD-L1; wherein the binding region comprises:
(a) a heavy chain variable region (VH) comprising:
   (1) a CDR1 comprising the amino acid sequence EYTMH (SEQ ID NO:27),
   (2) a CDR2 comprising the amino acid sequence GINPNNGGTWYNQKFKG (SEQ ID NO:29), and
   (3) a CDR3 comprising the amino acid sequence PYYYGSREDYFDY (SEQ ID NO:32);
and
(b) a light chain variable region (VL) comprising:
   (1) a CDR1 comprising the amino acid sequence SASSSVSYMY (SEQ ID NO:19),
   (2) a CDR2 comprising the amino acid sequence LTSNLAS (SEQ ID NO:20), and
   (3) a CDR3 comprising the amino acid sequence QQWSSNPPT (SEQ ID NO:26); and

(iii) at least one CD8+ T-cell epitope that is heterologous to Shiga-like toxin A subunits.

Embodiment 2. The PD-L1 binding molecule of embodiment 1, wherein the CD8+ T-cell epitope comprises the sequence of SEQ ID NO: 300 or 301.

Embodiment 3. The PD-L1 binding molecule of embodiment 1, wherein the at least one CD8+ T-cell epitope is an antigen recognized by HLA subtypes HLA-A. HLA-B, or HLA-C.

Embodiment 4. The PD-L1 binding molecule of embodiment 1, wherein the CD8+ T-cell epitope comprises the sequence of SEQ ID NO: 78-84.

Embodiment 5. The PD-L1 binding molecule of embodiment 1, wherein the at least one CD8+ T-cell epitope is an HLA:A01 restricted antigen.

Embodiment 6. The PD-L1 binding molecule of embodiment 1, wherein the at least one CD8+ T-cell epitope is an HLA:A02 restricted antigen.

Embodiment 7. The PD-L1 binding molecule of embodiment 1, wherein the at least one CD8+ T-cell epitope is an HLA:A03 restricted antigen.

Embodiment 8. The PD-L1 binding molecule of embodiment 1, wherein the at least one CD8+ T-cell epitope is an HLA:A24 restricted antigen Embodiment 9. The PD-L1 binding molecule of embodiment 1, wherein the at least one CD8+ T-cell epitope is isolated or derived from Human Cytomegalovirus (HCMV).

Embodiment 10. The PD-L1 binding molecule of embodiment 1, wherein the at least one CD8+ T-cell epitope is embedded or inserted into the Shiga-like toxin A subunit effector polypeptide.

Embodiment 11. The PD-L1 binding molecule of embodiment 1, wherein the at least one CD8+ T-cell epitope is located at the C-terminus of the Shiga-like toxin A subunit effector polypeptide.

Embodiment 12. The PD-L1 binding molecule of embodiment 1, wherein the at least one CD8+ T-cell epitope is embedded or inserted into the binding region.

Embodiment 13. The PD-L1 binding molecule of embodiment 1, wherein the at least one CD8+ T-cell epitope is located at the C-terminus of the binding region.

Embodiment 14. The PD-L1 binding molecule of embodiment 1, wherein the at least one CD8+ T-cell epitope is located between the Shiga-like toxin A subunit effector polypeptide and the binding region.

Embodiment 15. The PD-L1 binding molecule of embodiment 1, wherein the molecule comprises at least two CD8+ T-cell epitopes that are each heterologous to Shiga-like toxin A subunits.

Embodiment 16. The PD-L1 binding molecule of embodiment 1, wherein the molecule comprises at least three CD8+ T-cell epitopes that are each heterologous to Shiga-like toxin A subunits.

Embodiment 17. The PD-L1 binding molecule of embodiment 1, wherein the molecule comprises at least four CD8+ T-cell epitopes that are each heterologous to Shiga-like toxin A subunits.

Embodiment 18. The PD-L1 binding molecule of embodiment 1, wherein the molecule comprises, in order from N-terminus to C-terminus the Shiga-like toxin A subunit effector polypeptide; the binding region; and the at least one CD8+ T-cell epitope.

Embodiment 19. The PD-L1 binding molecule of embodiment 1, wherein the molecule comprises, in order from N-terminus to C-terminus, the Shiga-like toxin A subunit effector polypeptide; the binding region; and at least two CD8+ T-cell epitopes.

Embodiment 20. The PD-L1 binding molecule of embodiment 1, wherein the molecule comprises, in order from N-terminus to C-terminus the Shiga-like toxin A subunit effector polypeptide; the at least one CD8+ T-cell epitope; and the binding region.

Embodiment 21. The PD-L1 binding molecule of embodiment 1, wherein the molecule comprises, in order from N-terminus to C-terminus the Shiga-like toxin A subunit effector polypeptide; a first CD8+ T-cell epitope; the binding region; and a second CD8+ T-cell epitope.

Embodiment 22. The PD-L1 binding molecule of embodiment 21, wherein the first and the second CD8+ T-cell epitopes are different.

Embodiment 23. The PD-L1 binding molecule of embodiment 1, wherein the molecule comprises, in order from N-terminus to C-terminus the Shiga-like toxin A subunit effector polypeptide; a first CD8+ T-cell epitope; the binding region; a second CD8+ T-cell epitope; and a third CD8+ T-cell epitope.

Embodiment 24. The PD-L1 binding molecule of embodiment 23, wherein at least one of the first, second, and third CD8+ T-cell epitopes is different from the others.

Embodiment 25. The PD-L1 binding molecule of embodiment 1, wherein the molecule comprises, in order from N-terminus to C-terminus the binding region; the Shiga-like toxin A subunit effector polypeptide; and the at least one CD8+ T-cell epitope.

Embodiment 26. The PD-L1 binding molecule of embodiment 1, wherein the molecule comprises, in order from N-terminus to C-terminus, the binding region; the Shiga-like toxin A subunit effector polypeptide; and at least two CD8+ T-cell epitopes.

Embodiment 27. The PD-L1 binding molecule of embodiment 1, wherein the molecule comprises, in order from N-terminus to C-terminus the binding region; the at least one CD8+ T-cell epitope; and the Shiga-like toxin A subunit effector polypeptide.

Embodiment 28. The PD-L1 binding molecule of embodiment 1, wherein the molecule comprises, in order from N-terminus to C-terminus, the binding region; a first CD8+ T-cell epitope; the Shiga-like toxin A subunit effector polypeptide; and a second CD8+ T-cell epitope.

Embodiment 29. The PD-L1 binding molecule of embodiment 28, wherein the first and the second CD8+ T-cell epitopes are different.

Embodiment 30. The PD-L1 binding molecule of embodiment 1, wherein the molecule comprises, in order from N-terminus to C-terminus the binding region; a first CD8+ T-cell epitope; the Shiga-like toxin A subunit effector polypeptide; a second CD8+ T-cell epitope; and a third CD8+ T-cell epitope.

Embodiment 31. The PD-L1 binding molecule of embodiment 30, wherein at least one of the first, second, and third CD8+ T-cell epitopes is different from the others.

Embodiment 32. The PDl-L1 binding molecule of embodiment 1, wherein the Shiga-like toxin A subunit effector polypeptide comprises the sequence of SEQ ID NO: 41, or a sequence at least 90% or at least 95% identical thereto.

Embodiment 33. The PD-L1 binding molecule of embodiment 1, wherein the VH comprises the sequence of SEQ ID NO: 34, or a sequence at least 90% or at least 95% identical thereto.

Embodiment 34. The PD-L1 binding molecule of embodiment 1, wherein the VL comprises the sequence of SEQ ID NO: 35, or a sequence at least 90% or at least 95% identical thereto.

Embodiment 35. The PD-L1 binding molecule of embodiment 1, wherein the VH comprises the sequence of SEQ ID NO: 34 and the VL comprises the sequence of SEQ ID NO: 35.

Embodiment 36. The PD-L1 binding molecule of embodiment 1, wherein the binding region comprises the sequence of SEQ ID NO: 106, or a sequence at least 90% or at least 95% identical thereto.

Embodiment 37. The PD-L1 binding molecule of embodiment 1, wherein the PD-L1 binding molecule comprises the sequence of any one of SEQ ID NOs: 303-313, or a sequence at least 90% or at least 95% identical thereto.

Embodiment 38. The PD-L1 binding molecule of embodiment 1, wherein the PD-L1 binding molecule is a single continuous polypeptide.

Embodiment 39. The PD-L1 binding molecule embodiment 1, wherein the PD-L1 binding molecule comprises two polypeptides.

Embodiment 40. The PD-L1 binding molecule of embodiment 39, wherein each of the two polypeptide comprises the sequence of any one of SEQ ID NO: 303-313.

Embodiment 41. The PD-L1 binding molecule of embodiment 39, wherein the two polypeptides are non-covalently linked to each other.

Embodiment 42. The PD-L1 binding molecule of embodiment 1, wherein the binding molecule is cytotoxic.

Embodiment 43. A cell binding molecule comprising:
(i) a Shiga-like toxin A subunit effector polypeptide;
(ii) a binding region capable of specifically binding an extracellular target on a cell; and
(iii) CD8+ T-cell epitope comprising the sequence of SEQ ID NO: 300 or 301.

Embodiment 44. The cell binding molecule of embodiment 43, wherein the at least one CD8+ T-cell epitope is embedded or inserted into the Shiga-like toxin A subunit effector polypeptide.

Embodiment 45. The cell binding molecule of embodiment 43, wherein the at least one CD8+ T-cell epitope is located at the C-terminus of the Shiga-like toxin A subunit effector polypeptide.

Embodiment 46. The cell binding molecule of embodiment 43, wherein the at least one CD8+ T-cell epitope is embedded or inserted into the binding region.

Embodiment 47. The cell binding molecule of embodiment 43, wherein the at least one CD8+ T-cell epitope is located at the C-terminus of the binding region.

Embodiment 48. The cell binding molecule of embodiment 43, wherein the at least one CD8+ T-cell epitope is located between the Shiga-like toxin A subunit effector polypeptide and the binding region.

Embodiment 49. The cell binding molecule of embodiment 43, wherein the molecule comprises at least two CD8+ T-cell epitopes that are each heterologous to Shiga-like toxin A subunits.

Embodiment 50. The cell binding molecule of embodiment 43, wherein the molecule comprises at least three CD8+ T-cell epitopes that are each heterologous to Shiga-like toxin A subunits.

Embodiment 51. The cell binding molecule of embodiment 43, wherein the molecule comprises at least four CD8+ T-cell epitopes that are each heterologous to Shiga-like toxin A subunits.

Embodiment 52. The cell binding molecule of embodiment 43, wherein the molecule comprises, in order from N-terminus to C-terminus the Shiga-like toxin A subunit effector polypeptide; the binding region; and the at least one CD8+ T-cell epitope.

Embodiment 53. The cell binding molecule of embodiment 43, wherein the molecule comprises, in order from N-terminus to C-terminus, the Shiga-like toxin A subunit effector polypeptide; the binding region; and at least two CD8+ T-cell epitopes.

Embodiment 54. The cell binding molecule of embodiment 43, wherein the molecule comprises, m order from N-terminus to C-terminus, the Shiga-like toxin A subunit effector polypeptide; the at least one CD8+ T-cell epitope; and the binding region.

Embodiment 55. The cell binding molecule of embodiment 43, wherein the molecule comprises, in order from N-terminus to C-terminus the Shiga-like toxin A subunit effector polypeptide; a first CD8+ T-cell epitope; the binding region; and a second CD8+ T-cell epitope.

Embodiment 56. The cell binding molecule of embodiment 43, wherein the first and the second CD8+ T-cell epitopes are different.

Embodiment 57. The cell binding molecule of embodiment 43, wherein the molecule comprises, in order from N-terminus to C-terminus the Shiga-like toxin A subunit effector polypeptide; a first CD8+ T-cell epitope; the binding region; a second CD8+ T-cell epitope; and a third CD8+ T-cell epitope.

Embodiment 58. The cell binding molecule of embodiment 57, wherein at least one of the first, second, and third CD8+ T-cell epitopes is different from the others.

Embodiment 59. The cell binding molecule of embodiment 43, wherein the molecule comprises, in order from N-terminus to C-terminus the binding region; the Shiga-like toxin A subunit effector polypeptide; and the at least one CD8+ T-cell epitope.

Embodiment 60. The cell binding molecule of embodiment 59, wherein the molecule comprises, in order from N-terminus to C-terminus, the binding region; the Shiga-like toxin A subunit effector polypeptide; and at least two CD8+ T-cell epitopes.

Embodiment 61. The cell binding molecule of embodiment 43, wherein the molecule comprises, in order from N-terminus to C-terminus, the binding region; the at least one CD8+ T-cell epitope; and the Shiga-like toxin A subunit effector polypeptide.

Embodiment 62. The cell binding molecule of embodiment 43, wherein the molecule comprises, in order from N-terminus to C-terminus, the binding region; a first CD8+ T-cell epitope; the Shiga-like toxin A subunit effector polypeptide; and a second CD8+ T-cell epitope.

Embodiment 63. The cell binding molecule of embodiment 62, wherein the first and the second CD8+ T-cell epitopes are different.

Embodiment 64. The cell binding molecule of embodiment 43, wherein the molecule comprises, in order from N-terminus to C-terminus the binding region; a first CD8+ T-cell epitope; the Shiga-like toxin A subunit effector polypeptide; a second CD8+ T-cell epitope; and a third CD8+ T-cell epitope.

Embodiment 65. The cell binding molecule of embodiment 54, wherein at least one of the first, second, and third CD8+ T-cell epitopes is different from the others.

Embodiment 66. The cell binding molecule of embodiment 43, wherein the Shiga-like toxin A subunit effector polypeptide comprises the sequence of SEQ ID NO: 41, or a sequence at least 90% or at least 95% identical thereto.

Embodiment 67. The cell binding molecule of embodiment 43, wherein the Shiga-like toxin A subunit effector polypeptide comprises the amino acids 1-251 of SEQ ID NO: 1, or a sequence at least 90% or at least 95% identical thereto.

Embodiment 68. A pharmaceutical composition comprising the binding molecule of any one of embodiments 1-67, and at least one pharmaceutically acceptable excipient or carrier.

Embodiment 69. A polynucleotide encoding the binding molecule of any one of embodiments 1-67, or a complement thereof.

Embodiment 70. An expression vector comprising a polynucleotide according to embodiment 69.

Embodiment 71. A host cell comprising a polynucleotide according to embodiment 69 or an expression vector according to embodiment 70.

Embodiment 72. A method for making the binding molecule of any one of embodiments 1-67, the method comprising (a) expressing the binding molecule and (b) recovering the binding molecule.

Embodiment 73. The method of embodiment 72, wherein expressing the binding molecule comprises culturing the host cell of embodiment 71 under conditions wherein the binding molecule is expressed.

Embodiment 74. A method of killing a cell, the method comprising the step of contacting the cell with a binding molecule according to any one of embodiments 1-67 or a pharmaceutical composition according to embodiment 66.

Embodiment 75. A method of treating a disease, disorder, or condition in a subject, the method comprising a step of administering to a subject in need thereof a therapeutically effective amount of a binding molecule according to any one of embodiments 1-67 or a pharmaceutical composition according to embodiment 66.

Embodiment 76. The method of embodiment 75, wherein the disease, disorder, or condition is an immune disorder or microbial infection.

Embodiment 77. A method of treating cancer, the method comprising administering to a subject in need thereof an effective amount of the binding molecule of any one of embodiments 1-65, or the pharmaceutical composition of embodiment 68.

Embodiment 78. The method of embodiment 77, wherein the cancer is characterized by a high mutational burden and/or a high frequency of indels.

Embodiment 79. The method of any one of embodiments 77-78, wherein the cancer is a solid tumor.

Embodiment 80. The method of any one of embodiments 77-79, wherein the cancer is bladder cancer, breast cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, or urothelial cancer.

Embodiment 81. The method of any one of embodiments 77-79, wherein the cancer is bladder cancer, and the bladder cancer is urothelial carcinoma.

Embodiment 82. The method of any one of embodiments 77-79, wherein the cancer is breast cancer, and the breast cancer is HER2 positive breast cancer or triple negative breast cancer.

Embodiment 83. The method of any one of embodiments 77-79, wherein the cancer is colon cancer, and the colon cancer is colorectal cancer.

Embodiment 84. The method of any one of embodiments 77-79, wherein the cancer is gastrointestinal cancer, and the gastrointestinal cancer is gastric cancer, biliary tract neoplasm, or gastroesophageal junction cancer.

Embodiment 85. The method of any one of embodiments 77-79, wherein the cancer is glioma, and the glioma is glioblastoma.

Embodiment 86. The method of any one of embodiments 77-79, wherein the cancer is head and neck cancer, and the head and neck cancer is squamous cell carcinoma of the head and neck.

Embodiment 87. The method of any one of embodiments 77-79, wherein the cancer is kidney cancer, and the kidney cancer is renal cell carcinoma.

Embodiment 88. The method of any one of embodiments 77-79, wherein the cancer is liver cancer, and the liver cancer is hepatocellular carcinoma.

Embodiment 89. The method of any one of embodiments 77-79, wherein the cancer is lung cancer, and the lung cancer is non-small cell lung cancer or small-cell lung cancer.

Embodiment 90. The method of any one of embodiments 77-79, wherein the cancer is lymphoma, and the lymphoma is Hodgkin lymphoma, non-Hodgkin lymphoma, primary mediastinal large B-cell lymphoma, or diffuse large B-cell lymphoma.

Embodiment 91. The method of any one of embodiments 77-79, wherein the cancer is mesothelioma, and the mesothelial carcinoma is pleural mesothelioma.

Embodiment 92. The method of any one of embodiments 77-79, wherein the cancer is myeloma, and the myeloma is multiple myeloma.

Embodiment 93. The method of any one of embodiments 77-79, wherein the cancer is skin cancer, and the skin cancer is squamous cell cancer of the skin or melanoma.

Embodiment 94. The method of any one of embodiments 77-93, wherein the cancer is relapsed or refractory to treatment with one or more checkpoint inhibitors.

Embodiment 95. The method of any one of embodiments 77-93, wherein the cancer is relapsed or refractory to a treatment involving at least one of ipilimumab, nivolumab, pembrolizumab, atezolizumab, durvalumab, avelumab, tremelimumab and cemiplimab.

Embodiment 96. The method of any one of embodiments 77-95, wherein the cancer is metastatic.

### EXAMPLES

The following examples demonstrate certain embodiments of the present invention. However, it is to be understood that these examples are for illustration purposes only and do not intend, nor should any be construed, to be wholly definitive as to conditions and scope of this invention. The experiments in the following examples were carried out using standard techniques, which are well known and routine to those of skill in the art, except where otherwise described.

### Example 1. Functional Characteristics of the PD-L1 Binding Molecule MT-6402

This example evaluates the functional characteristics of the PD-L1 binding molecule MT-6402. The ability to inhibit ribosomes, bind PD-L1, and induce cytotoxicity of PD-L1-expressing cells was examined in vitro.

### Catalytic Activity

The ribosome inhibition assay used a cell-free, in vitro protein translation assay using the TNT® Quick Coupled Transcription/Translation kit (L1170 Promega Madison, Wis., U.S.A.). The kit includes Luciferase T7 Control DNA (L4821 Promega Madison, Wis., U.S.A.) and TNT® Quick Master Mix. The ribosome activity reaction was prepared according to manufacturer instructions. A series (typically 10-fold) of dilutions were prepared in appropriate buffer and a series of identical TNT reaction mixture components were created for each dilution. The protein samples were combined with each of the TNT reaction mixtures along with the Luciferase T7 Control DNA. The test samples were incubated for 1.5 hours at 30° C. After the incubation, Luciferase Assay Reagent (E1483 Promega. Madison, Wis., U.S.A.) was added to all test samples and the amount of luciferase protein translation was measured by luminescence according to the manufacturer instructions. The level of translational inhibition was determined by non-linear regression analysis of log-transformed concentrations of total protein versus relative luminescence units. Using statistical software (GraphPad Prism, San Diego, Calif., U.S.A.), the half maximal inhibitory concentration (IC₅₀) value was calculated for each sample using the Prism software function of log(inhibitor) vs. response (three parameters) [Y=Bottom+((Top−Bottom)/(14+10\hat{ }(X−Log IC₅₀)))] under the heading dose-response-inhibition.

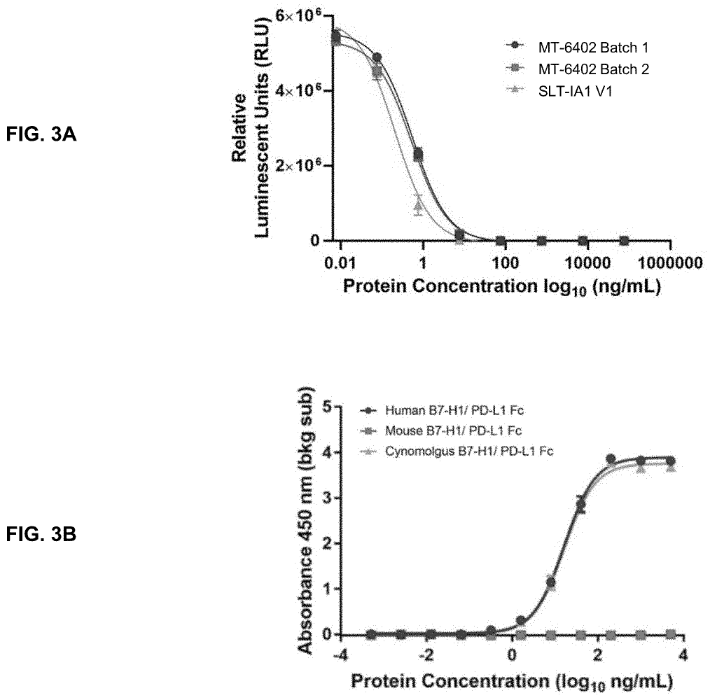

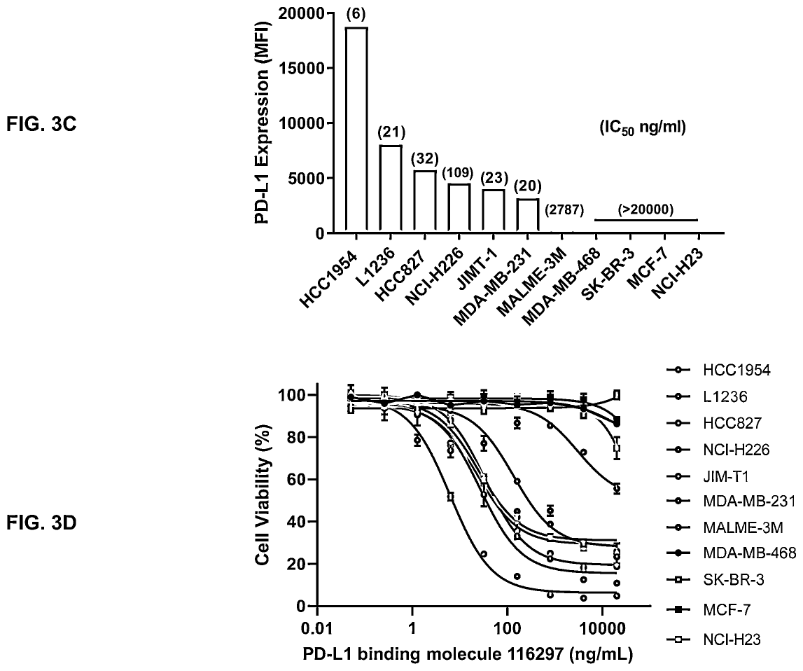

As shown in FIG. 3A, the PD-L1 binding molecule MT-6402 (SEQ ID NO:128) exhibited ribosome inhibition activities comparable to a positive “control” molecule, a Shiga toxin effector polypeptide (SLT-IA1 V1) not coupled with any targeting agent or binding region.

### Binding Kinetics

The PD-L1 binding molecule MT-6402 (SEQ ID NO:128) was tested for binding to recombinant PD-L1 proteins originating from human, cynomolgus macaque, or mouse using in an enzyme-linked immuno assay (ELISA) format. Background subtracted ELISA signals detected as absorbance values at 450 nanometer (nm) using a plate reader are shown on the Y-axis.

FIG. 3D shows results of a PD-L1 target binding assay for the PD-L1 binding molecule MT-6402 (SEQ ID NO:128). The PD-L1 binding molecule MT-6402 (SEQ ID NO:128) bound to recombinant human PD-L1 and cynomolgus macaque PD-L1 but did not exhibit high-affinity binding to recombinant mouse PD-L1 in this assay.

### Cytoxicity

PD-L expression was evaluated on a variety of clinically relevant tumor cell lines by flow cytometry. As shown in FIG. 3C, PD-L1 is expressed on the cell surface of various human tumor cells, including cell lines of human lung, skin, and breast cancer origin.

FIG. 3D shows cytotoxicity for the PD-L1 binding molecule MT-6402 (SEQ ID NO:128) in the tumor cell lines. MT-6402 (SEQ ID NO:128) exhibited broad anti-tumor cytotoxicity. MT-6402 (SEQ ID NO:128) specifically and potently kills target cells expressing PD-L1.

### Example 2. The PD-L1 Binding Molecule MT-6402 Effectively Delivers Antigens to PD-L1-Positive Target Cells

This example evaluates the ability of the PD-L1 binding molecule MT-6402 to deliver antigens to PD-L1 positive target cells in vitro.

Co-culture assays using CMV-restricted T cells and PD-L1, HLA:A02 positive target cells were used to assess IFN-γ secretion and cytotoxicity. CMV-restricted T cells and PD-L1, HLA:A02 positive target cells were cultured at a 1:1 effector to target cell ratio.

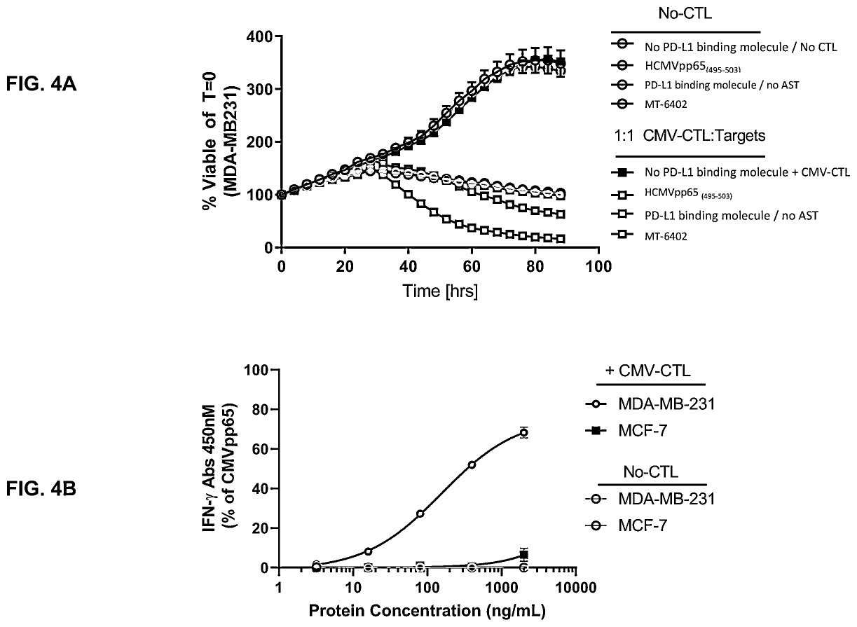

FIG. 4A shows the results of a co-culture cytotoxicity assay for the PD-L1 binding molecule MT-6402 comprising a CMV-restricted MHC-I peptide (NLVPMVATV, SEQ ID NO: 78) compared to PD-L1 binding molecules without a CMV-restricted MHC-I peptide. FIG. 4B shows the results of cytotoxic T cell (CTL) activation for the PD-L1 binding molecule MT-6402 comprising a CMV-restricted MHC-I peptide (NLVPMVATV, SEQ ID NO: 78).

### Example 3. Single and Multi-Antigen PD-L1 Binding Molecules Retain the Ability to Bind PD-L1 and Kill PD-L1-Expressing Cells In Vitro

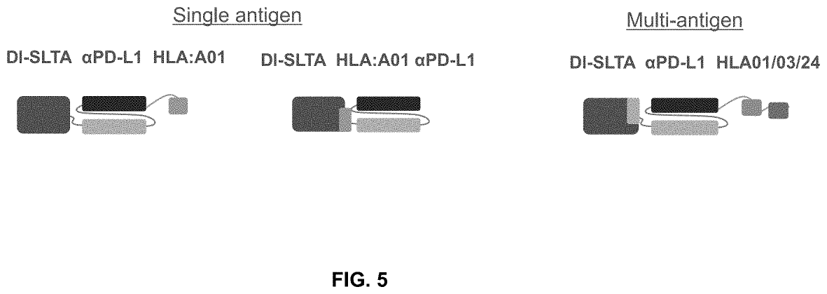

This example evaluates the functional characteristics of single and multi-antigen PD-L1 binding molecules. FIG. 5 is a schematic of PD-L1 binding molecules comprising single or multiple HLA:A01-restricted antigens in different locations of the PD-L1 binding molecule.

### In Vitro Binding Characteristics of Single and Multi-Antigen PD-L1 Binding Molecules

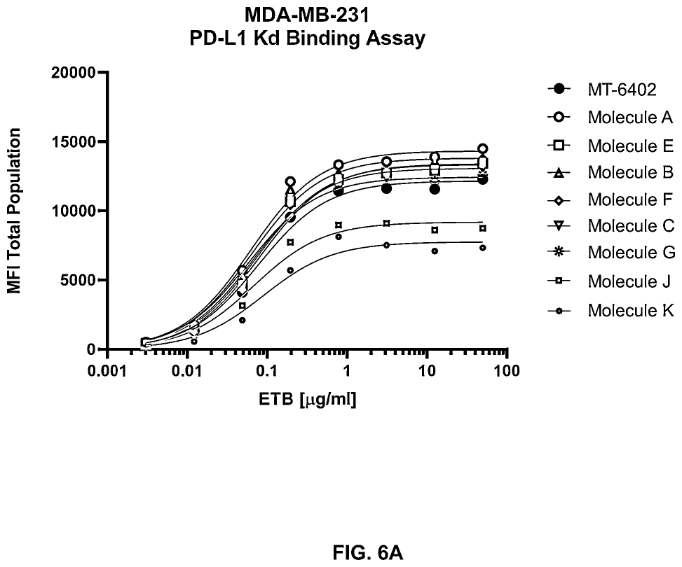

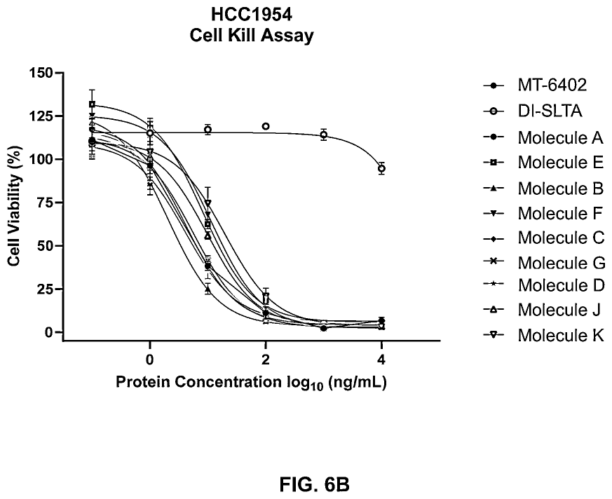

The binding kinetics of single and multi-antigen PD-L1 binding molecules was determined by flow-cytometry in PD-L1 high-expressing cancer cells (MDS-MB-231). As shown in FIG. 6A, the maximum median fluorescence intensity (Bₘₐₓ) of the PD-L1 binding molecules was approximately 8,000 to 15,000 MFI. Notably, the PD-L1 binding molecules with multiple antigens (Molecule J and Molecule K) had a Bmax of greater than 50% of the single antigen molecules (Molecule A, Molecule E, Molecule B, Molecule F, Molecule C, MT-6402, and Molecule G). All PD-L1 binding molecules had comparable K_{d} values within the range of 0.01 nM to 1 nM.

### Cytotoxicity of Single and Multi-Antigen PD-L1 Binding Molecules

The cytotoxic activities of single and multi-antigen PD-L1 binding molecules were measured using a tissue culture cell-based toxicity assay. The cytotoxicities of PD-L1 binding molecules were tested using cell-kill assays involving either PD-L1 positive or PD-L1 negative cells.

Human tumor cell line cells were plated (typically at 2×10³ cells per well for adherent cells the day prior to treatment, or 7.5×10³ cells per well for suspended cells, plated the same day as treatment) in 20 μL cell culture medium in 384-well plates. A series of 10-fold dilutions of the PD-L1 binding molecules was prepared in an appropriate buffer, and 5 μL of the dilutions or only buffer as a negative control were added to the cells. Control wells containing only cell culture medium were used for baseline correction. The cell samples were incubated with the proteins or buffer for 3 or 5 days at 37° C. and in an atmosphere of 5% carbon dioxide (CO₂). The total cell survival or percent viability was determined using a luminescent readout using the CellTiter-Glo® Luminescent Cell Viability Assay (G7573 Promega Madison, Wis., U.S.) according to the manufacturer's instructions as measured in relative light units (RLU).

The Percent Viability of experimental wells was calculated using the following equation: (Test RLU−Average Media RLU)/(Average Cells RLU−Average Media RLU)*100. Log protein concentration versus Percent Viability was plotted in Prism (GraphPad Prism, San Diego, Calif., U.S.) and log (inhibitor) versus response (3 parameter) analysis were used to determine the half-maximal cytotoxic concentration (CD₅₀) value for the tested proteins. The CD₅₀ values for each cell-targeting protein tested was calculated when possible.

The specificity of the cytotoxic activity of a given PD-L1 binding molecule was determined by comparing cytotoxicity of cells expressing PD-L1 with cytotoxicity of cells which do not exhibit any significant amount of PD-L1. This was accomplished by determining the CD₅₀ value of a given cell-targeting molecule toward cell populations which were positive for cell surface expression of the target biomolecule of the cell-targeting molecule being analyzed, and, then, using the same cell-targeting molecule concentration range to attempt to determine the CD₅₀ value toward cell populations which were negative for cell surface expression of the target biomolecule of the cell-targeting molecule. In some experiments, the PD-L1 negative cells treated with the maximum amount of the PD-L1 binding molecule did not show any change in viability as compared to a “buffer only” negative control. A molecule exhibiting an IC₅₀ value within 10-fold of a CD₅₀ value measured for a reference molecule is considered to exhibit cytotoxic activity comparable to that reference molecule. In particular, any cell-targeting molecule that exhibited a CD₅₀ value to a target positive cell population within 10-fold of the CD₅₀ value of a reference cell-targeting molecule comprising the same binding region and a wild-type, Shiga toxin effector polypeptide (e.g. SLT-1A-WT) but not comprising any fused, heterologous, T-cell epitope-peptide, toward the same cell-type is referred to herein as “comparable to wild-type.”

The cytotoxic activity of single and multi-antigen PD-L1 binding molecules is shown in FIG. 6B. All PD-L1 binding molecules exhibited potent cytotoxicity, although some PD-L1 binding molecules exhibited reduced cytotoxicity compared to other PD-L1 binding molecules. The single and multi-antigen PD-L1 binding molecules did not kill PD-L1 negative cells at the same concentrations (data not shown).

### Example 4. Single and Multi-Antigen PD-L1 Binding Molecules Induce Human T Cell Activation and Cytotoxicity of Tumor Cells in Vitro

This example examines the functional consequences of MHC class I presentation of T-cell epitopes delivered by single and multi-antigen PD-L1 binding molecules.

### Methods

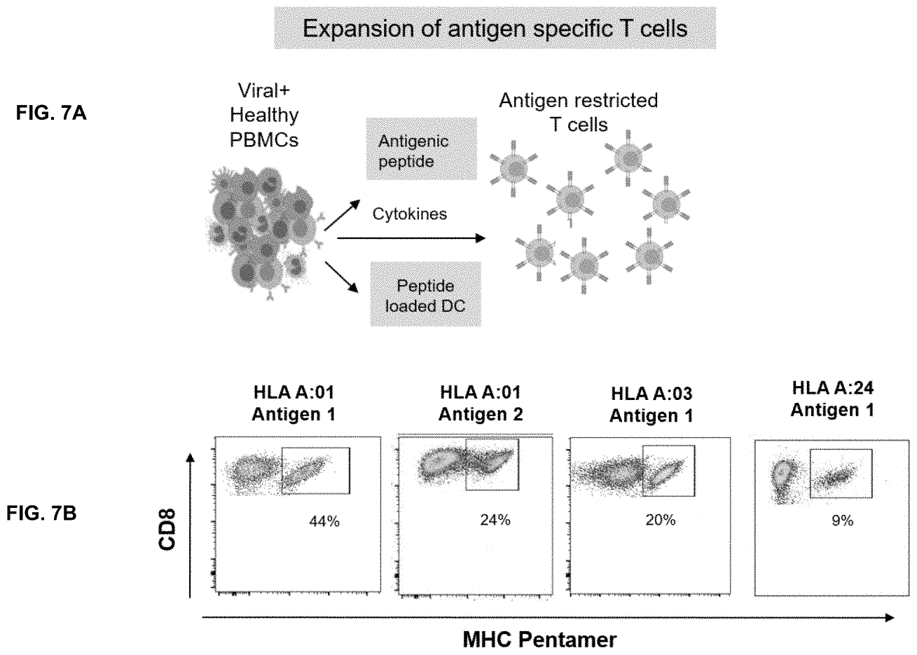

Peripheral blood mononuclear cells (PBMCs) were isolated from healthy donors and enriched for antigen-specific T cells by culturing the PBMCs in the presence of antigenic peptide, peptide loaded DCs, and cytokines (FIG. 7A). Antigen-restricted T cells specific were identified and sorted for specificity to the MHC-peptide complex using MHC tetramers and following standard cell staining and flow cytometry protocols (FIG. 7B).

Co-culture assays using antigen-specific T cells and PD-L1 positive target cells were used to assess IFN-γ secretion and cytotoxicity. PD-L1 target cells that are either HLA:A1 (A375 cell lines: clone U/PD-L1-high; clone D: PD-L1-low) or HLA:A24 (PC-3: PD-L1high; HepG2: PD-L1-negative) positive were incubated for 16 hours with 500 nM of the PD-L1 binding molecule at 37° C. and 5% CO₂. The PD-L1 positive target cells were washed and combined with media containing either antigen-restricted T cells or no antigen-restricted T cells and co-incubated for 40 hours at a ratio of two T cells to one target positive tumor cell (2:1) at 37° C. and 5% CO₂.

To measure IFN-γ secretion, supernatants were harvested from the co-culture following incubation and Supernatants were harvested and IFN-γ concentrations were measured using a cytokine-specific IFN-γ ELISA Kit (Biolegend, Inc., San Diego, Calif., U.S.) according to manufacturer's instructions.

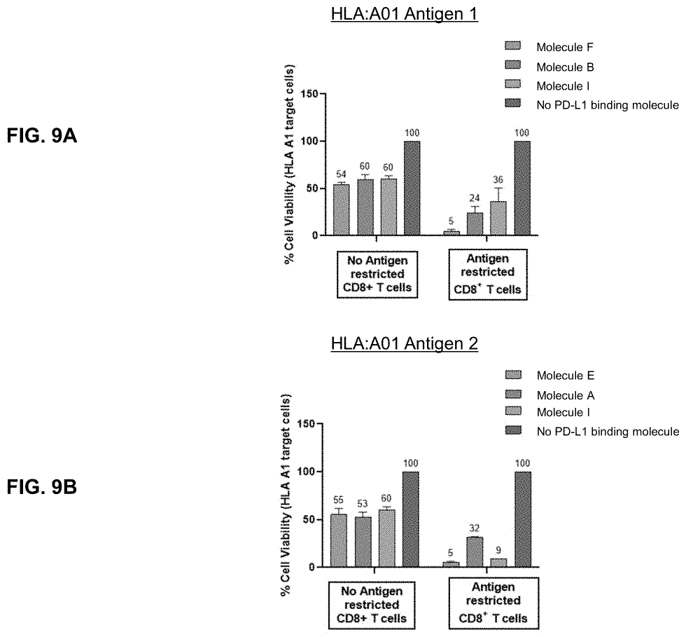

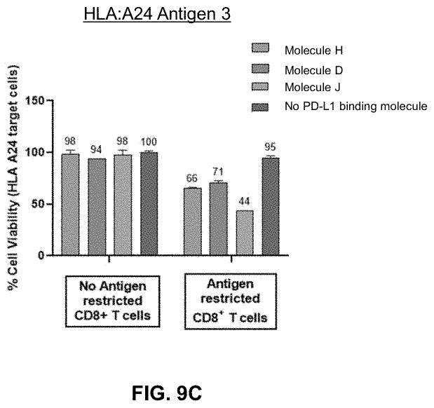

For the results shown in FIG. 9A and FIG. 9B, cytotoxicity was determined using the IncuCyte® S3 Live-Cell Analysis System (EssenBioscience, Ann Arbor, Mich., U.S.) normalized to time-point zero (baseline viability). Briefly, PD-L1, HLA:A1 positive cells (A375 cell line) were plated in standard 96-well tissue culture plates and cultured under standard conditions. Peptide-restricted T cells and A375 target cells were cultured at a 2:1 ratio (effector to target cell). Data was obtained from up to four images per well as readout by phase contrast via standard protocols provided by the manufacturer.

For the results shown in FIG. 9C, after harvesting of supernatants for the IFN-γ analysis, adherent PD-L1, HLA:A24 positive tumor cells (PC3 cell line) were washed to remove PBMCs, and cell viability of the remaining adherent cells was assessed by CellTiter-Glo® Luminescent Cell Viability Assay (G7573 Promega Madison, Wis., U.S.), according to the manufacturer's instructions.

### Results

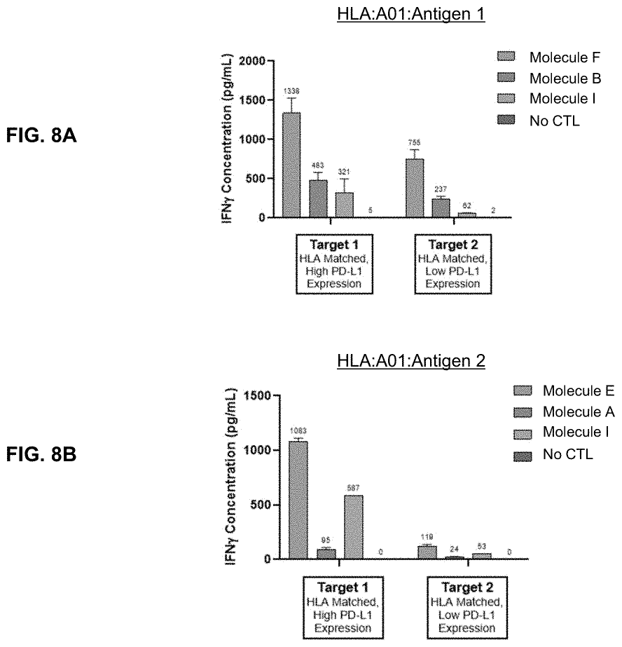

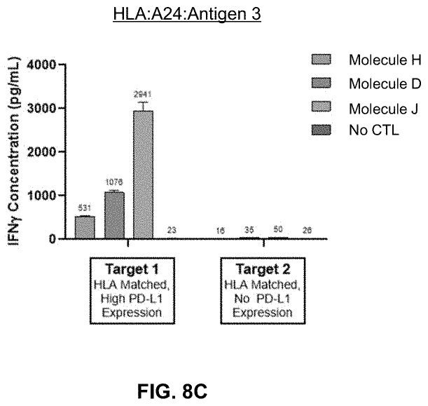

FIGS. 8A-8C show the results of the IFN-γ ELISA assay. FIG. 8A shows that the single and multi-antigen PD-L1 binding molecules Molecule F, Molecule B. and Molecule I induce IFN-γ secretion in peptide-stimulated T cells. FIG. 8B shows that the single and multi-antigen PD-L1 binding molecules Molecule E, Molecule A, and Molecule I induce IFN-γ secretion in peptide-stimulated T cells. FIG. 8C shows that the single and multi-antigen PD-L1 binding molecules Molecule D, Molecule H, and Molecule J induce IFN-γ secretion in peptide-stimulated T cells. Collectively, these data demonstrate that single and multi-antigen PD-L1 molecules result in cell-surface presentation of the antigen by target cells resulting in the activation of T cells to release effector cytokines.

FIG. 9A and FIG. 9B shows results from the IncuCyte® S3 Live-Cell Analysis System. The results show that coculture of PD-L1 binding molecule-treated A375 cells with antigen-restricted T cells reduced viability of A375 cells compared to the no CTL control.

FIG. 9C shows results from the CellTiter-Glo® Luminescent Cell Viability Assay. The results show that coculture of PD-L1 binding molecule-treated PC-3 cells with antigen-restricted T cells after incubation with single or multi-antigen PD-L1 binding molecules reduced viability of target cells compared to the control.

### Example 5. Single-Antigen PD-L1 Binding Molecules Induce Cytotoxicity In Vitro

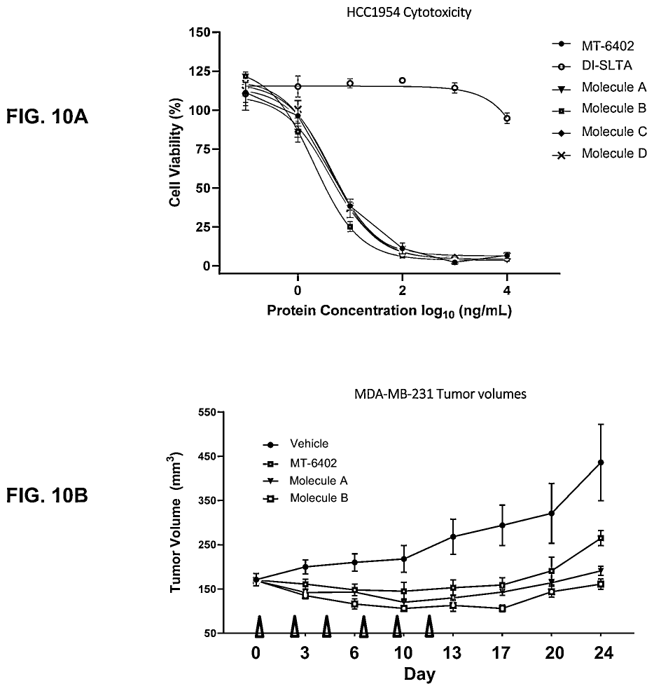

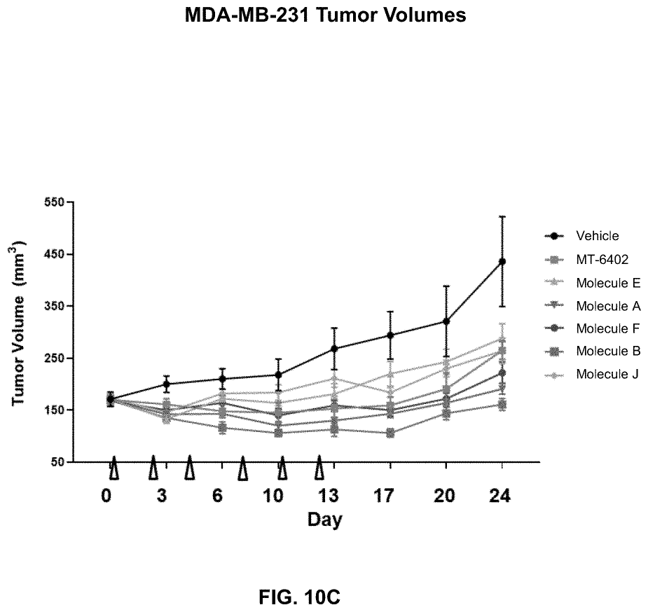

Cytotoxicity of single-antigen PD-L1 binding molecules (Molecule A, Molecule B, Molecule C, or Molecule D) on HCC1954 cells was measured in vitro. As shown in FIG. 10A and Table 8, in vitro direct cell kill potency was retained compared to MT-6402.

TABLE 8
IC50 values from HCC1954 cell killing assay

| | MT- | Molecule | Molecule | Molecule | Molecule |
|---|---|---|---|---|---|
| | 6402 | A | B | C | D |
| IC50 (ng/ml) | 4.57 | 4.13 | 2.05 | 4.07 | 4.49 |

### Example 6. Single and Multi-Antigen PD-L1 Binding Molecules Induce Anti-Tumor Responses In Vivo

This example determines the efficacy and pharmacokinetics of single and multi-antigen PD-L1 binding molecules in vivo.

### Methods

Immunocompetent NOG mice were injected with 0.1 mL of MDA-MB-231 cells with 50% Matrigel subcutaneously in the left flank. Pre-study tumor volumes were recorded beginning four to five days after injection. When tumors reached an average tumor volume of 50-150 mm³ animals were matched by tumor volume into treatment or control groups and were used for dosing of the single or multi-antigen PD-L1 binding molecules.

Mice were intravenously administered 6 mg/kg of the PD-L1 binding molecule in a 10 mL/kg volume on day 1 of the study followed by intravenous administration of 2 mg/kg of the PD-L1 binding molecule in a 10 mL/kg volume on days 2, 4, 7, 9, and 11 of the study. The vehicle control was a buffer diluted in saline and was administered on days 0, 2, 4, 7, 9, and 11 of the study.

To determine efficacy of the PD-L1 binding molecules, tumor dimensions were measured twice weekly by digital caliper and data including individual and mean estimated tumor volumes (Mean TV±SEM) recorded for each group; tumor volume was calculated using the formula (1): TV=width²×length×0.52. At study completion, percent tumor growth inhibition (% TGI) values was calculated and reported for each treatment group (T) versus control (C) using initial (i) and final (f) tumor measurements by the formula (2): % TGI=1−(Tf−Ti)/(Cf−Ci). Individual mice reporting a tumor volume less than or equal to 30% of the Day 0 measurement for two consecutive measurements were considered partial responders (PR). Individual mice lacking palpable tumors (0.00 mm³ for two consecutive measurements) were classified as complete responders (CR); a CR that persists until study completion will be considered a tumor-free survivor (TFS). Tumor doubling time (DT) was determined for the vehicle treated groups using the formula DT=(Df−Di)*log₂ (log TVf−log TVi) where D=Day and TV=Tumor Volume. Tumor volume will be monitored beginning on Day 0.

To determine the half-life of the PD-L1 binding molecules, approximately 70-100 uL blood was collected by submandibular bleed at various timepoints post dose 1 on Day 0 and post dose 4 on Day 7, from animals as shown below and processed for serum. All blood was transferred to serum separator tubes and allowed to clot at room temperature for at least 15 minutes. Samples were centrifuged at 3500 for 10 minutes at room temperature. The resultant serum was separated, transferred to uniquely labeled clear polypropylene tubes, and frozen immediately over dry ice or in a freezer set to maintain −80° C. until shipment.

To determine safety of the PD-L1 binding molecules, animals were observed daily and weighed twice weekly using a digital scale; data including individual and mean gram weights (Mean Weight±SEM), mean percent weight change versus Day 0 (% vD0) were recorded for each group and % vD0 plotted at study completion. Single agent or combination groups reporting a mean % vD0>20% and/or >10% mortality were considered above the maximum tolerated dose (MTD) for that treatment on the evaluated regimen. Maximum mean % vD0 (weight nadir) for each treatment group was reported at study completion. Animal weight will be monitored beginning on Day 0.

The study endpoint was when the mean tumor volume of the control group (uncensored) reaches 1500 mm³. If this occurs before Day 28, treatment groups and individual mice may be dosed and measured up to Day 28. If the mean tumor volume of the control group (uncensored) does not reach 1500 mm³ by Day 28, then the endpoint for all animals will be the day when the mean tumor volume of the control group (uncensored) reaches 1500 mm³ up to a maximum of Day 60. Studies extended beyond these endpoints are subject to additional charges.

### Results

As shown in FIG. 10B and FIG. 10C, tumor-bearing mice administered single-(Molecule A, Molecule B, Molecule E, or Molecule F) or multi-antigen (Molecule J) PD-L1 binding molecules exhibited reduced tumor volume compared to mice treated with a vehicle control. These data indicate that single or multi-antigen PD-L1 binding molecules elicit effective anti-tumor responses in vivo.

### Example 7. Single and Multi-Antigen PD-L1 Binding Molecules that Deliver Antigen Induce Human T Cell Specific Cytokine Release in an HLA Matched Manner

This example examines the functional consequences of MHC class I presentation of T-cell epitopes delivered by single and multi-antigen PD-L1 binding molecules.

### Methods

PBMCs were isolated from healthy donors and enriched for antigen-specific T cells by culturing the PBMCs in the presence of antigenic peptide, peptide loaded DCs, and cytokines (FIG. 7A). Antigen-restricted T cells specific were identified and sorted for specificity to the MHC-peptide complex using MHC tetramers and following standard cell staining and flow cytometry protocols (FIG. 7B).

Co-culture assays using antigen-specific T cells and PD-L1 positive target cells were used to assess cytokine release. PD-L1 target cells that are either HLA:A1 (A375 cell lines: clone I/PD-L1-high; clone D: PD-L1-low) or HLA:A2 (MDA-MB-231: PD-L1 positive, or MCF-7: PD-L1 low/negative) positive were incubated for 16 hours in triplicate with 500 nM of the PD-L1 binding molecule at 37° C. and 5% CO₂. The PD-L1 positive target cells were washed and combined with media containing antigen-restricted T cells and co-incubated for 40 hours at a ratio of two T cells to one target positive tumor cell (2:1) at 37° C. and 5% CO₂.

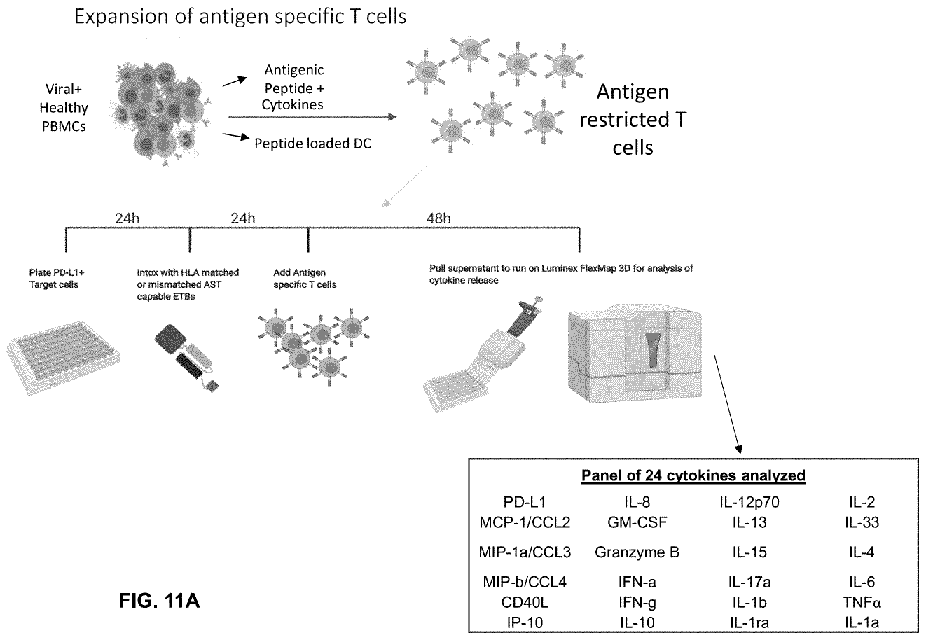

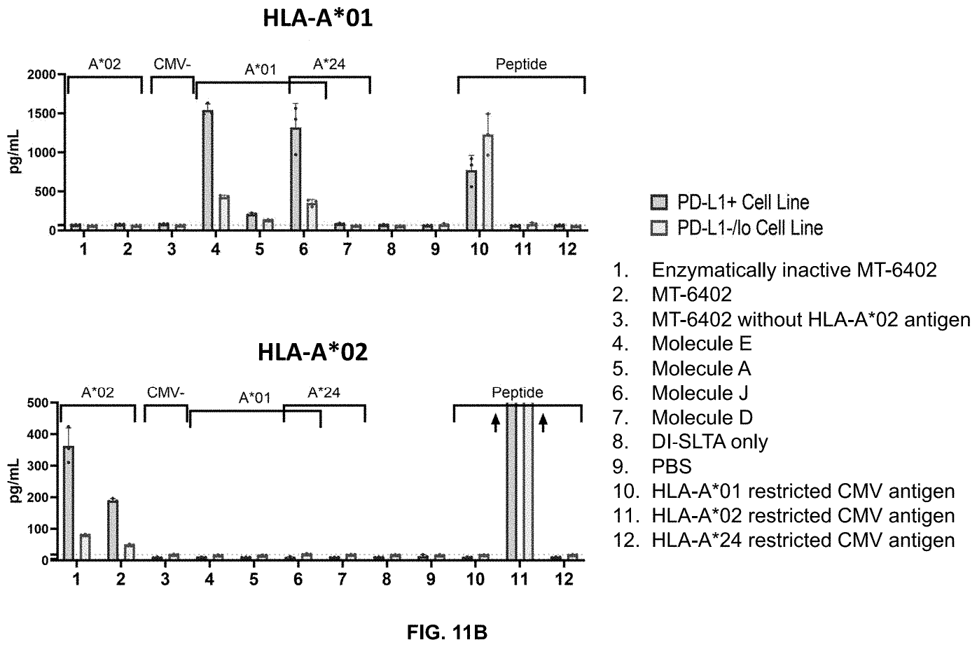

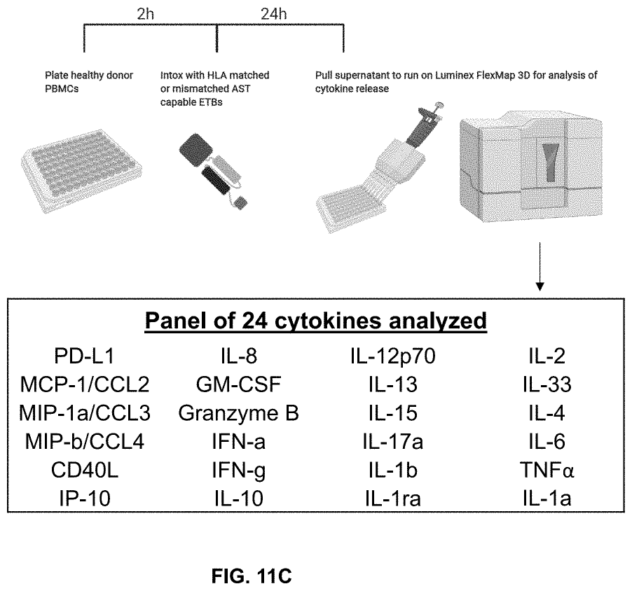

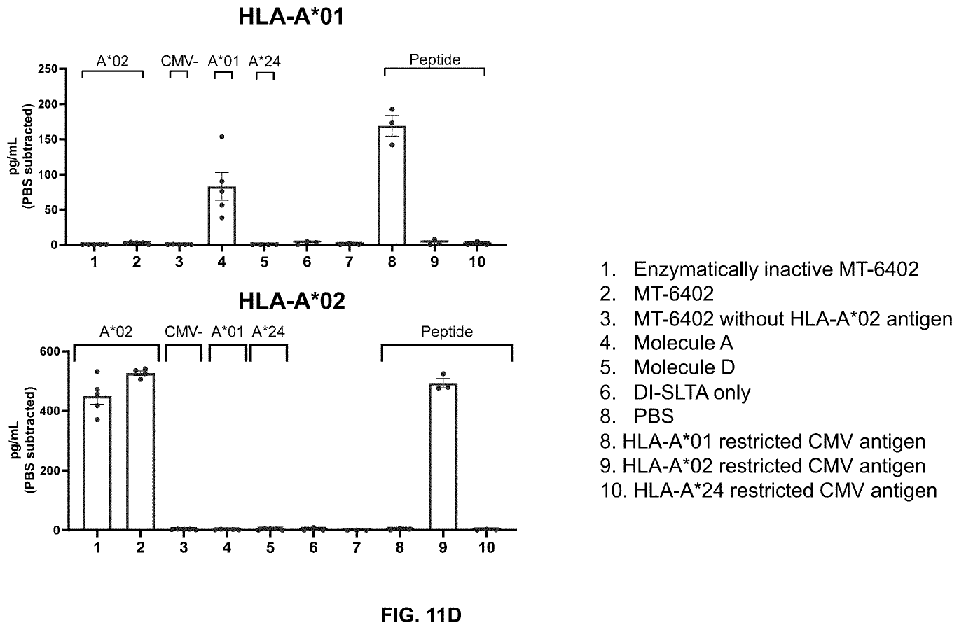

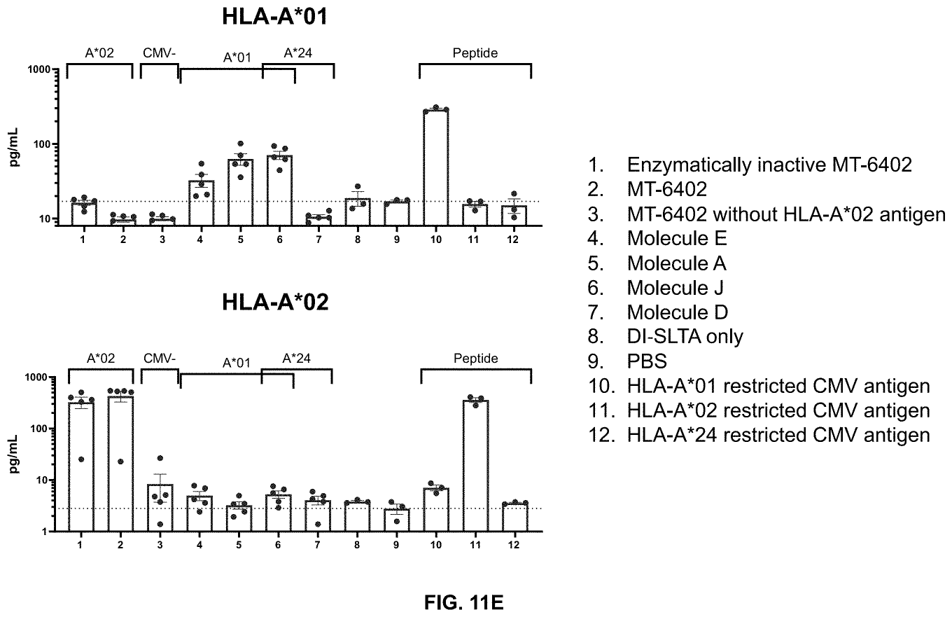

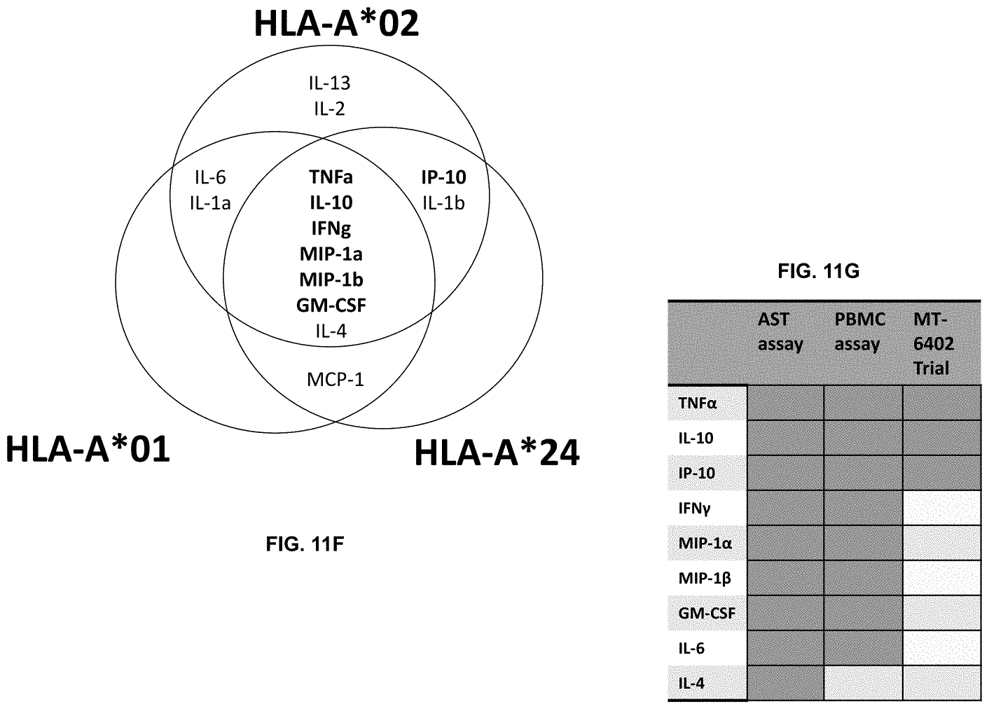

To measure cytokine release, supernatants were harvested from the co-culture following incubation and a panel of 24 cytokine analytes were assessed (FIG. 11A) by a Luminex FlexMap® 3D (Luminex Inc. Austin, Tex., US). Briefly. Luminex multiplex assays utilize color-coded superparamagnetic beads coated with analyte-specific capture antibodies. Beads recognizing different target analytes are mixed and incubated with the collected supernatant. Captured analytes are subsequently detected using a cocktail of biotinylated detection antibodies and a streptavidin-phycoerythrin conjugate. Bead conjugates are run on the dual-laser flow-based Luminex FlexMap® 3D, and results are quantified by interpolation with standard curves for determined for each analyte in the same assay.

### Results

As shown in FIG. 11B and FIG. 11F, TNFα was used as an example cytokine that was specific for HLA matched antigen delivery and T cell release in both HLA-A*01 and HLA-A*02 matched systems. Only PD-L1 binding molecules carrying the A1 antigen were able to deliver to the A1 target cell lines (A-375) and generate T cell mediated TNFα release, while PD-L1 binding molecules with mismatched antigens (A2 or A24) did not generate TNFα. The positive control matching peptide is also restricted to an HLA matched paring of PD-L1 binding molecule, Target cell, and CTL addition as seen in FIG. 113. FIG. 11F is a summary Venn diagram of overlapping cytokines released in this assay from co-cultures of matched HLA-A*01, HLA*A*02, and HLA-A*24 cell types.

### Example 8. Single and Multi-Antigen PD-L1 Binding Molecules that Deliver Antigen Induce Human T Cell Specific Cytokine Release in an HLA Matched Manner

This example examines the functional consequences, specifically PBMC mediated cytokine release, of MHC class I presentation of T-cell epitopes delivered by single and multi-antigen PD-L1 binding molecules to healthy donor PBMCs.

### Methods

HLA typed PBMCs were obtained from healthy donors to be used in these assays. Intoxication of these PBMCs by PD-L1 binding molecules with HLA matched or mis-matched antigen was performed to determine the consequence of antigen delivery in a matched HLA system. Briefly, PBMCs from healthy donors were plated in 96-well plates and allowed to rest for 2h at 37° C. and 5% CO₂(FIG. 11C). This was followed by intoxication of five replicates of donor PBMCs by PD-L1 binding molecules (10,000 ng/ml) with HLA matched or mis-matched antigens. Following a 24h incubation time at 37° C. and 5% CO2, supernatants were collected to measure cytokine release. LPS and an HLA matched restricted CMV antigen were used as positive controls.

To measure cytokine release, supernatants were harvested from the co-culture following incubation and a panel of 24 cytokine analytes were assessed (FIG. 11C) by a Luminex FlexMap® 3D (Luminex Inc, Austin, Tex., US). Briefly, Luminex multiplex assays utilize color-coded superparamagnetic beads coated with analyte-specific capture antibodies. Beads recognizing different target analytes are mixed and incubated with the collected supernatant. Captured analytes are subsequently detected using a cocktail of biotinylated detection antibodies and a streptavidin-phycoerythrin conjugate. Bead conjugates are run on the dual-laser flow-based Luminex FlexMap® 3D, and results are quantified by interpolation with standard curves for determined for each analyte in the same assay.

### Results

As shown in FIG. 11D, FIG. 11E and FIG. 11G, IP-10 was used as an example cytokine that was specific for HLA matched antigen delivery and T cell release in both HLA-A*01 and HLA-A*02 matched systems. Only PD-L1 binding molecules carrying the A1 antigen could deliver to the A1 target cell lines (A-375) and generate T cell mediated IP-10 release, while PD-L1 binding molecules with mismatched antigens (A2 or A24) did not generate IP-10 (FIG. 11D). Positive control peptides restricted to different HLA serotypes only released IP-10 when their HLA restriction matched the HLA type of PD-L1 positive target cells and PBMC donor cells (FIG. 11E). FIG. 11G is a table summary of cytokines that were released in matched HLA type setting for the AST assay, the PBMC assay, and the MT-6402 clinical trial. Shaded cells indicate HLA matched cytokine release for each respective assay.

### INCORPORATION BY REFERENCE

All publications, patents, and patent applications are herein incorporated by reference in their entirety to the same extent as if each individual publication, patent or patent application was specifically and individually indicated to be incorporated by reference in its entirety. The international patent application publications WO 2014/164680, WO 2014/164693, WO 2015/138435, WO 2015/138452, WO 2015/113005, WO 2015/113007, WO 2015/191764, WO 2016/196344, WO 2017/019623, WO 2018/106895, WO 2018/140427, WO 2019/183093, and WO 2020/154475, are each incorporated herein by reference in its entirety. The disclosures of U.S. patent applications US2015/259428, US2016/17784, US2017/143814, and U.S. 62/644,832, and PCT Application No. PCT/US2020/051589 are each incorporated herein by reference in their entirety. The complete disclosures of all electronically available biological sequence information from GenBank (National Center for Biotechnology Information, U.S.A.) for amino acid and nucleotide sequences cited herein are each incorporated herein by reference in their entirety.

1.-96. (canceled)

97. A cell binding molecule comprising:
(i) a Shiga-like toxin A subunit effector polypeptide comprising the sequence of SEQ ID NO: 41, or a sequence at least 90% or at least 95% identical thereto;
(ii) a binding region capable of specifically binding an extracellular target on a cell; and
(iii) a CD8+ T-cell epitope comprising the sequence of SEQ ID NO: 80, 81, 82, 300, 301, or 314.

98. The cell binding molecule of claim 97, wherein the CD8+ T-cell epitope is located at the C-terminus of the Shiga-like toxin A subunit effector polypeptide.

99. The cell binding molecule of claim 97, wherein the at least one CD8+ T-cell epitope is located at the C-terminus of the binding region.

100. The cell binding molecule of claim 97, wherein the at least one CD8+ T-cell epitope is located between the Shiga-like toxin A subunit effector polypeptide and the binding region.

101. The cell binding molecule of claim 97, wherein the molecule comprises a first CD8+ T-cell epitope and a second CD8+ T-cell epitope that are each heterologous to Shiga-like toxin A subunits.

102. The cell binding molecule of claim 101, wherein the first and the second CD8+ T-cell epitopes are different from each other.

103. The cell binding molecule of claim 97, wherein the molecule comprises a first CD8+ T-cell epitope, a second CD8+ T-cell epitope, and a third CD8+ T-cell epitope that are each heterologous to Shiga-like toxin A subunits.

104. The cell binding molecule of claim 103, wherein at least one of the first, second, and third CD8+ T-cell epitopes is different from the others.

105. The cell binding molecule of claim 103, wherein the molecule comprises:
(a) three CD8+ T-cell epitopes comprising the sequences of SEQ ID NOs: 78, 79, and 300; or
(b) three CD8+ T-cell epitopes comprising the sequences of SEQ ID NOs: 300, 301, and 302.

106. The cell binding molecule of claim 105, wherein the molecule comprises three CD8+ T-cell epitopes, comprising, in N-terminal to C-terminal order, the sequences of:
(a) SEQ ID NOs: 300, 301, and 302; or
(b) SEQ ID NOs: 300, 302, and 301.

107. A pharmaceutical composition comprising the binding molecule of claim 97, and at least one pharmaceutically acceptable excipient or carrier.

108. A polynucleotide encoding the binding molecule claim 97, or a complement thereof.

109. An expression vector comprising the polynucleotide according to claim 108.

110. A host cell comprising the polynucleotide according to claim 108.

111. A method of treating a disease, disorder, or condition in a subject, the method comprising a step of administering to a subject in need thereof a therapeutically effective amount of the binding molecule according to claim 97.

112. The method of claim 111, wherein the disease, disorder, or condition is an immune disorder or microbial infection.

113. A method of treating cancer, the method comprising administering to a subject in need thereof a therapeutically effective amount of the binding molecule of claim 97.

114. The method of claim 113, wherein the cancer is a solid tumor.

115. The method of claim 113, wherein the cancer is bladder cancer, breast cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, or urothelial cancer.

116. A PD-L1 binding molecule comprising:
(i) a Shiga-like toxin A subunit effector polypeptide comprising the sequence of SEQ ID NO: 41, or a sequence at least 90% or at least 95% identical thereto;
(ii) a binding region capable of specifically binding an extracellular part of PD-L1; wherein the binding region comprises:
(a) a heavy chain variable region (VH) comprising:
(1) a CDR1 comprising the amino acid sequence EYTMH (SEQ ID NO:27),
(2) a CDR2 comprising the amino acid sequence GINPNNGGTWYNQKFKG (SEQ ID NO:29), and
(3) a CDR3 comprising the amino acid sequence PYYYGSREDYFDY (SEQ ID NO:32);
and
(b) a light chain variable region (VL) comprising:
(1) a CDR1 comprising the amino acid sequence SASSSVSYMY (SEQ ID NO:19),
(2) a CDR2 comprising the amino acid sequence LTSNLAS (SEQ ID NO:20),
and
(3) a CDR3 comprising the amino acid sequence QQWSSNPPT (SEQ ID NO:26); and
(iii) a CD8+ T-cell epitope comprising the sequence of SEQ ID NO: 80, 81, 82, 300, 301, or 314.

117. The PD-L1 binding molecule of claim 116, wherein the CD8+ T-cell epitope is located at the C-terminus of the Shiga-like toxin A subunit effector polypeptide.

118. The PD-L1 binding molecule of claim 116, wherein the at least one CD8+ T-cell epitope is located at the C-terminus of the binding region.

119. The PD-L1 binding molecule of claim 116, wherein the at least one CD8+ T-cell epitope is located between the Shiga-like toxin A subunit effector polypeptide and the binding region.

120. The PD-L1 binding molecule of claim 116, wherein the molecule comprises a first CD8+ T-cell epitope and a second CD8+ T-cell epitope that are each heterologous to Shiga-like toxin A subunits.

121. The PD-L1 binding molecule of claim 120, wherein the first and the second CD8+ T-cell epitopes are different from each other.

122. The PD-L1 binding molecule of claim 116, wherein the molecule comprises a first CD8+ T-cell epitope, a second CD8+ T-cell epitope, and a third CD8+ T-cell epitope that are each heterologous to Shiga-like toxin A subunits.

123. The PD-L1 binding molecule of claim 122, wherein at least one of the first, second, and third CD8+ T-cell epitopes is different from the others.

124. The PD-L1 binding molecule of claim 122, wherein the molecule comprises:
(a) three CD8+ T-cell epitopes comprising the sequences of SEQ ID NOs: 78, 79, and 300; or
(b) three CD8+ T-cell epitopes comprising the sequences of SEQ ID NOs: 300, 301, and 302.

125. The PD-L1 binding molecule of claim 124, wherein the molecule comprises three CD8+ T-cell epitopes, comprising, in N-terminal to C-terminal order, the sequences of:
(a) SEQ ID NOs: 300, 301, and 302; or
(b) SEQ ID NOs: 300, 302, and 301.

126. The PD-L1 binding molecule of claim 116, wherein the PD-L1 binding molecule is a continuous polypeptide.

127. The PD-L1 binding molecule of claim 116, wherein the PD-L1 binding molecule comprises two polypeptides.

128. The PD-L1 binding molecule of claim 116, wherein the PD-L1 binding molecule comprises the sequence of any one of SEQ ID NO: 303-313.

129. A pharmaceutical composition comprising the binding molecule of claim 116, and at least one pharmaceutically acceptable excipient or carrier.

130. A polynucleotide encoding the binding molecule claim 116, or a complement thereof.

131. An expression vector comprising the polynucleotide according to claim 130.

132. A host cell comprising the polynucleotide according to claim 130.

133. A method of treating a disease, disorder, or condition in a subject, the method comprising a step of administering to a subject in need thereof a therapeutically effective amount of the binding molecule according to claim 116.

134. The method of claim 133, wherein the disease, disorder, or condition is an immune disorder or microbial infection.

135. A method of treating cancer, the method comprising administering to a subject in need thereof a therapeutically effective amount of the binding molecule of claim 116.

136. The method of claim 135, wherein the cancer is a solid tumor.

137. The method of claim 135, wherein the cancer is bladder cancer, breast cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, or urothelial cancer.
